(19) United States
(12) Patent Application Publication
(10) Pub. No.: US 2013/0202634 A1
(43) Pub. Date: Aug. 8, 2013

(54) Dengue Virus (DV) Polypeptide Sequences, T Cell Epitopes and Methods and Uses Thereof

(71) Applicant: La Jolla Institute For Allergy And Immunology, La Jolla, CA (US)

(72) Inventors: Sujan Shresta, San Diego, CA (US); Lauren Yauch, La Jolla, CA (US); Alessandro Sette, San Diego, CA (US); Daniela Weiskopf, San Diego, CA (US)

(73) Assignee: LA JOLLA INSTITUTE FOR ALLERGY AND IMMUNOLOGY, La Jolla, CA (US)

(21) Appl. No.: 13/719,227

(22) Filed: Dec. 18, 2012

Related U.S. Application Data
(63) Continuation-in-part of application No. PCT/US2011/041889, filed on Jun. 24, 2011.
(60) Provisional application No. 61/358,142, filed on Jun. 24, 2010.
(60) Provisional application No. 61/391,882, filed on Oct. 11, 2010.

(51) Int. Cl. C07K 7/08; C07K 7/06

(52) U.S. Cl. CPC C07K 7/08; C07K 7/06
USPC 424/186.1; 530/326; 530/328

(57) ABSTRACT

Dengue virus (DV) peptides, including T cell epitopes, structural and non-structural (NS) polypeptide sequences, subsequences and modifications thereof, nucleotide sequences encoding such peptides, and compositions including such peptides and encoding nucleotide sequences, and cells expressing such peptides, are provided. Such DV peptides, nucleotide sequences and compositions, can be used to elicit, stimulate, induce, promote, increase, enhance or activate an anti-DV CD8⁺ T cell response or an anti-DV CD4⁺ T cell response. Such peptides, nucleotide sequences and compositions can also be used for and in methods of vaccination/immunization of a subject against Dengue virus (DV) (e.g., to provide protection against DV infection and/or pathology), and for treatment of a subject in need thereof, for example, treatment of the subject for a Dengue virus (DV) infection or pathology.

### RELATED APPLICATIONS

This application is a continuation in part of International Application No. PCT/US2011/041889, filed Jun. 24, 2011, which claims the benefit of priority to provisional application Ser. No. 61/358,142, filed Jun. 24, 2010, and provisional application Ser. No. 61/391,882, filed Oct. 11, 2010, all of which are expressly incorporated herein by reference in their entirety.

### GOVERNMENT SUPPORT

This invention received government support under National Institutes of Health grants AI060989, AI077099, U54 AI057157 and National Institutes of Health contract HHSN272200900042C. The government has certain rights in the invention.

### SEQUENCE LISTING

The instant application contains a Sequence Listing which has been submitted in ASCII format via EFS-Web and hereby incorporated by reference in its entirety. Said ASCII copy, created on Apr. 1, 2013, is named 2008-06-09_SEQ_ST25.txt and is 212,000 bytes in size.

### INTRODUCTION

Dengue virus (DENV, DV) is a mosquito-borne RNA virus in the Flaviviridae family, which also includes West Nile Virus (WNV), Yellow Fever Virus (YFV), and Japanese Encephalitis Virus (JEV). The four serotypes of DENV (DENV1-4) share approximately 65-75% homology at the amino acid level (Fu, et al. Virology 188:953 (1992)). Infections with DENV can be asymptomatic, or cause disease ranging from dengue fever (DF) to dengue hemorrhagic fever (DHF) and dengue shock syndrome (DSS) (WHO, Dengue: Guidelines for diagnosis, treatment, prevention and control (2009)). DF is a self-limiting illness with symptoms that include fever, headache, myalgia, retro-orbital pain, nausea, and vomiting. DHF and DSS are characterized by increased vascular permeability, thrombocytopenia, hemorrhagic manifestations, and in the case of DSS, shock, which can be fatal. The incidence of DENV infections has increased 30-fold in the past 50 years (WHO, Dengue: Guidelines for diagnosis, treatment, prevention and control (2009)). DF and DHF/DSS are a significant cause of morbidity and mortality worldwide, and therefore a DENV vaccine is a global public health priority. However, vaccine development has been challenging, as a vaccine should protect against all four DENV serotypes (Whitehead, et al. Nat Rev Microbiol 5:518 (2007)).

Severe dengue disease (DHF/DSS) most often occurs in individuals experiencing a secondary infection with a heterologous DENV serotype, suggesting the immune response contributes to the pathogenesis (Sangkawibha, et al. Am J Epidemiol 120:653 (1984); Guzman, et al. Am J Epidemiol 152:793 (1997)). One hypothesis is that serotype cross-reactive antibodies enhance infection of FcγR⁺ cells during a secondary infection resulting in higher viral loads and more severe disease via a phenomenon known as antibody-dependent enhancement (ADE) (Morens, et al. Clin Infect Dis 19:500 (1994); Halstead, Adv Virus Res 60:421 (2003)). Recent studies have demonstrated DENV-specific antibody can enhance disease in mice (Zellweger, et al. Cell Host Microbe 7:128 (2010); Balsitis, et al. PLoS Pathog 6:e1000790 (2010)). It has also been proposed that serotype cross-reactive memory T cells may respond sub-optimally during secondary infection and contribute to the pathogenesis (Mathew, et al. Immunol Rev 225:300 (2008)). Accordingly, studies have shown serotype cross-reactive T cells can exhibit an altered phenotype in terms of cytokine production and degranulation (Mangada, et al. J Immunol 175:2676 (2005); Mongkolsapaya, et al. Nat Med 9:921 (2003); Mongkolsapaya, et al. J Immunol 176:3821 (2006)). However, another study found the breadth and magnitude of the T cell response during secondary DENV infection was not significantly associated with disease severity (Simmons, et al. J Virol 79:5665 (2005)). Although many studies have investigated the role of T cells in DENV pathogenesis, few studies have examined the contribution of T cells to protection against DENV. Consequently, the role of T cells in protection versus pathogenesis during DENV infections is presently unknown. This is primarily due to the lack of an adequate animal model, as mice are resistant to infection with this human pathogen (Yauch, et al. Antiviral Res 80:87 (2008)). A mouse-passaged DENV2 strain, S221, does not replicate to detectable levels in wild-type C57BL/6 mice, but does replicate in IFN-α/βR^{-/-} mice (Yauch, et al. J Immunol 182:4865 (2009)). Using S221 and IFN-α/βR^{-/-} mice, a protective role for CD8⁺ T cells in the response to primary DENV2 infection was reported (Yauch, et al. J Immunol 182:4865 (2009)).

CD4⁺ T cells can contribute to the host response to pathogens in a variety of ways. They produce cytokines and can mediate cytotoxicity. They also help B cell responses by inducing immunoglobulin class switch recombination (CSR), and help prime the CD8⁺ T cell response. CD4⁺ T cells can help the CD8⁺ T cell response indirectly by activating APCs, for example via CD40L/CD40 (Bevan, Nat Rev Immunol 4:595 (2004)). CD40L on CD4⁺ T cells is important in activating B cells as well (Elgueta, et al. Immunol Rev 229:152 (2009)). CD4⁺ T cells can also induce chemokine production that attracts CD8⁺ T cells to sites of infection (Nakanishi, et al. Nature 462:510 (2009)). However, the requirement for CD4⁺ T cell help for antibody and CD8⁺ T cell responses is not absolute, and may be specific to the pathogen and/or experimental system. For instance, it has been shown that CSR can occur in the absence of CD4⁺ T cells (Stavnezer, et al. Annu Rev Immunol 26:261 (2008)), and the primary CD8⁺ T cell response is CD4-independent under inflammatory conditions (Bevan, Nat Rev Immunol 4:595 (2004)).

Numerous studies have investigated the phenotype of DENV serotype cross-reactive T cells, which have been hypothesized to contribute to the pathogenesis of secondary heterologous infections, yet the actual contribution of T cells during DENV infection is unknown.

This suspected dual role of T cells in protection and pathogenesis is difficult to study in humans, since in most donor cohorts the time point and in case of secondary infections the sequence of infection is unknown, and does not allow direct correlations with T cell responses. A mouse model, which allows investigation of adaptive immune responses restricted by human histocompatibility complex (MHC) molecules to DENV infection, would shed light on the role of T cells in protection and/or pathogenesis. Mice transgenic for human leukocyte antigens (HLA) are widely used to study T cell responses restricted by human MHC molecules and studies in other viral systems have shown the valuable impact of HLA transgenic mice in epitope identification (Kotturi, et al. Immunome Res 6:4 (2010); Kotturi, et al. Immunome Res 5:3 (2009); Pasquetto, et al. J Immunol 175:5504 (2005)). It has been reported that mice lacking the IFNR-α/β support a productive DENV infection and allow the study of T cell responses after DENV infection (Yauch, et al. J Immunol 185:5405 (2010); Yauch, et al. J Immunol 182:4865 (2009); Shresta, et al. J Virol 78:2701 (2004)). To cover a wide range of HLA phenotypes, IFN-α/βR^{-/-} mice were backcrossed with HLA, A*0201, A*0101, A*1101, B*0701 and DRB1*0101 transgenic mice and the T cell response against infection with DENV was determined.

### SUMMARY

As disclosed herein, the contribution of CD4⁺ T cells to the host response to primary DENV2 infection using IFN-α/βR^{-/-} mice is defined. Infection with DENV2 resulted in CD4⁺ T cell expansion and activation. The DENV-specific CD4⁺ T cells expressed intracellular IFN-γ, TNF, IL-2, and CD40L, and could mediate in vivo cytotoxicity (e.g., kill peptide-pulsed target cells in vivo). Surprisingly, depletion of CD4⁺ T cells before DENV infection had no effect on viral loads, and CD4⁺ T cells were not required for the induction of the DENV2-specific antibody or CD8⁺ T cell responses. Candidate MHC class II (I-A^{b})-binding peptides from the entire proteome of DENV2, which is approximately 3390 amino acids and encodes three structural (core (C), envelope (E), and membrane (M)), and seven non-structural (NS) (NS1, NS2A, NS2B, NS3, NS4A, NS4B, NS5) proteins, were identified. Numerous additional CD4⁺ T cell and CD8+ T cell epitopes from the structural and non-structural (NS) proteins are also disclosed herein (e.g., Tables 1-4, 8 & 10 (SEQ ID Nos: 11-1163, respectively, in order of appearance)) Immunization with T cell epitopes, such as CD8⁺ or CD4⁺ T cell epitopes, before DENV infection resulted in significantly lower viral loads. While CD4⁺ T cells do not appear to be required for controlling primary DENV infection, immunization contributes to viral clearance.

By way of example, 42 epitopes derived from 9 of the 10 DENV proteins were identified. 80% of the epitopes identified were able to elicit a T cell response in human donors, previously exposed to DENV. The mouse model described herein also reflected response patterns observed in humans. These findings indicate that inducing anti-DENV CD4⁺ T and/or CD8+ T cell responses by immunization/vaccination will be an effective prophylactic or therapeutic treatment for DENV infection and/or pathology.

By way of a further example, HLA class I binding predicted epitopes for 16 HLA A and 11 HLA B alleles for all four DENV serotypes were tested in ex vivo IFNγ ELISPOT assays in HLA-matched PBMC of 250 blood donors from Sri Lanka where DENV is hyper-epidemic. This proteome-wide screen h identified a total of 408 epitopes across all 10 DENV proteins. Clustering epitopes that share more than 80% sequence homology resulted in the definition of 267 antigenic regions, with the 25 most immunodominant regions accounting for 50% of the total response observed.

In accordance with the invention, there are provided peptides, methods and uses, in which the peptides include or consist of a subsequence, portion, or an amino acid modification of Dengue virus (DV) structural or non-structural (NS) polypeptide sequence, and the peptide elicits, stimulates, induces, promotes, increases, or enhances an anti-DV CD8⁺ T cell response or an anti-DV CD4⁺ T cell response. In one embodiment, a peptide includes or consists of a subsequence, portion, or an amino acid modification of Dengue virus (DV) structural core (C), membrane (M) or envelope (E) polypeptide sequence, for example, based upon or derived from a DENV1, DENV2, DENV3 or DENV4 serotype. In another embodiment, a peptide includes or consists of a subsequence, portion, or an amino acid modification of Dengue virus (DV) NS1, NS2A, NS2B, NS3, NS4A, NS4B or NS5 polypeptide sequence, for example, based upon or derived from a DENV1, DENV2, DENV3 or DENV4 serotype.

In particular aspects, a peptide includes or consists of a sequence set forth in Tables 1-4, 8 & 10 (SEQ ID Nos: 11-1163, respectively, in order of appearance), or a subsequence thereof or a modification thereof. Exemplary modifications include 1, 2, 3, 4, 5 or 6, 7, 8, 9, 10 or more conservative, non-conservative, or conservative and non-conservative amino acid substitutions.

In certain embodiments, a peptide elicits an anti-DV response. In particular aspects, an anti-DV response includes a CD8⁺ T cell response and/or a CD4⁺ T cell response. Such responses can be ascertained, for example, by increased IFN-gamma, TNF-alpha, IL-1alpha, IL-6 or IL-8 production by CD8⁺ T cells in the presence of the peptide; and/or increased CD4⁺ T cell production of IFN-gamma, TNF, IL-2, or CD40L in the presence of the peptide, or killing of peptide-pulsed target cells.

The invention also provides compositions including the peptides (e.g., T cell epitopes), such as pharmaceutical compositions. Compostions can include one or more peptides selected from Tables 1-4, 8 & 10 (SEQ ID Nos: 11-1163, respectively, in order of appearance) or a subsequence thereof or a modification thereof, as well as optionally adjuvants.

Peptides, and subsequences, portions, and modifications thereof (e.g., T cell epitopes) can be used for stimulating, inducing, promoting, increasing, or enhancing an immune response against Dengue virus (DV) in a subject. In one embodiment, a method includes administering to a subject an amount of a peptide sufficient to stimulate, induce, promote, increase, or enhance an immune response against Dengue virus (DV) in the subject, and/or provide the subject with protection against a Dengue virus (DV) infection or pathology, or one or more physiological conditions, disorders, illness, diseases or symptoms caused by or associated with DV infection or pathology.

Peptides, and subsequences, portions, and modifications thereof (e.g., T cell epitopes) can also be used for treating a subject for a Dengue virus (DV) infection. In one embodiment, a method includes administering to a subject an amount of a peptide sufficient to treat the subject for the Dengue virus (DV) infection.

Exemplary responses, in vitro, ex vivo or in vivo, elicited by T cell epitopes include, stimulating, inducing, promoting, increasing, or enhancing an anti-DV CD8⁺ T cell response or an anti-DV CD4⁺ T cell response. In particular aspects, CD8⁺ T cells produce IFN-gamma, TNF-alpha, IL-1alpha, IL-6 or IL-8 in response to T cell epitope, and/or CD4⁺ T cells produce IFN-gamma, TNF, IL-2 or CD40L, or kill peptide-pulsed target cells in response to a T cell epitope. Accordingly, peptides, and subsequences, portions, and modifications thereof (e.g., T cell epitopes) can also be used for inducing, increasing, promoting or stimulating anti-Dengue virus (DV) activity of CD8⁺ T cells or CD4+ T cells in a subject.

In various embodiments, multiple peptides, for example, multiple Dengue virus (DV) T cell epitopes are employed in the methods and uses of the invention. In particular aspects, the Dengue virus (DV) T cell epitope includes or consists of one or more sequences set forth in Tables 1-4, 8 &10, (SEQ ID Nos: 11-1163, respectively, in order of appearance) or a subsequence thereof or a modification thereof.

### DESCRIPTION OF DRAWINGS

The patent or application file contains at least one drawing executed in color. Copies of this patent or patent application publication with color drawing(s) will be provided by the Office upon request and payment of the necessary fee.

FIGS. 1A-1D show that DENV2 infection results in CD4⁺ T cell activation and expansion in IFN-α/βR^{-/-} mice. A) The numbers of splenic CD4⁺ T cells in naïve IFN-α/βR^{-/-} mice (n=6) and IFN-α/βR^{-/-} mice infected with 10¹⁰ genomic equivalents (GE) of DENV2 (n=11) are shown. *** p<0.001 for naïve versus infected mice. B) The percentage of CD62L^{lo}CD44^{hi} cells (gated on CD4⁺ cells) is shown for naïve (n=4) and IFN-α/βR^{-/-} mice infected with 10¹⁰ GE of DENV2 (n=8). ** p<0.01 for naïve versus infected mice. C) Blood lymphocytes were obtained from IFN-α/βR^{-/-} mice on days 3, 5, 7, 10, and 14 after infection with 10¹⁰ GE of DENV2. The percentage of CD44^{hi}CD62L^{lo} cells (gated on CD4⁺ T cells)±SEM (n=6) is shown. D) The percentage and number of splenic Foxp3⁺ cells (gated on CD4⁺ cells) are shown for naïve (n=4) and infected IFN-α/βR^{-/-} mice (n=4).

FIGS. 2A-2B show the identification of DENV2-derived epitopes recognized by CD4⁺ T cells. A) Splenocytes were obtained from IFN-α/βR^{-/-} mice 7 days after infection with 10¹⁰ GE of DENV2 and re-stimulated in vitro with DENV2-derived 15-mer peptides predicted to bind I-A^{b}. Cells were then stained for surface CD4 and intracellular IFN-γ and analyzed by flow cytometry. The 4 positive peptides identified are shown. In the dot plots, the percentage of CD4⁺ T cells producing IFN-γ is indicated. The responses of individual mice as well as the mean and SEM are also shown (n=7-11). The response of unstimulated cells was subtracted from the response to each DENV2 peptide, and the net percentage and number of splenic CD4⁺ T cells producing IFN-γ are indicated. B, Splenocytes were obtained from wild-type C57BL/6 mice 7 days after infection with 10¹⁰ GE of DENV2 and stimulated and stained as in A (n=6).

FIG. 3 shows that DENV2-specific CD4⁺ T cells are polyfunctional. Splenocytes were obtained from IFN-α/βR^{-/-} mice 7 days after infection with 10¹⁰ GE of DENV2 and stimulated in vitro with individual peptides. Cells were then stained for surface CD4, and intracellular IFN-γ, TNF, IL-2, and CD40L, and analyzed by flow cytometry. The response of unstimulated cells was subtracted from the response to each DENV2 peptide, and the net percentages of the CD4⁺ T cells that are expressing at least one molecule are indicated. The mean and SEM of 3 mice is shown.

FIG. 4 shows that depletion of CD4⁺ T cells prior to DENV2 infection does not affect viral RNA levels. IFN-α/βR^{-/-} mice were depleted of CD4⁺ or CD8⁺ cells, or both, by administration of GK1.5 or 2.43 Ab, respectively, (or given an isotype control Ab) 2 days before and 1 day after infection with 10¹⁰ GE of DENV2. Mice were sacrificed 5 days later, and DENV2 RNA levels in the serum, spleen, small intestine, brain, and kidney were quantified by real-time RT-PCR. Data are expressed as DENV2 copies per ml of sera, or DENV2 units normalized to 18S rRNA levels for the organs. Each symbol represents one mouse, the bar represents the geometric mean, and the dashed line is the limit of detection. * p<0.05, ** p<0.01, and *** p<0.001 for viral RNA levels comparing T cell-depleted mice with control mice.

FIGS. 5A-5C show that CD4⁺ T cells are not required for the anti-DENV2 antibody response. IFN-α/βR^{-/-} mice (control or CD4-depleted) were infected with 10¹⁰ GE of DENV2. A) IgM and IgG titers in the sera at day 7 were measured by ELISA (n=5 control and 6 CD4-depleted mice). Data are combined from two independent experiments. B) Neutralizing activity of sera from naïve (n=4) and control (n=6) or CD4-depleted mice (n=6) obtained 7 days after infection was determined by measuring the ability of the sera to reduce DENV2 infection of C6/36 cells. C) The percentage of germinal center B cells (GL7⁺Fas⁺, gated on B220⁺ cells) in the spleen 7 days after infection is shown. The plots are representative of 5 control and 5 CD4-depleted mice.

FIGS. 6A-6C show that CD4⁺ T cells are not required for the primary DENV2-specific CD8⁺ T cell response. A) Splenocytes were obtained from IFN-α/βR^{-/-} mice (control or CD4-depleted) 7 days after infection with 10¹⁰ GE of DENV2, and stimulated in vitro with immunodominant DENV2-derived H-2^{b}-restricted CD8⁺ T cell epitopes. Cells were then stained for CD8 and IFN-γ and analyzed by flow cytometry, and the number of CD8⁺ T cells producing IFN-γ is shown. Results are expressed as the mean±SEM of 4 mice per group. ** p<0.01. B) Splenocytes were obtained as in A and stimulated with NS4B₉₉₋₁₀₇ in the presence of an anti-CD 107 Ab, and then stained for CD8, IFN-γ, TNF, and IL-2. The response of unstimulated cells was subtracted from the response to each DENV2 peptide, and the net percentages of the CD8⁺ T cells that are expressing at least one molecule are indicated. The mean and SEM of 3 mice is shown. C) CD8⁺ T cell-mediated killing. IFN-α/βR^{-/-} mice (control or CD4-depleted) infected 7 days previously with 10¹⁰ GE of DENV2 were injected i.v. with CFSE-labeled target cells pulsed with a pool of DENV2-derived immunodominant H-2^{b}-restricted peptides (C₅₁₋₅₉, NS2A₈₋₁₅, NS4B₉₉₋₁₀₇, and NS5₂₃₇₋₂₄₅) at the indicated concentrations (n=3-6 mice per group). After 4 h, splenocytes were harvested, analyzed by flow cytometry, and the percentage killing was calculated.

FIG. 7 shows cytotoxicity mediated by DENV2-specific CD4⁺ T cells. In vivo killing of DENV2-derived I-A^{b}-restricted peptide-pulsed cells. IFN-α/βR^{-/-} mice (control, CD4-depleted, or CD8-depleted) infected 7 days previously with 10¹⁰ GE of DENV2 were injected i.v. with CFSE-labeled target cells pulsed with the three epitopes that contain only CD4⁺ T cell epitopes (NS2B₁₀₈₋₁₂₂, NS3₁₉₈₋₂₁₂, and NS3₂₃₇₋₅₁) (n=6 control, 3 CD4-depleted, and 3 CD8-depleted mice). After 16 h, splenocytes were harvested, analyzed by flow cytometry, and the percentage killing was calculated.

FIG. 8 shows that peptide immunization with CD4⁺ T cell epitopes results in enhanced DENV2 clearance. IFN-α/βR^{-/-} mice were immunized s.c. with 50 μg each of the three DENV peptides that contain only CD4⁺ T cell epitopes (NS2B₁₀₈₋₁₂₂, NS3₁₉₈₋₂₁₂, NS3₂₃₇₋₅₁) in CFA, or mock-immunized with DMSO in CFA. Mice were boosted 11 days later with peptide in IFA, then challenged with 10¹¹ GE of DENV2 13 days later, and sacrificed 4 days after infection. Separate groups of peptide-immunized mice were depleted of CD4⁺ or CD8⁺ T cells prior to infection. DENV2 RNA levels in the tissues were quantified by real-time RT-PCR and are expressed as DENV2 units normalized to 18S rRNA. Each symbol represents one mouse and the bar represents the geometric mean. * p<0.05, ** p<0.01.

FIG. 9 shows identification of DENV-derived epitopes recognized by CD8⁺ T cells. DENV specific epitope identification was performed in four different HLA transgenic mouse strains (A) A*0201; (B) A *1101; (C) A*0101; (D) B*0702. For all strains tested, IFNγ ELISPOT was performed using splenic T cells isolated from HLA transgenic IFN-α/βR^{-/-} mice (black bars) and HLA transgenic IFN-α/βR^{-/-} mice (white bars). Mice were infected i.v. retro-orbitally with 1×10¹⁰ GE of DENV2 (S221) in 100 μl PBS. Seven days post-infection, CD8⁺ T cells were purified and tested against a panel of S221 predicted peptides. The data are expressed as mean number of SFC/10⁶ CD8⁺ T cells of two independent experiments. Error bars represent SEM. Responses against peptides were considered positive if the stimulation index (SI) exceeded double the mean negative control wells (effector cells plus APCs without peptide) and net spots were above the threshold of 20 SFCs/10⁶ CD8⁺ T cells in two independent experiments. Asterisks indicate peptides, which were able to elicit a significant IFNγ response in each individual experiment, according to the criteria described above.

FIG. 10 shows identification of DENV-derived epitopes recognized by CD4⁺ T cells. IFNγ ELISPOT was performed using CD4⁺ T cells isolated from DRB1*0101 transgenic IFN-α/βR^{-/-} (black bars) and IFN-α/βR^{-/-} (white bars) mice. Mice were infected i.v. retro-orbitally with 1×10¹⁰ GE of DENV2 (S221) in 100 μl PBS. Seven days postinfection, CD4⁺ T cells were purified and tested against a panel of S221 predicted peptides. The data are expressed as mean number of SFC/10⁶ CD4⁺ T cells of two independent experiments. Error bars represent SEM. Responses against peptides were considered positive if the stimulation index (SI) exceeded double the mean negative control wells (effector cells plus APCs without peptide) and net spots were above the threshold of 20 SFCs/10⁶ CD4⁺ T cells in two individual experiments. Asterisks indicate peptides, which were able to elicit a significant IFNγ response, according to the criteria described above.

FIGS. 11A-11B show the determination of optimal epitope studies. To determine the dominant epitope, HLA-transgenic IFN-α/βR^{-/-} mice were infected with 1×10¹⁰ GE of DENV2 (S221) and spleens harvested 7 days post infection. CD8⁺ T cells were purified and incubated for 24 hours with ascending concentrations of nested peptides. A) shows pairs of peptides where the 9-mer and the 10mer were able to elicit a significant T cell response; B) shows the 3 B*0702 restricted peptides which did show an IC₅₀>1000 nM in the respective binding assay. Peptides were retested in parallel with their corresponding 8-, 10- and 11-mers. The peptides, which were able to elicit stronger IFNγ responses at various concentrations, were then considered the dominant epitope.

FIGS. 12A-12B show MHC-restriction of identified epitopes. HLA A*0201 (A) and HLA A* 1101 (B) transfected 0.221 cells, as well as the non-transfected cell line as a control, were used as antigen presenting cells in titration experiments to determine MHC restriction. Purified CD8⁺ T cells from DENV2 (S221) infected HLA A*A0201 and HLA A* 110 IFN-α/βR^{-/-} mice were incubated with increasing concentrations of peptides and tested for IFNγ production in an ELISPOT assay. Representative graphs of CD8⁺ T cell responses are shown, when incubated with HLA transfected cell lines (A and B; black lines) and non-transfected cell lines (A and B, grey lines) are shown. The dotted line indicates the 25 net SFCs/10⁶cells threshold used to define positivity.

FIGS. 13A-13F show antigenicity of identified epitopes in human donors. Epitopes (1 μg/ml individual peptide for 7 days) identified in the HLA-transgenic IFN-α/βR^{-/-} mice were validated by their capacity to stimulate PBMC (2×10⁶ PBMC/ml) from human donors and then tested in an IFNγ ELISPOT assay. A-E) show IFNγ responses/10⁶ PBMC after stimulation with A*0101, A*0201, A*1101, B*0702 and DRB1*0101 restricted peptides, respectively. Donors, seropositive for DENV, were grouped in HLA matched and non-HLA matched cohorts, as shown in panels 1 and 2 of each figure. All epitopes identified were further tested in DENV seronegative individuals. The average IFNγ responses elicited by PBMC from DENV seropositive non-HLA matched and DENV seronegative donors plus 3 times the standard deviation (SD) was set as a threshold for positivity, as indicated by the dashed line. F) shows the mean IFNγ response/10⁶ T cells from HLA transgenic mice (black bars) and HLA matched donors (white bars) grouped by HLA restriction of the epitopes tested.

FIG. 14 shows subproteinlocation of identified epitopes from Table 2. All identified epitopes were grouped according to the DENV subprotein they are derived from. Black bars show the total IFNγ response all epitopes of a certain protein could elicit. Numbers in parenthesis indicate the number of epitopes that have been detected for this protein.

FIGS. 15A-15C show HLA coverage and serological characteristics of the study population. (A) HLA allele coverage in the Sri Lankan cohort is shown. Bars represent the relative number of donors where the donor specific HLA alleles have been exactly matched (black bars) or matched within the same supertype (white bars) with one of the 27 alleles selected for our study. The black line represents the cumulative number of donors where at least 1 allele has been matched exactly. (B) The relative number of donors with neutralizing antibodies against one, two, three or all four serotypes is shown. Donors have experienced either primary (white bars) or secondary infection (black bars). (C) Summary of all donors experiencing primary infection (n=55). Neutralization titers against donors infected with DENV1 (n=14), DENV2 (n=18), DENV3 (n=20) or DENV4 (n=3) are shown.

FIGS. 16A-16B show immunodominant regions of the dengue virus polyprotein. (A) The genomic position of DENV encoded proteins ((capsid (C), pre-membrane (prM/M), envelope (E), NS1, NS2A, NS2B, NS3, NS4A, NS4B, NS5) and the total observed response magnitude for every amino acid along the proteome is shown (black bars). The data are expressed as total number of IFNγ SFC/10⁶ PBMC. The heat map indicates the number of donors that showed a positive cytokine response to peptides within these regions. (B) Identified antigenic regions were plotted as a function of the percentage of the total response. Lines indicate the number of regions needed to account for 25, 75 and 90% of the total response, respectively.

FIGS. 17A-17D show differences between serotype specific responses. (A) Magnitude of serotype specific and conserved T cell responses. Total of responses observed against serotype specific regions (black bars) or regions being conserved/homologous (white bars) are shown. Conserved regions are defined as sequences found in two or more serotypes, also allowing one residue substitutions to account for potential cross-reactivity of highly homologous sequences. The data are expressed as total number of IFNγ SFC/10⁶ PBMC. Responses magnitudes (as SFC/10⁶ PBMC values) directed against conserved regions (B), specific for DENV2 (C) or DENV3 (D) were plotted as a function of the genomic position in the DENV polyprotein.

FIGS. 18A-18H show deciphering antigenic sin. Responses form donors either exhibiting responses against DENV2 (A) or DENV3 (B) specific epitopes are compiled. All responses observed in these donors were then plotted dependent on their origin from serotype specific (white bars) or conserved regions (black bars). Representative donors were incubated with donor-specific peptide pools [1 μg/ml] originated either from regions serotype specific for DENV3 (white circles) or regions conserved between two or more serotypes (black circles) for 6 hours in the presence of BFA. Cells were then stained with mAB against surface markers CD3, CD8, CD45RA, CD27, and mAB against intracellular C107a, IFNγ, TNFα and IL2. Magnitude of response (C), and phenotype of responding cell (D) of the individual donors (n=7) based on gating of the IFN′ producing cells is shown. The average CD45RA, CCR7, CD27 and CD107a expression for all responding cells is shown in panel (E). Multifunctional responses are shown for individual donors (F) and as average of all donors studied (G, n=6). Avidity of responding T cells was determined by incubating PBMC with ascending concentrations of peptide pools [0.001; 0.01; 0.1; 1; and 10 μg/ml]. The peptide concentration, which was necessary to induce 50% of the maximum responses (EC50) was calculated and compared between normalized samples (H).

FIGS. 19A-19C show HLA linked T cell responses. (A) Differential frequency and magnitude of HLA restricted responses. Frequency (black bars) and the magnitude (white bars) of T cell responses (as total SFC/10⁶ PBMC values) sorted according to their restriction element is shown. (B) Association of HLA restricted T cell responses with disease susceptibility. A meta-analysis of all data associating HLA allele with disease susceptibility available in the literature was performed. For each of the studies, all investigated alleles were ranked according to their association with disease (dengue fever [DF], dengue hemorrhagic fever [DHF] and dengue shock syndrome [DSS]). A calculated percentile ranking across all studies for the 18 alleles detected in significant frequencies in our own cohort was performed and correlated the rankings with T cell responses. Disease susceptibility was correlated with the average magnitudes of HLA restricted responses (left panel), with the frequency and the magnitude of T cell responses per donor (middle panel) and with the magnitude per epitope as well as breath of (right panel) One-tailed Spearman test was then used to calculate correlations using Prism Graph Pad Software, Inc (La Jolla, Calif.). (C) Multi-functionality of HLA restricted responses. Representative donors were stimulated with HLA restricted donor-specific peptide pools. [1μg/ml] for 6 hours in the presence of BFA. Cells were then stained with mAB against surface markers CD3, CD8 and mAB against intracellular IFNγ, TNFα and IL2. Pie charts represent cytokine profiles of individual donors. The relative number of cells producing one (dark grey areas), two (light grey areas) or three (white areas) of the measured cytokines are shown. Percentages in the pie charts represent the number of cells, which produce 2 or more cytokines. Numbers under the pie charts represent the T cell responses (as IFNγ SFC/106 PBMC) of this specific donor/allele combination in the ELISPOT assay. Responses hierarchy was IFNγ>TNFα>IL2 in all experiments (n=4).

FIGS. 20A-20D show a correlation between T cell and antibody responses. (A) DENV specific IgG titers of all donors investigated in this study are shown. Results are grouped according to the immune status of the donor (dengue negative, primary or secondary infection). Donors with history of dengue infection have additionally been grouped into responding donors (R) with non-responding donors (NR) referring to a detectable T cell response in the IFNγ ELISPOT assay. Serum sample from 80 donors were investigated and total dengue specific IgG titers (B), enhancement titers (C) and neutralization titers (D) were measured. These parameters were then compared to T cell responses observed in these specific donors. Data were analyzed using Prism Graph Pad Software, Inc (La Jolla, Calif.).

### DETAILED DESCRIPTION

The invention is based at least in part on Dengue virus (DV) peptides, subsequences and portions, and amino acid modifications of DV peptides, subsequences and portions. Invention Dengue virus (DV) peptides, subsequences, portions and modifications thereof, including T cell epitopes that elicit, stimulate, induce, promote, increase, enhance, or activate an anti-DV CD4⁺ T cell and/or an anti-DV CD8⁺ T cell response in vitro or in vivo, are useful in vaccination and immunization (e.g., prophylactic), as well as treatment uses and methods. For example, an invention Dengue virus (DV) peptide, subsequence, portion or modification thereof, can be used to immunize or vaccinate a subject, or to treat a subject having or at risk of having Dengue virus (DV) infection or pathology.

Dengue virus (DV) peptide, subsequences, portions and modifications thereof include T cell epitopes. A T cell epitope can elicit, stimulate, induce, promote, increase, enhance, or activate an anti-DV CD4⁺ T cell and/or an anti-DV CD8⁺ T cell response in vitro (e.g., in solution, in solid phase, in tissue culture) or in vivo. Such anti-DV CD4⁺ T cell and/or an anti-DV CD8⁺ T cell responses can be detected using various assays disclosed herein or known to the skilled artisan. For example, an anti-DV CD8⁺ T cell response can include one or more of increased IFN-gamma, TNF-alpha, IL-1alpha, IL-6 or IL-8 production by CD8⁺ T cells in the presence of the peptide; and an anti-DV CD4⁺ T cell response can include one or more of increased IFN-gamma, TNF, IL-2, or CD40L production by CD4⁺ T cells in the presence of the peptide, or CD4⁺ T cell killing of peptide-pulsed target cells.

Exemplary T cell epitopes can include or consist of a subsequence, a portion or modification of Dengue virus (DV) structural Core, Membrane or Envelope polypeptide sequence, or a subsequence or portion of a Dengue virus (DV) non-structural (NS)NS1, NS2A, NS2B, NS3, NS4A, NS4B or NS5 polypeptide sequence. Specific non-limiting examples of Dengue virus (DV) structural protein and non-structural (NS) protein subsequences, portions and modifications include or consist of a sequence set forth in Tables 1-4, 8 & 10 (SEQ ID Nos: 11-1163, respectively, in order of appearance), as well as subsequences and portions, and amino acid modifications of sequences set forth in Tables 1-4, 8 & 10 (SEQ ID Nos: 11-1163, respectively, in order of appearance).

Additional Dengue virus (DV) peptide, subsequences and portions thereof can be based upon or derived from DENV serotypes, such as DENV1, DENV2, DENV3 or DENV4 serotypes. For example, a subsequence or portion of a Dengue virus (DV) structural polypeptide such as a core (C), membrane (M) or envelope (E) polypeptide, or a non-structural (NS)NS1, NS2A, NS2B, NS3, NS4A, NS4B or NS5 polypeptide, can be a sequence having 75% or more (e.g., 80%, 85%, 90%, 95%, 96%, 97%, 98%, 99%, 100%) sequence identity to all or a region of a structural or non-structural (NS) Dengue virus (DV) serotype, such as a DENV1, DENV2, DENV3 or DENV4 serotype (e.g., a peptide listed in any of Tables 1-4, 8 & 10 (SEQ ID Nos: 11-1163, respectively, in order of appearance)).

Thus, in accordance with the invention, there are also provided Dengue virus (DV) peptides, subsequences, portions and modifications thereof that exhibit sequence identity to a reference Dengue virus (DV) peptide, subsequence or portion, or modification thereof (e.g., as set forth in Tables 1-4, 8 & 10 (SEQ ID Nos: 11-1163, respectively, in order of appearance)). In one embodiment, an Dengue virus (DV) peptide, subsequence, portion or modification thereof includes or consists of a sequence at least 60% or more (e.g., 65%, 70%, 75%, 80%, 85%, 90%, 95%, etc.) identical to a reference Dengue virus (DV) peptide, subsequence, portion or modification thereof (e.g., a subsequence, portion or modification of any peptide listed in Tables 1-4, 8 & 10 (SEQ ID Nos: 11-1163, respectively, in order of appearance)).

In another embodiment, Dengue virus (DV) peptides, subsequences and portions thereof include or consist of a Dengue virus (DV) peptide, subsequence or portion thereof set forth as any peptide listed in Tables 1-4, 8 & 10 (SEQ ID Nos: 11-1163, respectively, in order of appearance) wherein the Dengue virus (DV) peptide, subsequence or portion thereof has one or more modifications, such as an amino acid addition to, deletion of, or substitution of any amino acid residue in any peptide listed in Tables 1-4, 8 & 10 (SEQ ID Nos: 11-1163, respectively, in order of appearance). In particular aspects, a modified sequence is at least 80% or more, e.g., 80-85%, 85-90%, 90-95%, 95-100% identical, to Dengue virus (DV) peptide, subsequence or portion thereof set forth any peptide listed in Tables 1-4, 8 & 10 (SEQ ID Nos: 11-1163, respectively, in order of appearance), or has 1, 2, 3, 4, 5, 6, 7, 8, 9, 10, 1, 12, 13, 14, 15, 16, 17, 18, 19, 20, 20-25, 25-30, 30-50, 50-100, or more, additions to, deletions of, or substitutions.

T cell epitopes typically are short amino acid sequences, e.g. about five to 15 amino acids in length (or 5-10 amino acids in length). Linear or contiguous T cell epitopes include a continuous amino acid sequence, such as a 5 to 15 amino acid sequence, which can elicit an anti-DV CD4⁺ T cell or anti-DV CD8⁺ T cell response in vitro or in vivo.

A non-limiting example of a subsequence or portion of a Dengue virus (DV) polypeptide sequence includes or consists of a subsequence or portion of Dengue virus (DV) structural Core, Membrane or Envelope polypeptide sequence. A non-limiting example of a subsequence or portion of a Dengue virus (DV) polypeptide sequence includes or consists of a subsequence or portion of Dengue virus (DV) non-structural (NS)NS 1, NS2A, NS2B, NS3, NS4A, NS4B or NS5 polypeptide sequence.

A non-limiting Core sequence from which a subsequence, portion or modification can be based upon is a sequence set forth as:

(SEQ ID NO: 1)
MNNQRKKARNTPFNMLKRERNRVSTVQQLTKRFSLGMLQGRGPLKLFMALVAFLRFLTIPP
TAGILKRWGTIKKSKAINVLRGFRKEIGRMLNILNRRRRTAGMIIMLIPTVMA.

A non-limiting Membrane sequence from which a subsequence, portion or modification can be based upon is a sequence set forth as:

(SEQ ID NO: 2)
FHLTTRNGEPHMIVSRQEKGKSLLFKTGDGVNMCTLMAMDLGELCEDTITYKCPLLRQNEP
EDIDCWCNSTSTWVTYGTCTTTGEHRREKRSVALVPHVGMGLETRTETWMSSEGAWKHA
QRIETWILRHPGFTIMAAILAYTIGTTHFQRALIFILLTAVAPSMT.

A non-limiting Envelope sequence from which a subsequence, portion or modification can be based upon is a sequence set forth as:

(SEQ ID NO: 3)
MRCIGISNRDFVEGVSGGSWVDIVLEHGSCVTTMAKNKPTLDFELIKTEAKQSATLRKYCIE
AKLTNTTTESRCPTQGEPSLNEEQDKRFVCKHSMVDRGWGNGCGLFGKGGIVTCAMFTCK
KNMKGKVVQPENLEYTIVITPHSGEEHAVGNDTGKHGKEIKITPQSSITEAELTGYGTVTME
CSPRTGLDFNEMVLLQMENKAWLVHRQWFLDLPLPWLPGADTQGSNWIQKETLVTFKNP
HAKKQDVVVLGSQEGAMHTALTGATEIQMSSGNLLFTGHLKCRLRMDKLQLKGMSYSMC
TGKFKVVKEIAETQHGTIVIRVQYEGDGSPCKIPFEIMDLEKRHVLGRLITVNPIVTEKDSPV
NIEAEPPFGDSYIIIGVEPGQLKLNWFKKGSSIGQMLETTMRGAKRMAILGDTAWDFGSLGG
VFTSIGKALHQVFGAIYGAAFSGVSWTMKILIGVIITWIGMNSRSTSLSVSLVLVGVVTLYLG
VMVQA.

A non-limiting non-structural NS 1 sequence from which a subsequence, portion or modification can be based upon is a sequence set forth as:

(SEQ ID NO: 4)
ADSGCVVSWKNKELKCGSGIFITDNVHTWTEQYKFQPESPSKLASAIQKAHEEGICGIRSVT
RLENLMWKQITPELNHILSENEVKLTIMTGDIKGIMQAGKRSLRPQPTELKYSWKTWGKAK
MLSTESHNQTFLIDGPETAECPNTNRAWNSLEVEDYGFGVFTTNIWLKLREKQDVFCDSKL
MSAAIKDNRAVHADMGYWIESALNDTWKIEKASFIEVKSCHWPKSHTLWSNEVLESEMIIP
KNFAGPVSQHNYRPGYHTQTAGPWHLGKLEMDFDFCEGTTVVVTEDCGNRGPSLRTTTAS
GKLITEWCCRSCTLPPLRYRGEDGCWYGMEIRPLKEKEENLVNSLVTA.

A non-limiting non-structural NS2A sequence from which a subsequence, portion or modification can be based upon is a sequence set forth as:

(SEQ ID NO: 5)
GHGQIDNFSLGVLGMALFLEEMLRTRVGTKHAILLVAVSFVTLITGNMSFRDLGRVMVMV
GATMTDDIGMGVTYLALLAAFKVRPTFAAGLLLRKLTSKELMMTTIGIVLLSQSTIPETILEL
TDALALGMMVLKMVRKMEKYQLAVTIMAILCVPNAVILQNAWKVSCTILAVVSVSPLFLT
SSQQKADWIPLALTIKGLNPTAIFLTTLSRTNKKR.

A non-limiting non-structural NS2B sequence from which a subsequence, portion or modification can be based upon is a sequence set forth as:

(SEQ ID NO: 6)
SWPLNEAIMAVGMVSILASSLLKNDIPMTGPLVAGGLLTVCYVLTGRSADLELERAADVK
WEDQAEISGSSPILSITISEDGSMSIKNEEEEQTLTILIRTGLLVISGLFPVSLPITAAAWYLWEV
KKQR.

A non-limiting non-structural NS3 sequence from which a subsequence, portion or modification can be based upon is a sequence set forth as:

(SEQ ID NO: 7)
AGVLWDVPSPPPVGKAELEDGAYRIKQKGILGYSQIGAGVYKEGTFHTMWHVTRGAVLM
HKGKRIEPSWADVKKDLISYGGGWKLEGEWKEGEEVQVLALEPGKNPRAVQTKPGLFKTN
AGTIGAVSLDFSPGTSGSPIIDKKGKVVGLYGNGVVTRSGAYVSAIAQTEKSIEDNPEIEDDIF
RKRKLTIMDLHPGAGKTKRYLPAIVREAIKRGLRTLILAPTRVVAAEMEEALRGLPIRYQTP
AIRAEHTGREIVDLMCHATFTMRLLSPVRVPNYNLIIMDEAHFTDPASIAARGYISTRVEMG
EAAGIFMTATPPGSRDPFPQSNAPIMDEEREIPERSWSSGHEWVTDFKGKTVWFVPSIKAGN
DIAACLRKNGKKVIQLSRKTFDSEYVKTRTNDWDFVVTTDISEMGANFKAERVIDPRRCMK
PVILTDGEERVILAGPMPVTHSSAAQRRGRIGRNPKNENDQYIYMGEPLENDEDCAHWKEA
KMLLDNINTPEGIIPSMFEPEREKVDAIDGEYRLRGEARKTFVDLMRRGDLPVWLAYRVAA
EGINYADRRWCFDGIKNNQILEENVEVEIWTKEGERKKLKPRWLDARIYSDPLALKEFKEFA
AGRK.

A non-limiting non-structural NS4A sequence from which a subsequence, portion or modification can be based upon is a sequence set forth as:

(SEQ ID NO: 8)
SLTLSLITEMGRLPTFMTQKARDALDNLAVLHTAEAGGRAYNHALSELPETLETLLLLTLLA
TVTGGIFLFLMSGRGIGKMTLGMCCIITASILLWYAQIQPHWIAASIILEFFLIVLLIPEPEKQR
TPQDNQLTYVVIAILTVVAATMA.

A non-limiting non-structural NS4B sequence from which a subsequence, portion or modification can be based upon is a sequence set forth as:

(SEQ ID NO: 9)
NEMGFLEKTKKDLGLGSITTQQPESNILDIDLRPASAWTLYAVATTFVTPMLRHSIENSSVN
VSLTAIANQATVLMGLGKGWPLSKMDIGVPLLAIGCYSQVNPITLTAALFLLVAHYAIIGPG
LQAKATREAQKRAAAGIMKNPTVDGITVIDLDPIPYDPKFEKQLGQVMLLVLCVTQVLMM
RTTWALCEALTLATGPISTLWEGNPGRFWNTTIAVSMANIFRGSYLAGAGLLFSIMKNTTNT
RR.

A non-limiting non-structural NS5 sequence from which a subsequence, portion or modification can be based upon is a sequence set forth as:

(SEQ ID NO: 10)
GTGNIGETLGEKWKSRLNALGKSEFQIYKKSGIQEVDRTLAKEGIKRGETDHHAVSRGSAK
LRWFVERNMVTPEGKVVDLGCGRGGWSYYCGGLKNVREVKGLTKGGPGHEEPIPMSTYG
WNLVRLQSGVDVFFTPPEKCDTLLCDIGESSPNPTVEAGRTLRVLNLVENWLNNNTQFCIK
VLNPYMPSVIEKMEALQRKYGGALVRNPLSRNSTHEMYWVSNASGNIVSSVNMISRMLINR
FTMRHKKATYEPDVDLGSGTRNIGIESEIPNLDIIGKRIEKIKQEHETSWHYDQDHPYKTWA
YHGSYETKQTGSASSMVNGVVRLLTKPWDVVPMVTQMAMTDTTPFGQQRVFKEKVDTRT
QEPKEGTKKLMKITAEWLWKELGKKKTPRMCTREEFTRKVRSNAALGAIFTDENKWKSAR
EAVEDSRFWELVDKERNLHLEGKCETCVYNMMGKREKKLGEFGKAKGSRAIWYMWLGA
RFLEFEALGFLNEDHWFSRENSLSGVEGEGLHKLGYILRDVSKKEGGAMYADDTAGWDTR
ITLEDLKNEEMVTNHMEGEHKKLAEAIFKLTYQNKVVRVQRPTPRGTVMDIISRRDQRGSG
QVGTYGLNTFTNMEAQLIRQMEGEGVFKSIQHLTVTEEIAVQNWLARVGRERLSRMAISGD
DCVVKPLDDRFASALTALNDMGKVRKDIQQWEPSRGWNDWTQVPFCSHHFHELIMKDGR
VLVVPCRNQDELIGRARISQGAGWSLRETACLGKSYAQMWSLMYFHRRDLRLAANAICSA
VPSHWVPTSRTTWSIHAKHEWMTAEDMLTVWNRVWIQENPWMEDKTPVESWEEIPYLGK
REDQWCGSLIGLTSRATWAKNIQTAINQVRSLIGNEEYTDYMPSMKRFRREEEEAGVLW.

As disclosed herein, Dengue virus (DV) peptides, subsequences, portions and modifications thereof of the invention include those having at least partial sequence identity to one or more exemplary Dengue virus (DV) peptides, subsequences, portions or modifications thereof (e.g., sequences set forth in Tables 1-4, 8 & 10 (SEQ ID Nos: 11-1163, respectively, in order of appearance)). The percent identity of such sequences can be as little as 60%, or can be greater (e.g., 65%, 70%, 75%, 80%, 85%, 90%, 95%, 96%, 97%, 98%, 99%, etc.). The percent identity can extend over the entire sequence length or a portion of the sequence. In particular aspects, the length of the sequence sharing the percent identity is 2, 3, 4, 5 or more contiguous amino acids, e.g., 6, 7, 8, 9, 10, 11, 12, 13, 14, 15, 16, 17, 18, 19, 20, etc. contiguous amino acids. In additional particular aspects, the length of the sequence sharing the percent identity is 20 or more contiguous amino acids, e.g., 20, 21, 22, 23, 24, 25, 26, 27, 28, 29, 30, 31, 32, 33, 34, 35, etc. contiguous amino acids. In further particular aspects, the length of the sequence sharing the percent identity is 35 or more contiguous amino acids, e.g., 35, 36, 37, 38, 39, 40, 41, 42, 43, 44, 45, 45, 47, 48, 49, 50, etc., contiguous amino acids. In yet further particular aspects, the length of the sequence sharing the percent identity is 50 or more contiguous amino acids, e.g., 50-55, 55-60, 60-65, 65-70, 70-75, 75-80, 80-85, 85-90, 90-95, 95-100, 100-110, etc. contiguous amino acids.

The term “identity” and grammatical variations thereof, mean that two or more referenced entities are the same. Thus, where two Dengue virus (DV) peptides, subsequences, portions and modifications thereof are identical, they have the same amino acid sequence. The identity can be over a defined area (region or domain) of the sequence. “Areas, regions or domains” of homology or identity mean that a portion of two or more referenced entities share homology or are the same.

The extent of identity between two sequences can be ascertained using a computer program and mathematical algorithm known in the art. Such algorithms that calculate percent sequence identity (homology) generally account for sequence gaps and mismatches over the comparison region or area. For example, a BLAST (e.g., BLAST 2.0) search algorithm (see, e.g., Altschul et al., J. Mol. Biol. 215:403 (1990), publicly available through NCBI) has exemplary search parameters as follows: Mismatch −2; gap open 5; gap extension 2. For polypeptide sequence comparisons, a BLASTP algorithm is typically used in combination with a scoring matrix, such as PAM100, PAM 250, BLOSUM 62 or BLOSUM 50. FASTA (e.g., FASTA2 and FASTA3) and SSEARCH sequence comparison programs are also used to quantitate the extent of identity (Pearson et al., Proc. Natl. Acad. Sci. USA 85:2444 (1988); Pearson, Methods Mol Biol. 132:185 (2000); and Smith et al., J. Mol. Biol. 147:195 (1981)). Programs for quantitating protein structural similarity using Delaunay-based topological mapping have also been developed (Bostick et al., Biochem Biophys Res Commun. 304:320 (2003)).

In accordance with the invention, modified and variant forms of Dengue virus (DV) peptides, subsequences and portions there are provided. Such forms, referred to as “modifications” or “variants” and grammatical variations thereof, mean a Dengue virus (DV) peptide, subsequence or portion thereof that deviates from a reference sequence. For example, certain sequences set forth in Tables 1-4, 8 & 10 (SEQ ID Nos: 11-1163, respectively, in order of appearance) are considered a modification or variant of Dengue virus (DV) peptide, subsequence or portion thereof. Such modifications may have greater or less activity or function than a reference Dengue virus (DV) peptide, subsequence or portion thereof, such as ability to elicit, stimulate, induce, promote, increase, enhance or activate a CD4+ or a CD8+ T cell response. Thus, Dengue virus (DV) peptides, subsequences and portions thereof include sequences having substantially the same, greater or less relative activity or function as a T cell epitope than a reference T cell epitope (e.g., any of the sequences in Tables 1-4, 8 & 10 (SEQ ID Nos: 11-1163, respectively, in order of appearance)), for example, an ability to elicit, stimulate, induce, promote, increase, enhance or activate an anti-DV CD4⁺ T cell or anti-DV CD8⁺ T cell response in vitro or in vivo.

Non-limiting examples of modifications include one or more amino acid substitutions (e.g., 1, 2, 3, 4, 5, 6, 7, 8, 9, 10, 1, 12, 13, 14, 15, 16, 17, 18, 19, 20, 20-25, 25-30, 30-50, 50-100, or more residues), additions and insertions (e.g., 1, 2, 3, 4, 5, 6, 7, 8, 9, 10, 1, 12, 13, 14, 15, 16, 17, 18, 19, 20, 20-25, 25-30, 30-50, 50-100, or more residues) and deletions (e.g., 1, 2, 3, 4, 5, 6, 7, 8, 9, 10, 1, 12, 13, 14, 15, 16, 17, 18, 19, 20, 20-25, 25-30, 30-50, 50-100) of a reference Dengue virus (DV) peptide, subsequence or portion thereof. In particular embodiments, a modified or variant sequence retains at least part of a function or an activity of unmodified sequence, which can have less than, approximately the same, or greater, but at least a part of, a function or activity of a reference sequence, for example, the ability to elicit, stimulate, induce, promote, increase, enhance or activate an anti-DV CD4⁺ T cell or anti-DV CD8⁺ T cell response in vitro or in vivo. Such CD4⁺ T cell and CD8⁺ T cell responses elicited include, for example, among others, induced, increased, enhanced, stimulate or activate expression or production of a cytokine (e.g., IFN-gamma, TNF, IL-2 or CD40L), release of a cytotoxin (perforin or granulysin), or apoptosis of a target (e.g., DV infected) cell.

Specific non-limiting examples of substitutions include conservative and non-conservative amino acid substitutions. A “conservative substitution” is the replacement of one amino acid by a biologically, chemically or structurally similar residue. Biologically similar means that the substitution does not destroy a biological activity. Structurally similar means that the amino acids have side chains with similar length, such as alanine, glycine and serine, or a similar size. Chemical similarity means that the residues have the same charge or are both hydrophilic or hydrophobic. Particular examples include the substitution of one hydrophobic residue, such as isoleucine, valine, leucine or methionine for another, or the substitution of one polar residue for another, such as the substitution of arginine for lysine, glutamic for aspartic acids, or glutamine for asparagine, serine for threonine, and the like.

An addition can be the covalent or non-covalent attachment of any type of molecule to the sequence. Specific examples of additions include glycosylation, acetylation, phosphorylation, amidation, formylation, ubiquitination, and derivatization by protecting/blocking groups and any of numerous chemical modifications. Additional specific non-limiting examples of an addition is one or more additional amino acid residues. Accordingly, DV sequences including T cell epitopes, subsequences, portions, and modifications thereof can be a part of or contained within a larger molecule, such as another peptide sequence, such as a fusion or chimera with a different DV sequence, or a non-DV peptide sequence. In particular embodiments, an addition is a fusion (chimeric) sequence, an amino acid sequence having one or more molecules not normally present in a reference native (wild type) sequence covalently attached to the sequence.

The term “chimeric” and grammatical variations thereof, when used in reference to a sequence, means that the sequence contains one or more portions that are derived from, obtained or isolated from, or based upon other physical or chemical entities. For example, a chimera of two or more different proteins may have one part a Dengue virus (DV) peptide, subsequence, portion or modification, and a second part of the chimera may be from a different Dengue virus (DV) peptide sequence, or a non-Dengue virus (DV) sequence.

Another particular example of a modified sequence having an amino acid addition is one in which a second heterologous sequence, i.e., heterologous functional domain is attached (covalent or non-covalent binding) that confers a distinct or complementary function. Heterologous functional domains are not restricted to amino acid residues. Thus, a heterologous functional domain can consist of any of a variety of different types of small or large functional moieties. Such moieties include nucleic acid, peptide, carbohydrate, lipid or small organic compounds, such as a drug (e.g., an antiviral), a metal (gold, silver), and radioisotope. For example, a tag such as T7 or polyhistidine can be attached in order to facilitate purification or detection of a T cell epitope. Thus, in other embodiments the invention provides Dengue virus (DV) peptides, subsequences, portions and modifications thereof and a heterologous domain, wherein the heterologous functional domain confers a distinct function, on the Dengue virus (DV) peptides, subsequences, portions and modifications thereof. Such constructs containing Dengue virus (DV) peptides, subsequences, portions and modifications thereof and a heterologous domain are also referred to as chimeras.

Linkers, such as amino acid or peptidomimetic sequences may be inserted between the sequence and the addition (e.g., heterologous functional domain) so that the two entities maintain, at least in part, a distinct function or activity. Linkers may have one or more properties that include a flexible conformation, an inability to form an ordered secondary structure or a hydrophobic or charged character which could promote or interact with either domain. Amino acids typically found in flexible protein regions include Gly, Asn and Ser. Other near neutral amino acids, such as Thr and Ala, may also be used in the linker sequence. The length of the linker sequence may vary without significantly affecting a function or activity of the fusion protein (see, e.g., U.S. Pat. No. 6,087,329). Linkers further include chemical moieties and conjugating agents, such as sulfo-succinimidyl derivatives (sulfo-SMCC, sulfo-SMPB), disuccinimidyl suberate (DSS), disuccinimidyl glutarate (DSG) and disuccinimidyl tartrate (DST).

Further non-limiting examples of additions are detectable labels. Thus, in another embodiment, the invention provides Dengue virus (DV) peptides, subsequences and portions thereof that are detectably labeled. Specific examples of detectable labels include fluorophores, chromophores, radioactive isotopes (e.g., S³⁵, P³², I¹²⁵), electron-dense reagents, enzymes, ligands and receptors. Enzymes are typically detected by their activity. For example, horseradish peroxidase is usually detected by its ability to convert a substrate such as 3,3-',5,5-'-tetramethylbenzidine (TMB) to a blue pigment, which can be quantified.

Another non-limiting example of an addition is an insertion of an amino acid within any Dengue virus (DV) sequence, subsequence, portions or modification thereof (e.g., any sequence set forth in Tables 1-4, 8 & 10 (SEQ ID Nos: 11-1163, respectively, in order of appearance)). In particular embodiments, an insertion is of one or more amino acid residues inserted into a Dengue virus (DV) peptide, subsequence portion or modification thereof, such as any sequence set forth in Tables 1-4, 8 & 10 (SEQ ID Nos: 11-1163, respectively, in order of appearance).

Modified and variant Dengue virus (DV) peptides, subsequences and portions thereof also include one or more D-amino acids substituted for L-amino acids (and mixtures thereof), structural and functional analogues, for example, peptidomimetics having synthetic or non-natural amino acids or amino acid analogues and derivatized forms. Modifications include cyclic structures such as an end-to-end amide bond between the amino and carboxy-terminus of the molecule or intra- or inter-molecular disulfide bond. Dengue virus (DV) peptides, subsequences and portions thereof may be modified in vitro or in vivo, e.g., post-translationally modified to include, for example, sugar residues, phosphate groups, ubiquitin, fatty acids, lipids, etc.

Specific non-limiting examples of substitutions include at least one amino acid deletion from full length Dengue virus (DV) peptide sequence. In particular embodiments, a peptide subsequence or portion is from about 5 to 300 amino acids in length, provided that said subsequence or portion is at least one amino acid less in length than the full-length Dengue virus (DV) structural sequence or the non-structural (NS) sequence. In additional particular embodiments, a peptide subsequence or portion is from about 2 to 5, 5 to 10, 10 to 15, 15 to 20, 20 to 25, 25 to 50, 50 to 100, 100 to 150, 150 to 200, or 200 to 300 amino acids in length, provided that said subsequence or portion is at least one amino acid less in length than the full-length Dengue virus (DV) structural sequence or non-structural (NS) sequence.

Dengue virus (DV) peptides, subsequences and portions thereof including modified forms can be produced by any of a variety of standard protein purification or recombinant expression techniques. For example, a Dengue virus (DV) peptide, subsequence, portion or modification thereof can be produced by standard peptide synthesis techniques, such as solid-phase synthesis. A portion of the protein may contain an amino acid sequence such as a T7 tag or polyhistidine sequence to facilitate purification of expressed or synthesized protein. The protein may be expressed in a cell and purified. The protein may be expressed as a part of a larger protein (e.g., a fusion or chimera) by recombinant methods.

Dengue virus (DV) peptides, subsequences and portions thereof including modified forms can be made using recombinant DNA technology via cell expression or in vitro translation. Polypeptide sequences including modified forms can also be produced by chemical synthesis using methods known in the art, for example, an automated peptide synthesis apparatus (see, e.g., Applied Biosystems, Foster City, Calif.).

The invention provides isolated and/or purified Dengue virus (DV) peptides, including or consisting of a subsequence, portion or modification of a structural core (C), membrane (M) or envelope (E) polypeptide sequence, or a non-structural (NS)NS1, NS2A, NS2B, NS3, NS4A, NS4B or NS5 polypeptide sequence. In particular embodiments, an isolated and/or purified subsequence, portion or modification of the Dengue virus (DV) polypeptide sequence includes a T cell epitope, e.g., as set forth in Tables 1-4, 8 & 10 (SEQ ID Nos: 11-1163, respectively, in order of appearance).

The term “isolated,” when used as a modifier of a composition (e.g., Dengue virus (DV) peptides, subsequences, portions and modifications thereof, nucleic acids encoding same, etc.), means that the compositions are made by the hand of man or are separated, completely or at least in part, from their naturally occurring in vivo environment. Generally, isolated compositions are substantially free of one or more materials with which they normally associate with in nature, for example, one or more protein, nucleic acid, lipid, carbohydrate, cell membrane. The term “isolated” does not exclude alternative physical forms of the composition, such as fusions/chimeras, multimers/oligomers, modifications (e.g., phosphorylation, glycosylation, lipidation) or derivatized forms, or forms expressed in host cells produced by the hand of man.

An “isolated” composition (e.g., Dengue virus (DV) peptide, subsequence, portion or modification thereof) can also be “substantially pure” or “purified” when free of most or all of the materials with which it typically associates with in nature. Thus, an isolated Dengue virus (DV) peptide, subsequence, portion or modification thereof, that also is substantially pure or purified does not include polypeptides or polynucleotides present among millions of other sequences, such as peptides of an peptide library or nucleic acids in a genomic or cDNA library, for example.

A “substantially pure” or “purified” composition can be combined with one or more other molecules. Thus, “substantially pure” or “purified” does not exclude combinations of compositions, such as combinations of Dengue virus (DV) peptides, subsequences, portions and modifications thereof (e.g., multiple, T cell epitopes), and other antigens, agents, drugs or therapies.

The invention also provides nucleic acids encoding Dengue virus (DV) peptides, subsequences, portions and modifications thereof. Such nucleic acid sequences encode a sequence at least 60% or more (e.g., 65%, 70%, 75%, 80%, 85%, 90%, 95%, etc.) identical to a Dengue virus (DV) peptide, subsequence or portion thereof. In an additional embodiment, a nucleic acid encodes a sequence having a modification, such as one or more amino acid additions (insertions), deletions or substitutions of a Dengue virus (DV) peptide, subsequences or portion thereof, such as any sequence set forth in Tables 1-4, 8 & 10 (SEQ ID Nos: 11-1163, respectively, in order of appearance).

The terms “nucleic acid” and “polynucleotide” and the like refer to at least two or more ribo- or deoxy-ribonucleic acid base pairs (nucleotides) that are linked through a phosphoester bond or equivalent. Nucleic acids include polynucleotides and polynucleosides. Nucleic acids include single, double or triplex, circular or linear, molecules. Exemplary nucleic acids include but are not limited to: RNA, DNA, cDNA, genomic nucleic acid, naturally occurring and non naturally occurring nucleic acid, e.g., synthetic nucleic acid.

Nucleic acids can be of various lengths. Nucleic acid lengths typically range from about 20 nucleotides to 20 Kb, or any numerical value or range within or encompassing such lengths, 10 nucleotides to 10 Kb, 1 to 5 Kb or less, 1000 to about 500 nucleotides or less in length. Nucleic acids can also be shorter, for example, 100 to about 500 nucleotides, or from about 12 to 25, 25 to 50, 50 to 100, 100 to 250, or about 250 to 500 nucleotides in length, or any numerical value or range or value within or encompassing such lengths. In particular aspects, a nucleic acid sequence has a length from about 10-20, 20-30, 30-50, 50-100, 100-150, 150-200, 200-250, 250-300, 300-400, 400-500, 500-1000, 1000-2000, nucleotides, or any numerical value or range within or encompassing such lengths. Shorter polynucleotides are commonly referred to as “oligonucleotides” or “probes” of single- or double-stranded DNA. However, there is no upper limit to the length of such oligonucleotides.

Nucleic acid sequences further include nucleotide and nucleoside substitutions, additions and deletions, as well as derivatized forms and fusion/chimeric sequences (e.g., encoding recombinant polypeptide). For example, due to the degeneracy of the genetic code, nucleic acids include sequences and subsequences degenerate with respect to nucleic acids that encode Dengue virus (DV) peptides, subsequences and portions thereof, as well as variants and modifications thereof (e.g., substitutions, additions, insertions and deletions).

Nucleic acids can be produced using various standard cloning and chemical synthesis techniques. Techniques include, but are not limited to nucleic acid amplification, e.g., polymerase chain reaction (PCR), with genomic DNA or cDNA targets using primers (e.g., a degenerate primer mixture) capable of annealing to the encoding sequence. Nucleic acids can also be produced by chemical synthesis (e.g., solid phase phosphoramidite synthesis) or transcription from a gene. The sequences produced can then be translated in vitro, or cloned into a plasmid and propagated and then expressed in a cell (e.g., a host cell such as eukaryote or mammalian cell, yeast or bacteria, in an animal or in a plant).

Nucleic acid may be inserted into a nucleic acid construct in which expression of the nucleic acid is influenced or regulated by an “expression control element.” An “expression control element” refers to a nucleic acid sequence element that regulates or influences expression of a nucleic acid sequence to which it is operatively linked. Expression control elements include, as appropriate, promoters, enhancers, transcription terminators, gene silencers, a start codon (e.g., ATG) in front of a protein-encoding gene, etc.

An expression control element operatively linked to a nucleic acid sequence controls transcription and, as appropriate, translation of the nucleic acid sequence. Expression control elements include elements that activate transcription constitutively, that are inducible (i.e., require an external signal for activation), or derepressible (i.e., require a signal to turn transcription off; when the signal is no longer present, transcription is activated or “derepressed”), or specific for cell-types or tissues (i.e., tissue-specific control elements).

Nucleic acid may be inserted into a plasmid for propagation into a host cell and for subsequent genetic manipulation. A plasmid is a nucleic acid that can be propagated in a host cell, plasmids may optionally contain expression control elements in order to drive expression of the nucleic acid encoding Dengue virus (DV) peptides, subsequences, portions and modifications thereof in the host cell. A vector is used herein synonymously with a plasmid and may also include an expression control element for expression in a host cell (e.g., expression vector). Plasmids and vectors generally contain at least an origin of replication for propagation in a cell and a promoter. Plasmids and vectors are therefore useful for genetic manipulation and expression of Dengue virus (DV) peptides, subsequences and portions thereof. Accordingly, vectors that include nucleic acids encoding or complementary to Dengue virus (DV) peptides, subsequences, portions and modifications thereof, are provided.

In accordance with the invention, there are provided transformed and host cells that are transformed with a nucleic acid that encodes and/or express Dengue virus (DV) peptides, subsequences, portions and modifications thereof. Transformed and host cells include but are not limited to prokaryotic and eukaryotic cells such as bacteria, fungi (yeast), plant, insect, and animal (e.g., mammalian, including primate and human, CHO cells and hybridomas) cells. For example, bacteria transformed with recombinant bacteriophage nucleic acid, plasmid nucleic acid or cosmid nucleic acid expression vectors; yeast transformed with recombinant yeast expression vectors; plant cell systems infected with recombinant virus expression vectors (e.g., cauliflower mosaic virus, CaMV; tobacco mosaic virus, TMV) or transformed with recombinant plasmid expression vectors (e.g., Ti plasmid); insect cell systems infected with recombinant virus expression vectors (e.g., baculovirus); and animal cell systems infected with recombinant virus expression vectors (e.g., retroviruses, adenovirus, vaccinia virus), or transformed animal cell systems engineered for stable expression. The cells may be a primary cell isolate, cell culture (e.g., passaged, established or immortalized cell line), or part of a plurality of cells, or a tissue or organ ex vivo or in a subject (in vivo).

The term “transformed” or “transfected” when used in reference to a cell (e.g., a host cell) or organism, means a genetic change in a cell following incorporation of an exogenous molecule, for example, a protein or nucleic acid (e.g., a transgene) into the cell. Thus, a “transfected” or “transformed” cell is a cell into which, or a progeny thereof in which an exogenous molecule has been introduced by the hand of man, for example, by recombinant DNA techniques.

The nucleic acid or protein can be stably or transiently transfected or transformed (expressed) in the host cell and progeny thereof. The cell(s) can be propagated and the introduced protein expressed, or nucleic acid transcribed. A progeny of a transfected or transformed cell may not be identical to the parent cell, since there may be mutations that occur during replication.

Introduction of Dengue virus (DV) peptides, subsequences, portions and modifications thereof, and nucleic acid into target cells (e.g., host cells) can also be carried out by methods known in the art such as osmotic shock (e.g., calcium phosphate), electroporation, microinjection, cell fusion, etc. Introduction of nucleic acid and polypeptide in vitro, ex vivo and in vivo can also be accomplished using other techniques. For example, a polymeric substance, such as polyesters, polyamine acids, hydrogel, polyvinyl pyrrolidone, ethylene-vinylacetate, methylcellulose, carboxymethylcellulose, protamine sulfate, or lactide/glycolide copolymers, polylactide/glycolide copolymers, or ethylenevinylacetate copolymers. A nucleic acid can be entrapped in microcapsules prepared by coacervation techniques or by interfacial polymerization, for example, by the use of hydroxymethylcellulose or gelatin-microcapsules, or poly(methylmethacrolate) microcapsules, respectively, or in a colloid system. Colloidal dispersion systems include macromolecule complexes, nano-capsules, microspheres, beads, and lipid-based systems, including oil-in-water emulsions, micelles, mixed micelles, and liposomes.

Liposomes for introducing various compositions into cells are known in the art and include, for example, phosphatidylcholine, phosphatidylserine, lipofectin and DOTAP (e.g., U.S. Pat. Nos. 4,844,904, 5,000,959, 4,863,740, and 4,975,282; and GIBCO-BRL, Gaithersburg, Md.). piperazine based amphilic cationic lipids useful for gene therapy also are known (see, e.g., U.S. Pat. No. 5,861,397). Cationic lipid systems also are known (see, e.g., U.S. Pat. No. 5,459,127). Polymeric substances, microcapsules and colloidal dispersion systems such as liposomes are collectively referred to herein as “vesicles.” Accordingly, viral and non-viral vector means delivery into cells are included.

Dengue virus (DV) peptides, subsequences, portions and modifications thereof can be employed in various methods and uses. Such methods and uses include contact or administration of one or more peptides set forth in Tables 1-4, 8 & 10 (SEQ ID Nos: 11-1163, respectively, in order of appearance), or contact or administration of a subsequence, portion or a modification of one or more peptides set forth in Tables 1-4, 8 & 10 (SEQ ID Nos: 11-1163, respectively, in order of appearance), in vitro and in vivo.

In accordance with the invention, methods of stimulating, inducing, promoting, increasing, or enhancing an immune response against Dengue virus (DV) in a subject are provided. In one embodiment, a method includes administering to a subject an amount of a Dengue virus (DV) T cell epitope sufficient to stimulate, induce, promote, increase, or enhance an immune response against Dengue virus (DV) in the subject. Such immune response methods can in turn be used to provide a subject with protection against a Dengue virus (DV) infection or pathology, or one or more physiological conditions, disorders, illness, diseases or symptoms caused by or associated with DV infection or pathology.

In accordance with the invention, treatment methods are provided that include therapeutic (following Dengue virus (DV) infection) and prophylactic (prior to Dengue virus (DV) exposure, infection or pathology) methods. For example, therapeutic and prophylactic methods of treating a subject for a Dengue virus (DV) infection include treatment of a subject having or at risk of having a Dengue virus (DV) infection or pathology, treating a subject with a Dengue virus (DV) infection, and methods of protecting a subject from a Dengue virus (DV) infection (e.g., provide the subject with protection against Dengue virus (DV) infection), to decrease or reduce the probability of a Dengue virus (DV) infection in a subject, to decrease or reduce susceptibility of a subject to a Dengue virus (DV) infection, or to inhibit or prevent a Dengue virus (DV) infection in a subject, and to decrease, reduce, inhibit or suppress transmission of the Dengue virus (DV) from a host (e.g., a mosquito) to a subject.

Such methods include administering Dengue virus (DV) peptide, subsequence, portion or modification thereof to therapeutically or prophylactically treat (vaccinate or immunize) a subject having or at risk of having a Dengue virus (DV) infection or pathology. Accordingly, methods can treat the Dengue virus (DV) infection or pathology, or provide the subject with protection from infection (e.g., prophylactic protection).

In one embodiment, a method includes administering to a subject an amount of Dengue virus (DV) peptide, subsequence, portion or modification thereof sufficient to treat the subject for the Dengue virus (DV) infection or pathology. In another embodiment, a method includes administering to a subject an amount of a Dengue virus (DV) T cell epitope sufficient to provide the subject with protection against the Dengue virus (DV) infection or pathology, or one or more physiological conditions, disorders, illness, diseases or symptoms caused by or associated with the virus infection or pathology. In a further embodiment, a method includes administering a subject an amount of a Dengue virus (DV) T cell epitope sufficient to treat the subject for the Dengue virus (DV) infection.

Dengue virus (DV) peptides, subsequences, portions and modifications thereof include T cell epitopes. In one embodiment, a method includes administering an amount of Dengue virus (DV) peptide, subsequence, portion or modification thereof (e.g., a T cell epitope) to a subject in need thereof, sufficient to provide the subject with protection against Dengue virus (DV) infection or pathology. In another embodiment, a method includes administering an amount of a Dengue virus (DV) peptide, subsequence, portion or modification thereof (e.g., a T cell epitope) to a subject in need thereof sufficient to treat, vaccinate or immunize the subject against the Dengue virus (DV) infection or pathology.

In accordance with the invention, methods of inducing, increasing, promoting or stimulating anti-Dengue virus (DV) activity of CD8⁺ T cells or CD4⁺ T cells in a subject are provided. In one embodiment, a method includes administering to a subject an amount of a Dengue virus (DV) T cell epitope sufficient to induce, increase, promote or stimulate anti-Dengue virus (DV) activity of CD8⁺ T cells or CD4⁺ T cells in the subject.

In methods of the invention, any appropriate Dengue virus (DV) peptide, subsequence, portion or modification thereof can be administered. Non-limiting examples include Dengue virus (DV) peptide, subsequence, portion or modification thereof of a DENV1, DENV2, DENV3 or DENV4 serotype. Additional non-limiting examples include a Dengue virus structural protein (e.g., C, M or E) or non-structural (NS) protein (e.g., NS1, NS2A, NS2B, NS3, NS4A, NS4B or NS5) T cell epitope, such as a subsequence, portion or modification of a sequence in such structural and non-structural (NS) proteins. Particular non-limiting examples include a peptide sequence set forth in Tables 1-4, 8 & 10 (SEQ ID Nos: 11-1163, respectively, in order of appearance), a subsequence thereof or a modification thereof.

In particular methods embodiments, one or more disorders, diseases, physiological conditions, pathologies and symptoms associated with or caused by a Dengue virus (DV) infection or pathology will respond to treatment. In particular methods embodiments, treatment methods reduce, decrease, suppress, limit, control or inhibit Dengue virus (DV) numbers or titer; reduce, decrease, suppress, limit, control or inhibit pathogen proliferation or replication; reduce, decrease, suppress, limit, control or inhibit the amount of a pathogen protein; or reduce, decrease, suppress, limit, control or inhibit the amount of a Dengue virus (DV) nucleic acid. In additional particular methods embodiments, treatment methods include an amount of a Dengue virus (DV) peptide, subsequence or portion thereof sufficient to increase, induce, enhance, augment, promote or stimulate an immune response against a Dengue virus (DV); increase, induce, enhance, augment, promote or stimulate Dengue virus (DV) clearance or removal; or decrease, reduce, inhibit, suppress, prevent, control, or limit transmission of Dengue virus (DV) to a subject (e.g., transmission from a host, such as a mosquito, to a subject). In further particular methods embodiments, treatment methods include an amount of Dengue virus (DV) peptide, subsequence or portion thereof sufficient to protect a subject from a Dengue virus (DV) infection or pathology, or reduce, decrease, limit, control or inhibit susceptibility to Dengue virus (DV) infection or pathology.

Methods of the invention include treatment methods, which result in any therapeutic or beneficial effect. In various methods embodiments, Dengue virus (DV) infection, proliferation or pathogenesis is reduced, decreased, inhibited, limited, delayed or prevented, or a method decreases, reduces, inhibits, suppresses, prevents, controls or limits one or more adverse (e.g., physical) symptoms, disorders, illnesses, diseases or complications caused by or associated with Dengue virus (DV) infection, proliferation or replication, or pathology (e.g., fever, rash, headache, pain behind the eyes, muscle or joint pain, nausea, vomiting, loss of appetite). In additional various particular embodiments, treatment methods include reducing, decreasing, inhibiting, delaying or preventing onset, progression, frequency, duration, severity, probability or susceptibility of one or more adverse symptoms, disorders, illnesses, diseases or complications caused by or associated with Dengue virus (DV) infection, proliferation or replication, or pathology (e.g., fever, rash, headache, pain behind the eyes, muscle or joint pain, nausea, vomiting, loss of appetite). In further various particular embodiments, treatment methods include improving, accelerating, facilitating, enhancing, augmenting, or hastening recovery of a subject from a Dengue virus (DV) infection or pathogenesis, or one or more adverse symptoms, disorders, illnesses, diseases or complications caused by or associated with Dengue virus (DV) infection, proliferation or replication, or pathology (e.g., fever, rash, headache, pain behind the eyes, muscle or joint pain, nausea, vomiting, loss of appetite). In yet additional various embodiments, treatment methods include stabilizing infection, proliferation, replication, pathogenesis, or an adverse symptom, disorder, illness, disease or complication caused by or associated with Dengue virus (DV) infection, proliferation or replication, or pathology, or decreasing, reducing, inhibiting, suppressing, limiting or controlling transmission of Dengue virus (DV) from a host (e.g., mosquito) to an uninfected subject.

A therapeutic or beneficial effect of treatment is therefore any objective or subjective measurable or detectable improvement or benefit provided to a particular subject. A therapeutic or beneficial effect can but need not be complete ablation of all or any particular adverse symptom, disorder, illness, disease or complication caused by or associated with Dengue virus (DV) infection, proliferation or replication, or pathology (e.g., fever, rash, headache, pain behind the eyes, muscle or joint pain, nausea, vomiting, loss of appetite). Thus, a satisfactory clinical endpoint is achieved when there is an incremental improvement or a partial reduction in an adverse symptom, disorder, illness, disease or complication caused by or associated with Dengue virus (DV) infection, proliferation or replication, or pathology, or an inhibition, decrease, reduction, suppression, prevention, limit or control of worsening or progression of one or more adverse symptoms, disorders, illnesses, diseases or complications caused by or associated with Dengue virus (DV) infection, Dengue virus (DV) numbers, titers, proliferation or replication, Dengue virus (DV) protein or nucleic acid, or Dengue virus (DV) pathology, over a short or long duration (hours, days, weeks, months, etc.).

A therapeutic or beneficial effect also includes reducing or eliminating the need, dosage frequency or amount of a second active such as another drug or other agent (e.g., anti-viral) used for treating a subject having or at risk of having a Dengue virus (DV) infection or pathology. For example, reducing an amount of an adjunct therapy, for example, a reduction or decrease of a treatment for a Dengue virus (DV) infection or pathology, or a vaccination or immunization protocol is considered a beneficial effect. In addition, reducing or decreasing an amount of a Dengue virus (DV) antigen used for vaccination or immunization of a subject to provide protection to the subject is considered a beneficial effect.

Adverse symptoms and complications associated with Dengue virus (DV) infection and pathology include, for example, e.g., fever, rash, headache, pain behind the eyes, muscle or joint pain, nausea, vomiting, loss of appetite, etc. Other symptoms of Dengue virus (DV) infection or pathogenesis are known to one of skill in the art and treatment thereof in accordance with the invention is provided. Thus, the aforementioned symptoms and complications are treatable in accordance with the invention.

Methods and compositions of the invention also include increasing, stimulating, promoting, enhancing, inducing or augmenting an anti-DENV CD4⁺ and/or CD8⁺ T cell responses in a subject, such as a subject with or at risk of a Dengue virus infection or pathology. In one embodiment, a method includes administering to a subject an amount of Dengue virus (DV) peptide, subsequence, portion or modification thereof sufficient to increase, stimulate, promote, enhance, augment or induce anti-DENV CD4⁺ or CD8⁺ T cell response in the subject. In another embodiment, a method includes administering to a subject an amount of Dengue virus (DV) peptide, subsequence, portion or modification thereof and administering a Dengue virus (DV) antigen, live or attenuated Dengue virus (DV), or a nucleic acid encoding all or a portion (e.g., a T cell epitope) of any protein or proteinaceous Dengue virus (DV) antigen sufficient to increase, stimulate, promote, enhance, augment or induce anti-Dengue virus (DV) CD4⁺ T cell or CD8⁺ T cell response in the subject.

Methods of the invention additionally include, among other things, increasing production of a Th1 cytokine (e.g., IFN-gamma, TNF-alpha, IL-1alpha, IL-2, IL-6, IL-8, etc.) or other signaling molecule (e.g., CD40L) in vitro or in vivo. In one embodiment, a method includes administering to a subject in need thereof an amount of Dengue virus (DV) peptide, subsequence or portion thereof sufficient to increase production of a Th1 cytokine in the subject (e.g., IFN-gamma, TNF-alpha, IL-1alpha, IL-2, IL-6, IL-8, etc.) or other signaling molecule (e.g., CD40L).

Methods and compositions of the invention include administration of Dengue virus (DV) peptide, subsequence, portion or modification thereof to a subject prior to contact, exposure or infection by a Dengue virus, administration prior to, substantially contemporaneously with or after a subject has been contacted by, exposed to or infected with a Dengue virus (DV), and administration prior to, substantially contemporaneously with or after Dengue virus (DV) pathology or development of one or more adverse symptoms. Methods, compositions and uses of the invention also include administration of Dengue virus (DV) peptide, subsequence, portion or modification thereof to a subject prior to, substantially contemporaneously with or following a Dengue virus (DV) peptide, subsequence or portion thereof or adverse symptom, disorder, illness or disease caused by or associated with a Dengue virus (DV) infection, or pathology. A subject infected with a Dengue virus (DV) may have an infection over a period of 1-5, 5-10, 10-20, 20-30, 30-50, 50-100 hours, days, months, or years.

Invention compositions (e.g., Dengue virus (DV) peptide, subsequence or portion thereof, including T cell epitopes) and uses and methods can be combined with any compound, agent, drug, treatment or other therapeutic regimen or protocol having a desired therapeutic, beneficial, additive, synergistic or complementary activity or effect. Exemplary combination compositions and treatments include multiple T cell epitopes as set for the herein, second actives, such as anti-Dengue virus (DV) compounds, agents and drugs, as well as agents that assist, promote, stimulate or enhance efficacy. Such anti-Dengue virus (DV) drugs, agents, treatments and therapies can be administered or performed prior to, substantially contemporaneously with or following any other method of the invention, for example, a therapeutic method of treating a subject for a Dengue virus (DV) infection or pathology, or a method of prophylactic treatment of a subject for a Dengue virus (DV) infection.

Dengue virus (DV) peptides, subsequences, portions and modifications thereof can be administered as a combination composition, or administered separately, such as concurrently or in series or sequentially (prior to or following) administering a second active, to a subject. The invention therefore provides combinations in which a method or use of the invention is used in a combination with any compound, agent, drug, therapeutic regimen, treatment protocol, process, remedy or composition, such as an anti-viral (e.g., Dengue virus (DV)) or immune stimulating, enhancing or augmenting protocol, or pathogen vaccination or immunization (e.g., prophylaxis) set forth herein or known in the art. The compound, agent, drug, therapeutic regimen, treatment protocol, process, remedy or composition can be administered or performed prior to, substantially contemporaneously with or following administration of one or more Dengue virus (DV) peptides, subsequences, portions or modifications thereof, or a nucleic acid encoding all or a portion (e.g., a T cell epitope) of a Dengue virus (DV) peptide, subsequence, portion or modification thereof, to a subject. Specific non-limiting examples of combination embodiments therefore include the foregoing or other compound, agent, drug, therapeutic regimen, treatment protocol, process, remedy or composition.

An exemplary combination is a Dengue virus (DV) peptide, subsequence, portion or modification thereof (e.g., a CD4⁺ or CD8⁺ T cell epitope) and a different Dengue virus (DV) peptide, subsequence, portion or modification thereof (e.g., a different T cell epitope) such as a T cell epitope, antigen (e.g., Dengue virus (DV) extract), or live or attenuated Dengue virus (DV) (e.g., inactivated Dengue virus (DV)). Such Dengue virus (DV) antigens and T cell epitopes set forth herein or known to one skilled in the art include a Dengue virus (DV) antigen that increases, stimulates, enhances, promotes, augments or induces a proinflammatory or adaptive immune response, numbers or activation of an immune cell (e.g., T cell, natural killer T (NKT) cell, dendritic cell (DC), B cell, macrophage, neutrophil, eosinophil, mast cell, CD4⁺ or a CD8⁺ cell, B220⁺ cell, CD14⁺, CD11b⁺ or CD11c⁺ cells), an anti-Dengue virus (DV) CD4⁺ or CD8⁺ T cell response, production of a Th1 cytokine, a T cell mediated immune response, etc.

Combination methods and use embodiments include, for example, second actives such as anti-pathogen drugs, such as protease inhibitors, reverse transcriptase inhibitors, virus fusion inhibitors and virus entry inhibitors, antibodies to pathogen proteins, live or attenuated pathogen, or a nucleic acid encoding all or a portion (e.g., an epitope) of any protein or proteinaceous pathogen antigen, immune stimulating agents, etc., and include contact with, administration in vitro or in vivo, with another compound, agent, treatment or therapeutic regimen appropriate for pathogen infection, vaccination or immunization

Methods of the invention also include, among other things, methods that result in a reduced need or use of another compound, agent, drug, therapeutic regimen, treatment protocol, process, or remedy. For example, for a Dengue virus (DV) infection or pathology, vaccination or immunization, a method of the invention has a therapeutic benefit if in a given subject a less frequent or reduced dose or elimination of an anti-Dengue virus (DV) treatment results. Thus, in accordance with the invention, methods of reducing need or use of a treatment or therapy for a Dengue virus (DV) infection or pathology, or vaccination or immunization, are provided.

In invention methods in which there is a desired outcome, such as a therapeutic or prophylactic method that provides a benefit from treatment, vaccination or immunization Dengue virus (DV) peptide, subsequence, portion or modification thereof can be administered in a sufficient or effective amount. As used herein, a “sufficient amount” or “effective amount” or an “amount sufficient” or an “amount effective” refers to an amount that provides, in single (e.g., primary) or multiple (e.g., booster) doses, alone or in combination with one or more other compounds, treatments, therapeutic regimens or agents (e.g., a drug), a long term or a short term detectable or measurable improvement in a given subject or any objective or subjective benefit to a given subject of any degree or for any time period or duration (e.g., for minutes, hours, days, months, years, or cured).

An amount sufficient or an amount effective can but need not be provided in a single administration and can but need not be achieved by Dengue virus (DV) peptide, subsequence, portion or modification thereof alone, in a combination composition or method that includes a second active. In addition, an amount sufficient or an amount effective need not be sufficient or effective if given in single or multiple doses without a second or additional administration or dosage, since additional doses, amounts or duration above and beyond such doses, or additional antigens, compounds, drugs, agents, treatment or therapeutic regimens may be included in order to provide a given subject with a detectable or measurable improvement or benefit to the subject. For example, to increase, enhance, improve or optimize immunization and/or vaccination, after an initial or primary administration of one or more Dengue virus (DV) peptides, subsequences, portions or modifications thereof to a subject, the subject can be administered one or more additional “boosters” of one or more Dengue virus (DV) peptides, subsequences, portions or modifications thereof. Such subsequent “booster” administrations can be of the same or a different formulation, dose or concentration, route, etc.

An amount sufficient or an amount effective need not be therapeutically or prophylactically effective in each and every subject treated, nor a majority of subjects treated in a given group or population. An amount sufficient or an amount effective means sufficiency or effectiveness in a particular subject, not a group of subjects or the general population. As is typical for such methods, different subjects will exhibit varied responses to treatment.

The term “subject” refers to an animal, typically a mammalian animal (mammal), such as a non human primate (apes, gibbons, gorillas, chimpanzees, orangutans, macaques), a domestic animal (dogs and cats), a farm animal (poultry such as chickens and ducks, horses, cows, goats, sheep, pigs), experimental animal (mouse, rat, rabbit, guinea pig) and humans. Subjects include animal disease models, for example, mouse and other animal models of pathogen (e.g., DV) infection known in the art.

Subjects appropriate for treatment include those having or at risk of having Dengue virus infection or pathology. Target subjects therefore include subjects that have been exposed to or contacted with Dengue virus (DV), or that have an ongoing infection or have developed one or more adverse symptoms caused by or associated with Dengue virus (DV) infection or pathology, regardless of the type, timing or degree of onset, progression, severity, frequency, duration of the symptoms.

Target subjects also include those at risk of Dengue virus (DV) exposure, contact, infection or pathology or at risk of having or developing a Dengue virus (DV) infection or pathology. The invention methods are therefore applicable to treating a subject who is at risk of Dengue virus (DV) exposure, contact, infection or pathology, but has not yet been exposed to or contacted with Dengue virus (DV). Prophylactic methods are therefore included. Target subjects for prophylaxis can be at increased risk (probability or susceptibility) of exposure, contact, infection or pathology, as set forth herein. Such subjects are considered in need of treatment due to being at risk.

Target subjects for prophylaxis need not be at increased risk but may be from the general population in which it is desired to vaccinate or immunize a subject against a Dengue virus (DV) infection, for example. Such a subject that is desired to be vaccinated or immunized against a Dengue virus (DV) can be administered Dengue virus (DV) peptide, subsequence, portion or modification thereof. In another non-limiting example, a subject that is not specifically at risk of exposure to or contact with a Dengue virus (DV), but nevertheless desires protect against infection or pathology, can be administered a Dengue virus (DV) peptide, subsequence, portion or modification thereof. Such subjects are also considered in need of treatment.

At risk subjects appropriate for treatment also include subjects exposed to environments in which subjects are at risk of a Dengue virus (DV) infection due to mosquitos. Subjects appropriate for treatment therefore include human subjects exposed to mosquitos, or travelling to geographical regions or countries in which Dengue virus (DV) is know to infect subjects due, for example, an individual who risks exposure due to the presence of DENV in a particular geographical region or country or population, or transmission from mosquitos present in the region or country. At risk subjects appropriate for treatment also include subjects where the risk of Dengue virus (DV) infection or pathology is increased due to changes in infectivity or the type of region of Dengue virus (DV) carrying mosquitos. Such subjects are also considered in need of treatment due to such a risk.

“Prophylaxis” and grammatical variations thereof mean a method in which contact, administration or in vivo delivery to a subject is prior to contact with or exposure to or infection. In certain situations it may not be known that a subject has been contacted with or exposed to Dengue virus (DV), but administration or in vivo delivery to a subject can be performed prior to infection or manifestation of pathology (or an associated adverse symptom, condition, complication, etc. caused by or associated with a Dengue virus (DV)). For example, a subject can be immunized or vaccinated with a Dengue virus (DV) peptide, subsequence, portion or modification thereof. In such case, a method can eliminate, prevent, inhibit, suppress, limit, decrease or reduce the probability of or susceptibility towards a Dengue virus (DV) infection or pathology, or an adverse symptom, condition or complication associated with or caused by or associated with a Dengue virus (DV) infection or pathology.

Treatment of an infection can be at any time during the infection. Dengue virus (DV) peptide, subsequence or portion thereof can be administered as a combination (e.g., with a second active), or separately concurrently or in sequence (sequentially) in accordance with the methods as a single or multiple dose e.g., one or more times hourly, daily, weekly, monthly or annually or between about 1 to 10 weeks, or for as long as appropriate, for example, to achieve a reduction in the onset, progression, severity, frequency, duration of one or more symptoms or complications associated with or caused by Dengue virus (DV) infection, pathology, or an adverse symptom, condition or complication associated with or caused by a Dengue virus (DV). Thus, a method can be practiced one or more times (e.g., 1-10, 1-5 or 1-3 times) an hour, day, week, month, or year. The skilled artisan will know when it is appropriate to delay or discontinue administration. A non-limiting dosage schedule is 1-7 times per week, for 1, 2, 3, 4, 5, 6, 7, 8, 9, 10, 15, 20 or more weeks, and any numerical value or range or value within such ranges.

Methods of the invention may be practiced by any mode of administration or delivery, or by any route, systemic, regional and local administration or delivery. Exemplary administration and delivery routes include intravenous (i.v.), intraperitoneal (i.p.), intrarterial, intramuscular, parenteral, subcutaneous, intra-pleural, topical, dermal, intradermal, transdermal, transmucosal, intra-cranial, intra-spinal, rectal, oral (alimentary), mucosal, inhalation, respiration, intranasal, intubation, intrapulmonary, intrapulmonary instillation, buccal, sublingual, intravascular, intrathecal, intracavity, iontophoretic, intraocular, ophthalmic, optical, intraglandular, intraorgan, or intralymphatic.

Doses can be based upon current existing protocols, empirically determined, using animal disease models or optionally in human clinical trials. Initial study doses can be based upon animal studies set forth herein, for a mouse, which weighs about 30 grams, and the amount of Dengue virus (DV) peptide, subsequence, portion or modification thereof administered that is determined to be effective. Exemplary non-limiting amounts (doses) are in a range of about 0.1 mg/kg to about 100 mg/kg, and any numerical value or range or value within such ranges. Greater or lesser amounts (doses) can be administered, for example, 0.01-500 mg/kg, and any numerical value or range or value within such ranges. The dose can be adjusted according to the mass of a subject, and will generally be in a range from about 1-10 ug/kg, 10-25 ug/kg, 25-50 ug/kg, 50-100 ug/kg,100-500 ug/kg, 500-1,000 ug/kg, 1-5 mg/kg, 5-10 mg/kg, 10-20 mg/kg, 20-50 mg/kg, 50-100 mg/kg, 100-250 mg/kg, 250-500 mg/kg, or more, two, three, four, or more times per hour, day, week, month or annually. A typical range will be from about 0.3 mg/kg to about 50 mg/kg, 0-25 mg/kg, or 1.0-10 mg/kg, or any numerical value or range or value within such ranges.

Doses can vary and depend upon whether the treatment is prophylactic or therapeutic, whether a subject has been previously exposed to, infected with our suffered from Dengue virus (DV), the onset, progression, severity, frequency, duration probability of or susceptibility of the symptom, condition, pathology or complication, or vaccination or immunization to which treatment is directed, the clinical endpoint desired, previous or simultaneous treatments, the general health, age, gender, race or immunological competency of the subject and other factors that will be appreciated by the skilled artisan. The skilled artisan will appreciate the factors that may influence the dosage and timing required to provide an amount sufficient for providing a therapeutic or prophylactic benefit.

Typically, for therapeutic treatment, Dengue virus (DV) peptide, subsequence, portion or modification thereof will be administered as soon as practical, typically within 1-2, 2-4, 4-12, 12-24 or 24-72 hours after a subject is exposed to or contacted with a Dengue virus (DV), or within 1-2, 2-4, 4-12, 12-24 or 24-48 hours after onset or development of one or more adverse symptoms, conditions, pathologies, complications, etc., associated with or caused by a Dengue virus (DV) infection or pathology. For prophylactic treatment in connection with vaccination or immunization, Dengue virus (DV) peptide, subsequence, portion or modification thereof can be administered for a duration of 0-4 weeks, e.g., 2-3 weeks, prior to exposure to, contact or infection with Dengue virus (DV), or at least within 1-2, 2-4, 4-12, 12-24, 24-48 or 48-72 hours prior to exposure to, contact or infection with Dengue virus (DV). For an acute infection, Dengue virus (DV) peptide, subsequence, portion or modification thereof is administered at any appropriate time.

The dose amount, number, frequency or duration may be proportionally increased or reduced, as indicated by the status of the subject. For example, whether the subject has a pathogen infection, whether the subject has been exposed to, contacted or infected with pathogen or is merely at risk of pathogen contact, exposure or infection, whether the subject is a candidate for or will be vaccinated or immunized. The dose amount, number, frequency or duration may be proportionally increased or reduced, as indicated by any adverse side effects, complications or other risk factors of the treatment or therapy.

Dengue virus (DV) peptides, subsequences, portions and modifications thereof can be incorporated into compositions, including pharmaceutical compositions, e.g., a pharmaceutically acceptable carrier or excipient. Such pharmaceutical compositions are useful for, among other things, administration to a subject in vivo or ex vivo.

As used herein the term “pharmaceutically acceptable” and “physiologically acceptable” mean a biologically acceptable formulation, gaseous, liquid or solid, or mixture thereof, which is suitable for one or more routes of administration, in vivo delivery or contact. Such formulations include solvents (aqueous or non-aqueous), solutions (aqueous or non-aqueous), emulsions (e.g., oil-in-water or water-in-oil), suspensions, syrups, elixirs, dispersion and suspension media, coatings, isotonic and absorption promoting or delaying agents, compatible with pharmaceutical administration or in vivo contact or delivery. Aqueous and non-aqueous solvents, solutions and suspensions may include suspending agents and thickening agents. Such pharmaceutically acceptable carriers include tablets (coated or uncoated), capsules (hard or soft), microbeads, powder, granules and crystals. Supplementary active compounds (e.g., preservatives, antibacterial, antiviral and antifungal agents) can also be incorporated into the compositions.

Pharmaceutical compositions can be formulated to be compatible with a particular route of administration. Thus, pharmaceutical compositions include carriers, diluents, or excipients suitable for administration by various routes. Exemplary routes of administration for contact or in vivo delivery which a composition can optionally be formulated include inhalation, respiration, intranasal, intubation, intrapulmonary instillation, oral, buccal, intrapulmonary, intradermal, topical, dermal, parenteral, sublingual, subcutaneous, intravascular, intrathecal, intraarticular, intracavity, transdermal, iontophoretic, intraocular, opthalmic, optical, intravenous (i.v.), intramuscular, intraglandular, intraorgan, or intralymphatic.

Formulations suitable for parenteral administration comprise aqueous and non-aqueous solutions, suspensions or emulsions of the active compound, which preparations are typically sterile and can be isotonic with the blood of the intended recipient. Non-limiting illustrative examples include water, saline, dextrose, fructose, ethanol, animal, vegetable or synthetic oils.

To increase an immune response, immunization or vaccination, Dengue virus (DV) peptides, subsequences, portions and modifications thereof can be coupled to another protein such as ovalbumin or keyhole limpet hemocyanin (KLH), thyroglobulin or a toxin such as tetanus or cholera toxin. Dengue virus (DV) peptides, subsequences, portions and modifications thereof can also be mixed with adjuvants.

Adjuvants include, for example: Oil (mineral or organic) emulsion adjuvants such as Freund's complete (CFA) and incomplete adjuvant (IFA) (WO 95/17210; WO 98/56414; WO 99/12565; WO 99/11241; and U.S. Pat. No. 5,422,109); metal and metallic salts, such as aluminum and aluminum salts, such as aluminum phosphate or aluminum hydroxide, alum (hydrated potassium aluminum sulfate); bacterially derived compounds, such as Monophosphoryl lipid A and derivatives thereof (e.g., 3 De-O-acylated monophosphoryl lipid A, aka 3D-MPL or d3-MPL, to indicate that position 3 of the reducing end glucosamine is de-O-acylated, 3D-MPL consisting of the tri and tetra acyl congeners), and enterobacterial lipopolysaccharides (LPS); plant derived saponins and derivatives thereof, for example Quil A (isolated from the Quilaja Saponaria Molina tree, see, e.g., “Saponin adjuvants”, Archiv. fur die gesamte Virusforschung, Vol. 44, Springer Verlag, Berlin, p 243-254; U.S. Pat. No. 5,057,540), and fragments of Quil A which retain adjuvant activity without associated toxicity, for example QS7 and QS21 (also known as QA7 and QA21), as described in WO96/33739, for example; surfactants such as, soya lecithin and oleic acid; sorbitan esters such as sorbitan trioleate; and polyvinylpyrrolidone; oligonucleotides such as CpG (WO 96/02555, and WO 98/16247), polyriboA and polyriboU; block copolymers; and immunostimulatory cytokines such as GM-CSF and IL-1, and Muramyl tripeptide (MTP). Additional examples of adjuvants are described, for example, in “Vaccine Design—the subunit and adjuvant approach” (Edited by Powell, M. F. and Newman, M. J.; 1995, Pharmaceutical Biotechnology (Plenum Press, New York and London, ISBN 0-306-44867-X) entitled “Compendium of vaccine adjuvants and excipients” by Powell, M. F. and Newman M.

Cosolvents may be added to a Dengue virus (DV) peptide, subsequence, portion or modification composition or formulation. Non-limiting examples of cosolvents contain hydroxyl groups or other polar groups, for example, alcohols, such as isopropyl alcohol; glycols, such as propylene glycol, polyethyleneglycol, polypropylene glycol, glycol ether; glycerol; polyoxyethylene alcohols and polyoxyethylene fatty acid esters. Non-limiting examples of cosolvents contain hydroxyl groups or other polar groups, for example, alcohols, such as isopropyl alcohol; glycols, such as propylene glycol, polyethyleneglycol, polypropylene glycol, glycol ether; glycerol; polyoxyethylene alcohols and polyoxyethylene fatty acid esters.

Supplementary compounds (e.g., preservatives, antioxidants, antimicrobial agents including biocides and biostats such as antibacterial, antiviral and antifungal agents) can also be incorporated into the compositions. Pharmaceutical compositions may therefore include preservatives, anti-oxidants and antimicrobial agents.

Preservatives can be used to inhibit microbial growth or increase stability of ingredients thereby prolonging the shelf life of the pharmaceutical formulation. Suitable preservatives are known in the art and include, for example, EDTA, EGTA, benzalkonium chloride or benzoic acid or benzoates, such as sodium benzoate. Antioxidants include, for example, ascorbic acid, vitamin A, vitamin E, tocopherols, and similar vitamins or provitamins.

An antimicrobial agent or compound directly or indirectly inhibits, reduces, delays, halts, eliminates, arrests, suppresses or prevents contamination by or growth, infectivity, replication, proliferation, reproduction, of a pathogenic or non-pathogenic microbial organism. Classes of antimicrobials include antibacterial, antiviral, antifungal and antiparasitics. Antimicrobials include agents and compounds that kill or destroy (-cidal) or inhibit (-static) contamination by or growth, infectivity, replication, proliferation, reproduction of the microbial organism.

Exemplary antibacterials (antibiotics) include penicillins (e.g., penicillin G, ampicillin, methicillin, oxacillin, and amoxicillin), cephalosporins (e.g., cefadroxil, ceforanid, cefotaxime, and ceftriaxone), tetracyclines (e.g., doxycycline, chlortetracycline, minocycline, and tetracycline), aminoglycosides (e.g., amikacin, gentamycin, kanamycin, neomycin, streptomycin, netilmicin, paromomycin and tobramycin), macrolides (e.g., azithromycin, clarithromycin, and erythromycin), fluoroquinolones (e.g., ciprofloxacin, lomefloxacin, and norfloxacin), and other antibiotics including chloramphenicol, clindamycin, cycloserine, isoniazid, rifampin, vancomycin, aztreonam, clavulanic acid, imipenem, polymyxin, bacitracin, amphotericin and nystatin.

Particular non-limiting classes of anti-virals include reverse transcriptase inhibitors; protease inhibitors; thymidine kinase inhibitors; sugar or glycoprotein synthesis inhibitors; structural protein synthesis inhibitors; nucleoside analogues; and viral maturation inhibitors. Specific non-limiting examples of anti-virals include nevirapine, delavirdine, efavirenz, saquinavir, ritonavir, indinavir, nelfinavir, amprenavir, zidovudine (AZT), stavudine (d4T), larnivudine (3TC), didanosine (DDI), zalcitabine (ddC), abacavir, acyclovir, penciclovir, ribavirin, valacyclovir, ganciclovir, 1,-D-ribofuranosyl-1,2,4-triazole-3 carboxamide, 9->2-hydroxy-ethoxy methylguanine, adamantanamine, 5-iodo-2′-deoxyuridine, trifluorothymidine, interferon and adenine arabinoside.

Pharmaceutical formulations and delivery systems appropriate for the compositions and methods of the invention are known in the art (see, e.g., Remington: The Science and Practice of Pharmacy (2003) 20^{th} ed., Mack Publishing Co., Easton, Pa.; Remington's Pharmaceutical Sciences (1990) 18^{th} ed., Mack Publishing Co., Easton, Pa.; The Merck Index (1996) 12^{th} ed., Merck Publishing Group, Whitehouse, N.J.; Pharmaceutical Principles of Solid Dosage Forms (1993), Technonic Publishing Co., Inc., Lancaster, Pa.; Ansel ad Soklosa, Pharmaceutical Calculations (2001) 11^{th} ed., Lippincott Williams & Wilkins, Baltimore, Md.; and Poznansky et al., Drug Delivery Systems (1980), R. L. Juliano, ed., Oxford, N.Y., pp. 253-315).

Dengue virus (DV) peptides, subsequences, portions, and modifications thereof, along with any adjunct agent, compound drug, composition, whether active or inactive, etc., can be packaged in unit dosage form (capsules, tablets, troches, cachets, lozenges) for ease of administration and uniformity of dosage. A “unit dosage form” as used herein refers to physically discrete units suited as unitary dosages for the subject to be treated; each unit containing a predetermined quantity of active ingredient optionally in association with a pharmaceutical carrier (excipient, diluent, vehicle or filling agent) which, when administered in one or more doses, is calculated to produce a desired effect (e.g., prophylactic or therapeutic effect). Unit dosage forms also include, for example, ampules and vials, which may include a composition in a freeze-dried or lyophilized state; a sterile liquid carrier, for example, can be added prior to administration or delivery in vivo. Unit dosage forms additionally include, for example, ampules and vials with liquid compositions disposed therein. Individual unit dosage forms can be included in multi-dose kits or containers. Pharmaceutical formulations can be packaged in single or multiple unit dosage form for ease of administration and uniformity of dosage.

The invention provides kits that include Dengue virus (DV) peptide, subsequences, portions, and modifications thereof, optionally with a second active, and pharmaceutical formulations thereof, packaged into suitable packaging material. A kit typically includes a label or packaging insert including a description of the components or instructions for use in vitro, in vivo, or ex vivo, of the components therein. A kit can contain a collection of such components, e.g., Dengue virus (DV) peptide, subsequence, portion, or modification thereof, and optionally a second active, such as another compound, agent, drug or composition.

The term “packaging material” refers to a physical structure housing the components of the kit. The packaging material can maintain the components sterilely, and can be made of material commonly used for such purposes (e.g., paper, corrugated fiber, glass, plastic, foil, ampules, vials, tubes, etc.).

Kits of the invention can include labels or inserts. Labels or inserts include “printed matter,” e.g., paper or cardboard, or separate or affixed to a component, a kit or packing material (e.g., a box), or attached to an ampule, tube or vial containing a kit component. Labels or inserts can additionally include a computer readable medium, such as a disk (e.g., hard disk, flash memory), optical disk such as CD- or DVD-ROM/RAM, DVD, MP3, magnetic tape, or an electrical storage media such as RAM and ROM or hybrids of these such as magnetic/optical storage media, FLASH media or memory type cards.

Labels or inserts can include identifying information of one or more components therein, dose amounts, does frequency or timing, clinical pharmacology of the active ingredient(s) including mechanism of action, pharmacokinetics and pharmacodynamics. Labels or inserts can include information identifying manufacturer information, lot numbers, manufacturer location and date.

Labels or inserts can include information on a condition, disorder or disease (e.g., viral infection, vaccination or immunization) for which a kit component may be used. Labels or inserts can include instructions for the clinician or subject for using one or more of the kit components in a method, or treatment protocol or therapeutic regimen. Instructions can include dosage amounts, frequency or duration, and instructions for practicing any of the methods, treatment protocols or prophylactic or therapeutic regimes described herein. Exemplary instructions include, instructions for treating a Dengue virus (DV) infection or pathology, and instructions for providing a subject with protection against Dengue virus (DV) infection or pathology, e.g., immune response stimulating, vaccination or immunization.

Labels or inserts can include information on any benefit that a component may provide, such as a prophylactic or therapeutic benefit. Labels or inserts can include information on potential adverse side effects, complications or reactions, such as warnings to the subject or clinician regarding situations where it would not be appropriate to use a particular composition. Adverse side effects or complications could also occur when the subject has, will be or is currently taking one or more other medications that may be incompatible with the composition, or the subject has, will be or is currently undergoing another treatment protocol or therapeutic regimen which would be incompatible with the composition and, therefore, instructions could include information regarding such incompatibilities.

Methods of the invention also include, among other things, methods of diagnosing DV infection in a subject, and DV exposure of a subject. In one embodiment, a method includes contacting cells from a subject to one or more DV T cell epitopes; and determining if the T cell epitope elicits a response (e.g., anti-DV response) from the contacted cells. A response identifies the cells as having been exposed to the T cell epitope, thereby diagnosing the subject from which the cells were obtained as having a DV infection or as having been exposed to DV. Exemplary cells include CD8⁺ T cells and/or CD4+ T cells. Exemplary responses include inducing, increasing, promoting or stimulating an anti-Dengue virus (DV) activity of CD8⁺ T cells or CD4+ T cells; and/or increased or stimulated IFN-gamma, TNF-alpha, IL-1alpha, IL-6 or IL-8 production by CD8⁺ T cells in the presence of the T cell epitope; and/or increased or stimulated IFN-gamma, TNF, IL-2, or CD40L in the presence of the T cell epitope, or killing T cell epitope-pulsed target cells.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described herein.

All applications, publications, patents and other references, GenBank citations and ATCC citations cited herein are incorporated by reference in their entirety. In case of conflict, the specification, including definitions, will control.

As used herein, the singular forms “a,” “and,” and “the” include plural referents unless the context clearly indicates otherwise. Thus, for example, reference to a “Dengue virus (DV) peptide, subsequence, portion, or modification thereof,” or a “Dengue virus (DV)” includes a plurality of Dengue virus (DV) peptides, subsequences, portions, and modifications thereof, such as CD4⁺ and/or CD8⁺ T cell epitopes, or serotypes of Dengue virus (DV), and reference to an “activity or function” can include reference to one or more activities or functions of a Dengue virus (DV) peptide, subsequence, portion, or modification thereof, including function as a T cell epitopes, an ability to elicit, stimulate, induce, promote, increase, enhance or activate a measurable or detectable anti-DV CD4⁺ T cell response or anti-DV CD8⁺ T cell response, and so forth.

As used herein, numerical values are often presented in a range format throughout this document. The use of a range format is merely for convenience and brevity and should not be construed as an inflexible limitation on the scope of the invention. Accordingly, the use of a range expressly includes all possible subranges, all individual numerical values within that range, and all numerical values or numerical ranges include integers within such ranges and fractions of the values or the integers within ranges unless the context clearly indicates otherwise. This construction applies regardless of the breadth of the range and in all contexts throughout this patent document. Thus, to illustrate, reference to a range of 90-100% includes 91-99%, 92-98%, 93-95%, 91-98%, 91-97%, 91-96%, 91-95%, 91-94%, 91-93%, and so forth. Reference to a range of 90-100%, includes 91%, 92%, 93%, 94%, 95%, 95%, 97%, etc., as well as 91.1%, 91.2%, 91.3%, 91.4%, 91.5%, etc., 92.1%, 92.2%, 92.3%, 92.4%, 92.5%, etc., and so forth. Reference to a range of 1-5 fold therefore includes 1, 2, 3, 4, 5, 6, 7, 8, 9, 10, 11, 12, 13, 14, 15, 16, 17, 18, 19, 20, fold, etc., as well as 1.1, 1.2, 1.3, 1.4, 1.5, fold, etc., 2.1, 2.2, 2.3, 2.4, 2.5, fold, etc., and so forth. Further, for example, reference to a series of ranges of 2-72 hours, 2-48 hours, 4-24 hours, 4-18 hours and 6-12 hours, includes ranges of 2-6 hours, 2, 12 hours, 2-18 hours, 2-24 hours, etc., and 4-27 hours, 4-48 hours, 4-6 hours, etc.

As also used herein a series of range formats are used throughout this document. The use of a series of ranges includes combinations of the upper and lower ranges to provide a range. Accordingly, a series of ranges include ranges which combine the values of the boundaries of different ranges within the series. This construction applies regardless of the breadth of the range and in all contexts throughout this patent document. Thus, for example, reference to a series of ranges such as 5-10, 10-20, 20-30, 30-40, 40-50, 50-75, 75-100, 100-150, and 150-171, includes ranges such as 5-20, 5-30, 5-40, 5-50, 5-75, 5-100, 5-150, 5-171, and 10-30, 10-40, 10-50, 10-75, 10-100, 10-150, 10-171, and 20-40, 20-50, 20-75, 20-100, 20-150, 20-171, and so forth.

The invention is generally disclosed herein using affirmative language to describe the numerous embodiments and aspects. The invention also specifically includes embodiments in which particular subject matter is excluded, in full or in part, such as substances or materials, method steps and conditions, protocols, procedures, assays or analysis. For example, in certain embodiments or aspects of the invention, antibodies or other materials and method steps are excluded. In certain embodiments and aspects of the invention, for example, a Dengue virus (DV) peptide, subsequence, portion, or modification thereof, is excluded. Thus, even though the invention is generally not expressed herein in terms of what is not included, embodiments and aspects that expressly exclude compositions (e.g., antibodies or pathogen antigens) or method steps are nevertheless disclosed and included in the invention.

A number of embodiments of the invention have been described. Nevertheless, it will be understood that various modifications may be made without departing from the spirit and scope of the invention. Accordingly, the following examples are intended to illustrate but not limit the scope of invention described in the claims.

### EXAMPLES

### Example 1

This example includes a description of various materials and methods.

### Mice and Infections

C57BL/6 (H-2^{b}) mice were obtained from The Jackson Laboratory and subsequently bred. IFN-α/βR^{-/-} mice on the C57BL/6 background were obtained from Dr. Wayne Yokoyama (Washington University, St. Louis, Mo.) via Dr. Carl Ware. HLA-A*0201/Kb, A*1101/Kb, A*0101, B*0702 and DRB1*0101 transgenic mice were bred at LIAI as previously described (Kotturi et al., Immunome Res 6:4 (2010); Pasquetto et al., J Immunol 175:5504 (2005); Alexander et al., J Immunol 159:4753 (1997); Alexander et al., Hum Immunol 64:211 (2003)). All transgenic mouse strains were subsequently backcrossed with the IFN-α/βR^{-/-} mice at the animal facility at LIAI.B6.SJL mice were purchased from Taconic. Mice were used between 5 and 10 weeks of age.

Mice were infected intravenously (i.v.) in the lateral tail vein or retro-orbitally (r.o.) with 200 μl of the DENV2 strain, S221, in 5% FBS/PBS. Blood was obtained from anesthetized mice by r.o. puncture. For experiments with transgenic mice, mice were infected i.v.r.o. with 10¹⁰ genomic equivalents (GE) of S221 in 100 uL PBS. On day 7 post-infection, mice were sacrificed and splenic CD8+ or CD4+ T cells, respectively, were used in mouse IFNγ ELISPOT assays. All mouse experiments were approved by the Animal Care Committee.

### Cell Culture and Viral Stocks

The hybridoma clones SFR3, GK1.5, and 2.43, which produce rat anti-human HLA-DRS, anti-mouse CD4, and anti-mouse CD8 IgG2b Ab, respectively, were from the American Type Culture Collection, and were grown in Protein-Free Hybridoma Medium supplemented with penicillin, streptomycin, HEPES, GlutaMAX, and 2-ME (all from Invitrogen) at 37° C., 5% CO₂. C6/36, an A. albopictus mosquito cell line, was cultured in Leibovitz's L-15 Medium (Invitrogen) supplemented with 10% FBS (Gemini Bio-Products), penicillin, streptomycin, and HEPES at 28° C. in the absence of CO₂. S221, a plaque-purified DENV2 strain, was derived from the clinical isolate, PL046 (Lin et al., J Virol 72:9729 (1998)), as described previously (Yauch et al., J Immunol 182:4865 (2009)). Viral stocks were amplified in C6/36 cells and purified over a sucrose gradient as previously described (Prestwood et al., J Virol 82:8411 (2008)). Infectious doses were determined based on GE, which were quantified by real-time RT-PCR. There are approximately 5×10⁴ GE/PFU for S221, based on plaque assay on baby hamster kidney cells.

### Bioinformatic Analyses

Candidate epitopes were identified using a consensus approach (Wang et al., PLoS Comput Biol 4:e1000048 (2008)). Briefly, all 15-mer peptides that are encoded in the DENV2 PL046 polyprotein were predicted for binding to H-2 I-A^{b}. Two independent algorithms (Zhang et al., Nucleic Acids Res 36:W513 (2008)) were used to rank the peptides by predicted binding affinity. The median of the two ranks was used to select the top 73 out of 3383 peptides, corresponding to the top 2% of all peptides.

For human MHC class I binding predictions all 9 and 10mer peptides were predicted for their binding affinity to their respective alleles. Binding predictions were performed using the command-line version of the consensus prediction tool available on the IEDB web site (Zhang et al., Nucleic Acids Res 36:W513 (2008)). Peptides were selected if they are in the top 1% of binders in a given strain. For human MHC class II binding predictions all 15mer peptides were predicted for their binding affinity to the DRB1*0101 allele. As with class I, binding predictions were performed using the command-line version of the consensus prediction tool available on the IEDB web site. The top 2% of predicted binders were then selected for synthesis. All peptides evaluated in this study were derived from the DENV2 virus strain S221, which was also used as infectious agent in this study, as described above. For the conservancy analysis, full-length DENV polyprotein sequences were retrieved for each serotype from the NCBI Protein database using the following query: txid11053 AND polyprotein AND 3000:5000[slen]. The number of isolates from any one country was limited to 10 to eliminate geographical bias. Sequences were considered “unique” if they varied by at least 1 amino acid from all other sequences. In summary, 171 DENV2, 162 DENV1, 169 DENV3 and 53 DENV4 sequences from the NCBI protein database were investigated for conservancy of the identified epitopes within the respective serotypes.

### Selection of DENV Sequences for Human HLAs

Full-length DENV polyprotein sequences were retrieved for each serotype from the NCBI Protein database using the following query: txid11053 AND polyprotein AND 3000:5000(44). Table 5 shows the DENV sequences that were retrieved for DENV3 as an example. In summary 162 DENV1, 171 DENV2, 169 DENV3 and 53 DENV4 sequences have been retrieved. Sequences were considered “unique” if they varied by at least 1 amino acid from all other sequences. The number of isolates from any one country was limited to 10 to avoid geographical bias. Polyproteins were broken down into all possible 9, 10mer sequences for binding predictions as described below.

### MHC Class I Binding Predictions and Peptide Selection

All 9 and 10mer peptides were predicted for their binding affinity to 27 MHC class I alleles (Table 6). Binding predictions were performed using the command-line version of the consensus prediction tool available on the Immune Epitope Data Base (IEDB), web site (www.iedb.org, (45)). For each allele and length combination, peptides from each included polyprotein were selected if they are in the top 1% of binders in a given strain and they exist in at least 30% of the isolates for that serotype. If the same peptide is selected from two or more serotypes, then it was placed in the “conserved” group. If two or more peptides at the same position in the alignment (i.e. completely overlapping) are selected from one serotype, the peptide that is more common is designated the representative and the less-common peptides were placed in the “variant” group. This resulted in a set of 8,088 peptides to be synthesized. Table 6 summarizes the peptide groups resulting from this selection.

### Peptide Synthesis

Peptides utilized in initial screening studies were synthesized as crude material by A and A Labs. A total of 73 15-mer peptides were ordered and synthesized twice in different (alphabetical vs. predicted IC₅₀) order. Positive peptides were re-synthesized by A and A Labs and purified to >90% homogeneity by reverse-phase HPLC. Purity of these peptides was determined using mass spectrometry. The HPLC-purified peptides were used for all subsequent experiments.

All peptides using human MHC class I or II sequences were synthesized by Mimotopes (Victoria, Australia). A total of 8088 9-mer and 10-mer peptides were identified by MHC class I predictions and synthesized as crude material. Peptides were combined into pools of 10 individual peptides, according to their predicted HLA restriction.

### Flow Cytometric Analyses

For surface staining of germinal center B cells, splenocytes were stained with anti-B220-Alexa Fluor 647 (Biolegend), anti-CD4-PerCP (BD Biosciences), GL7-FITC (BD Biosciences), anti-IgD-eFluor 450 (eBioscience), and anti-Fas-PE (BD Biosciences). For intracellular cytokine staining (ICS) of CD4⁺ T cells, 2×10⁶splenocytes were plated in 96-well U-bottom plates and stimulated with individual DENV2 peptides (3 μg/ml) for 2 h (hours). Brefeldin A (GolgiPlug, BD Biosciences) was then added and cells were incubated for another 5 h (hours). Cells were washed, incubated with supernatant from 2.4G2-producing hybridoma cells, and labeled with anti-CD4-eFluor 450 (eBioscience) and anti-CD8α-PerCP-eFluor 710 (eBioscience) or PE-Cy7 (BD Biosciences). The cells were then fixed and permeabilized using the BD Cytofix/Cytoperm Kit, and stained with various combinations of anti-IFN-γ-APC (eBioscience), anti-TNF-PE-Cy7 (BD Biosciences), anti-IL-2-Alexa Fluor 488 (BD Biosciences) or —PE (Biolegend), and anti-CD40L-PE (eBioscience). Foxp3 staining was done using the mouse regulatory T cell staining kit from eBioscience. The criteria for positivity in CD4⁺ T cell epitope identification were: 2× the percentage of IFN-γ produced by stimulated cells compared with unstimulated cells, positive in two independent crude peptide orders, and positive when ordered as HPLC-purified (>90% pure). For CD8⁺ T cell ICS, splenocytes (2×10⁶) were stimulated in 96-well U-bottom plates for 5 h (hours) in the presence of 1 μg/ml H-2^{b}-restricted epitopes identified previously: M₆₀₋₆₇, NS2A₈₋₁₅, and NS4B₉₉₋₁₀₇ (Yauch et al., J Immunol 182:4865 (2009)). Anti-CD107a-FITC (BD Biosciences) was added to the wells during the stimulation. Cells were then stained as described for CD4⁺ T cell ICS. Samples were read on an LSR II (BD Biosciences) and analyzed using FloJo software (Tree Star).

### Immunohistochemistry

Tissues were embedded in O.C.T. compound (Sakura). Sections (6 μm) were cut and stored at −80° C. Frozen sections were thawed and fixed for 10 minutes in acetone at 25° C., followed by 8 minutes in 1% paraformaldehyde (EMS) in 100 mM dibasic sodium phosphate containing 60 mM lysine and 7 mM sodium periodate pH 7.4 at 4° C. Sections were blocked first using the Avidin/Biotin Blocking Kit (Vector Labs) followed by 5% normal goat serum (Invitrogen) and 1% BSA (Sigma) in PBS. Sections were stained overnight with anti-F4/80-biotin (clone BM8, Biolegend), anti-CD4-PE (clone RM4-5, eBioscience), anti-CD813-Alexa Fluor 647 (clone YTS156.7.7, Biolegend), and anti-B220-FITC (clone RA3-6B2, BD Pharmingen). Sections were then washed and stained with streptavidin-Alexa Fluor 750 and rabbit anti-FITC-Alexa Fluor 488 (Invitrogen). Images were recorded using a Leica TCS SP5 confocal microscope, processed using Leica Microsystems software, stitched together using Adobe Illustrator, and adjusted using ImageJ.

### T Cell Depletions

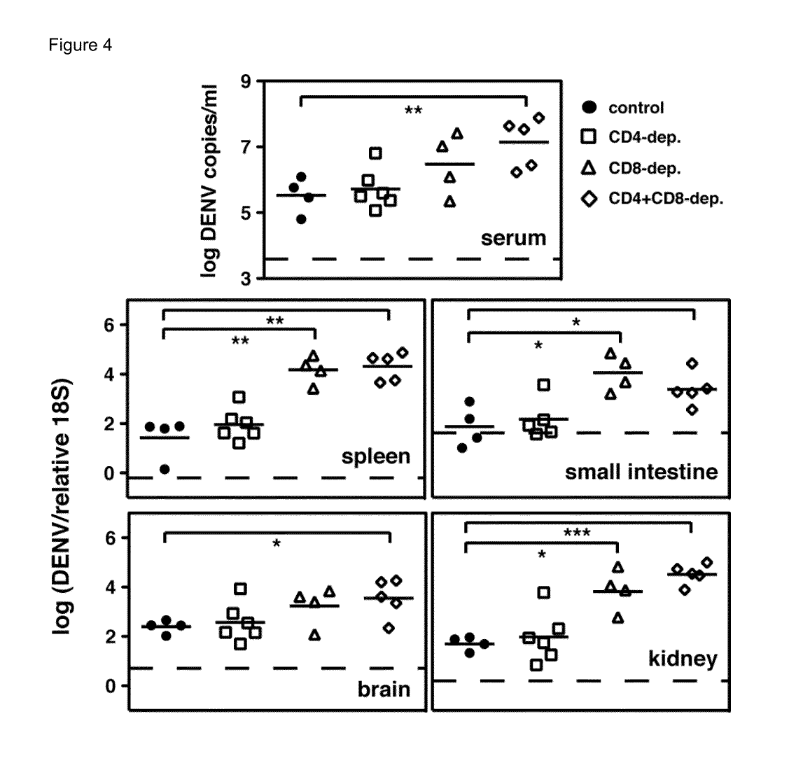

Hybridoma supernatants were clarified by centrifugation, dialyzed against PBS, sterile-filtered, and quantified by BCA Protein Assay Reagent (Thermo Scientific). IFN-α/βR^{-/-} mice were injected i.p. with 250 μg of SFR3, or GK1.5, or 2.43 in PBS (250 μl total volume) 3 days and 1 day before or 1 day before and 1 day after infection, which resulted in depletion of ≧90% of CD8⁺ cells and ≧97% of CD4⁺ cells. In FIG. 4, one CD4-depleted mouse received GK1.5 only on day 1, which still resulted in ≧97% depletion.

### DENV2-Specific Antibody ELISA

Serum was harvested from control and CD4-depleted IFN-α/βR^{-/-} mice 7 days after infection with 10¹⁰ GE of DENV2, or naïve mice. EIA/RIA 96-well plates (Costar) were coated with DENV2 (10⁹ GE per well) in 50 μl 0.1M NaHCO₃. The virus was UV-inactivated and plates left overnight at 4° C. The plates were then washed to remove unbound virus using 0.05% (v/v) Tween 20 (Sigma) in PBS. After blocking with Blocker Casein Blocking Buffer (Thermo Scientific) for 1 h at room temperature, 1:3 serial dilutions of serum in a total volume of 100 μl were added to the wells. After 1.5 h, wells were washed and bound antibody was detected using HRP-conjugated goat anti-mouse IgG Fc portion or HRP-conjugated donkey anti-mouse IgMμ chain (Jackson Immunoresearch) and TMB (eBioscience).

### Antibody-Virus Neutralization Assay

Serum was heat-inactivated at 56° C. for 30 min. Three-fold serial dilutions of serum were then incubated with 5×10⁸ GE of DENV2 for 1 h at room temperature in a total volume of 100 μl PBS. Next, approximately 6×10⁵ C6/36 cells per well of a 24-well plate were infected with 100 μl of the virus-antibody mix for one hour at 28° C. Cells were washed twice with 500 μl of PBS, and then incubated at 28° C. in 500 μl L-15 Medium containing 5% FBS, penicillin, and streptomycin for 24 h. For each antibody dilution, the percentage of infected cells was determined by flow cytometry as previously described (Lambeth et al., J Clin Microbiol 43:3267 (2005)) using 2H2-biotin (IgG2a anti-prM/M, DENV1-4 reactive) and streptavidin-APC (Biolegend). The percentage of infected cells was normalized to 100% (infection without serum).

### CD8 In Vivo Cytotoxicity Assay

IFN-α/βR^{-/-} mice (recipients) were infected with 10¹⁰ GE of DENV2. Some mice were depleted of CD4⁺ T cells before infection. Splenocytes (targets) were harvested from donor B6.5JL congenic mice (CD45.1) 7 days later. RBC were lysed, and the target cells were pulsed with varying concentrations of a pool of 4 H-2^{b}-restricted DENV2 peptides (M₆₀₋₆₇, NS2A₈₋₁₅, NS4B₉₉₋₁₀₇, NS5₂₃₇₋₂₄₅) or DMSO for 1 h at 37° C. The cells were then washed and labeled with CFSE (Invitrogen) in PBS/0.1% BSA for 10 min at 37° C. Cells were labeled with 1 μM CFSE (CFSE^{high}) or 100 nM CFSE (CFSE^{low}) or left unlabeled. After washing, the cell populations were mixed and 5×10⁶ cells from each population were injected i.v. into naïve or infected recipient mice. After 4 h, the mice were sacrificed and splenocytes stained with anti-CD45.1-APC (eBioscience) and analyzed by flow cytometry, gating on CD45.1⁺ cells. The percentage killing was calculated as follows: 100−((percentage DENV peptide-pulsed in infected mice/percentage DMSO-pulsed in infected mice)/(percentage DENV peptide-pulsed in naïve mice/percentage DMSO-pulsed in naïve mice)×100).

### CD4 In Vivo Cytotoxicity Assay

IFN-α/βR^{-/-} mice (recipients) were infected with 10¹⁰ GE of DENV2. Some mice were depleted of CD4⁺ or CD8⁺ cells before infection. Splenocytes (targets) were harvested from donor B6.5JL congenic mice (CD45.1) 7 days later. RBC were lysed and the target cells were pulsed with 1.7 μg (approximately 1 μM) each of NS2B₁₀₈₋₁₂₂, NS3₁₉₈₋₂₁₂, and NS3₂₃₇₋₂₅₁ (or DMSO) for 1 h at 37° C. The cells were then washed and labeled with CFSE in PBS/0.1% BSA for 10 min at 37° C. DENV2 peptide-pulsed cells were labeled with 1 μM CFSE (CFSE^{high}) and DMSO-pulsed cells with 100 nM CFSE (CFSE^{low}). After washing, the two cell populations were mixed and 5×10⁶ cells from each population were injected i.v. into naïve or infected recipient mice. After 16 h, the mice were sacrificed and splenocytes stained and the percentage killing calculated as described for the CD8 in vivo cytotoxicity assay.

### Quantitation of DENV Burden in Mice

Mice were euthanized by isoflurane inhalation and blood was collected via cardiac puncture. Serum was separated from whole blood by centrifugation in serum separator tubes (Starsted). Small intestines were put into PBS, flushed, filleted, chopped into small pieces, and put into RNAlater (Qiagen). Other organs were immediately placed into RNAlater and all organs were subsequently homogenized for 3 min in 1 ml tissue lysis buffer (Qiagen Buffer RLT) using a Mini-Beadbeater-8 (BioSpec Products) or QiagenTissueLyser Immediately after homogenization, all tissues were centrifuged (5 min, 4° C., 16,000×g) to pellet debris, and RNA was isolated using the RNeasy Mini Kit (Qiagen). Serum RNA was isolated using the QIAamp Viral RNA Mini Kit (Qiagen). After elution, viral RNA was stored at −80° C. Quantitative RT-PCR was performed according to a published protocol (Houng et al., J Virol Methods 86:1-11 (2000)), except a MyiQ Single-Color Real-Time PCR Detection System (Bio-Rad) with One-Step qRT-PCR Kit (Quanta BioSciences) were used. The DENV2 standard curve was generated with serial dilutions of a known concentration of DENV2 genomic RNA which was in vitro transcribed (MAXIscriptKit, Ambion) from a plasmid containing the cDNA template of S221 3′UTR. After transcription, DNA was digested with DNase I, and RNA was purified using the RNeasy Mini Kit and quantified by spectrophotometry. To control for RNA quality and quantity when measuring DENV in tissues, the level of 18S rRNA was measured using 18S primers described previously (Lacher, et al., Cancer Res 66:1648 (2006)) in parallel real-time RT-PCR reactions. A relative 18S standard curve was made from total splenic RNA.

### Peptide Immunizations

IFN-α/βR^{-/-} mice were immunized s.c. with 50 μg each of NS2B₁₀₈₋₁₂₂, NS3₁₉₈₋₂₁₂, and NS3₂₃₇₋₂₅₁ emulsified in CFA (Difco). After 11 days, mice were boosted with 50 μg peptide emulsified in IFA (Difco). Mock-immunized mice received PBS/DMSO emulsified in CFA or IFA. Mice were infected 13 days after the boost with 10¹¹ GE of DENV2 (some mice were depleted of CD4⁺ or CD8⁺ T cells 3 days and 1 day before infection). Four days later, the mice were sacrificed and tissues harvested, RNA isolated, and DENV2 RNA levels measured as described above. For Example 7, mice were immunized instead with 50 μg each of C₅₁₋₅₉, NS2A₈₋₁₅, NS4B₉₉₋₁₉₇, and NS5₂₃₇₋₂₄₅ as described in Yauch et al., J Immunol 182:4865 (2009).

### MHC Peptide-Binding and Restriction Assays

MHC purification and quantitative assays to measure the binding affinity of peptides to purified A*0201, A*0101, A*1101, B*0702 and DRB1*0101 molecules were performed as described elsewhere(Sidney et al., Immunome Res 4:2 (2008); Sidney et al., Curr Protoc Immunol Chapter 18:Unit 18 13 (2001)). Briefly, after a 2-day incubation, binding of the radiolabeled peptide to the corresponding MHC molecule was determined by capturing MHC/peptide complexes on Greiner Lumitrac 600 microplates (Greiner Bio-One, Monroe, N.C.) coated with either the W6/32 (HLA class I specific) or L243 (HLA DR specific) monoclonal antibodies. Bound cpm were then measured using the Topcount microscintillation counter (Packard Instrument, Meriden, Conn.). The concentration of peptide yielding 50% inhibition of the binding of the radiolabeled probe peptide (IC₅₀) was then calculated.

The tumor cell line 721.221 (Shimizu et al., J Immunol 142:3320 (1989), which lacks expression of HLA-A, -B and C class I genes, was transfected with the HLA-A*0201/Kb or HL-A*1101 chimeric genes, and was used as APC in the restriction assays. The non-transfected cell line was used as a negative control.

### Human Blood Samples

250 peripheral blood samples were obtained from healthy adult blood donors from the National Blood Center, Ministry of Health, Colombo, Sri Lanka in an anonymous fashion. The institutional review boards of both the La Jolla Institute for and Genetech approved all protocols described in this study and donors gave written informed consent. Donors were of both sexes and between 18 and 60 years of age. Samples have been collected over a time course of 19 month between February 2010 and August 2011. PBMC were purified by density gradient centrifugation (Ficoll-Paque Premium, GE Healthcare Biosciences, Kowloon, Hong Kong) re-suspended in fetal bovine serum (Gemini Bio-products, Sacramento, Calif.) containing 10% dimethyl sulfoxide, and cryo-preserved in liquid nitrogen. 23 of the 250 blood samples obtained from the National Blood Center had to be excluded from the study due to poor viability of cells after shipment to LIAI as determined by trypan blue exclusion. For the remaining 227 samples DENV seropositivity was determined by dengue IgG ELISA as previously described (26). Flow cytometry-based neutralization assays were performed for further characterization of seropositve donors, as previously described (46).

Genomic DNA isolated from PBMC of the study subjects by standard techniques (QIAmp. Qiagen, Valencia, Calif.) was used for HLA typing. High resolution Luminex-based typing for HLA Class I was utilized according the manufacturer's protocol (Sequence-Specific Oligonucleotides (SSO) typing; One Lambda, Canoga Park, Calif.). Where needed, PCR based methods were used to provide high resolution sub-typing. (Sequence-Specific Primer (SSP) typing; One Lambda, Canoga Park, Calif.).

### IFNγ ELISPOT Assay

For all murine studies, splenic CD4⁺ or CD8⁺ T cells were isolated by magnetic bead positive selection (MiltenyiBiotec, BergischGladbach, Germany) 7 days after infection. 2×10⁵ T cells were stimulated with 1×10⁵ uninfected splenocytes as APCs and 10 μg/ml of individual DENV peptides in 96-well flat-bottom plates (Immobilon-P; Millipore, Bedford, Mass.) coated with anti-IFNγ mAb (clone AN18; Mabtech, Stockholm, Sweden). Each peptide was evaluated in triplicate. Following a 20-h incubation at 37° C., the wells were washed with PBS/0.05% Tween 20 and then incubated with biotinylated IFNγ mAb (clone R4-6A2; Mabtech) for 2 h. The spots were developed using Vectastain ABC peroxidase (Vector Laboratories, Burlingame, Calif.) and 3-amino-9-ethylcarbazole (Sigma-Aldrich, St. Louis, Mo.) and counted by computer-assisted image analysis (KS-ELISPOT reader, Zeiss, Munich, Germany). Responses against peptides were considered positive if the net spot-forming cells (SFC) per 10⁶ were 0.20, a stimulation index of 0.2, and p<0.05 in at test comparing replicates with those from the negative control.

To evaluate the antigenicity of the epitopes in humans, 2×10⁶ PBMC/ml were stimulated in the presence of 1μg/ml individual peptide for 7 days. Cells were cultured at 37° C., 5% CO₂, and recombinant IL2 (10 U/mL, eBiosciences, San Diego, Calif.) was added 3 days after antigenic stimulation. After one week, PBMC were harvested and tested at a concentration of 1×10⁵/well in an IFNγ ELISPOT assay, as described above. The mAb 1-D1K and mAb 7-B6-1 (Mabtech) were used as coating and biotinylated secondary Ab, respectively. To be considered positive, IFNγ responses needed to exceed the threshold set as the mean responses of HLA non-matched and DENV seronegative donors plus 3 times the standard deviation.

### Ex Vivo IFNγ ELISPOT Assay

96-well multiScreen plates (Immobilon-P; Millipore, Bedford, Mass.) were coated with anti-IFNγ mab (clone 1-DIK; [5 μg/ml]: Mabtech, Stockholm, Sweden) at 4° C. overnight. 2×10⁵ PBMC were then incubated in triplicates with 0.1 ml complete RPMI 1640 in the presence of HLA-matched peptide pools [2 μg/ml]. Following a 20 h incubation at 37° C., the cells were washed with PBS/0.05% Tween 20 and then incubated with biotinylated IFNγ mAb (mAb 7-B6-1 Mabtech, Stockholm, Sweden) for 2 h. The spots were developed using Vectastain ABC peroxidase (Vector Laboratories, Burlingame, Calif.) and 3-amino-9-ethylcarbazole (Sigma-Aldrich, St. Louis, Mo.) and counted by computer-assisted image analysis (KS-ELISPOT reader, Zeiss, Munich, Germany). Responses were expressed as the number of IFNγ secreting cells per 10⁶ PBMC and were considered positive if the net spot-forming cells (SFC) per 10⁶ were ≧20, had a stimulation index of ≧2, and a p<0.05 in at test comparing replicates with those from the negative control. Positive pools were subsequently deconvoluted and a peptide was considered positive according to the criteria described above.

### Flow Cytometry and Intracellular Cytokine Staining (ICS)

The following monoclonal antibodies from eBioscience (eBioscience, San Diego, Calif.) were used in this study: anti-CD8a PerCP-Cy5.5 (clone RPA-T8), anti-CD3 efluor 450 (clone UCHT1), anti-CD107a FITC (clone ebioH4A3), anti-CD45RA PE-CY7 (clone H100), anti-CD27 PE (clone 0323), anti-CD197 (CCR7) APC efluor 780 (clone 3D12), anti-IFNγ APC (clone 4SB3), anti-IL 2 PE (clone MQ1-17H12), anti-TNFα (clone MAD 110).

PBMC were cultured in the presence of peptide pools [10 μg/ml] and GolgiPlug containing brefeldin A [1 μg/ml] (BD Biosciences, San Diego, Calif.) in 0.2 ml complete RPMI medium for 6 h. To determine the avidity of DENV specific T cells, PBMC were incubated with descending concentrations of peptide pools (10 1, 0.1, 0.01, 0.001, and 0.0001 μg/ml] and the half maximal effective concentration (EC₅₀) was calculated. Cells stimulated with PMA/Ionomycin (Sigma Aldrich, St. Louis, Mo.) or media alone were used as positive and negative control respectively. After incubation, cells were washed and stained with directly conjugated anti-CD3 mAb, anti-CD8 mAb, anti-CD27 mAb, anti-CD45RA mAb and anti-CCR7 mAb for 30 minutes on ice, fixed with 1% of paraformaldehyde (Sigma-Aldrich, St. Louis, Mo.) and were kept at 4° C. over night. Cells were washed, incubated in Cytofix/cytoperm solution (BD Biosciences) for 20 minutes on ice, washed with Perm/Wash (BD Biosciences) and then stained for IL 2, IFNγ, TNFα and CD107a with directly conjugated Abs for 30 minutes on ice. Samples were acquired on a LSR II flow cytometer (BD Immunocytometry Systems), and analyzed with FlowJo software (Tree Star, San Carlos, Calif.).

### Statistical Analyses

Data were analyzed with Prism software version 5.0 (GraphPad Software, Inc.). Statistical significance was determined using the unpaired t-test with Welch's correction.

### Example 2

This example includes data demonstrating CD4⁺ T cell activation and expansion following DENV2 infection.

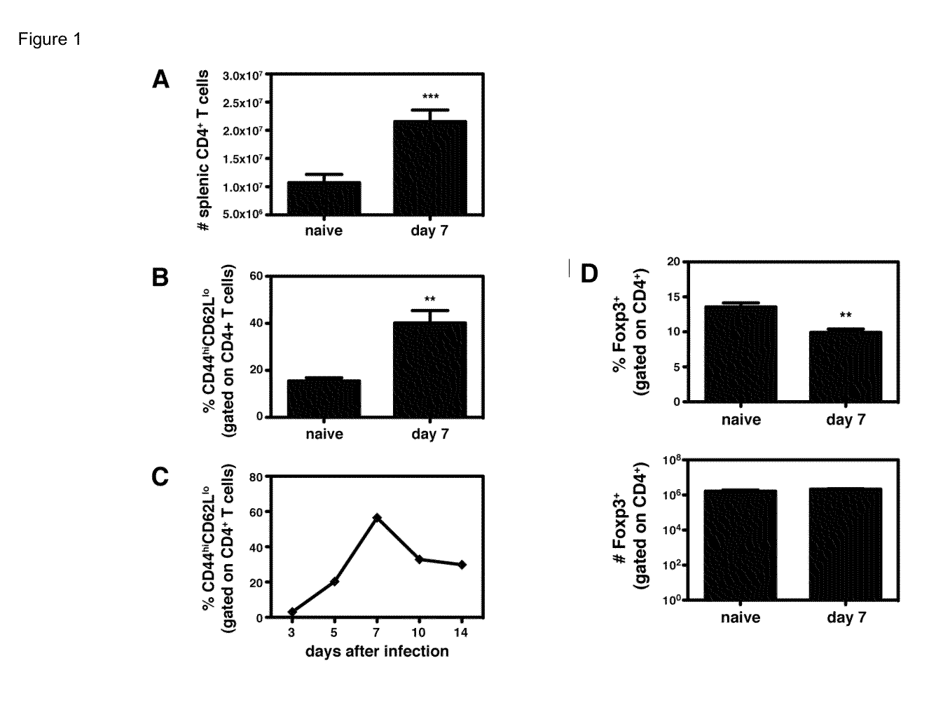

DENV2 (10¹⁰ GE of S221) infection of IFN-α/βR^{-/-} mice results in an acute infection, with viral replication peaking between 2 and 4 days after infection (Yauch, et al. J Immunol 182:4865 (2009)). At this time the mice show signs of disease including hunched posture and ruffled fur, and the virus is subsequently cleared from the serum by day 6. To determine the role of CD4⁺ T cells during the course of this primary DENV2 infection, the expansion of CD4⁺ T cells in the spleens of IFN-α/βR^{-/-} mice 7 days after infection with DENV2 was examined, and a 2-fold increase in CD4⁺ T cell numbers was observed (FIG. 1A). The cells were activated, as measured by CD44 upregulation and CD62L downregulation on splenic CD4⁺ T cells (FIG. 1B) and on circulating blood CD4⁺ T cells, with the peak on day 7 after infection (FIG. 1C). To study the CD4⁺ T cell response in the spleen in more detail, immunohistochemistry on spleen sections obtained from naïve mice and mice 3, 5, and 7 days after DENV2 infection was performed. Sections were stained for CD4, CD8, B220 to highlight B cell follicles, and F4/80 to show red pulp macrophages. As expected, in naïve mice, CD4⁺ and CD8⁺ T cells were dispersed throughout the spleen, but preferentially in T cell areas, also known as the periarteriolar lymphoid sheath (PALS). By day 3 after DENV2 infection, most of the CD4⁺ and CD8⁺ T cells had migrated to the PALS, with very few T cells observed in the red pulp. At day 5, the CD4⁺ cells were still concentrated in the PALS, at the border between the T cell area and B cell follicles, and also in the B cell follicles. At day 7 after infection, the spleen had increased in size dramatically, and CD4⁺ T cells were found primarily in the PALS and B cell follicles. The localization of CD8⁺ T cells differed from the CD4⁺ T cells mainly in that at day 5 after infection, many of the CD8⁺ T cells had left the T cell area and were found distributed throughout the red pulp and marginal zone (MZ). By day 7, the CD8⁺ T cells were observed in the PALS, MZ, and also the red pulp. These images illustrate the kinetics of the adaptive immune response to DENV2 in the spleen, and show CD4⁺ T cells in close proximity to both CD8′ T cells and B cells after DENV2 infection.

Regulatory T cells (Tregs) are a subset of CD4⁺ T cells that are characterized by the expression of the transcription factor, Foxp3 (Josefowicz, et al. Immunity 30:616 (2009)), and have been found to facilitate the early host response to HSV-2 (Lund, et al. Science 320:1220 (2008)) and help control WNV infection (Lanteri, et al. J Clin Invest 119:3266 (2009)). To determine if DENV2 infection resulted in an expansion of Tregs, the number of CD4⁺Foxp3⁺ cells in the spleen 7 days after infection was determined. There was a decrease in the percentage of Tregs among total CD4⁺ cells, and no change in the number of Tregs, demonstrating that DENV2 infection does not lead to an expansion of Tregs in the spleen (FIG. 1D).

### Example 3

This example includes data for the identification of DENV2 CD4⁺ T cell epitopes and phenotype of DENV2-specific CD4⁺ T cells.

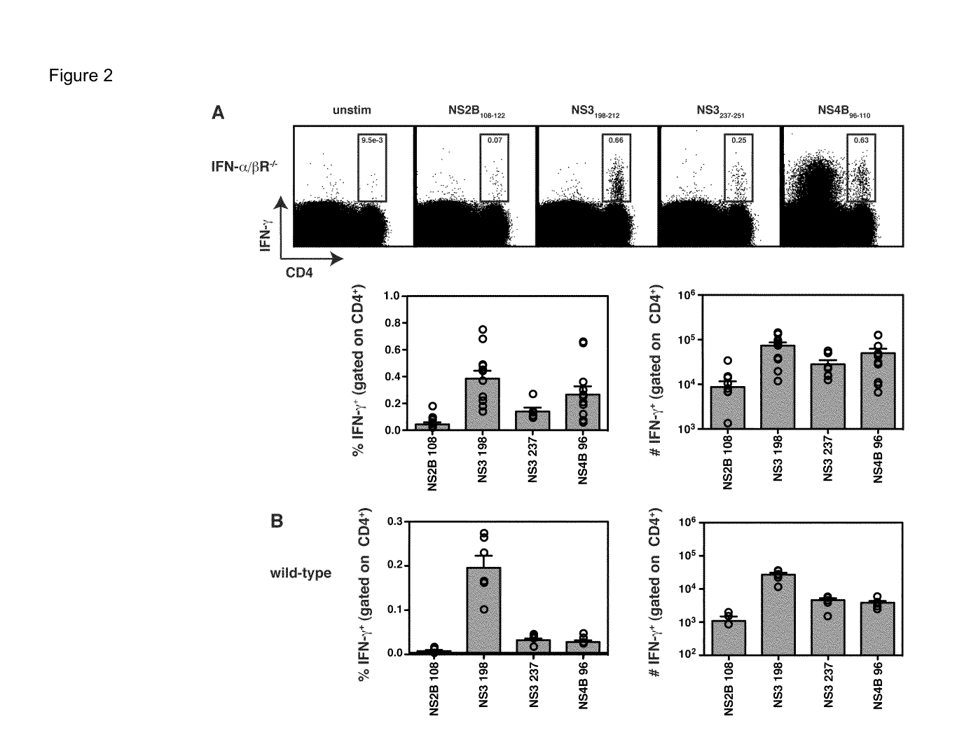

In order to study the DENV2-specific CD4⁺ T cell response, the identity of MHC class II (I-A^{b})-restricted CD4⁺ T cell epitopes using a bioinformatics prediction method previously reported to map the CD4⁺ T cell response to mouse cytomegalovirus (Arens, et al. J Immunol 180:6472 (2008)) was employed. Briefly, the proteome of DENV2 was screened and 73 15-mer peptides predicted to bind I-A^{b} were identified. The peptides were tested by IFN-γ ICS using splenocytes from DENV2-infected IFN-α/βR^{-/-} mice. Positive peptides (2× background) were then re-ordered as HPLC-purified (>90%) and re-tested. Four positive peptides were identified: NS2B₁₀₈₋₁₂₂, NS3₁₉₈₋₂₁₂, NS3₂₃₇₋₂₅₁, and NS4B₉₆₋₁₁₀ (FIG. 2A and Table 1). Similar to the DENV2-specific CD8⁺ T cell response (Yauch, et al. J Immunol 182:4865 (2009)), the epitopes identified in IFN-α/βR^{-/-} mice were also recognized by CD4⁺ T cells from DENV2-infected wild-type mice (FIG. 2B), and the magnitude of the CD4⁺ T cell response was higher in IFN-α/βR^{-/-} mice compared with wild-type mice, likely due to increased viral replication. Notably, NS3₂₀₀₋₂₁₄ has been identified as a human HLA-DR15-restricted CD4⁺ T cell epitope (Simmons, et al. J Virol 79:5665 (2005); Zeng, et al. J Virol 70:3108 (1996)). It was also of interest that NS4B₉₆₋₁₁₀ contains a CD8⁺ T cell epitope (NS4B₉₉₋₁₀₇) that was identified as the immunodominant epitope in both wild-type and IFN-α/βR^{-/-} C57BL/6 mice infected with DENV2 (Yauch, et al. J Immunol 182:4865 (2009)).

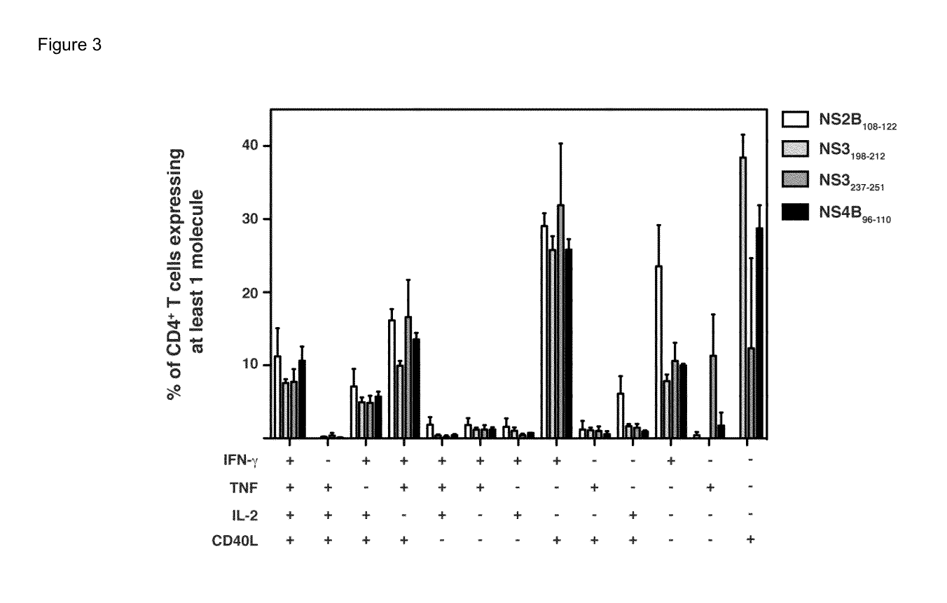

Multicolor flow cytometry was performed to study the phenotype of DENV2-specific CD4⁺ T cells. These cells produced IFN-γ, TNF, and IL-2 (FIG. 3). No intracellular IL-4, IL-5, IL-17, or IL-10 were detected. The DENV2-specific CD4⁺ T cells also expressed CD40L, suggesting they are capable of activating CD40-expressing cells, which include DCs and B cells. The four DENV2-derived CD4⁺ T cell epitopes induced responses that differed in magnitude, but were similar in terms of phenotype. The most polyfunctional cells (those expressing IFN-γ, TNF, IL-2, and CD40L) also expressed the highest levels of the cytokines and CD40L. These results demonstrate that DENV2 infection elicits a virus-specific Th1 CD4⁺ T cell response in IFN-α/βR^{-/-} mice.

TABLE 1
DENV2-derived CD4⁺ T cell epitopes

| Epitope | Sequence |
|---|---|
| NS2B₁₀₈₋₁₂₂ | GLFPVSLPITAAAWY |
| | (SEQ ID NO: 11) |
| NS3₁₉₈₋₂₁₂ | GKTKRYLPAIVREAI |
| | (SEQ ID NO: 12) |
| NS3₂₃₇₋₅₁ | GLPIRYQTPAIRAEH |
| | (SEQ ID NO: 13) |
| NS4B₉₆₋₁₁₀ | IGCYSQVNPITLTAA |
| | (SEQ ID NO: 14) |

### Example 4

This example includes a description of studies of the effects of CD4⁺ and/or CD8⁺ T cell depletions on DENV2 viral RNA levels, and data showing that CD4⁺ T cells are not required for the anti-DENV2 antibody response, and are also not necessary for the primary DENV2-specific CD8⁺ T cell response.

To determine how CD4⁺ T cells contribute to controlling DENV2 infection, CD4⁺ T cells, CD8⁺ T cells, or both were depleted from IFN-α/βR^{-/-} mice and DENV2 RNA levels 5 days after infection with 10¹⁰ GE of DENV2 was measured. No difference in viral RNA levels between control undepleted mice and CD4-depleted mice in the serum, kidney, small intestine, spleen, or brain was observed (FIG. 4). CD8-depleted mice had significantly higher viral loads than control mice. Depletion of both CD4⁺ and CD8⁺ T cells resulted in viral RNA levels that were significantly higher than those in control mice in all tissues examined, and equivalent to the viral RNA levels in CD8-depleted mice. These data show that CD4⁺ T cells are not required to control primary DENV2 infection in IFN-α/βR^{-/-} mice, and confirm an important role for CD8⁺ T cells in viral clearance.

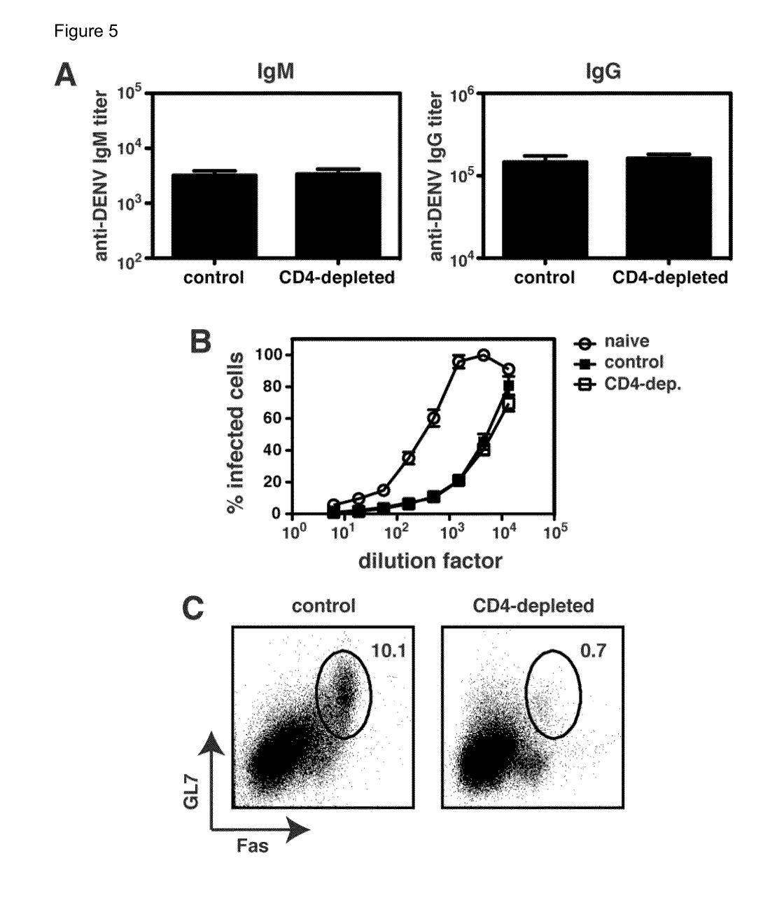

Although CD4⁺ T cells were not required for controlling DENV2 infection, the contribution to the anti-DENV immune response, for example by helping the B cell and/or CD8⁺ T cell responses, was investigated. CSR, the process by which the immunoglobulin heavy chain constant region is switched so the B cell expresses a new isotype of Ab, can be induced when CD40L-expressing CD4⁺ T cells engage CD40 on B cells (Stavnezer, et al. Annu Rev Immunol 26:261 (2008)). However, CSR can also occur in the absence of CD4⁺ T cell help. To determine whether the anti-DENV2 antibody response depends on CD4⁺ T cells, DENV2-specific IgM and IgG titers in the sera of control and CD4-depleted mice was measured 7 days after infection with 10¹⁰ GE of DENV2. As expected, there was no difference in IgM titers at day 7 between control and CD4-depleted mice (FIG. 5A). There was also no difference in IgG titers between control and CD4-depleted mice. To measure the functionality of these DENV2-specific antibody, a flow cytometry-based neutralization assay was performed, in which C6/36 mosquito cells were infected with DENV2 in the presence of heat-inactivated sera obtained from control and CD4-depleted mice 7 days after infection. The sera from control and CD4-depleted mice could neutralize DENV2 equally well (FIG. 5B). As reported previously (Zellweger, et al. Cell Host Microbe 7:128 (2010)), naïve serum was able to prevent DENV infection of C6/36 cells, although not as efficiently as DENV-immune serum. The presence of germinal center (GC) B cells, as the GC reaction is generally thought to be CD4⁺ T cell-dependent (Allen, et al. Immunity 27:190 (2007)), was also evaluated. As expected, GC B cells were absent in the CD4-depleted mice (FIG. 5C). Based on the lack of GC B cells in the DENV2-infected CD4-depleted mice, the early anti-DENV2 antibody response is CD4- and GC-independent.

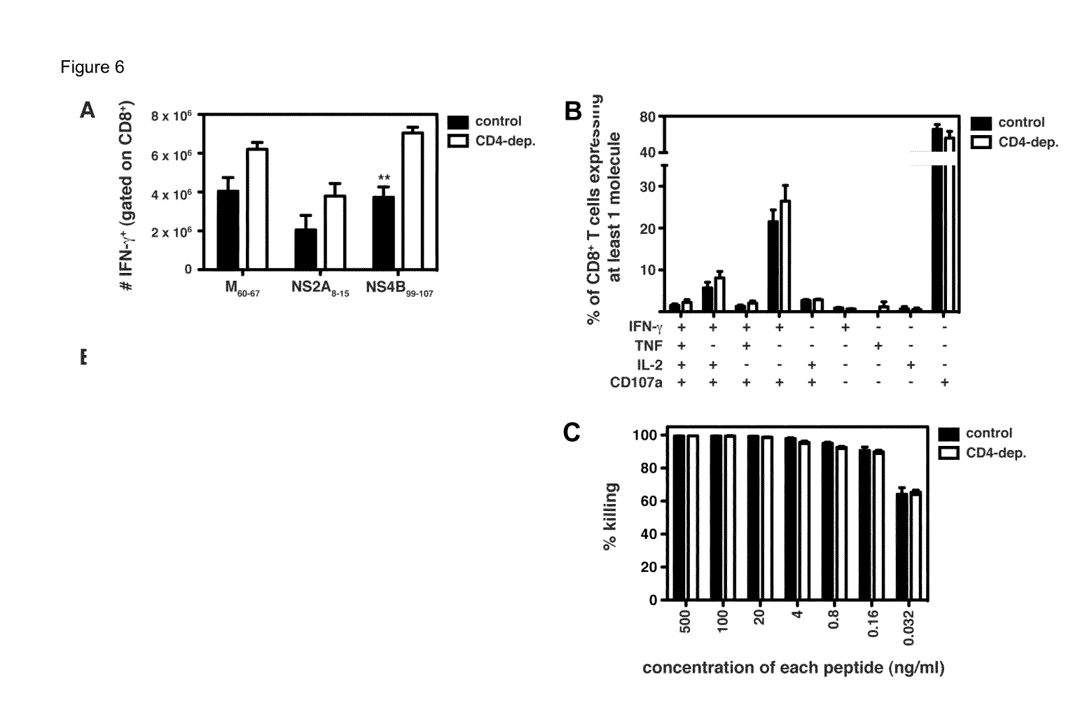

Next, the role of CD4⁺ T cells in helping the CD8⁺ T cell response was assessed by examining the DENV2-specific CD8⁺ T cell response in control and CD4-depleted DENV2-infected mice. The numbers of splenic CD8⁺ T cells were equivalent in control and CD4-depleted mice. IFN-γ ICS was performed using DENV2-derived H-2^{b}-restricted immunodominant peptides identified (M₆₀₋₆₇, NS2A₈₋₁₅, and NS4B₉₉₋₁₀₇) (Yauch, et al. J Immunol 182:4865 (2009)). Somewhat surprisingly, there was an increase in the number of DENV2-specific IFN-γ⁺CD8⁺ T cells in CD4-depleted mice compared with control mice (FIG. 6A). To further characterize the phenotype of the CD8⁺ T cells generated in the absence of CD4⁺ T cells, expression of TNF, IL-2, and CD107a (a marker for degranulation) in cells stimulated with NS4B₉₉₋₁₀₇ was examined (FIG. 6B). As also shown in FIG. 6A, the magnitude of the CD8⁺ T cell response was larger in the CD4-depleted mice, but the cytokine and CD 107a expression profiles were comparable. Similar results were observed when cells were stimulated with M₆₀₋₆₇ or NS2A₈₋₁₅. Next, the functionality of the DENV2-specific CD8′ T cells using an in vivo cytotoxicity assay, in which splenocytes were pulsed with a pool of 4 H-2^{b}-restricted immunodominant peptides and CFSE-labeled before injection into control or CD4-depleted DENV2-infected mice, was examined. CD8⁺ T cell-mediated-cytotoxicity was very efficient; almost 100% killing was observed at peptide concentrations of 500 ng/ml (FIG. 6C). Therefore, the peptide concentrations were titrated down, and no difference in killing was observed between control and CD4-depleted mice at any concentration tested. These data reveal that the primary anti-DENV2 CD8⁺ T cell response, in terms of expansion, cytokine production, degranulation, and cytotoxicity, does not depend on CD4⁺ T cell help.

### Example 5

This example is a description of studies of in vivo killing of I-A^{b}-restricted peptide-pulsed target cells in DENV2-infected mice, and data showing that vaccination with DENV2 CD4⁺ T cell epitopes controls viral load.

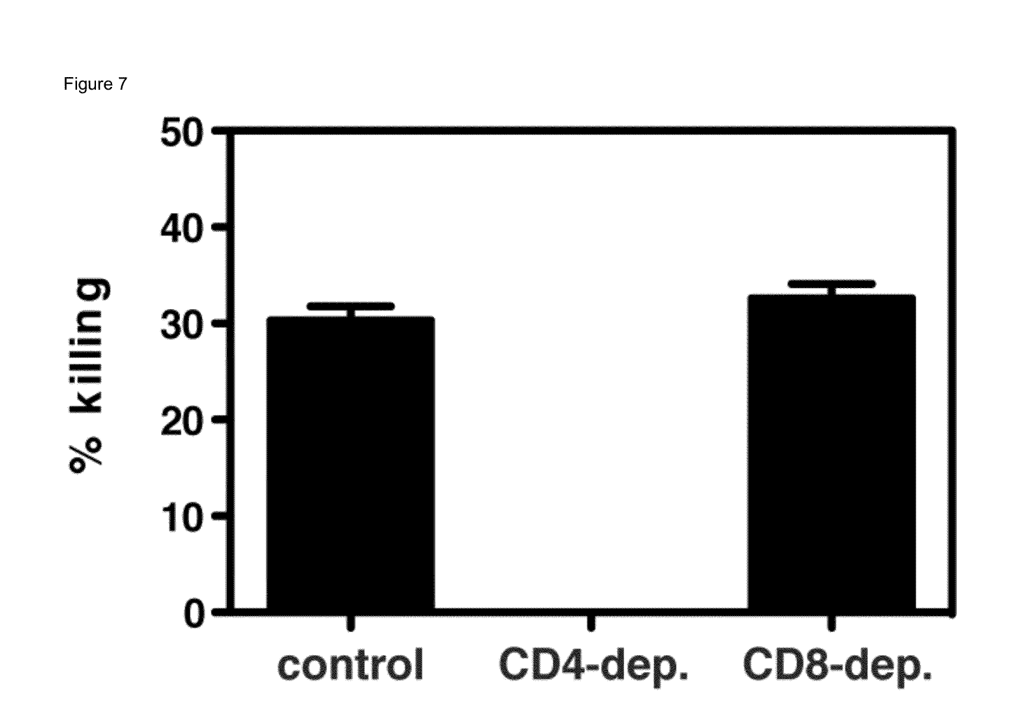

Although the absence of CD4⁺ T cells had no effect on viral RNA levels on day 5 after infection, it was possible that CD4⁺ T cells could still be contributing to the anti-DENV2 host response by killing infected cells. In vivo cytotoxicity assay was performed using splenocytes pulsed with the three peptides that contain only CD4⁺ T cell epitopes (NS2B₁₀₈₋₁₂₂, NS3₁₉₈₋₂₁₂, and NS3₂₃₇₋₂₅₁) and not NS4B₉₆₋₁₁₀ to measure only CD4′, not CD8⁺ T cell-mediated killing. Approximately 30% killing of target cells was observed (FIG. 7). No cytolytic activity was observed when CD4⁺ T cells were depleted, whereas depletion of CD8⁺ T cells had no effect on killing, demonstrating that the cytotoxicity was CD4⁺ T cell-mediated. Thus, DENV2-specific CD4⁺ T cells exhibit in vivo cytolytic activity, although this effector function does not appear to significantly contribute to controlling primary DENV2 infection.

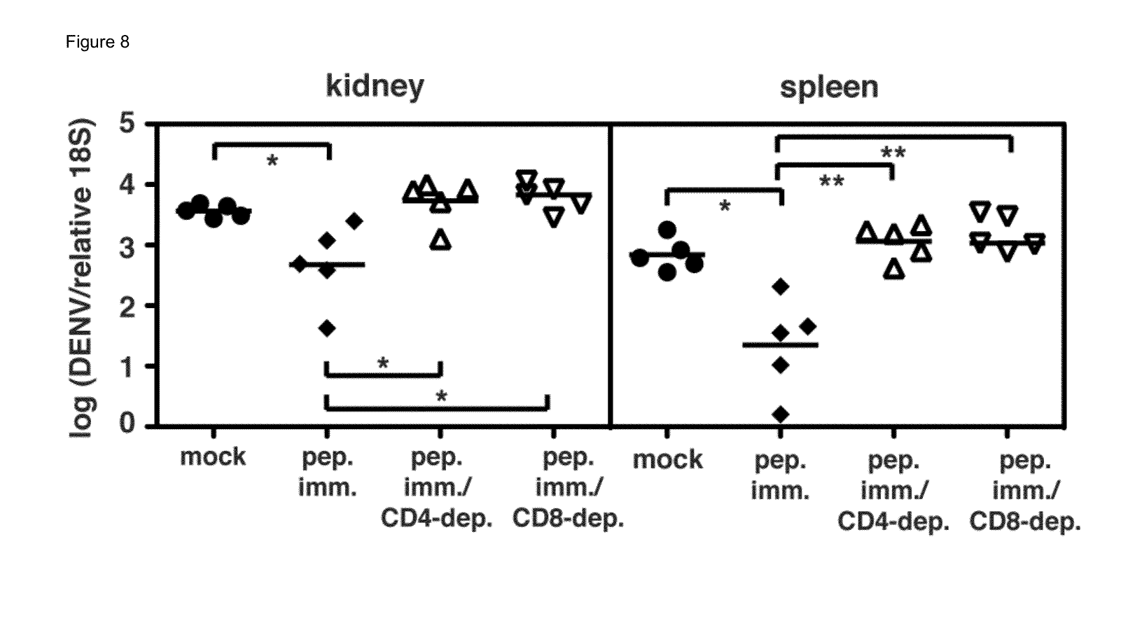

Having found that DENV2-specific CD4⁺ T cells can kill target cells, immunization with CD4⁺ T cell epitopes was assessed for control of DENV2 infection. Mice were immunized with NS2B₁₀₈₋₁₂₂, NS3₁₉₈₋₂₁₂, and NS3₂₃₇₋₂₅₁ before DENV2 infection, and CD4⁺ T cell responses by ICS and viral RNA levels 4 days after infection measured. Peptide immunization resulted in enhanced CD4⁺ T cell cytokine responses, and significantly lower viral loads in the kidney and spleen (FIG. 8). The protective effect was mediated by CD4⁺ T cells, as CD4-depletion before infection abrogated the protective effect. Similarly, CD8-depletion resulted in no protection, demonstrating that protection after CD4⁺ T cell peptide immunization requires both CD4⁺ and CD8⁺ T cells. These data suggest that CD4⁺ T cells elicited by immunization protect by helping the CD8⁺ T cell response. Thus, although CD4⁺ T cells are not required for the primary CD8⁺ T cell or antibody response, and the absence of CD4⁺ T cells had no effect on viral RNA levels, vaccination with CD4⁺ T cell epitopes can reduce viral loads.

### Example 6

This example includes a discussion of the data and a summary of the implications.

The data reveal that CD8⁺ T cells play an important protective role in the response to primary DENV2 infection, whereas CD4⁺ T cells do not. CD4⁺ T cells expanded, were activated, and were located near CD8⁺ T cells and B cells in the spleen after DENV2 infection, yet they did not seem to affect the induction of the DENV2-specific CD8⁺ T cell or antibody responses. In fact, CD4⁺ T cell depletion had no effect on viral clearance. However, the data demonstrate that vaccination with CD4⁺ T cell epitopes prior to DENV infection can provide significant protection, demonstrating that T cell peptide vaccination is a strategy for DENV immunization without the risk of ADE.

The DENV2-specific CD4⁺ T cells recognized epitopes from the NS2B, NS3, and NS4B proteins, and displayed a Th1 phenotype. CD4⁺ T cell epitopes have been identified in mice infected with other flaviviruses, including YFV, for which an I-A^{b}-restricted peptide from the E protein was identified (van der Most, et al. Virology 296:117 (2002)), and WNV, for which six epitopes from the E and NS3 proteins were identified (Brien, et al. J Immunol 181:8568 (2008)). DENV-derived epitopes recognized by human CD4⁺ T cells have been identified, primarily from NS proteins, including the highly conserved NS3 (Mathew, et al. Immunol Rev 225:300 (2008)). One study identified numerous epitopes from the NS3₂₀₀₋₃₂₄ region, and alignment of consensus sequences for DENV1-4 revealed that this region is more conserved (78%) than NS3 as a whole (68%) (Simmons, et al. J Virol 79:5665 (2005)), suggesting that the region contains good candidates for a DENV T cell epitope-based vaccine. Interestingly, one of the NS3-derived epitopes identified herein is also a human CD4⁺ T cell epitope, which may bind human HLAs promiscuously, making it a good vaccine candidate. Another finding was that one of the CD4⁺ T cell epitopes identified in this study contained the most immunodominant of the CD8⁺ T cell epitopes identified previously. Overlapping epitopes have also been found in LCMV (Homann, et al. Virology 363:113 (2007); Mothe, et al. J Immunol 179:1058-1067 (2007); Dow, et al. J Virol 82:11734 (2008)). The significance of overlapping epitopes is unknown, but is likely related to homology between MHC class I and MHC class II, and may be associated with proteasomal processing. Overlapping epitopes may turn out to be common once the complete CD4⁺ and CD8⁺ T cell responses to other pathogens are mapped.

CD4⁺ T cells are classically defined as helper cells, as they help B cell and CD8⁺ T cell responses. However, inflammatory stimuli can override the need for CD4⁺ T cell help, and therefore, the responses to many acute infections are CD4-independent (Bevan, Nat Rev Immunol 4:595 (2004)). DENV2 replicates to high levels in IFN-α/βR^{-/-} mice, the mice appear hunched and ruffled at the time of peak viremia, and they have intestinal inflammation, suggesting that there is a significant inflammatory response to DENV2. Accordingly, CD4⁺ T cells did not play a critical role in the immune response to primary DENV2 infection. The contribution of CD4⁺ T cells has been examined during infections with other flaviviruses. The reports suggest that the contribution of CD4⁺ T cells to protection against flavivirus infection varies depending on the virus and experimental system (Brien, et al. J Immunol 181:8568 (2008); Murali-Krishna, et al. J Gen Virol 77 (Pt 4):705 (1996); Sitati, et al. J Virol. 80:12060 (2006)).

Antibody responses can be T cell-dependent or T cell-independent. In particular, the formation of GCs is thought to be CD4⁺ T cell-dependent, and is where high-affinity plasma cells and memory B cells are generated and where CSR can occur (Stavnezer, et al. Annu Rev Immunol 26:261 (2008); Allen, et al. Immunity 27:190 (2007); Fagarasan et al. Science 290:89 (2000)). T-independent antibody responses to viruses have been demonstrated for vesicular stomatitis virus (Freer, et al. J Virol 68:3650 (1994)), rotavirus (Franco, et al. Virology 238:169 (1997)), and polyomavirus (Szomolanyi-Tsuda, et al. Virology 280:160 (2001)). In addition, EBV (via LMP1) can induce CD40-independent CSR (He, et al. J Immunol 171:5215 (2003)), and mice deficient for CD40 or CD4⁺ T cells are able to mount an influenza-specific IgG response that is protective (Lee, et al. J Immunol 175:5827 (2005)).

The data herein demonstrate that the DENV2-specific IgG response at day 7 is CD4-independent. The lack of GC B cells in CD4-depleted mice shows that CD4-depletions have a functional effect, and indicate anti-DENV IgG is being produced by extrafollicular B cells. It is possible that the absence of CD4⁺ T cells would have an effect on DENV2-specific antibody titers and/or neutralizing activity at later time points, however, the goal of this study was to determine how CD4⁺ T cells contribute to clearance of primary DENV2 infection, and the early anti-DENV2 antibody response is CD4-independent.

Like pathogen-specific antibody responses, primary CD8⁺ T cell responses to many acute infections are also CD4-independent. CD4-independent CD8⁺ T cell responses have been demonstrated for Listeria monocytogenes (Sun, et al. Science 300:339 (2003); Shedlock, et al. J Immunol 170:2053 (2003)), LCMV (Ahmed, et al. J Virol 62:2102 (1988)), and influenza (Belz, et al. J Virol 76:12388 (2002)). Recently a mechanism for how DCs can activate CD8⁺ T cells in the absence of CD4⁺ T cell help has been described (Johnson, et al. Immunity 30:218 (2009)). In accordance with the studies herein, the primary CD8⁺ T cell response to DENV2 did not depend on CD4′T cells. In fact, an enhanced DENV2-specific CD8⁺ T cell response in CD4-deficient mice compared with control mice at day 7 was observed, which has also been reported for influenza-(Belz, et al. J Virol 76:12388 (2002)) and WNV- (Sitati, et al. J Virol. 80:12060 (2006)) specific CD8⁺ T cell responses. This could be due to the depletion of Tregs, or an increased availability of cytokines (e.g. IL-2) in mice lacking CD4⁺ T cells. This enhanced CD8⁺ T cell response may explain why CD4-depleted mice have no differences in viral titers despite the fact that DENV2-specific CD4⁺ T cells demonstrate in vivo cytotoxicity.

Although CD4⁺ T cells did not play an important role in helping antibody or CD8⁺ T cell responses, DENV2-specific CD4⁺ T cells could kill peptide-pulsed target cells in vivo. CD4⁺ T cells specific for other pathogens, including HIV (Norris, et al. J Virol 78:8844 (2004)) and influenza (Taylor, et al. Immunol Lett 46:67 (1995)) demonstrate in vitro cytotoxicity. In vivo cytotoxicity assays have been used to show CD4⁺ T cell-mediated killing following infection with LCMV (Jellison, et al. J Immunol 174:614 (2005)) and WNV (Brien, et al. J Immunol 181:8568 (2008)). DENV-specific cytolytic human CD4⁺ T cell clones (Gagnon, et al. J Virol 70:141 (1996); Kurane, et al. J Exp Med 170:763 (1989)) and a mouse (H-2^{d}) CD4⁺ T cell clone (Rothman, et al. J Virol 70:6540 (1996)) have been reported. Whether CD4⁺ T cells actually kill infected cells during DENV infection remains to be determined, but is possible, as MHC class II-expressing macrophages are targets of DENV infection (Zellweger, et al. Cell Host Microbe 7:128 (2010)). Based on the fact that CD4-depletion did not have a significant effect on viral clearance, it is unlikely that CD4⁺ T cell-mediated killing plays a major role in the anti-DENV2 response in this model.

A caveat to using the IFN-α/βR^{-/-} mice is that type I IFNs are known to help T cell and B cell responses through their actions on DCs, and can act directly on T cells (Iwasaki, et al. Nat Immunol 5:987 (2004)). Type I IFNs were found to contribute to the expansion of CD4⁺ T cells following infection with LCMV, but not Listeria monocytogenes (Havenar-Daughton, et al. J Immunol 176:3315 (2006)). Type I IFNs can induce the development of Th1 IFN-γ responses in human CD4⁺ T cells, but cannot substitute for IL-12 in promoting Th1 responses in mouse CD4⁺ T cells (Rogge, et al. J Immunol 161:6567 (1998)). Following Listeria infection, IL-12 synergized with type I IFN to induce IFN-γ production by CD4⁺ T cells (Way, et al. J Immunol 178:4498 (2007)). Although DENV does not replicate to detectable levels in wild-type mice, examining the CD4⁺ T cell response in these mice revealed that the same epitopes were recognized as in the IFN-α/βR^{-/-} mice, but the magnitude of the epitope-specific response was greater in the IFN-α/βR^{-/-} mice. This suggests that the high levels of viral replication in the IFN-α/βR^{-/-} mice are sufficient to drive a DENV2-specific CD4⁺ IFN-γ response. The results demonstrate a DENV2-specific CD4⁺ T cell response, including Th1-type cytokine production and cytotoxicity, in the absence of IFN-α/βR signaling; however, this response is not required for clearance of infection. It is possible that CD4⁺ T cells contribute to protection during DENV infection of hosts with intact IFN responses.

The results herein demonstrate that immunization with CD4⁺ T epitopes is also protective. These results have significant implications for DENV vaccine development, since designing a vaccine is challenging, as, ideally, a vaccine needs to protect against all four serotypes. DENV vaccine candidates in development, some of which are in phase II trials, focus on eliciting an antibody response. The challenge is to induce and maintain a robust neutralizing antibody response against all four serotypes, as it is becoming increasingly clear that non-neutralizing antibodies (or sub-neutralizing quantities of antibodies) can actually worsen dengue disease (Zellweger, et al. Cell Host Microbe 7:128 (2010); Balsitis, et al. PLoS Pathog 6:e1000790 (2010)). An alternative approach would be a peptide vaccine that induces cell-mediated immunity, including both CD4⁺ and CD8⁺ T cell responses, which, although not able to prevent infection, would reduce viral loads and disease severity, and would eliminate the risk of ADE. Such a vaccine should target highly conserved regions of the proteome, for example NS3, NS4B, and/or NS5, and ideally include epitopes conserved across all four serotypes. A vaccine containing only peptides from these particular NS proteins would also preclude the induction of any antibody against epitopes on the virion, which could enhance infection, or antibody against NS1, which could potentially contribute to pathogenesis (Lin, et al. Viral Immunol 19:127 (2006)). Peptide vaccination was given along with CFA, which is commonly used in mice to induce Th1 responses (Billiau, et al. J Leukoc Biol 70:849 (2001)), which was the type of response observed after natural DENV infection. CFA is not a vaccine adjuvant approved for human use, and thus, any peptide vaccine developed against DENV will be formulated with an adjuvant that is approved for human use.

Although the results herein indicate that CD4⁺ T cells do not make a significant contribution to controlling primary DENV2 infection, the characterization of the primary CD4⁺ T cell response and epitope identification allows the determination of the role of CD4⁺ T cells during secondary homologous and heterologous infections. CD4⁺ T cells are often dispensable for the primary CD8⁺ T cell response to infection, but have been shown to be required for the maintenance of memory CD8⁺ T cell responses after acute infection (Sun, et al. Nat Immunol 5:927 (2004)). Finally, the data herein support a DENV vaccine strategy that induces CD4⁺ T cell, in addition to CD8⁺ T cell, responses.

### Example 7

This example includes a description of additional studies showing that vaccination with DENV CD8⁺ T cell epitopes controls viral load.

Since depleting CD8⁺ T cells resulted in increased viral loads and DENV-specific CD8⁺ T cells demonstrated in vivo cytotoxic activity, studies were performed to determine whether enhancing the anti-DENV CD8⁺ T cell response through peptide immunization would contribute to protection against a subsequent DENV challenge. Specifically, the effect of peptide vaccination on viremia was determined by immunizing IFN-α/βR^{-/-} mice with DENV-2 derived H-2^{b} peptides prior to infection with S221. Mice were immunized with four dominant DENV epitopes (C₅₁₋₅₉, NS2A₈₋₁₅, NS4B₉₉₋₁₉₇, and NS5₂₃₇₋₂₄₅) (Yauch et al., J Immunol 182:4865 (2009)) in an attempt to induce a multispecific T cell response, which is desirable to prevent possible viral escape through mutation (Welsh et al., Nat Rev Microbiol 5:555 (2007)). At day 4 after infection, viremia in the serum was measured by real-time RT-PCR, as described above. The peptide-immunization resulted in enhanced control of DENV infection, with 350-fold lower serum DENV RNA levels in peptide-immunized mice than mock-immunized mice (Yauch et al., J Immunol 182:4865 (2009)). To confirm that the protection was mediated by CD8⁺ T cells, CD8⁺ T cells were depleted from a group of peptide-immunized mice prior to infection, and it was found that this abrogated the protective effect (Yauch et al., J Immunol 182:4865 (2009)). Thus, the data demonstrate that a preexisting DENV-specific CD8⁺ T cell response induced by peptide vaccination enhances viral clearance.

Most dengue infections are asymptomatic or classified as DF, whereas DHF/DSS accounts for a small percentage of dengue cases, indicating that in most infections the host immune response is protective. These data indicate that CD8⁺ T cells contribute to protection during primary infection by reducing viral load and that CD8⁺ T cells are an important component to a protective immune response.

This study shows that immunization with four dominant epitopes prior to infection resulted in enhanced DENV clearance, and this protection was mediated by CD8⁺ T cells. These results indicate that vaccination with T cell epitopes can reduce viremia.

Results from the Examples described herein reveal a critical role for CD8⁺ T cells in the immune response to an important human pathogen, and provide a rationale for the inclusion of CD8′ T cell epitopes in DENV vaccines. Furthermore, identification of the CD8⁺ T cell epitopes recognized during DENV infection in combination with the disclosed mouse model can provide the foundation for elucidating the protective versus pathogenic role of CD8⁺ T cells during secondary infections.

### Example 8

This example is a description of a novel system to identify DENV specific HLA*0201 epitopes.

Mouse-passaged DENV is able to replicate to significant levels in IFN-α/βR^{-/-} mice. HLA*0201 transgenic and IFN-α/βR^{-/-} mice strains were backcrossed to study DENV-specific HLA restricted T cell responses. These mice were then infected with mouse adapted DENV2 strain S221, and purified splenic T cells were used to study the anti-DENV CD8⁺ T cell responses.

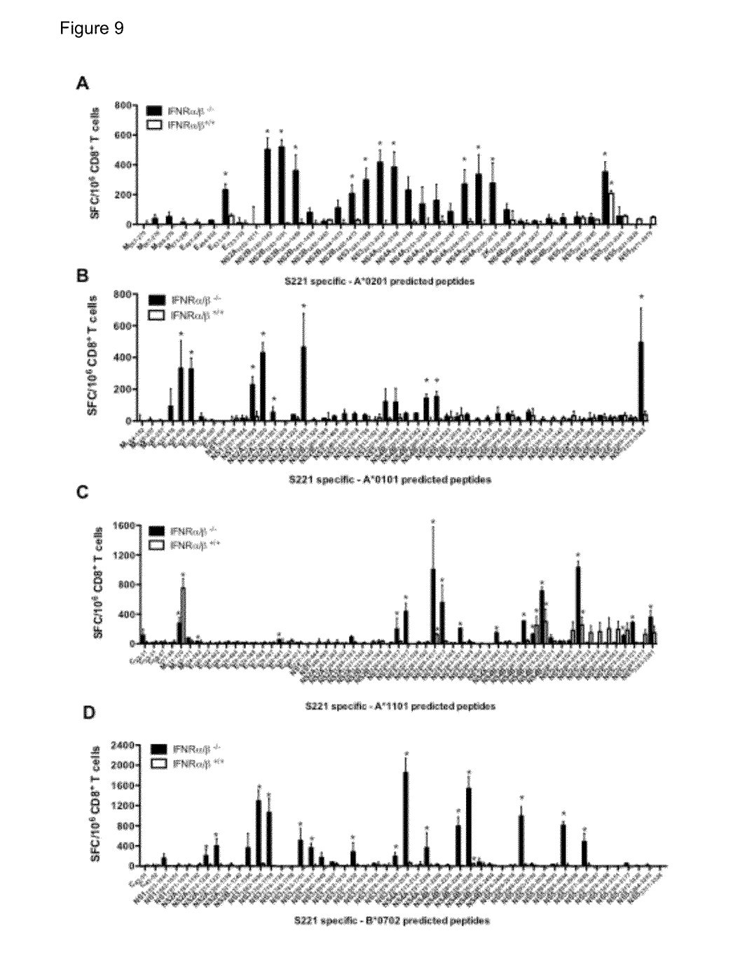

A panel of 116 predicted A*0201 binding peptides were generated using bioinformatics (Moutaftsi, et al. Nat Biotechnol 24:817 (2006)). Predicted HLA A*0201 binding peptides were combined into pools of 10 individual peptides and tested in an IFNγ ELISPOT assay using CD8⁺ T cells from HLA transgenic IFN-α/βR^{-/-} and IFN-α/βR^{+/+}, S221 infected mice, respectively. Positive pools were deconvoluted and the individual peptides were tested in two independent experiments. Using this approach, a single peptide in the HLA*A0201 IFN-α/βR^{+/+} mice was identified (NTS5₃₀₅₈₋₃₀₆₆, FIG. 9A, white bars) whereas screening in IFN-α/βR^{-/-} mice lead to identification of ten additional epitopes. (FIG. 9A, black bars.) These results demonstrate that the HLA A transgenic IFN-α/βR^{-/-} has a stronger and broader T cell response.

### Example 9

This example describes population coverage by additional HLA transgenic mice IFN-α/13R−/− strains.

To address whether similar observations could be made by assessing responses in other HLA-transgenic IFN-α/βR^{-/-} and IFN-α/βR^{+/+} mice, IFN-α/βR^{-/-} mice were backcrossed with HLA A*0101, A*1101, and B*0702 transgenic mice. These alleles were chosen as representatives of three additional HLA class I supertypes (A1, A3 and B7, respectively).

Screening in HLA A*0101 and A*1101 transgenic IFN-α/βR^{-/-} mice revealed 9 HLA A*0101 restricted (FIG. 9B, black bars), and 16 A*1101 restricted epitopes (FIG. 9C, black bars), respectively. In case of the HLA A*0101 transgenic wildtype mice, no epitope could be detected, whereas the HLA A* 1101 transgenic mice showed an overlap of 5 epitopes with the corresponding IFN-α/βR^{-/-} strain (M₁₁₁₋₁₂₀, NS3₁₆₀₈₋₁₆₁₇, NS4B₂₂₈₇₋₂₂₉₆, NS4B₂₃₁₅₋₂₃₂₃ and NS5₃₁₁₂₋₃₁₂₁). Two of these epitopes were able to elicit a stronger response in the HLA A* 1101 IFN-α/βR^{-/-} mice compared to the IFN-α/βR^{-/-} strain (M₁₁₁₋₁₂₀ and NS4B₂₂₈₇₋₂₂₉₆). All other responses observed were stronger in the IFN-α/βR^{-/-} mice.

To extend the observations to mice transgenic for an HLA B allele HLA B*0702 transgenic IFN-α/βR^{-/-} and IFN-α/βR^{-/-} mice were infected and epitope recognition was compared between the two strains. 15 B*0702 restricted epitopes in the IFN-α/βR^{-/-} strain (FIG. 9D, black bars) were identified. 1 of these has also been detected in the corresponding IFN-α/βR^{+/+} mice (NS4B₂₂₈₀₋₂₂₈₉; FIG. 9D, white bars). Similar to the other HLA transgenic mouse strains, the responses observed in the HLA B*0702 transgenic IFN-α/βR^{-/-} mice were not only broader but also more than ten-fold higher in magnitude. The one epitope recognized in the IFN-α/βR^{+/+} strain elicited an IFNγ response of 50 SFC/10⁶ CD8⁺ T cells compared to an average of 857 SFC/10⁶ CD8⁺ T cells in the IFN-α/βR^{-/-} mice.

### Example 10

This example describes Dengue virus specific T cell responses in an MHC class II transgenic mouse model.

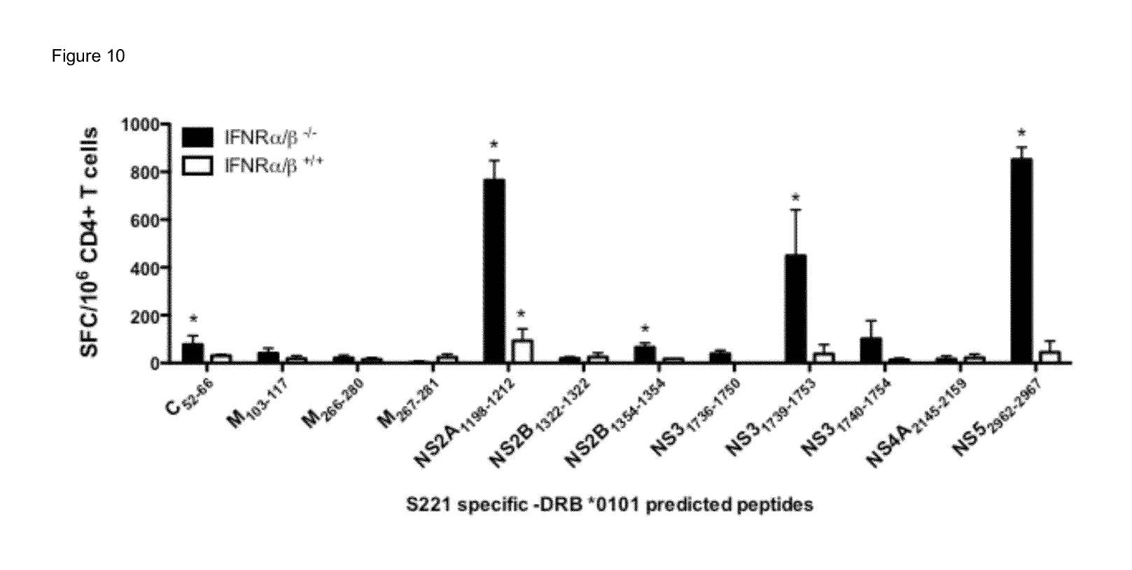

To determine if the observations made in the case of MHC class I transgenic mice were also applicable to MHC class II molecules, the antigenicity of HLA DRB1*0101 DENV predicted binding peptides in HLA DRB1*0101, IFN-α/βR^{-/-} and IFN-α/βR^{+/+} mice, respectively, was determined. Using the same study conditions described above for the MHC class I transgenic mice, HLA DRB1*0101, IFN-α/βR^{-/-} and IFN-α/βR^{+/+} mice were infected with DENV2 (S221), and CD4⁺ T cells were isolated 7 days post infection. A panel of 12 predicted S221 specific peptides was then analyzed for IFNγ production by ELISPOT. Five epitopes in the DRB1*0101, IFN-α/βR^{-/-} mice were identified from these assays which could elicit a significant IFNγ response in two independent experiments (FIG. 10; black bars). As seen above in the MHC class I transgenic mice, only one peptide could be detected in the corresponding DRB1*0101, IFN-α/βR^{+/+} mice (NS2A₁₁₉₉₋₁₂₁₃; FIG. 10, white bars). This identified epitope in the IFN-α/βR^{+/+} did not represent a novel epitope as it was also observed in the corresponding IFN-α/βR^{-/-} mice. Similarly to the MHC class I transgenic mice all observed responses were stronger in the IFN-α/βR^{-/-} mice.

In summary, a total of 55 epitopes were identified in the HLA transgenic IFN-α/βR^{-/-} mice, whereas the same screen in HLA transgenic IFN-α/βR^{+/+} mice only revealed 8 epitopes. All of these 8 epitopes have also been detected in the HLA transgenic IFN-α/βR^{-/-} mice. The broader repertoire seen in IFN-α/βR^{-/-} mice as well as the stronger and more robust IFNγ responses, suggest that HLA transgenic mice, backcrossed with IFN-α/βR^{-/-} mice are a more suitable model to study T cell responses to DENV infection than HLA transgenic wildtype mice.

### Example 11

This example is a description of mapping optimal epitopes with respect to peptide length, and further characterization of the identified epitopes.

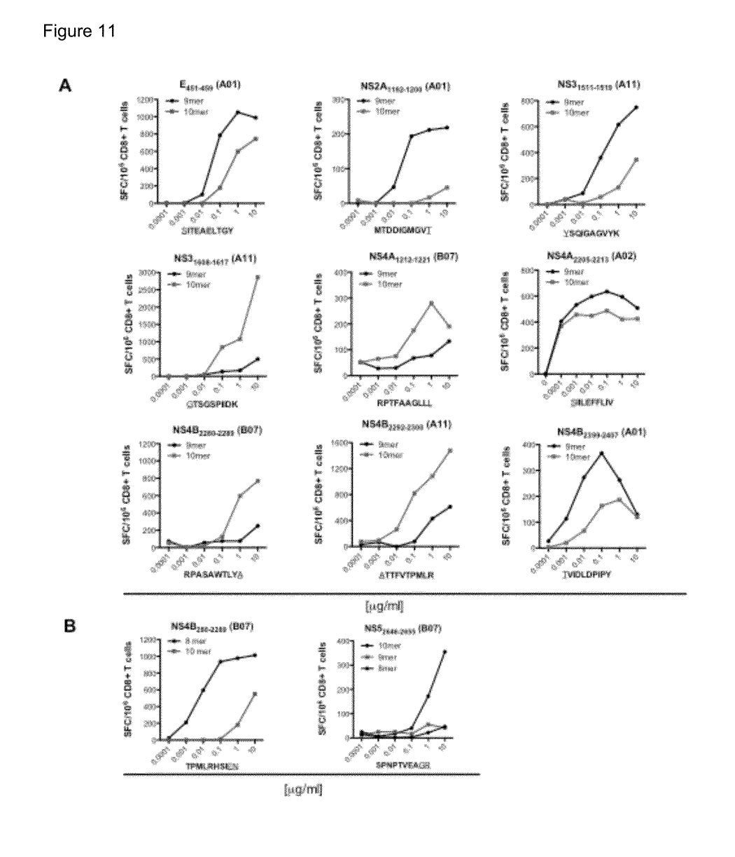

For all HLA alleles tested in this study, class I 9- and 10-mer peptide predictions were performed using the consensus prediction tool as described in greater detail in Example 1. Within the 50 MHC class I restricted epitopes identified, 9 pairs of nested epitopes were identified, where the 9-mer as well as the 10-mer peptide was able to elicit an immune response. To determine which specific peptide within each nested epitope pair was the optimal epitope, peptide titration assays were employed (FIG. 11A). For one epitope (NS4A₂₂₀₅₋₂₂₁₃), both the 9- and the 10-mer displayed similar kinetics upon peptide titration (FIG. 11A). Since the 9-mer was able to elicit slightly higher responses in all conditions tested, the 9-mer version of this epitope was used for further studies. In all other cases an optimal epitope length peptide could be unequivocally identified.

Similarly, for two of the identified B*0702 restricted epitopes (NS4B₂₂₉₆₋₂₃₀₅ and NS5₂₆₄₆₋₂₆₅₅) which showed low binding affinity (IC₅₀>1000 nM) 8-, and 9-mers carrying alternative dominant B7 motifs were synthesized and tested them for T cell recognition and binding affinity. In one case the corresponding 8-mer (NS4B₂₂₉₆₋₂₃₀₄) showed dominant IFNγ responses as well as higher binding affinity compared to the 9-mer. In the other case, the 10mer originally identified (NS5₂₆₄₆₋₂₆₅₅) was able to elicit higher responses than the newly synthesized 8- and 9-mer. In both cases the optimal epitope length could be identified and was considered further in the study, as shown in FIG. 11B.

Of all five HLA transgenic mouse strains analyzed, two strains, namely the A*0201 and the A* 1101 transgenic strains, co-expressed murine MHC molecules together with the respective HLA molecule. Thus it was necessary to address that the observed responses were restricted by the human HLA class I molecule and not by murine Class I. Accordingly, purified T cells were studied for their capacity to recognize the specific epitopes when pulsed on antigen presenting cells expressing only human HLA class and not any murine class I molecule. For this purpose, the tumor cell line 721.221 was utilized, which is negative for expression of any human or murine Class I molecule, and was transfected with either HLA A*0201 or HLA* 1101.

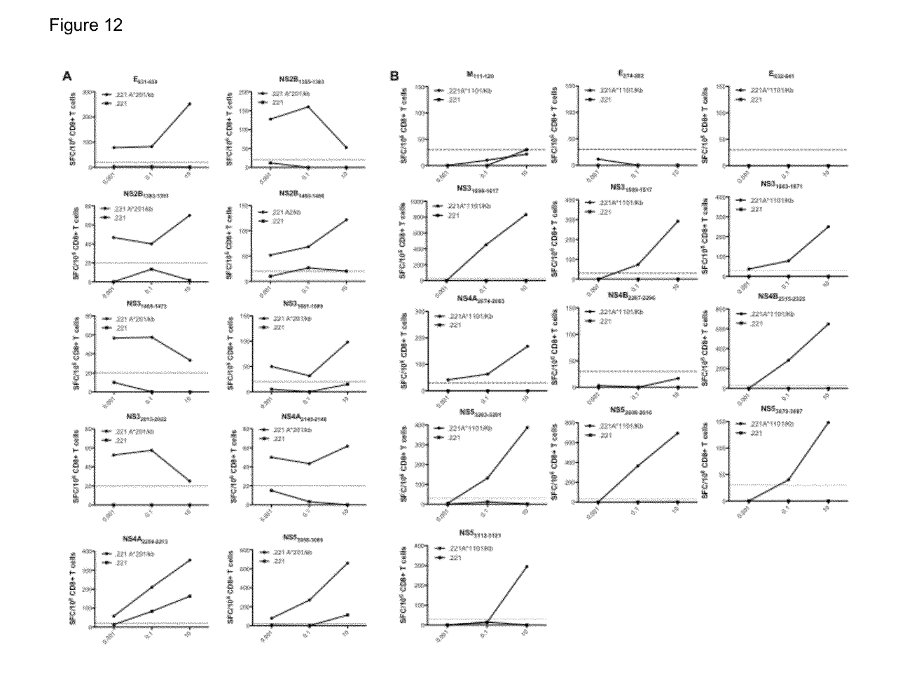

As shown in FIG. 12A, all ten HLA*A0201 restricted epitopes were recognized when presented by APC exclusively expressing HLA*A0201 molecules. Nine out of thirteen of the HLA*A1101 restricted epitopes identified did stimulate a CD8⁺ T cell response when presented exclusively on HLA* 1101 molecules (FIG. 12B). When the four remaining epitopes were tested in non-HLA transgenic IFN-α/βR^{-/-} mice as described above, all elicited a significant T cell response. Furthermore, one of the epitopes has already been described to be recognized by T cells from DENV2 infected Balb/c mice (E₆₃₃₋₆₄₂ (Roehrig, et al. J Virol 66:3385 (1992))). These epitopes (M₁₁₁₋₁₂₀, E₂₇₄₋₂₈₂, E₆₃₃₋₆₄₂, NS4B₂₂₈₇₋₂₂₉₆) are therefore considered solely mouse MHC restricted, and were excluded from further study. Among those epitopes were also the two epitopes which elicited a stronger response in the HLA A* 1101 IFN-α/βR^{+/+} mice compared to the IFN-α/βR^{-/-} strain (M₁₁₁₋₁₂₀ and NS4B₂₂₈₇₋₂₂₉₆).

To further confirm the MHC restriction of the identified epitopes, MHC-binding capacity to their predicted allelic molecule was measured using purified HLA molecules in an in vitro binding assay. The results of these assays are also shown in Table 2. 32 of the 42 tested peptides (67%) bound the corresponding predicted allele with high affinity as indicated by an IC₅₀<50 nM. 16 out of these even showed an IC₅₀<10 nM and can therefore be considered as very strong binders. Of the remaining peptides, 7 (17%) were able to bind the predicted allele with intermediate affinities as indicated by IC₅₀<150 nM. Only three of the identified epitopes (7%) bound with low affinity, showing an IC₅₀>500 nM. A summary of all epitopes identified, after conclusion of the studies and elimination of redundancies, is shown in Table 2 (SEQ ID NOs: 15-56).

TABLE 2
Identified DENV2 derived epitopes in HLA-transgenic IFN-α/βR^{-/-} mice

| | Sequence | | | | | | | | | | |
|---|---|---|---|---|---|---|---|---|---|---|---|
| | (SEQ ID | | T cell | | | | Conservancy within | | | | |
| | NOs: 15-56, | | responses | | frequency | HLA | serotypes | | | | |
| | in order of | Restric- | [SFC] | | in | binding | [%] | | | | Refer- |
| Epitope | appearance) | tion | mouse | human | humans | [IC₅₀] | DENV2 | DENV1 | DENV3 | DENV4 | ences |
| E₄₅₁₋₄₅₉ | ITEAELTGY | | 327 | 67 | 20% | 25 | 85 | 0 | 0 | 0 | |
| | | | | | (1 out of 5) | | | | | | |
| NS1₁₀₉₀₋₁₀₉₉ | RSCTLPPLRY | | 228 | 104 | 20% | 5.9 | 100 | 0 | 100 | 0 | |
| | | | | | (1 out of 5) | | | | | | |
| NS2A₁₁₉₂₋₁₂₀₀ | MTDDIGMGV | A*0101 | 430 | 163 | 20% | 19 | 84 | 0 | 0 | 0 | |
| | | | | | (1 out of 5) | | | | | | |
| NS2A₁₂₅₁₋₁₂₅₉ | LTDALALGM | | 465 | 143 | 40% | 129 | 91 | 0 | 0 | 0 | |
| | | | | | (2 out of 5) | | | | | | |
| NS4B₂₃₉₉₋₂₄₀₇ | VIDLDPIPY | | 153 | 92 | 20% | 17 | 53 | 0 | 0 | 0 | |
| | | | | | (1 out of 5) | | | | | | |
| NS5₃₃₇₅₋₃₃₈₃ | YTDYMPSMK | | 495 | 143 | 20% | 37 | 98 | 0 | 0 | 0 | |
| | | | | | (1 out of 5) | | | | | | |
| E₆₃₁₋₆₃₉ | RLITVNPIV | | 265 | 393 | 43% | 2.8 | 98 | 0 | 0 | 0 | |
| | | | | | (3 out of 7) | | | | | | |
| NS2B₁₃₅₅₋₁₃₆₃ | IMAVGMVSI | | 503 | 417 | 43% | 1.9 | 92 | 0 | 0 | 0 | |
| | | | | | (3 out of 7) | | | | | | |
| NS2B₁₃₈₃₋₁₃₉₁ | GLLTVCYVL | | 519 | 434 | 57% | 6.0 | 100 | 0 | 0 | 0 | |
| | | | | | (4 out of 7) | | | | | | |
| NS2B₁₄₅₀₋₁₄₅₉ | LLVISGLFPV | | 361 | 588 | 43% | 26 | 50 | 0 | 0 | 0 | |
| | | | | | (3 out of 7) | | | | | | |
| NS3₁₄₆₅₋₁₄₇₃ | AAAWYLWEV | A*0201 | 207 | 495 | 57% | 0.39 | 92 | 0 | 0 | 0 | |
| | | | | | (4 out of 7) | | | | | | |
| NS3₁₆₈₁₋₁₆₈₉ | YLPAIVREA | | 299 | 401 | 71% | 18 | 99 | 0 | 0 | 0 | [76¹] |
| | | | | | (5 out of 7) | | | | | | |
| NS3₂₀₁₃₋₂₀₂₂ | DLMRRGDLPV | | 417 | 312 | 71% | 6.3 | 92 | 0 | 0 | 0 | |
| | | | | | (5 out of 7) | | | | | | |
| NS4A₂₁₄₀₋₂₁₄₈ | ALSELPETL | | 384 | 297 | 14% | 61 | 99 | 0 | 0 | 0 | [77²] |
| | | | | | (1 out of 7) | | | | | | |
| NS4A₂₂₀₅₋₂₂₁₃ | IILEFFLIV | | 336 | 301 | 28% | 18 | 99 | 0 | 0 | 0 | |
| | | | | | (2 out of 7) | | | | | | |
| NS5₃₀₅₈₋₃₀₆₆ | KLAEAIFKL | | 353 | 597 | 43% | 2.2 | 95 | 0 | 0 | 0 | [77] |
| | | | | | (3 out of 7) | | | | | | |
| NS3₁₅₀₉₋₁₅₁₇ | SQIGAGVYK | | 436 | 0 | 0% | 33 | 98 | 0 | 0 | 0 | |
| | | | | | (0 out of 5) | | | | | | |
| NS3₁₆₀₈₋₁₆₁₇ | GTSGSPIIDK | | 1003 | 880 | 20% | 12 | 30 | 0 | 0 | 0 | [78³] |
| | | | | | (1 out of 5) | | | | | | |
| NS3₁₈₆₃₋₁₈₇₁ | KTFDSEYVK | | 208 | 0 | 0% | 140 | 75 | 0 | 0 | 0 | [76] |
| | | | | | (0 out of 5) | | | | | | |
| NS4A₂₀₇₄₋₂₀₈₃ | RIYSDPLALK | | 148 | 3087 | 20% | 51 | 89 | 0 | 0 | 0 | [76] |
| | | | | | (1 out of 5) | | | | | | |
| NS4B₂₃₁₅₋₂₃₂₃ | ATVLMGLGK | A*1101 | 712 | 0 | 0% | 16 | 98 | 0 | 0 | 0 | |
| | | | | | (0 out of 5) | | | | | | |
| NS5₂₆₀₈₋₂₆₁₆ | STYGWNLVR | | 1030 | 0 | 0% | 22 | 100 | 0 | 0 | 0 | |
| | | | | | (0 out of 5) | | | | | | |
| NS5₃₀₇₉₋₃₀₈₇ | TVMDIISRR | | 105 | 0 | 0% | 71 | 91 | 0 | 0 | 0 | |
| | | | | | (0 out of 5) | | | | | | |
| NS5₃₁₁₂₋₃₁₂₁ | RQMEGEGVFK | | 284 | 0 | 0% | 118 | 43 | 0 | 0 | 0 | |
| | | | | | (0 out of 5) | | | | | | |
| NS5₃₂₈₃₋₃₂₉₁ | RTTWSIHAK | | 358 | 800 | 20% | 83 | 65 | 0 | 0 | 0 | |
| | | | | | (1 out of 5) | | | | | | |
| NS2A₁₂₁₂₋₁₂₂₁ | RPTFAAGLLL | | 400 | 335 | 20% | 4.8 | 92 | 0 | 0 | 0 | |
| | | | | | (1 out of 5) | | | | | | |
| NS3₁₆₈₂₋₁₆₉₀ | LPAIVREAI | | 1293 | 207 | 20% | 6.5 | 100 | 98 | 96 | 0 | [76] |
| | | | | | (1 out of 5) | | | | | | |
| NS3₁₇₀₀₋₁₇₀₉ | APTRVVAAEM | | 1064 | 1426 | 40% | 4.6 | 99 | 0 | 100 | 100 | [76] |
| | | | | | (2 out of 5) | | | | | | |
| NS3₁₇₅₃₋₁₇₆₁ | VPNYNLIIM | | 509 | 410 | 20% | 43 | 100 | 0 | 89 | 0 | [76] |
| | | | | | (1 out of 5) | | | | | | |
| NS3₁₈₀₈₋₁₈₁₇ | APIMDEEREI | | 364 | 232 | 20% | 572 | 77 | 0 | 0 | 0 | |
| | | | | | (1 out of 5) | | | | | | |
| NS3₁₉₇₈₋₁₉₈₇ | TPEGIIPSMF | B*0702 | 194 | 1825 | 20% | 589 | 99 | 0 | 0 | 0 | [76] |
| | | | | | (1 out of 5) | | | | | | |
| NS3₂₀₇₀₋₂₀₇₈ | KPRWLDARI | | 1853 | 1633 | 40% | 6.8 | 91 | 0 | 0 | 0 | [76] |
| | | | | | (2 out of 5) | | | | | | |
| NS4B₂₂₈₀₋₂₂₈₉ | RPASAWTLYA | | 1539 | 0 | 0% | 7.4 | 100 | 37 | 0 | 100 | |
| | | | | | (0 out of 5) | | | | | | |
| NS4B₂₂₉₆₋₂₃₀₄ | TPMLRHSI | | 1013 | 460 | 20% | 1.1 | 100 | 0 | 0 | 0 | |
| | | | | | (1 out of 5) | | | | | | |
| NS5₂₆₄₆₋₂₆₅₅ | SPNPTVEAGR | | 994 | 0 | 0% | 1332 | 54 | 0 | 0 | 0 | |
| | | | | | (0 out of 5) | | | | | | |
| NS5₂₈₈₅₋₂₈₉₄ | TPRMCTREEF | | 811 | 1341 | 60% | 13 | 89 | 0 | 0 | 0 | |
| | | | | | (3 out of 5) | | | | | | |
| NS5₃₀₇₇₋₃₀₈₅ | RPTPRGTVM | | 487 | 390 | 40% | 1.5 | 97 | 0 | 0 | 0 | |
| | | | | | (2 out of 5) | | | | | | |
| C₅₃₋₆₇ | AFLRFLTIPPTAGIL | | 77 | 314 | 75% | 9.7 | 99 | 0 | 0 | 0 | [79⁴] |
| | | | | | (3 out of 4) | | | | | | |
| NS2A₁₁₉₉₋₁₂₁₃ | GVTYLALLAAFKVRP | | 764 | 249 | 75% | 10 | 91 | 0 | 0 | 0 | |
| | | | | | (3 out of 4) | | | | | | |
| NS2B₁₃₅₆₋₁₃₇₀ | MAVGMVSILASSLLK | DRB1*0101 | 65 | 279 | 75% | 34 | 100 | 0 | 0 | 0 | |
| | | | | | (3 out of 4) | | | | | | |
| NS3₁₇₄₂₋₁₇₅₆ | TFTMRLLSPVRVPNY | | 448 | 336 | 75% | 1.5 | 70 | 100 | 99 | 0 | [76] |
| | | | | | (3 out of 4) | | | | | | |
| NS5₂₉₆₆₋₂₉₈₀ | SRAIWYMWLGARFLE | | 851 | 729 | 75% | 17 | 100 | 99 | 0 | 100 | |
| | | | | | (3 out of 4) | | | | | | |

¹[76] Simmons et al., J Virol 79: 5665 (2005)
²[77] Appanna et al., Clin Vaccine Immunol 14: 969 (2007)
³[78] Mongkolsapaya et al., J Immunol 176: 3821
⁴[79] Wen et al., Virus Res 132: 42 (2008)

### Example 12

This example includes a description of validation studies of the identified epitopes in human DENV seropositive donors.

To validate the epitopes identified in the HLA-transgenic IFN-α/βR^{-/-} mice, the capacity of these epitopes to stimulate PBMC from human donors, previously exposed to DENV, was analyzed. Since the IFNγ response to these peptides was not detectable ex vivo, HLA-matched PBMC were re-stimulated for 7 days in presence of the respective peptides and IL2. As a control PBMC from donors which neither expressed the exact HLA-molecule nor one from the same supertype, as well as PBMC from DENV seronegative donors were re-stimulated. The average IFNγ response from these donors plus 3 times the standard deviation (SD) was set as a threshold of positivity.

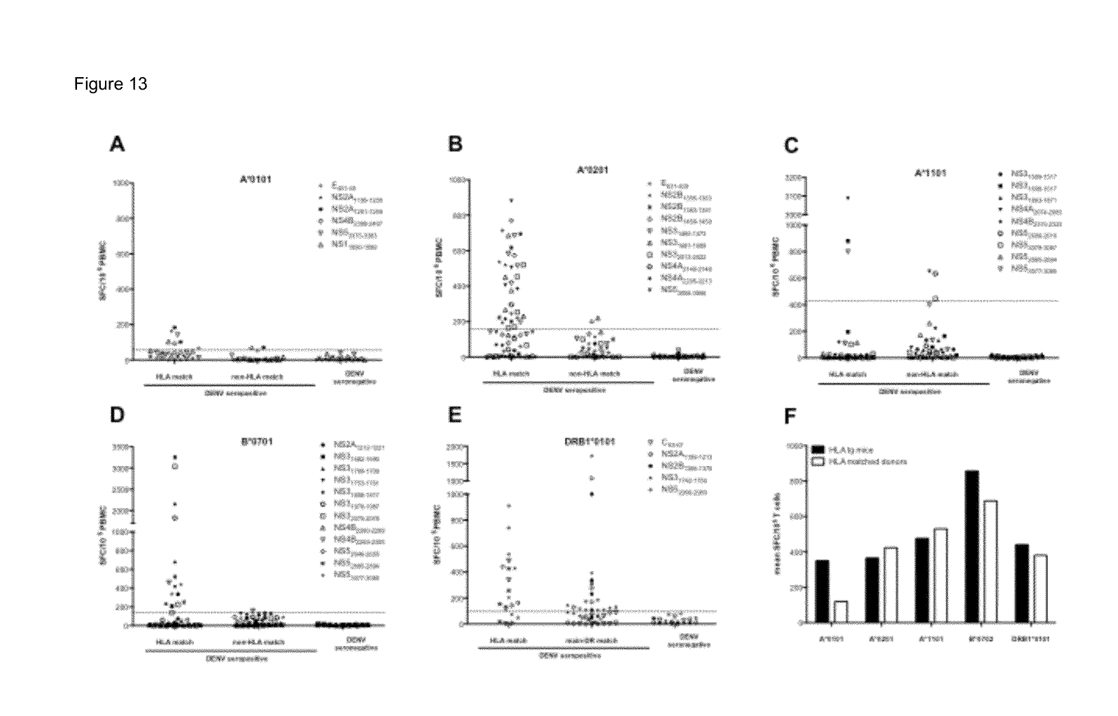

FIGS. 13A-13D (HLA A*0101, A*0201, A*1101, and B*0702) show the capacity of the identified epitopes to stimulate PBMC from the various donor categories. Each of the A*0101 and A*0201 epitopes was detected at least once in an HLA matched donor, although the magnitude as well as the frequency of responses was higher for the A*0201 restricted epitopes (FIGS. 13A-13B and Table 2). Out of the 9 A* 1101 restricted epitopes, 3 have been detected once in HLA matched donors. These three epitopes though have been able to stimulate a robust IFNγ response, as indicated by net SFCs >800 (FIG. 13C). In case of the B*0702 restricted epitopes, 10 out of the 12 have been detected in one or more HLA matched donors as shown in FIG. 13D and Table 1. No significant responses could be detected in non-HLA matched donors studied, as shown for A1, A2, A3 and B7 molecules. In contrast, all four restricted DRB1*0101 epitopes have been detected in 3 out of 4 HLA matched donors tested and were also able to elicit significant IFNγ responses in non-HLA matched donors. This is in accordance with recent reports, demonstrating a high degree of repertoire sharing across MHC class II molecules (Greenbaum, et al. Immunogenetics 63:325 (2011)). Overall, responses to 34 of the 42 epitopes were detected in at least one donor, which corresponds to an overlap of 81% between the murine and human system. In addition to the experimental approach, an IEDB query was performed with the epitopes identified in the mouse model. Here, 13 of the 42 epitopes previously described to elicit an IFNγ in DENV seropositive individuals were identified, as indicated in Table 2. The 30% overlap with known epitopes contributes to the validation of our mouse model and shows on the other hand that 70% of the epitopes identified are novel, contributing to an extended knowledge of T cell mediated responses to DENV.

### Example 13

This example includes studies showing dominance of B7 responses.

A notable observation here was that out of all HLA transgenic mouse strains tested the strongest CD8⁺ T cell responses could be detected in the B*0702 transgenic IFN-α/βR^{-/-} mice. Four B*0702 restricted epitopes were able to elicit an IFNγ response above a thousand SFC/10⁶ CD8⁺ T cells. On average B*0702 epitopes were able to elicit an IFNγ response of 857 SFC/10⁶ CD8⁺ T cells, compared to an average of 350, 365, and 476 SFC/10⁶ CD8⁺ T cells for the HLA A*0101, A*0201 and A* 1101 restricted epitopes, respectively (FIG. 13F, black bars). Most interestingly, the exact same response pattern could be observed testing PBMC from HLA matched donors, previously exposed to DENV (FIG. 13F, white bars). As seen in mice, B*0702 restricted epitopes were able to elicit the strongest IFNγ responses, reaching an average of 688 SFC/10⁶ CD8⁺ T cells, followed by an average of 530, 423 and 119 SFC/10⁶ CD8⁺ T cells for HLA* 1101, A*0202 and A*0101 restricted epitopes, respectively. The fact that the mouse model described herein reflects response patterns observed in humans makes it an especially suitable model to identify and study epitopes of human relevance to DENV infection.

### Example 14

This example includes a description of studies showing the subprotein location of identified epitopes, and the conservancy of identified epitopes within the DENV2 serotype.

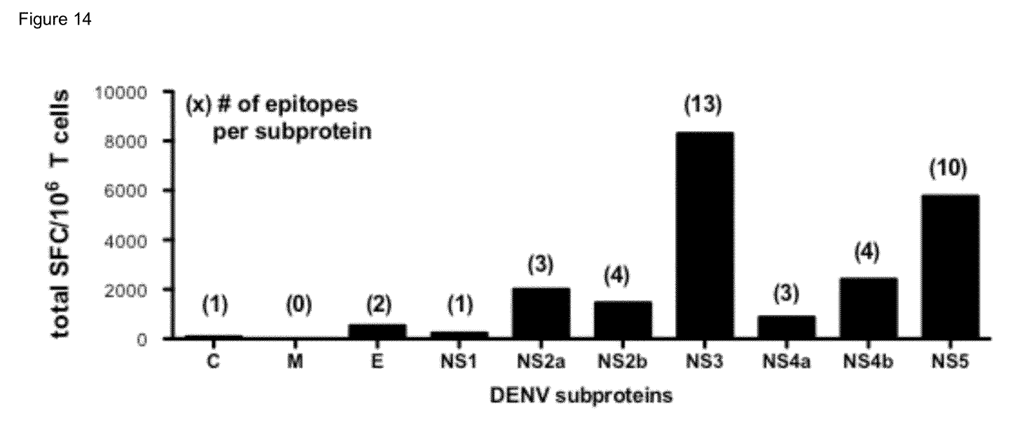

The identified epitopes are derived from 9 of the 10 DENV proteins, with the membrane protein being the only protein, no epitope could be detected (FIG. 14). The majority of epitopes are derived from the seven nonstructural proteins. 39 out of 42 of the identified epitopes (93%) originate from the nonstructural proteins, accounting for 97% of the total IFNγ response observed. Within the nonstructural proteins, however, NS3 and NS5 alone account for 67% of the total response, representing a total number of 23 epitopes detected from these two proteins. NS5 is furthermore the only subprotein where at least one derived epitope has been identified in all five HLA transgenic mouse strains. These results are consistent with the immunodominance of NS3, but also identify NS5 as a major target of T cell responses.

Cross-reactivity of T cells is a well-described phenomenon in DENV infection (Mathew, et al. Immunol Rev 225:300 (2008))). To circumvent this issue, T cell reactivity was exclusively tested to S221 derived peptides, which was also used as infectious agent in this study. However, to asses the relevance for infections with other DENV2 strains, conservancy of these epitopes within the DENV2 serotype was analyzed. 171 full-length DENV2 polyprotein sequences from the NCBI Protein database were analyzed for conservancy. Of the epitopes identified, 30 out of the 42 epitopes were conserved in >90% of all DENV2 strains; 8 epitopes were even conserved in all 171 strains analyzed. Of the remaining 12 epitopes, 6 were conserved in >75% of all strains analyzed and the other half was found in the 30-65% range. This accounts for an average conservancy of 92% for the epitopes identified, which is significantly higher than the average conservancy of non-epitopes (73%; p<0.001).

To determine if the epitopes identified were also conserved in serotypes, other than DENV2, 162 DENV1, 169 DENV3 and 53 DENV4 sequences from the NCBI protein database were studied for conservancy. In contrast to a high degree of conservancy within the DENV2 serotype, 35 out of the 42 epitopes did not occur in any of the 384 DENV-1, 3 and 4 sequences tested and only 7 epitopes had sequence homologues in one or more of the other serotypes. Interestingly, most of the epitopes which show conservancy across serotypes have been identified in the B*0702 transgenic mice. 4 of the identified B*0702 restricted epitopes (NS3₁₆₈₂₋₁₆₉₀, NS3₁₇₀₀₋₁₇₀₉, NS3₁₇₅₃₋₁₇₆₁, NS4B₂₂₈₀₋₂₈₉₂) were additionally conserved in 89-100% of sequences derived from serotypes other than DENV2. The same has been observed for two DRB1*0101 restricted epitopes which were conserved across serotypes (NS3₁₇₄₂₋₁₇₅₆, NS5₂₉₆₆₋₂₉₈₀). Here, the epitopes were conserved in >99% of polyprotein sequences of two serotypes other than DENV2. Finally, one of the A*0101 restricted epitopes (NS1₁₀₉₀₋₁₀₉₉) is also conserved in 100% of DENV3 sequences. All results from this analysis are shown in Tables 2 and 3 (SEQ ID NOs: 15-554).

The DENV2 epitopes identified in Table 2 were analyzed for their respective homologues in DENV1, DENV3 and DENV4. 162 DENV1, 171 DENV2, 169 DENV3 and 53 DENV4 sequences from the NCBI Protein database were analyzed for conservancy. Table 3 (SEQ ID NOs: 57-554) shows the sequences of the epitopes identified after infection with DENV2 (bold letters). “Counts” indicate the number of strains in which the epitope is conserved within the respective serotype. Listed for each epitope are variants of the epitope in the DENV1, 3 and 4 serotypes and their respective counts. Epitopes are sorted according to their appearance in Table 2 (SEQ ID NOs: 15-56). These sequences help determine the cross-reactivity patterns of the identified epitopes.

TABLE 3
Conservancy and Variants of Epitopes
Identified - CD8 Epitopes

| Epitope | | Serotype | Counts |
|---|---|---|---|
| | Sequence | | |
| | SEQ ID NOs: 57- | | |
| | 300, in order of | | |
| | appearnce | | |
| E₄₅₁₋₄₅₉ | ITEAELTGY | DENV2 | 146 |
| | STEIQLTDY | DENV1 | 5 |
| | TTEIQLTDY | DENV1 | 37 |
| | TSEIQLIDY | DENV1 | 1 |
| | TSEIQLTDY | DENV1 | 119 |
| | IAEAELTGY | DENV2 | 3 |
| | IAEAELTDY | DENV2 | 6 |
| | ITDAELTGY | DENV2 | 2 |
| | STEAELTGY | DENV2 | 2 |
| | TTEAELTGY | DENV2 | 10 |
| | ISEAELTDY | DENV2 | 2 |
| | ITEAELTGY | DENV2 | 146 |
| | TVEAVLLEY | DENV3 | 1 |
| | TVEAVLPEY | DENV3 | 40 |
| | TVEAILPEY | DENV3 | 44 |
| | TAEAILPEY | DENV3 | 4 |
| | THEALLPEY | DENV3 | 1 |
| | ITEAILPEY | DENV3 | 3 |
| | TTEVILPEY | DENV3 | 1 |
| | TTEAILPEY | DENV3 | 75 |
| | SVEVELPDY | DENV4 | 2 |
| | SVEVKLPDY | DENV4 | 51 |
| NS1₁₀₉₀₋₁₀₉₉ | RSCTLPPLRY | DENV2 | 171 |
| | RSCTLPPLRF | DENV1 | 162 |
| | RSCTLPPLRY | DENV2 | 171 |
| | RSCTLPPLRY | DENV3 | 169 |
| | RSCTMPPLRF | DENV4 | 53 |
| NS2A₁₁₉₂₋₁₂₀₀ | MTDDIGMGV | DENV2 | 143 |
| | ASDRMGMGM | DENV1 | 1 |
| | ASDMMGMGT | DENV1 | 2 |
| | ASDKMGMGT | DENV1 | 24 |
| | ASDNMGMGT | DENV1 | 11 |
| | VSDRMGMGT | DENV1 | 6 |
| | ASDRMGMGT | DENV1 | 118 |
| | MADDIGMGV | DENV2 | 12 |
| | MTDEMGMGV | DENV2 | 14 |
| | ITDDIGMGV | DENV2 | 2 |
| | MTDDIGMGV | DENV2 | 143 |
| | ASDRTGMGV | DENV3 | 1 |
| | ASDKMGMGV | DENV3 | 4 |
| | ATDRMGMGV | DENV3 | 1 |
| | ASDRMGMGV | DENV3 | 163 |
| NS2A₁₂₅₁₋₁₂₅₉ | LTDALALGM | DENV2 | 156 |
| | LGDGLAIGI | DENV1 | 1 |
| | LGDGFAMGI | DENV1 | 1 |
| | LGDGLAMGI | DENV1 | 160 |
| | LTDAIALGI | DENV2 | 13 |
| | LTDAWALGM | DENV2 | 1 |
| | LTDALALGI | DENV2 | 1 |
| | LTDALALGM | DENV2 | 156 |
| | MANGVALGL | DENV3 | 2 |
| | MANGIALGL | DENV3 | 167 |
| | LISGISLGL | DENV4 | 1 |
| | FIDGLSLGL | DENV4 | 1 |
| | LIDGISLGL | DENV4 | 45 |
| | LIDGIALGL | DENV4 | 1 |
| | FIDGISLGL | DENV4 | 5 |
| NS4B₂₃₉₉₋₂₄₀₇ | VIDLDPIPY | DENV2 | 90 |
| | TIDLDPVVY | DENV1 | 6 |
| | AIDLDPVVY | DENV1 | 156 |
| | VIDLEPIPY | DENV2 | 81 |
| | VIDLDPIPY | DENV2 | 90 |
| | TIDLDSVIF | DENV3 | 1 |
| | TIDLDPVIY | DENV3 | 167 |
| | TIALDPVIY | DENV3 | 1 |
| | VIDLEPISY | DENV4 | 53 |
| NS5₃₃₇₅₋₃₃₈₃ | YTDYMPSMK | DENV2 | 168 |
| | YSDYMTSMK | DENV1 | 8 |
| | YLDYMASMK | DENV1 | 1 |
| | YIDYMTSMK | DENV1 | 1 |
| | YLDFMTSMK | DENV1 | 6 |
| | YLDYMTSMK | DENV1 | 143 |
| | YLDYMISMK | DENV1 | 2 |
| | YIDYMPSMK | DENV2 | 1 |
| | YMDYMPSMK | DENV2 | 2 |
| | YTDYMPSMK | DENV2 | 168 |
| | FLDYMPSMK | DENV3 | 169 |
| | YADYMPVMK | DENV4 | 1 |
| | YMDYMPVMK | DENV4 | 1 |
| | YVDYMPAMK | DENV4 | 5 |
| | YVDYMPVMR | DENV4 | 2 |
| | YVDYMPVMK | DENV4 | 44 |
| E₆₃₁₋₆₃₉ | RLITVNPIV | DENV2 | 168 |
| | RVITANPIV | DENV1 | 7 |
| | RLVTANPIV | DENV1 | 11 |
| | RLITANPIV | DENV1 | 144 |
| | RLITVNPVV | DENV2 | 1 |
| | RLITVNPII | DENV2 | 1 |
| | RLITVNPIV | DENV2 | 168 |
| | RLTTVNPIV | DENV2 | 1 |
| | RLITANPIV | DENV3 | 11 |
| | RLITANPVV | DENV3 | 158 |
| | RVISATPLA | DENV4 | 11 |
| | RVISSTPLA | DENV4 | 15 |
| | RIISSTPLA | DENV4 | 9 |
| | RVISSTPFA | DENV4 | 1 |
| | RIISSTPFA | DENV4 | 16 |
| | RIISSIPFA | DENV4 | 1 |
| NS2B₁₃₅₅₋₁₃₆₃ | IMAVGMVSI | DENV2 | 157 |
| | IMAVGVVSI | DENV1 | 2 |
| | VMAVGIVSI | DENV1 | 1 |
| | IMAIGIVSI | DENV1 | 64 |
| | IMAVGIVSI | DENV1 | 95 |
| | VMAVGMVSI | DENV2 | 14 |
| | IMAVGMVSI | DENV2 | 157 |
| | VMAIGLVSI | DENV3 | 3 |
| | VMAVGLVSI | DENV3 | 166 |
| | MMAVGLVSL | DENV4 | 1 |
| | IMAVGLVSL | DENV4 | 52 |
| NS2B₁₃₈₃₋₁₃₉₁ | GLLTVCYVL | DENV2 | 170 |
| | GMLITCYVI | DENV1 | 1 |
| | GMLIACYVI | DENV1 | 161 |
| | GPLTVCYVL | DENV2 | 1 |
| | GLLTVCYVL | DENV2 | 170 |
| | GMLIACYVI | DENV3 | 2 |
| | GLLIACYVI | DENV3 | 167 |
| | GLLLAAYMM | DENV4 | 1 |
| | GLLLAAYVM | DENV4 | 52 |
| NS4A₂₀₇₄₋₂₀₈₃ | RIYSDPLALK | DENV2 | 153 |
| | RTYSDPQALR | DENV1 | 1 |
| | RTYSDPLALR | DENV1 | 161 |
| | RTYSDPLALK | DENV2 | 13 |
| | RIYSDPLTLK | DENV2 | 2 |
| | KIYSDPLALK | DENV2 | 2 |
| | RIYSEPRALK | DENV2 | 1 |
| | RIYSDPLALK | DENV2 | 153 |
| | RTYSDPLAPK | DENV3 | 1 |
| | RTYSDPLALK | DENV3 | 167 |
| | RIYSDPLALK | DENV3 | 1 |
| | RVYADPMALQ | DENV4 | 1 |
| | RVYADPMALK | DENV4 | 52 |
| NS4B₂₃₁₅₋₂₃₂₃ | ATVLMGLGK | DENV2 | 168 |
| | AAILMGLDK | DENV1 | 162 |
| | ATVLMGLGK | DENV2 | 168 |
| | ATVLMGLGR | DENV2 | 3 |
| | AVVLMGLNK | DENV3 | 1 |
| | AVVLMGLDK | DENV3 | 168 |
| | AAVLMGLGK | DENV4 | 53 |
| NS5₂₆₀₈₋₂₆₁₆ | STYGWNLVR | DENV2 | 171 |
| | AAYGWNLVK | DENV1 | 1 |
| | ATYGWNLVK | DENV1 | 161 |
| | STYGWNLVR | DENV2 | 171 |
| | STYGWNLVK | DENV3 | 3 |
| | STYGWNVVK | DENV3 | 1 |
| | STYGWNIVK | DENV3 | 165 |
| | ATYGWNLVK | DENV4 | 53 |
| NS5₃₀₇₉₋₃₀₈₇ | TVMDIISRR | DENV2 | 155 |
| | TVMDIISRR | DENV1 | 1 |
| | TVMDVISRR | DENV1 | 161 |
| | TVLDIISRR | DENV2 | 1 |
| | TVMDIISRK | DENV2 | 15 |
| | TVMDIISRR | DENV2 | 155 |
| | TVMDIISRK | DENV3 | 169 |
| | AVMDIISRK | DENV4 | 53 |
| NS5₃₁₁₂₋₃₂₉₁ | RQMEGEGVFK | DENV2 | 74 |
| | RQMESEEIFS | DENV1 | 1 |
| | RQMESEGIVS | DENV1 | 1 |
| | RQMESEGIFF | DENV1 | 5 |
| | RQMESEGIIL | DENV1 | 1 |
| | RQMESEGIFS | DENV1 | 87 |
| | RQMESEGIFL | DENV1 | 67 |
| | RQMEGEGVFR | DENV2 | 1 |
| | RQMEGEGIFR | DENV2 | 1 |
| | RQMEGEGLFK | DENV2 | 13 |
| | RQMEGEEVFK | DENV2 | 1 |
| | RQMEGEGVFK | DENV2 | 74 |
| | RQMEGEGIFK | DENV2 | 81 |
| | RQMEGEGVLT | DENV3 | 12 |
| | RQMEGEGVLS | DENV3 | 155 |
| | RQMEGEDVLS | DENV3 | 2 |
| | RQMEAEGVIT | DENV4 | 53 |
| NS5₃₂₈₃₋₃₂₉₁ | RTTWSIHAK | DENV2 | 111 |
| | RTTWSIHAH | DENV1 | 162 |
| | RTTWSIHAR | DENV2 | 8 |
| | RTTWSIHAT | DENV2 | 31 |
| | RTTWSIHAS | DENV2 | 21 |
| | RTTWSIHAK | DENV2 | 111 |
| | RTTWSIHAH | DENV3 | 169 |
| | RTTWSIHAH | DENV4 | 53 |
| NS2A₁₂₁₂₋₁₂₂₁ | RPTFAAGLLL | DENV2 | 158 |
| | RPMLAVGLLF | DENV1 | 1 |
| | RPMFAMGLLF | DENV1 | 1 |
| | RPMFAVGLLI | DENV1 | 4 |
| | RPMFAVGLLF | DENV1 | 156 |
| | RPTFAAGLFL | DENV2 | 1 |
| | RPTFAVGLVL | DENV2 | 1 |
| | RPTFAVGLLL | DENV2 | 11 |
| | RPTFAAGLLL | DENV2 | 158 |
| | QPFLALGFFM | DENV3 | 1 |
| | QPFLTLGFFL | DENV3 | 1 |
| | QPFLALGFFL | DENV3 | 167 |
| | SPRYVLGVFL | DENV4 | 1 |
| | SPGYVLGVFL | DENV4 | 46 |
| | SPGYVLGIFL | DENV4 | 6 |
| NS3₁₆₈₂₋₁₆₉₀ | LPAIVREAI | DENV2 | 171 |
| | LPAIIREAI | DENV1 | 1 |
| | LPAIVREAI | DENV1 | 158 |
| | LPAMVREAI | DENV1 | 3 |
| | LPAIVREAI | DENV2 | 171 |
| | LPTIVREAI | DENV3 | 2 |
| | LPAVVREAI | DENV3 | 1 |
| | LPAIVREAI | DENV3 | 163 |
| | LPAIIREAI | DENV3 | 3 |
| | LPSIVREAL | DENV4 | 53 |
| NS3₁₇₀₀₋₁₇₀₉ | APTRVVAAEM | DENV2 | 170 |
| | APTRVVASET | DENV1 | 1 |
| | APTRVVAAEM | DENV1 | 1 |
| | APTRVVASEM | DENV1 | 160 |
| | APPRVVPAEM | DENV2 | 1 |
| | APTRVVAAEM | DENV2 | 170 |
| | APTRVVAAEM | DENV3 | 169 |
| | APTRVVAAEM | DENV4 | 53 |
| NS3₁₇₅₃₋₁₇₆₁ | VPNYNLIIM | DENV2 | 171 |
| | VPNYNMIIV | DENV1 | 1 |
| | VPNYNMIIM | DENV1 | 160 |
| | VPNYNMIVM | DENV1 | 1 |
| | VPNYNLIIM | DENV2 | 171 |
| | VPNYNLIVM | DENV3 | 11 |
| | VPNYNLVVM | DENV3 | 1 |
| | VPNYNLVIM | DENV3 | 6 |
| | VSNYNLIIM | DENV3 | 1 |
| | VPNYNLIIM | DENV3 | 150 |
| | VPNYNLIVM | DENV4 | 53 |
| NS3₁₈₀₈₋₁₈₁₇ | APIMDEEREI | DENV2 | 131 |
| | AIIQDEERDI | DENV1 | 1 |
| | AVIQDEEKDI | DENV1 | 13 |
| | AAIQDEERDI | DENV1 | 3 |
| | AVIQDEERDI | DENV1 | 145 |
| | APIMDDEREI | DENV2 | 1 |
| | APIIDEEREI | DENV2 | 30 |
| | APIVDEEREI | DENV2 | 9 |
| | APIMDEEREI | DENV2 | 131 |
| | APIQDEEKDI | DENV3 | 2 |
| | SPIQDEERDI | DENV3 | 1 |
| | APIQDEERDI | DENV3 | 164 |
| | APIQDKERDI | DENV3 | 2 |
| | SPIEDIEREI | DENV4 | 53 |
| NS3₁₉₇₈₋₁₉₈₇ | TPEGIIPSMF | DENV2 | 170 |
| | TPEGIIPALY | DENV1 | 1 |
| | TPEGIIPALF | DENV1 | 161 |
| | TPEGIIPSLF | DENV2 | 1 |
| | TPEGIIPSMF | DENV2 | 170 |
| | TPEGIIPALF | DENV3 | 169 |
| | TPEGIIPTLF | DENV4 | 53 |
| | Sequence | | |
| | SEQ ID NOs 301- | | |
| | 554, in order of | | |
| | appearnce | | |
| NS2B₁₃₈₃₋₁₃₉₁ | LLVISGLFPV | DENV2 | 85 |
| | LLAISGVYPL | DENV1 | 1 |
| | LLAVSGMYPL | DENV1 | 5 |
| | LLAVSGVYPL | DENV1 | 49 |
| | LLVISGVYPM | DENV1 | 1 |
| | LLAVSGVYPI | DENV1 | 2 |
| | LLAASGVYPM | DENV1 | 1 |
| | LLAISGVYPM | DENV1 | 27 |
| | LLAVSGVYPM | DENV1 | 76 |
| | LLVVSGLFPV | DENV2 | 1 |
| | LLVISGLFPA | DENV2 | 1 |
| | LLVISGLFPI | DENV2 | 15 |
| | LLVISGVFPV | DENV2 | 69 |
| | LLVISGLFPV | DENV2 | 85 |
| | LLIVSGIFPC | DENV3 | 1 |
| | LLIVSGIFPY | DENV3 | 151 |
| | LLIVSGVFPY | DENV3 | 17 |
| | LITVSGLYPL | DENV4 | 53 |
| NS3₁₄₆₅₋₁₄₇₃ | AAAWYLWEV | DENV2 | 157 |
| | LFVWCFWQK | DENV1 | 1 |
| | LFLWYFWQK | DENV1 | 1 |
| | LFVWHFWQK | DENV1 | 6 |
| | FFVWYFWQK | DENV1 | 1 |
| | PFVWYFWQK | DENV1 | 1 |
| | LFVWYFWQK | DENV1 | 152 |
| | AAAWYLWET | DENV2 | 13 |
| | AAAWYLWEA | DENV2 | 1 |
| | AAAWYLWEV | DENV2 | 157 |
| | LLVWHAWQK | DENV3 | 1 |
| | MLVWHTWQK | DENV3 | 1 |
| | LLVWHTWQK | DENV3 | 167 |
| | MALWYIWQV | DENV4 | 9 |
| | MTLWYMWQV | DENV4 | 42 |
| | MALWYMWQV | DENV4 | 2 |
| NS3₁₆₈₁₋₁₆₈₉ | YLPAIVREA | DENV2 | 170 |
| | YLPAIIREA | DENV1 | 1 |
| | YLPAIVREA | DENV1 | 158 |
| | YLPAMVREA | DENV1 | 3 |
| | SLPAIVREA | DENV2 | 1 |
| | YLPAIVREA | DENV2 | 170 |
| | YLPTIVREA | DENV3 | 2 |
| | YLPAVVREA | DENV3 | 1 |
| | YLPAIVREA | DENV3 | 163 |
| | YLPAIIREA | DENV3 | 3 |
| | ILPSIVREA | DENV4 | 53 |
| NS3₂₀₁₃₋₂₀₂₂ | DLMRRGDLPV | DENV2 | 157 |
| | DLLRRGDLPV | DENV1 | 1 |
| | ELMRRGDLPV | DENV1 | 161 |
| | DLMKRGDLPV | DENV2 | 11 |
| | ELMRRGDLPV | DENV2 | 3 |
| | DLMRRGDLPV | DENV2 | 157 |
| | ELMRRGHLPV | DENV3 | 2 |
| | ELMRRGDLPV | DENV3 | 167 |
| | ELMKRGDLPV | DENV4 | 2 |
| | ELMRRGDLPV | DENV4 | 51 |
| NS4A₂₁₄₀₋₂₁₄₈ | ALSELPETL | DENV2 | 169 |
| | ALEELPDTI | DENV1 | 5 |
| | AVEELPDTI | DENV1 | 1 |
| | AMEELPDTI | DENV1 | 156 |
| | ALSELAETL | DENV2 | 1 |
| | ALGELPETL | DENV2 | 1 |
| | ALSELPETL | DENV2 | 169 |
| | AVEELPETM | DENV3 | 169 |
| | ALNELTESL | DENV4 | 1 |
| | ALNELPESL | DENV4 | 52 |
| NS4A₂₂₀₅₋₂₂₁₃ | IILEFFLIV | DENV2 | 170 |
| | IILKFFLMV | DENV1 | 1 |
| | IILEFLLMV | DENV1 | 1 |
| | IMLEFFLMV | DENV1 | 1 |
| | IILEFFLMV | DENV1 | 159 |
| | IILEFFLMV | DENV2 | 1 |
| | IILEFFLIV | DENV2 | 170 |
| | IILEFFMMV | DENV3 | 1 |
| | IVLEFFMMV | DENV3 | 168 |
| | IILEFFLMV | DENV4 | 53 |
| NS5₃₀₅₈₋₃₀₆₆ | KLAEAIFKL | DENV2 | 162 |
| | LLAKAIFKL | DENV1 | 15 |
| | QLAKSIFKL | DENV1 | 1 |
| | LLATSVFKL | DENV1 | 1 |
| | LLAKSIFKL | DENV1 | 26 |
| | LLATAIFKL | DENV1 | 1 |
| | LLATSIFKL | DENV1 | 117 |
| | LLASSIFKL | DENV1 | 1 |
| | KLAEAIFRL | DENV2 | 6 |
| | RLAEAIFKL | DENV2 | 2 |
| | KLAEAVFKL | DENV2 | 1 |
| | KLAEAIFKL | DENV2 | 162 |
| | QLASAIFKL | DENV3 | 6 |
| | LLANAIFKL | DENV3 | 1 |
| | RLANAIFKL | DENV3 | 2 |
| | QLANAIFKL | DENV3 | 160 |
| | TLAKAIFKL | DENV4 | 9 |
| | ILAKAIFKL | DENV4 | 44 |
| NS3₁₅₀₉₋₁₅₁₇ | SQIGAGVYK | DENV2 | 168 |
| | SQVGVGVFQ | DENV1 | 162 |
| | SQIGAGVYR | DENV2 | 1 |
| | SQIGTGVYK | DENV2 | 1 |
| | SQIGVGVYK | DENV2 | 1 |
| | SQIGAGVYK | DENV2 | 168 |
| | TQVGVGIQK | DENV3 | 3 |
| | TQVGVGVHK | DENV3 | 2 |
| | TQVGVGVQK | DENV3 | 164 |
| | TQVGVGIHI | DENV4 | 4 |
| | TQVGVGIHT | DENV4 | 1 |
| | TQVGVGIHM | DENV4 | 47 |
| | TQVGVGVHV | DENV4 | 1 |
| NS3₁₆₀₈₋₁₆₁₇ | GTSGSPIIDK | DENV2 | 49 |
| | GTSGSPIVSR | DENV1 | 1 |
| | GTSGSPIVNR | DENV1 | 161 |
| | GTSGSPIIDK | DENV2 | 49 |
| | GTSGSPIADK | DENV2 | 1 |
| | GTSGSPIVDR | DENV2 | 75 |
| | GTSGSPIVDK | DENV2 | 46 |
| | GTSGSPIINK | DENV3 | 1 |
| | GTSGSPIINR | DENV3 | 168 |
| | GSSGSPIINR | DENV4 | 1 |
| | GTSGSPIVNR | DENV4 | 1 |
| | GTSGSPIINK | DENV4 | 13 |
| | GTSGSPIINR | DENV4 | 38 |
| NS3₁₈₆₃₋₁₈₇₁ | KTFDSEYVK | DENV2 | 129 |
| | KTFDTEYQK | DENV1 | 162 |
| | KTFDTEYTK | DENV2 | 5 |
| | KTFDTEYIK | DENV2 | 7 |
| | KTFDFEYIK | DENV2 | 1 |
| | KTFDSEYIK | DENV2 | 26 |
| | KTFDSEYAK | DENV2 | 3 |
| | KTFDSEYVK | DENV2 | 129 |
| | KTFDTEYQR | DENV3 | 1 |
| | KTFNTEYQK | DENV3 | 1 |
| | KTFDTEYQK | DENV3 | 167 |
| | KTFDTEYPK | DENV4 | 53 |
| NS3₂₀₇₀₋₂₀₇₈ | KPRWLDARI | DENV2 | 155 |
| | RPRWLDART | DENV1 | 162 |
| | KPRWLDART | DENV2 | 13 |
| | KPRWLDAKI | DENV2 | 2 |
| | KPRWLDPRI | DENV2 | 1 |
| | KPRWLDARI | DENV2 | 155 |
| | RPRWLDART | DENV3 | 168 |
| | RPRWLDARI | DENV3 | 1 |
| | RPRWLDARV | DENV4 | 24 |
| | RPKWLDARV | DENV4 | 29 |
| NS4B₂₂₈₀₋₂₂₈₉ | RPASAWTLYA | DENV2 | 171 |
| | HPASAWTLYA | DENV1 | 102 |
| | RPASAWTLYA | DENV1 | 60 |
| | RPASAWTLYA | DENV2 | 171 |
| | HPASAWILYA | DENV3 | 1 |
| | HPASAWTLYA | DENV3 | 168 |
| | RPASAWTLYA | DENV4 | 53 |
| NS4B₂₂₉₆₋₂₃₀₃ | TPMLRHSI | DENV2 | 171 |
| | TPMLRHTI | DENV1 | 1 |
| | TPMMRHTI | DENV1 | 161 |
| | TPMLRHSI | DENV2 | 171 |
| | TPMLRHTI | DENV3 | 169 |
| | TPMLRHTI | DENV4 | 53 |
| NS5₂₆₄₆₋₂₆₅₅ | SPNPTVEAGR | DENV2 | 92 |
| | SPNPTIEEGR | DENV1 | 162 |
| | SPSPTVEAGR | DENV2 | 1 |
| | SPNPTVDAGR | DENV2 | 1 |
| | SPNPTVEAGP | DENV2 | 1 |
| | SPNPTIEAGR | DENV2 | 76 |
| | SPNPTVEAGR | DENV2 | 92 |
| | SPSPTVEEGR | DENV3 | 1 |
| | SPSLTVEESR | DENV3 | 1 |
| | SPSPIVEESR | DENV3 | 1 |
| | SPSPTVEESR | DENV3 | 166 |
| | SSNPTIEEGR | DENV4 | 53 |
| NS5₂₈₈₅₋₂₈₉₄ | TPRMCTREEF | DENV2 | 152 |
| | KPRICTREEF | DENV1 | 162 |
| | RPRICTRAEF | DENV2 | 1 |
| | KPRICTRAEF | DENV2 | 12 |
| | TRRMCTREEF | DENV2 | 1 |
| | TPRICTREEF | DENV2 | 3 |
| | IPRMCTREEF | DENV2 | 2 |
| | TPRMCTREEF | DENV2 | 152 |
| | KPRLCPREEF | DENV3 | 1 |
| | KPRLCTREEF | DENV3 | 88 |
| | RPRLCTREEF | DENV3 | 80 |
| | NPRLCTKEEF | DENV4 | 1 |
| | SPRLCTREEF | DENV4 | 6 |
| | TPRLCTREEF | DENV4 | 2 |
| | SPRLCTKEEF | DENV4 | 2 |
| | NPRLCTREEF | DENV4 | 41 |
| | KPRLCTREEF | DENV4 | 1 |
| NS5₃₀₇₇₋₃₀₈₅ | RPTPRGTVM | DENV2 | 166 |
| | RPVKNGTVM | DENV1 | 1 |
| | RPARNGTVM | DENV1 | 1 |
| | RPAKNGTVM | DENV1 | 147 |
| | RPAKSGTVM | DENV1 | 13 |
| | RPTPRGTVL | DENV2 | 1 |
| | RPTPKGTVM | DENV2 | 2 |
| | RPTPIGTVM | DENV2 | 2 |
| | RPTPRGTVM | DENV2 | 166 |
| | RPTPKGTVM | DENV3 | 89 |
| | RPTPTGTVM | DENV3 | 80 |
| | RPTPRGAVM | DENV4 | 35 |
| | RPTPKGAVM | DENV4 | 18 |
| C₅₃₋₆₇ | AFLRFLTIPPTAGIL | DENV2 | 169 |
| | AFLRFLAIPPTAGIV | DENV1 | 1 |
| | ALLRFLAIPPTAGIL | DENV1 | 2 |
| | AFLTFLAIPPTAGIL | DENV1 | 1 |
| | AFLRFLAIPPTAGIL | DENV1 | 158 |
| | AFLRFLTISPTAGIL | DENV2 | 1 |
| | AFLRFLTIPPTVGIL | DENV2 | 1 |
| | AFLRFLTIPPTAGIL | DENV2 | 169 |
| | AFLRFLAIPPTAGIL | DENV3 | 20 |
| | AFLRFLAIPPTAGVL | DENV3 | 149 |
| | TFLRVLSIPPTAGIL | DENV4 | 53 |
| NS2A₁₁₉₉₋₁₂₁₃ | GVTYLALLAAFKVRP | DENV2 | 156 |
| | GTTYLALMATFRMRP | DENV1 | 27 |
| | GMTYLALMATFKMRP | DENV1 | 1 |
| | GTTYLALMATLKMRP | DENV1 | 1 |
| | GTTHLALMATFKMRP | DENV1 | 2 |
| | GTTYLALMATFKMRP | DENV1 | 131 |
| | GVTYLALLATFKVRP | DENV2 | 1 |
| | GVTYLALLAAYKVRP | DENV2 | 2 |
| | GVTYLALLAAFRVRP | DENV2 | 12 |
| | GVTYLALLAAFKVRP | DENV2 | 156 |
| | GVTYLALIATFEIQP | DENV3 | 1 |
| | GVTCLALIATFKIQP | DENV3 | 1 |
| | GVTYLALIATFKVQP | DENV3 | 1 |
| | GVTYLALIATFKIQP | DENV3 | 166 |
| | GQTHLAIMAVFKMSP | DENV4 | 23 |
| | GQIHLAIMAVFKMSP | DENV4 | 24 |
| | GQTHLAIMIVFKMSP | DENV4 | 2 |
| | GQVHLAIMAVFKMSP | DENV4 | 3 |
| | GQIHLAIMTMFKMSP | DENV4 | 1 |
| NS3₁₃₅₆₋₁₃₇₀ | MAVGMVSILASSLLK | DENV2 | 171 |
| | MAVGVVSILLSSLLK | DENV1 | 2 |
| | MAIGIVSILLSSLLK | DENV1 | 64 |
| | MAVGIVSILLSSLLK | DENV1 | 96 |
| | MAVGMVSILASSLLK | DENV2 | 171 |
| | MAVGLVSILASSFLR | DENV3 | 11 |
| | MAIGLVSILASSLLR | DENV3 | 3 |
| | MAVGLVSILASSLLR | DENV3 | 155 |
| | MAVGLVSLLGSALLK | DENV4 | 53 |
| NS3₁₇₄₂₋₁₇₅₆ | TFTMRLLSPVRVPNY | DENV2 | 120 |
| | TFTMRLLSPVRVPNY | DENV1 | 162 |
| | PFTMRLLSPVRVPNY | DENV2 | 1 |
| | TFTMRLLSPIRVPNY | DENV2 | 50 |
| | TFTMRLLSPVRVPNY | DENV2 | 120 |
| | TFTMRLLSPVRVSNY | DENV3 | 1 |
| | PFTMRLLSPVRVPNY | DENV3 | 1 |
| | TFTMRLLSPVRVPNY | DENV3 | 167 |
| | TFTTKLLSSTRVPNY | DENV4 | 1 |
| | TFTTRLLSSTRVPNY | DENV4 | 52 |
| NS5₂₉₆₆₋₂₉₈₀ | SRAIWYMWLGARFLE | DENV2 | 171 |
| | SRAIWYVWLGARFLE | DENV1 | 1 |
| | SRAIWYMWLGAAFLE | DENV1 | 1 |
| | SRAIWYMWLGARFLE | DENV1 | 160 |
| | SRAIWYMWLGARFLE | DENV2 | 171 |
| | SRAIWYMWLGARFLE | DENV3 | 5 |
| | SRAIWYMWLGVRYLE | DENV3 | 1 |
| | SRAIWYMWLGARYLE | DENV3 | 163 |
| | SRAIWYMWLGARFLE | DENV4 | 53 |

### Example 15

This example includes a discussion of the foregoing data and conclusions based upon the data.

Wild-type mice are resistant to DENV-induced disease, and therefore, development of mouse models for DENV infection to date has been challenging and has had to rely on infection of immunocompromised mice, non-physiologic routes of infection, and mouse-human chimeras (Yauch, et al. Antiviral Res 80:87 (2008)). Due to the importance of the IFN system in the host antiviral response, mice lacking the IFNR-α/β support a productive infection. A mouse-passaged DENV2 strain, S221, is highly immunogenic and also replicates to high levels in IFNR-α/β−/− mice, thus allowing the study of CD4⁺ and CD8⁺ T cell responses in DENV infection. In this murine model, vaccination with T cell epitopes prior to S221 infection provided significant protection (Yauch, et al. J Immunol 185:5405 (2010); Yauch, et al. J Immunol 182:4865 (2009)). While significant differences exist between human and murine TCR repertoires and processing pathways, HLA transgenic mice are fairly accurate models of human immune responses, especially when peptide immunizations are utilized. Numerous studies to date show that these mice develop T cell responses that mirror the HLA restricted responses observed in humans in context of various pathogens (Gianfrani, et al. Hum Immunol 61:438 (2000); Wentworth, et al. Eur J Immunol 26:97 (1996); Shirai, et al. J Immunol 154:2733 (1995); Ressing, et al. J Immunol 154:5934 (1995); Vitiello, et al. J Exp Med 173:1007 (1991); Diamond, et al. Blood 90:1751 (1997); Firat, et al. Eur J Immunol 29:3112 (1999); Le, et al. J Immunol 142:1366 (1989); Man, et al. Int Immunol 7:597 (1995)).

The data disclosed herein demonstrate that HLA transgenic IFNRα/β^{-/-} mice are a valuable model to identify DENV epitopes recognized in humans. Not only were a number of HLA-restricted T cell responses identified, but the genome wide screen provided further insight into the subproteins targeted by T cells during DENV infection. The majority of DENV responses (97%) was derived from the nonstructural proteins; more than half of the epitopes identified originate from the NS3 and NS5 protein. The data show the immunodominant role of the highly conserved NS3 protein (Rothman Adv Virus Res 60:397 (2003); Duangchinda, et al. Proc Natl Acad Sci USA 107:16922 (2010)), and also suggest NS5 as a major target of T cell responses. Interestingly, proteins previously described as antibody targets (prM, E and NS1) (Rothman J Clin Invest 113:946 (2004)) accounted for less than 5% of all responses, with only 3 epitopes identified from these proteins. The observation that T cell and B cell epitopes after primary DENV infection are not derived from the same proteins may factor in vaccine design, since immunizing with NS3 and NS5 T cell epitopes would induce a robust T cell response without the risk of antibody-dependent-enhancement (ADE).

Another unique challenge in vaccine development is the high degree of sequence variation in a pathogen, characteristically associated with RNA viruses. This is of particular relevance in the case of DENV infections, where it is well documented that prior exposure to a different serotype may lead to more severe disease and immunopathology (Sangkawibha, et al. Am J Epidemiol 120:653 (1984)). The fact that there is also significant genetic variation within each serotype adds to the complexity of successful vaccinations (Twiddy, et al. Virology 298:63 (2002); Holmes, et al. Trends Microbiol 8:74 (2000)). It is hypothesized that in certain cases, peptide variants derived from the original antigen in the primary infection, with substitutions at particular residues, can induce a response that is qualitatively different from the response induced by the original antigen (for example inducing a different pattern of lymphokine production; Partial agonism), or even actively suppressing the response (TCR antagonism). Variants associated with this phenotype are often collectively referred to as Altered Peptide Ligands (APLs) (Yachi, et al. Immunity 25:203 (2006)). During secondary infections, the T cell response directed at the APL may lead to altered or aberrant patterns of lymphokine production, and TCR antagonist mediated inhibition of T cell responses (Kast, et al. J Immunol 152:3904 (1994)). Therefore, immunity to all four serotypes would provide an optimal DENV vaccine. It is generally recognized that conserved protein sequences represent important functional domains (Valdar Proteins 48:227 (2002)), thus mutations at these important protein sites could be detrimental to the survival of the virus. T cell epitopes that target highly conserved regions of a protein are therefore likely to target the majority of genetic variants of a pathogen (Khan, et al. Cell Immunol 244:141 (2006)). Most interestingly in this context was that epitopes that are highly conserved within the DENV2 serotype are the major target for T cells. This data suggests, that immunizations with peptides from a given serotype would protect from the majority of genotypes within this serotype. In contrast, the DENV2 derived epitopes identified are not conserved in other serotypes. These findings point to an immunization strategy with a collection of multiple non-crossreactive epitopes derived from each of the major DENV serotypes. The induction of separate non-crossreactive responses would avoid issues arising from incomplete crossreaction and APL/TCR antagonism effects.

In addition to sequence variation, HLA polymorphism adds to the complexity of studying T cell responses to DENV. MHC molecules are extremely polymorphic, with several hundred different variants known in humans (Klein, Natural History of the Major Histocompatibility Complex (1986); Hughes, et al. Nature 355:402 (1992)). Therefore, selecting multiple peptides matching different MHC binding specificities will increase coverage of the patient population for diagnostic and vaccine applications alike. However, different MHC types are expressed at dramatically different frequencies in different ethnicities. To address this issue, IFNR-α/β−/− mice were backcrossed with mice transgenic for HLA A*0101, A*0201, A*1101, B*0702 and DRB1*0101. These four MHC class I alleles were chosen as representatives of four supertypes (A1, A2, A3 and B7, respectively) and allow a combined coverage of approximately 90% of the worldwide human population (Sette, et al. Immunogenetics 50:201 (1999)), with more than 50% expressing the specific alleles. HLA supertypes are not limited to class I molecules. Several studies have demonstrated the existence of HLA class II supertypes (Doolan, et al. J Immunol 165:1123 (2000); Wilson, et al. J Virol 75:4195 (2001); Southwood, et al. J Immunol 160:3363 (1998)) and functional classification have revealed a surprising degree of repertoire sharing across supertypes (Greenbaum, et al. Immunogenetics 63:325 (2011)). This is in accordance with the data, since the DRB1*0101 restricted epitopes were identified in almost every donor, regardless if the donor was expressing the actual allele. Overall, the mouse model significantly reflects the response pattern observed in humans and that HLA B restricted responses seem to be dominant in B*0702 transgenic mice as well as in human donors, expressing the B*0702 allele (FIG. 13F).

The dominance of HLA B responses has been shown in context of several other viruses, such as HIV, EBV, CMV, and Influenza (Kiepiela, et al. Nature 432:769 (2004); Bihl, et al. J Immunol 176:4094 (2006); Boon, et al. J Immunol 172:4435 (2004); Lacey, et al. Hum Immunol 64:440 (2003)), suggesting that this observation is not limited to RNA viruses, and in fact, it has even been described for an intracellular bacterial pathogen, Mycobacterium Tuberculosis (Lewinsohn, et al. PLoS Pathog 3:1240 (2007); Axelsson-Robertson, et al. Immunology 129:496 (2010)). Furthermore, HLA B restricted T cell responses have been described to be of higher magnitude (Bihl, et al. J Immunol 176:4094 (2006)) and to influence infectious disease course and outcome. In case of DENV, one particular B07 epitope was reported to elicit higher responses in patients with DHF compared to patients suffering from DF only and could therefore be associated with disease outcome (Zivna, et al. J Immunol 168:5959 (2002)). Other reports suggest a role for HLA B44, B62, B76 and B77 alleles in protection against developing clinical disease after secondary DENV infection, whereas other alleles were associated with contribution to pathology (Stephens, et al. Tissue Antigens 60:309 (2002); Appanna, et al. PLoS One 5 (2010). Accordingly, HLA alleles appear to be associated with clinical outcome of exposure to dengue virus, in previously exposed and immunologically primed individuals. The fact that the stronger B*07 response occurs in our human samples as well as in our mouse model of DENV infection validates the relevance of this mouse model, since it even mimics patterns of immuno-dominance observed in humans.

### Example 16

This example includes a description of studies showing the identification of T cell responses against additional DENV-derived peptides in human donors.

Peripheral blood samples were obtained from healthy adult blood donors from the National Blood Center in Colombo, Sri Lanka. DENV-seropositivity was determined by ELISA. Those samples that are positive for DENV-specific IgM or IgG are further examined by the FACS based neutralization assay to determine whether the donor may have been exposed to single or multiple DENV serotypes. For MHC class I binding predictions all 9- and 10-mer peptides were predicted for their binding affinity to their respective alleles. Binding predictions were performed using the command-line version of the consensus prediction tool available on the IEDB web site. Peptides were selected if they were in the top 1% of binders.

As HLA typing and ELISA results were available, donor samples were tested such that predicted peptides for all four serotypes were tested against all appropriate and available HLA types expressed by the donor. DENV specific T cell responses were detected directly ex vivo from our Sri Lankan donor cohort, as measured by an IFNγ ELISPOT assay. Epitopes that have been identified in one or more donors are listed in Table 4 (SEQ ID Nos: 555-763).

TABLE 4
Human Donor Table and DENV Epitopes

| | | | Sequence | | | | | | |
|---|---|---|---|---|---|---|---|---|---|
| | | | (SEQ ID | | | | | | |
| | | | NOs: | | | | | | |
| | Protein location | | 555-763, in | | | | | T cell | HLA- |
| | Start | End | order of | Super- | | | Sero- | response | Binding |
| # | position | position | appearance) | type | Allele | Length | type | [SFC] | [IC50] |
| 1 | 43 | 51 | GPMKLVMAF | B7 | B*0702 | 9 | DENV1 | 32 | 13 |
| 2 | 43 | 52 | GPMKLVMAFI | B7 | B*0702 | 10 | DENV1 | 62 | 86 |
| 3 | 49 | 57 | MAFIAFLRF | B7 | B*3501 | 9 | DENV1 | 82 | 3 |
| 4 | 75 | 83 | KSGAIKVLK | A3 | A*1101 | 9 | DENV3 | 823 | 151 |
| 5 | 104 | 113 | ITLLCLIPTV | A2 | A*0201 | 10 | DENV4 | 43 | 441 |
| 6 | 105 | 114 | CLMMMLPATL | A2 | A*0201 | 10 | DENV3 | 63 | 26 |
| 7 | 105 | 113 | TLLCLIPTV | A2 | A*0201 | 9 | DENV4 | 42 | 1 |
| 8 | 106 | 114 | LMMMLPATL | A2 | A*0201 | 9 | DENV3 | 78 | 22 |
| 9 | 106 | 115 | LMMMLPATLA | A2 | A*0201 | 10 | DENV3 | 50 | 14 |
| 10 | 107 | 115 | MMMLPATLA | A2 | A*0201 | 9 | DENV3 | 62 | 28 |
| 11 | 107 | 116 | MMMLPATLAF | B7 | B*3501 | 10 | DENV3 | 57 | 555 |
| 12 | 108 | 116 | MLIPTAMAF | B7 | B*3501 | 9 | DENV2 | 58 | 422 |
| 13 | 150 | 159 | TLMAMDLGEL | A2 | A*0201 | 10 | DENV2 | 67 | 15 |
| 14 | 164 | 172 | VTYECPLLV | A2 | A*0201 | 9 | DENV4 | 40 | 27 |
| 15 | 245 | 254 | HPGFTILALF | B7 | B*3501 | 10 | DENV3 | 63 | 118 |
| 16 | 248 | 257 | FTIMAAILAY | B7 | B*3501 | 10 | DENV2 | 53 | 4223 |
| 17 | 248 | 257 | FTILALFLAH | B7 | B*3501 | 10 | DENV3 | 32 | 24988 |
| 18 | 249 | 257 | TIMAAILAY | B7 | B*3501 | 9 | DENV2 | 123 | 82 |
| 19 | 274 | 282 | MLVTPSMTM | B7 | B*3501 | 9 | DENV3 | 115 | 3850 |
| 20 | 355 | 363 | CPTQGEATL | B7 | B*3501 | 9 | DENV1 | 143 | 26 |
| 21 | 355 | 363 | CPTQGEAVL | B7 | B*3501 | 9 | DENV3 | 135 | 19 |
| 22 | 363 | 371 | LPEEQDQNY | B7 | B*3501 | 9 | DENV3 | 28 | 1015 |
| 23 | 413 | 421 | YENLKYSVI | B44 | B*4402 | 9 | DENV1 | 37 | 90 |
| 24 | 537 | 545 | QEGAMHSAL | B44 | B*4001 | 9 | DENV4 | 22 | 16 |
| 25 | 537 | 545 | QEGAMHTAL | B44 | B*4001 | 9 | DENV1 | 120 | 5 |
| 26 | 578 | 586 | MSYTMCSGK | A3 | A*1101 | 9 | DENV4 | 48 | 27 |
| 27 | 578 | 587 | MSYSMCTGKF | B7 | B*3501 | 10 | DENV2 | 23 | 10625 |
| 28 | 612 | 621 | SPCKIPFEIM | B7 | B*3501 | 10 | DENV2 | 35 | 7486 |
| 29 | 616 | 625 | IPFEIMDLEK | B7 | B*3501 | 10 | DENV2 | 237 | 6012 |
| 30 | 664 | 673 | EPGQLKLNWF | B7 | B*3501 | 10 | DENV2 | 168 | 42066 |
| 31 | 721 | 729 | FGAIYGAAF | B7 | B*3501 | 9 | DENV2 | 28 | 7667 |
| 32 | 733 | 742 | SWMVRILIGF | A24 | A*2402 | 10 | DENV4 | 90 | 132 |
| 33 | 738 | 746 | IGIGILLTW | B58 | B*5801 | 9 | DENV1 | 23 | 3 |
| 34 | 814 | 823 | SPKRLATAIA | B7 | B*0702 | 10 | DENV3 | 102 | 34 |
| 35 | 845 | 853 | KQIANELNY | B62 | B*1501 | 9 | DENV3 | 22 | 9 |
| 36 | 950 | 959 | VYTQLCDHRL | A24 | A*2402 | 10 | DENV3 | 67 | 6 |
| 37 | 950 | 958 | VYTQLCDHR | A3 | A*3301 | 9 | DENV3 | 28 | 1902 |
| 38 | 968 | 977 | KAVHADMGYW | B58 | B*5801 | 10 | DENV1 | 85 | 1 |
| 39 | 990 | 999 | RASFIEVKTC | B58 | B*5801 | 10 | DENV1 | 138 | 54 |
| 40 | 1023 | 1032 | FAGPVSQHNY | B7 | B*3501 | 10 | DENV2 | 190 | 38 |
| 41 | 1033 | 1041 | RPGYHTQTA | B7 | B*0702 | 9 | DENV2 | 177 | 10 |
| 42 | 1042 | 1051 | GPWHLGKLEL | B7 | B*0702 | 10 | DENV1 | 53 | 18 |
| 43 | 1042 | 1051 | GPWHLGKLEM | B7 | B*3501 | 10 | DENV2 | 25 | 6069 |
| 44 | 1098 | 1107 | RYMGEDGCWY | A24 | A*2402 | 10 | DENV3 | 182 | 829 |
| 45 | 1136 | 1145 | FTMGVLCLAI | A2 | A*0201 | 10 | DENV3 | 33 | 18 |
| 46 | 1176 | 1185 | MSFRDLGRVM | B7 | B*3501 | 10 | DENV2 | 35 | 469 |
| 47 | 1201 | 1209 | TYLALIATF | A24 | A*2402 | 9 | DENV3 | 82 | 7 |
| 48 | 1211 | 1219 | IQPFLALGF | A24 | A*2402 | 9 | DENV3 | 27 | 268 |
| 49 | 1218 | 1227 | GFFLRKLTSR | A3 | A*3301 | 10 | DENV3 | 230 | 59 |
| 50 | 1230 | 1238 | MMATIGIAL | B7 | B*3501 | 9 | DENV2 | 38 | 1117 |
| 51 | 1298 | 1306 | MALSIVSLF | B7 | B*5101 | 9 | DENV1 | 340 | 605 |
| 52 | 1356 | 1364 | MAVGMVSIL | B7 | B*3501 | 9 | DENV2 | 172 | 10 |
| 53 | 1373 | 1382 | IPMTGPLVAG | B7 | B*3501 | 10 | DENV2 | 182 | 129 |
| 54 | 1377 | 1385 | GPLVAGGLL | B7 | B*0702 | 9 | DENV2 | 35 | 67 |
| 55 | 1418 | 1427 | SPILSITISE | B7 | B*3501 | 10 | DENV2 | 158 | 4189 |
| 56 | 1457 | 1466 | FPVSIPITAA | B7 | B*3501 | 10 | DENV2 | 35 | 14 |
| 57 | 1461 | 1469 | IPITAAAWY | B7 | B*3501 | 9 | DENV2 | 70 | 6 |
| 58 | 1519 | 1527 | MEGVFHTMW | B44 | B*4403 | 9 | DENV4 | 68 | 3 |
| 59 | 1519 | 1528 | MEGVFHTMWH | B44 | B*4403 | 10 | DENV4 | 107 | 73 |
| 60 | 1608 | 1616 | KPGTSGSPI | B7 | B*0702 | 9 | DENV1 | 350 | 2 |
| 61 | 1608 | 1617 | KPGTSGSPII | B7 | B*0702 | 10 | DENV3 | 365 | 35 |
| 62 | 1610 | 1619 | GTSGSPIIDK | A3 | A*1101 | 10 | DENV2 | 32 | 12 |
| 63 | 1614 | 1623 | SPIINREGKV | B7 | B*0702 | 10 | DENV3 | 105 | 313 |
| 64 | 1653 | 1661 | NPEIEDDIF | B7 | B*3501 | 9 | DENV2 | 110 | 518 |
| 65 | 1672 | 1681 | HPGAGKTKRY | B7 | B*3501 | 10 | DENV2 | 108 | 680 |
| 66 | 1682 | 1690 | LPAIVREAI | B7 | B*0702 | 9 | DENV1 | 137 | 7 |
| 67 | 1700 | 1709 | APTRVVAAEM | B7 | B*3501 | 10 | DENV2 | 135 | 20 |
| 68 | 1700 | 1709 | APTRVVASEM | B7 | B*0702 | 10 | DENV1 | 153 | 8 |
| 69 | 1700 | 1709 | APTRVVAAEM | B7 | B*0702 | 10 | DENV2 | 113 | 5 |
| 70 | 1707 | 1716 | SEMAEALKGM | B44 | B*4001 | 10 | DENV1 | 120 | 613 |
| 71 | 1716 | 1724 | LPIRYQTPA | B7 | B*0702 | 9 | DENV2 | 180 | 19 |
| 72 | 1716 | 1725 | LPIRYQTPAI | B7 | B*3501 | 10 | DENV2 | 195 | 52 |
| 73 | 1768 | 1777 | DPASIAARGY | B7 | B*3501 | 10 | DENV1 | 183 | 5623 |
| 74 | 1769 | 1778 | PASIAARGYI | B58 | B*5801 | 10 | DENV1 | 140 | 263 |
| 75 | 1795 | 1803 | TPPGSRDPF | B7 | B*3501 | 9 | DENV2 | 210 | 161 |
| 76 | 1803 | 1812 | FPQSNAPIMD | B7 | B*3501 | 10 | DENV2 | 107 | 1 |
| 77 | 1803 | 1811 | FPQSNAPIM | B7 | B*3501 | 9 | DENV2 | 127 | 13693 |
| 78 | 1813 | 1822 | EERDIPERSW | B44 | B*4403 | 10 | DENV1 | 190 | 410 |
| 79 | 1815 | 1824 | REIPERSWNT | B44 | B*4001 | 10 | DENV4 | 93 | 1488 |
| 80 | 1872 | 1881 | YPKTKLTDWD | B7 | B*3501 | 10 | DENV4 | 267 | 1317 |
| 81 | 1899 | 1908 | RVIDPRRCMK | A3 | A*1101 | 10 | DENV2 | 93 | 64 |
| 82 | 1899 | 1908 | RVIDPRRCLK | A3 | A*1101 | 10 | DENV1 | 117 | 58 |
| 83 | 1899 | 1908 | RVIDPRRCMK | A3 | A*3101 | 10 | DENV2 | 115 | 4 |
| 84 | 1899 | 1907 | RVIDPRRCL | B7 | B*0702 | 9 | DENV1 | 117 | 146 |
| 85 | 1899 | 1908 | RVIDPRRCMK | A3 | A*0301 | 10 | DENV2 | 160 | 13 |
| 86 | 1902 | 1910 | DPRRCLKPV | B7 | B*0702 | 9 | DENV1 | 115 | 225 |
| 87 | 1925 | 1933 | MPVTHSSAA | B7 | B*3501 | 9 | DENV2 | 60 | 73 |
| 88 | 1925 | 1934 | MPVTHSSAAQ | B7 | B*3501 | 10 | DENV2 | 25 | 933 |
| 89 | 1942 | 1950 | NPAQEDDQY | B7 | B*3501 | 9 | DENV4 | 118 | 136 |
| 90 | 1949 | 1957 | QYIFTGQPL | A24 | A*2402 | 9 | DENV3 | 78 | 271 |
| 91 | 1978 | 1986 | TPEGIIPSM | B7 | B*0702 | 9 | DENV2 | 108 | 254 |
| 92 | 1978 | 1987 | TPEGIIPSMF | B7 | B*0702 | 10 | DENV2 | 27 | 12953 |
| 93 | 1978 | 1986 | TPEGIIPAL | B7 | B*0702 | 9 | DENV1 | 57 | 1214 |
| 94 | 1978 | 1987 | TPEGIIPALF | B7 | B*0702 | 10 | DENV1 | 38 | 1392 |
| 95 | 1978 | 1986 | TPEGIIPSM | B7 | B*3501 | 9 | DENV2 | 295 | 8 |
| 96 | 1978 | 1987 | TPEGIIPSMF | B7 | B*3501 | 10 | DENV2 | 297 | 386 |
| 97 | 1978 | 1987 | TPEGIIPTLF | B7 | B*3501 | 10 | DENV4 | 90 | 94 |
| 98 | 1978 | 1987 | TPEGIIPALF | B7 | B*3501 | 10 | DENV1 | 20 | 160 |
| 99 | 1999 | 2008 | GEFRLRGEQR | B44 | B*4001 | 10 | DENV4 | 273 | 1407 |
| 100 | 2005 | 2014 | GEARKTFVEL | B44 | B*4001 | 10 | DENV1 | 95 | 7 |
| 101 | 2005 | 2014 | GEARKTFVDL | B44 | B*4001 | 10 | DENV2 | 87 | 5 |
| 102 | 2005 | 2014 | GESRKTFVEL | B44 | B*4001 | 10 | DENV3 | 92 | 4 |
| 103 | 2005 | 2014 | GEQRKTFVEL | B44 | B*4001 | 10 | DENV4 | 37 | 5 |
| 104 | 2013 | 2022 | ELMRRGDLPV | A2 | A*0201 | 10 | DENV1 | 28 | 22 |
| 105 | 2020 | 2029 | LPVWLAYKVA | B7 | B*3501 | 10 | DENV2 | 27 | 5097 |
| 106 | 2026 | 2035 | YKVASAGISY | B7 | B*3501 | 10 | DENV4 | 238 | 70 |
| 107 | 2038 | 2047 | REWCFTGERN | B44 | B*4001 | 10 | DENV4 | 48 | 502 |
| 108 | 2070 | 2078 | RPRWLDART | B7 | B*0702 | 9 | DENV1 | 113 | 2 |
| 109 | 2083 | 2091 | MALKDFKEF | B7 | B*3501 | 9 | DENV4 | 40 | 77 |
| 110 | 2087 | 2095 | EFKEFAAGR | A3 | A*3301 | 9 | DENV1 | 60 | 2 |
| 111 | 2091 | 2100 | FASGRKSITL | B58 | B*5801 | 10 | DENV4 | 72 | 5541 |
| 112 | 2109 | 2118 | LPTFMTQKAR | B7 | B*3501 | 10 | DENV2 | 53 | 176 |
| 113 | 2113 | 2121 | MTQKARNAL | B7 | B*0702 | 9 | DENV2 | 263 | 16 |
| 114 | 2129 | 2137 | TAEAGGRAY | B7 | B*3501 | 9 | DENV2 | 230 | 46 |
| 115 | 2144 | 2153 | LPETLETLLL | B7 | B*3501 | 10 | DENV2 | 512 | 1693 |
| 116 | 2148 | 2156 | LETLMLVAL | B44 | B*4001 | 9 | DENV4 | 112 | 3 |
| 117 | 2148 | 2157 | LETLMLVALL | B44 | B*4001 | 10 | DENV4 | 185 | 127 |
| 118 | 2150 | 2159 | TLMLLALIAV | A2 | A*0201 | 10 | DENV1 | 50 | 8 |
| 119 | 2151 | 2160 | LMLLALIAVL | A2 | A*0201 | 10 | DENV1 | 63 | 95 |
| 120 | 2152 | 2160 | MLLALIAVL | A2 | A*0201 | 9 | DENV1 | 85 | 9 |
| 121 | 2163 | 2172 | GAMLFLISGK | A3 | A*1101 | 10 | DENV3 | 212 | 43 |
| 122 | 2204 | 2213 | SIILEFFLMV | A2 | A*0201 | 10 | DENV1 | 737 | 10 |
| 123 | 2205 | 2213 | IILEFFLMV | A2 | A*0201 | 9 | DENV1 | 232 | 75 |
| 124 | 2205 | 2214 | IILEFFLMVL | A2 | A*0201 | 10 | DENV1 | 152 | 96 |
| 125 | 2210 | 2219 | FLMVLLIPEP | A2 | A*0201 | 10 | DENV1 | 98 | 31 |
| 126 | 2224 | 2233 | TPQDNQLAYV | B7 | B*0702 | 10 | DENV1 | 100 | 331 |
| 127 | 2224 | 2232 | TPQDNQLTY | B7 | B*3501 | 9 | DENV2 | 22 | 11 |
| 128 | 2254 | 2263 | TTKRDLGMSK | A3 | A*1101 | 10 | DENV3 | 75 | 116 |
| 129 | 2266 | 2279 | TETTILDVDL | B44 | B*4001 | 10 | DENV4 | 852 | 11 |
| 130 | 2280 | 2288 | RPASAWTLY | B7 | B*0702 | 9 | DENV1 | 118 | 159 |
| 131 | 2280 | 2289 | RPASAWTLYA | B7 | B*0702 | 10 | DENV1 | 115 | 7 |
| 132 | 2280 | 2288 | HPASAWTLY | B7 | B*3501 | 9 | DENV1 | 38 | 6 |
| 133 | 2281 | 2290 | PASAWTLYAV | B58 | B*5801 | 10 | DENV1 | 90 | 704 |
| 134 | 2290 | 2298 | VATTFVTPM | B7 | B*3501 | 9 | DENV2 | 268 | 205 |
| 135 | 2295 | 2303 | ITPMLRHTI | A24 | A*2402 | 9 | DENV3 | 193 | 138 |
| 136 | 2296 | 2305 | TPMLRHTIEN | B7 | B*0702 | 10 | DENV3 | 90 | 1037 |
| 137 | 2315 | 2323 | IANQATVLM | B7 | B*3501 | 9 | DENV2 | 220 | 16 |
| 138 | 2338 | 2346 | VPLLAIGCY | B7 | B*3501 | 9 | DENV2 | 213 | 168 |
| 139 | 2350 | 2358 | NPLTLTAAV | B7 | B*0702 | 9 | DENV1 | 92 | 32 |
| 140 | 2353 | 2362 | TLTAAVLLLV | A2 | A*0201 | 10 | DENV3 | 43 | 179 |
| 141 | 2356 | 2365 | AAVLLLVTHY | B58 | B*5801 | 10 | DENV3 | 102 | 4148 |
| 142 | 2358 | 2367 | VLLLVTHYAI | A2 | A*0201 | 10 | DENV3 | 260 | 219 |
| 143 | 2403 | 2411 | DPIPYDPKF | B7 | B*3501 | 9 | DENV2 | 77 | 166 |
| 144 | 2419 | 2428 | MLLILCVTQV | A2 | A*0201 | 10 | DENV2 | 103 | 4 |
| 145 | 2444 | 2452 | ATGPLTTLW | B58 | B*5801 | 9 | DENV1 | 350 | 7 |
| 146 | 2444 | 2452 | ATGPISTLW | B58 | B*5801 | 9 | DENV2 | 163 | 1 |
| 147 | 2444 | 2452 | ATGPITTLW | B58 | B*5801 | 9 | DENV3 | 110 | 5 |
| 148 | 2444 | 2452 | ATGPILTLW | B58 | B*5801 | 9 | DENV4 | 27 | 13 |
| 149 | 2444 | 2452 | ATGPVLTLW | B58 | B*5801 | 9 | DENV4 | 185 | 0 |
| 150 | 2451 | 2459 | LWEGSPGKF | A24 | A*2402 | 9 | DENV1 | 57 | 6165 |
| 151 | 2455 | 2464 | SPGKFWNTTI | B7 | B*0702 | 10 | DENV1 | 105 | 6 |
| 152 | 2464 | 2472 | IAVSMANIF | B7 | B*3501 | 9 | DENV1 | 118 | 143 |
| 153 | 2464 | 2472 | IAVSMANIF | B58 | B*5801 | 9 | DENV2 | 108 | 52 |
| 154 | 2464 | 2472 | IAVSTANIF | B58 | B*5801 | 9 | DENV4 | 135 | 196 |
| 155 | 2468 | 2476 | MANIFRGSY | B7 | B*3501 | 9 | DENV1 | 5982 | 553 |
| 156 | 2476 | 2484 | YLAGAGLAF | B7 | B*0702 | 9 | DENV1 | 72 | 98 |
| 157 | 2553 | 2562 | GSSKIRWIVE | B58 | B*5801 | 10 | DENV4 | 45 | 219 |
| 158 | 2602 | 2611 | GPGHEEPIPM | B7 | B*3501 | 10 | DENV1 | 53 | 1150 |
| 159 | 2609 | 2618 | IPMSTYGWNL | B7 | B*0702 | 10 | DENV2 | 203 | 59 |
| 160 | 2609 | 2618 | IPMATYGWNL | B7 | B*0702 | 10 | DENV1 | 450 | 20 |
| 161 | 2609 | 2618 | IPMSTYGWNL | B7 | B*3501 | 10 | DENV2 | 33 | 393 |
| 162 | 2611 | 2620 | MSTYGWNIVK | A3 | A*1101 | 10 | DENV3 | 30 | 146 |
| 163 | 2612 | 2620 | STYGWNIVK | A3 | A*1101 | 9 | DENV3 | 273 | 21 |
| 164 | 2622 | 2631 | QSGVDVFFTP | B58 | B*5801 | 10 | DENV2 | 387 | 2662 |
| 165 | 2658 | 2666 | RVLKMVEPW | B58 | B*5801 | 9 | DENV1 | 643 | 1 |
| 166 | 2676 | 2685 | KVLNPYMPSV | A2 | A*0201 | 10 | DENV2 | 48 | 8 |
| 167 | 2677 | 2685 | VLNPYMPSV | A2 | A*0201 | 9 | DENV2 | 987 | 1 |
| 168 | 2682 | 2691 | MPSVIEKMET | B7 | B*3501 | 10 | DENV2 | 1010 | 375 |
| 169 | 2724 | 2733 | VSSVNMVSRL | B58 | B*5801 | 10 | DENV3 | 820 | 95 |
| 170 | 2729 | 2737 | MVSRLLLNR | A3 | A*1101 | 9 | DENV3 | 992 | 50 |
| 171 | 2738 | 2747 | FTMRHKKATY | B7 | B*3501 | 10 | DENV2 | 103 | 7441 |
| 172 | 2787 | 2795 | WHYDQDHPY | B7 | B*3501 | 9 | DENV2 | 20 | 7598 |
| 173 | 2791 | 2800 | QENPYRTWAY | B44 | B*4001 | 10 | DENV4 | 992 | 1601 |
| 174 | 2798 | 2806 | WAYHGSYET | B7 | B*3501 | 9 | DENV2 | 265 | 873 |
| 175 | 2798 | 2806 | WAYHGSYEV | B7 | B*5101 | 9 | DENV1 | 97 | 11 |
| 176 | 2840 | 2848 | DTTPFGQQR | A3 | A*6801 | 9 | DENV1 | 40 | 91 |
| 177 | 2842 | 2850 | TPFGQQRVF | B7 | B*3501 | 9 | DENV1 | 48 | 47 |
| 178 | 2860 | 2869 | EPKEGTKKLM | B7 | B*3501 | 10 | DENV2 | 382 | 54438 |
| 179 | 2869 | 2877 | MEITAEWLW | B58 | B*5801 | 9 | DENV3 | 27 | 5 |
| 180 | 2885 | 2894 | KPRICTREEF | B7 | B*0702 | 10 | DENV1 | 133 | 72 |
| 181 | 2885 | 2894 | TPRMCTREEF | B7 | B*0702 | 10 | DENV2 | 60 | 13 |
| 182 | 2885 | 2894 | KPRLCTREEF | B7 | B*0702 | 10 | DENV3 | 48 | 13 |
| 183 | 2885 | 2894 | NPRLCTREEF | B7 | B*0702 | 10 | DENV4 | 25 | 45 |
| 184 | 2885 | 2894 | RPRLCTREEF | B7 | B*0702 | 10 | DENV3 | 102 | 7 |
| 185 | 2885 | 2894 | TPRMCTREEF | B7 | B*3501 | 10 | DENV2 | 38 | 2576 |
| 186 | 2918 | 2926 | RAAVEDEEF | B58 | B*5801 | 9 | DENV3 | 87 | 866 |
| 187 | 2919 | 2928 | EAVEDSRFWE | B58 | B*5801 | 10 | DENV2 | 140 | 1714 |
| 188 | 2964 | 2973 | KGSRAIWYMW | B58 | B*5801 | 10 | DENV1 | 335 | 2 |
| 189 | 2977 | 2986 | RYLEFEALGF | A24 | A*2402 | 10 | DENV3 | 130 | 38 |
| 190 | 2977 | 2986 | RFLEFEALGF | A24 | A*2402 | 10 | DENV1 | 37 | 14 |
| 191 | 2993 | 3002 | FSRENSLSGV | B7 | B*5101 | 10 | DENV1 | 103 | 7587 |
| 192 | 3004 | 3012 | GEGLHKLGY | B44 | B*4403 | 9 | DENV1 | 248 | 281 |
| 193 | 3057 | 3065 | RQLANAIFK | A3 | A*1101 | 9 | DENV3 | 277 | 89 |
| 194 | 3079 | 3088 | TPRGTVMDII | B7 | B*0702 | 10 | DENV2 | 505 | 6 |
| 195 | 3079 | 3088 | TPKGAVMDII | B7 | B*0702 | 10 | DENV4 | 422 | 127 |
| 196 | 3116 | 3124 | RQMEGEGIF | B62 | B*1501 | 9 | DENV2 | 583 | 6 |
| 197 | 3116 | 3124 | RQMEGEGVL | B62 | B*1501 | 9 | DENV3 | 382 | 19 |
| 198 | 3182 | 3190 | KVRKDIQQW | B58 | B*5701 | 9 | DENV2 | 115 | 15 |
| 199 | 3254 | 3262 | YAQMWSLMY | B62 | B*1501 | 9 | DENV2 | 27 | 6 |
| 200 | 3254 | 3263 | YAQMWSLMYF | B7 | B*3501 | 10 | DENV2 | 625 | 177 |
| 201 | 3275 | 3283 | ICSAVPVHW | B58 | B*5801 | 9 | DENV3 | 305 | 6 |
| 202 | 3291 | 3299 | WSIHAHHQW | B58 | B*5801 | 9 | DENV1 | 45 | 1 |
| 203 | 3317 | 3326 | NPNMIDKTPV | B7 | B*0702 | 10 | DENV4 | 207 | 403 |
| 204 | 3317 | 3326 | NPWMEDKTPV | B7 | B*0702 | 10 | DENV2 | 137 | 56 |
| 205 | 3332 | 3341 | VPYLGKREDQ | B7 | B*0702 | 10 | DENV1 | 425 | 1251 |
| 206 | 3338 | 3346 | REDLWCGSL | B44 | B*4001 | 9 | DENV4 | 503 | 2 |
| 207 | 3338 | 3346 | REDQWCGSL | B44 | B*4001 | 9 | DENV1 | 150 | 2 |
| 208 | 3379 | 3388 | MPSMKRFRRE | B7 | B*3501 | 10 | DENV2 | 208 | 30905 |
| 209 | 3387 | 3395 | APFESEGVL | B7 | B*0702 | 9 | DENV4 | 77 | 38 |

### Example 17

This example includes a description of studies showing a comprehensive approach to characterize Dengue virus T cell responses.

The role of CD8 T cells in DENV infection is not fully understood. A limitation of existing studies is that only a relatively small fraction of the epitopes derived from the four DENV serotypes and presented by common HLA Class I alleles expressed by populations in endemic areas have been defined, leading to a lack of comprehensiveness in the analyses feasible for investigators. Here the present inventors designed an approach to comprehensively characterize responses by, taking into account HLA polymorphism and the extensive sequence variability both between and amongst the four main DENV serotypes.

555 full-length unique DENV polyprotein sequences (162 DENV1, 171 DENV2, 169 DENV3 and 53 DENV4 sequences, respectively) available from the NCBI Protein database at the start of the study (2009) were retrieved. The number of sequences available varied drastically as a function of geographic locations. For example, in the case of DENV3 40% of the sequences were derived form Venezuela and Puerto Rico alone (Table 5). To ensure a balanced representation, the number of isolates by geographical region from any one country was limited to a maximum of 10. Table 5 illustrates the selection process for DENV3 sequences as an example.

Next, a panel of 16 HLA A and 11 HLA B alleles was selected, which it was estimated would account for 97% of HLA A and B allelic variants in most ethnicities (24). For all four serotypes, 9- and 10-mer sequences predicted to bind to each allelic molecule were generated as described herein. Peptides, which were predicted for two or more serotypes were placed in a “conserved peptides” group. Conversely, if two or more variant peptides at the same position were selected from one serotype, the less commonly encountered sequences were placed in a “variant” group. This resulted in a set of 8,088 peptides, subdivided in 162 different groups of 50 peptides on average (range from 25 to 72, Table 6).

TABLE 5
Selection of DENV3 specific polyprotein sequences

| Country | Count | Unique | Included |
|---|---|---|---|
| Anguilla | 1 | 1 | 1 |
| Bangladesh | 8 | 8 | 8 |
| Brazil | 19 | 18 | 10 |
| Cambodia | 23 | 22 | 10 |
| China | 3 | 2 | 2 |
| Colombia | 12 | 11 | 10 |
| Cook Islands | 1 | 1 | 1 |
| East Timor | 4 | 4 | 4 |
| Ecuador | 1 | 1 | 1 |
| French Polynesia | 9 | 9 | 9 |
| Guyana | 1 | 1 | 1 |
| India | 1 | 1 | 1 |
| Indonesia | 20 | 18 | 10 |
| Malaysia | 23 | 19 | 10 |
| Martinique | 2 | 1 | 1 |
| Mexico | 3 | 3 | 3 |
| Mozambique | 1 | 1 | 1 |
| Nicaragua | 17 | 15 | 10 |
| Peru | 1 | 1 | 1 |
| Philippines | 3 | 3 | 3 |
| Puerto Rico | 94 | 79 | 10 |
| Saint Lucia | 2 | 2 | 2 |
| Samoa | 1 | 1 | 1 |
| Singapore | 48 | 24 | 10 |
| Sri Lanka | 12 | 9 | 9 |
| Taiwan | 15 | 9 | 9 |
| Thailand | 28 | 19 | 10 |
| Trinidad and Tobago | 2 | 1 | 1 |
| Venezuela | 102 | 82 | 10 |
| Viet Nam | 36 | 33 | 10 |
| Total | 493 | 399 | 169 |

TABLE 6
Selection of HLA specific peptide sets

| | | | DENV | DENV | DENV | DENV | | | Row |
|---|---|---|---|---|---|---|---|---|---|
| | Supertype | Allele | 1 | 2 | 3 | 4 | Variants | Conserved | total |
| HLA A | A*01 | A*01:01 | 57 | 59 | 47 | 69 | 39 | 31 | 302 |
| | | A*26:01 | 58 | 65 | 60 | 64 | 31 | 26 | 304 |
| | | A*30:02 | 51 | 59 | 49 | 60 | 33 | 30 | 282 |
| | | A*32:01 | 58 | 65 | 56 | 65 | 46 | 25 | 315 |
| | A*02 | A*02:01 | 62 | 72 | 59 | 66 | 46 | 27 | 332 |
| | | A*02:03 | 64 | 68 | 58 | 70 | 46 | 23 | 329 |
| | | A*02:06 | 62 | 66 | 55 | 64 | 43 | 27 | 317 |
| | | A*68:02 | 58 | 68 | 56 | 70 | 36 | 30 | 318 |
| | A*24 | A*23:01 | 51 | 45 | 50 | 53 | 36 | 40 | 275 |
| | | A*24:02 | 51 | 51 | 55 | 58 | 34 | 36 | 285 |
| | A*03 | A*03:01 | 58 | 67 | 52 | 59 | 34 | 30 | 300 |
| | | A*11:01 | 57 | 71 | 53 | 60 | 29 | 30 | 300 |
| | | A*30:01 | 60 | 67 | 59 | 64 | 26 | 26 | 302 |
| | | A*31:01 | 55 | 61 | 46 | 57 | 37 | 33 | 289 |
| | | A*33:01 | 56 | 58 | 47 | 57 | 28 | 34 | 280 |
| | | A*68:01 | 54 | 58 | 54 | 63 | 24 | 30 | 283 |
| HLA B | B*44 | B*40:01 | 56 | 59 | 57 | 57 | 30 | 31 | 290 |
| | | B*44:02 | 60 | 61 | 63 | 61 | 26 | 30 | 301 |
| | | B*44:03 | 55 | 66 | 56 | 61 | 26 | 32 | 296 |
| | B*57 | B*57:01 | 58 | 57 | 54 | 54 | 40 | 28 | 291 |
| | | B*58:01 | 55 | 60 | 51 | 58 | 47 | 31 | 302 |
| | B*15 | B*15:01 | 54 | 55 | 47 | 61 | 35 | 31 | 283 |
| | B*07 | B*07:02 | 57 | 63 | 46 | 66 | 35 | 32 | 299 |
| | | B*35:01 | 57 | 57 | 52 | 66 | 32 | 31 | 295 |
| | | B*51:01 | 55 | 57 | 56 | 59 | 31 | 35 | 293 |
| | | B*53:01 | 65 | 62 | 55 | 67 | 38 | 27 | 314 |
| | B*08 | B*08:01 | 62 | 62 | 66 | 63 | 31 | 27 | 311 |

| | | | | | | | |
|---|---|---|---|---|---|---|---|
| Column total | 1546 | 1659 | 1459 | 1672 | 939 | 813 | 8088 |

### Example 18

This example includes a description of studies showing validation of the characterization of Dengue virus T cell responses in the general population from the Colombo endemic area

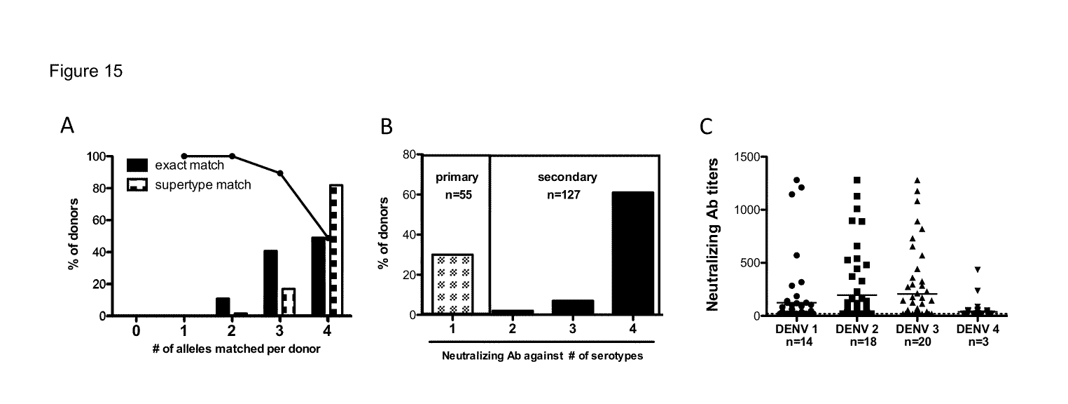

Next, it was sought to validate the approach described herein in the donor population derived from the Colombo (Sri Lanka) region endemic area. In this region, levels of seropositivity for DENV approach 50% by the age of 16 (WHO, dengue bulletin). To capture the features of natural immunity in the general population, buffy coats form the National Blood Bank were obtained. PBMC from a total of 250 blood donors were collected HLA typed. The 27 alleles selected allowed to exactly match 3 out of 4 possible HLA A and B alleles expressed per donor in 41% of our cohort and for 4 out of 4 in 49% (FIG. 15A, black bars). Cumulatively, the number of donors matching at least 3 out of 4 possible HLA A and B alleles is than 90% (FIG. 15A, solid line). Considering closely related HLA alleles from the same supertype allowed to match at least three of four MHC class I alleles in 99% of the donors (FIG. 15A, white bars).

The assumption that this general donor population would be associated with high levels of previous DENV infection as evidenced by positivity in ELISA and neutralization assays was tested. When serum from all donors was tested for the presence of DENV specific IgG antibodies, 80% of the donors in the cohort were seropositive, (182 out of 227) and only 20% (n=45) were negative. To determine whether primary or secondary infection may have occurred, all 182 DENV positive samples were tested in a FACS-based neutralization assay, which showed that 55 donors had experienced primary infection and 127 donors experienced secondary infections.

By definition, donors exposed to primary infection show neutralization antibodies to only one of the serotypes. Neutralization assays are however unable to distinguish which specific DENV serotypes were associated with secondary infections, and most donors had neutralizing antibodies to all four serotypes (FIG. 15B). Antibodies to all four serotypes were detected as infecting agents in our primary infection donors (14 DENV1, 18 DENV2, 20 DENV3 and 3 DENV4 donors; FIG. 15C) confirming a circulation of all four serotypes in Sri Lanka, as previously reported (25). The most frequently encountered primary titers were to DENV2 and DENV3, which have been reported to be the primary circulating serotypes in Sri Lanka (25, 26), followed by DENV1, for which an epidemic has been recently reported (27). Thus, as expected, the pattern of primary infection closely correlates with the DENV serotypes most prevalent in the recent years, close in time to the PBMC donations.

### Example 18

This example includes a description of studies showing determination of T cell reactivity and correlation between T cell and Antibody responses

Next, PBMCs from all donors were screened with HLA matched class I predicted peptide pools in ex vivo IFNγ ELISPOT assays. HLA matched peptide pools originated from all four serotypes. Responses against peptides were considered positive if the net spot-forming cells (SFC) per 10⁶ were ≧20, had a stimulation index of ≧2, and a p<0.05 in at test comparing replicates with those from the negative control in two independent experiments. Positive pools were subsequently deconvoluted and a peptide was considered positive according to the criteria described above.

Overall, ex-vivo T cell reactivity was detected for 22% of primary and 43% of secondary infection donors. In total, 753 total donor/peptide responses were identified. These resulted in the identification of 408 unique CD8⁺ T cell epitopes (Table 7). As a control all DENV negative donors (n=45) were screened and no significant responses were detected (data not shown).

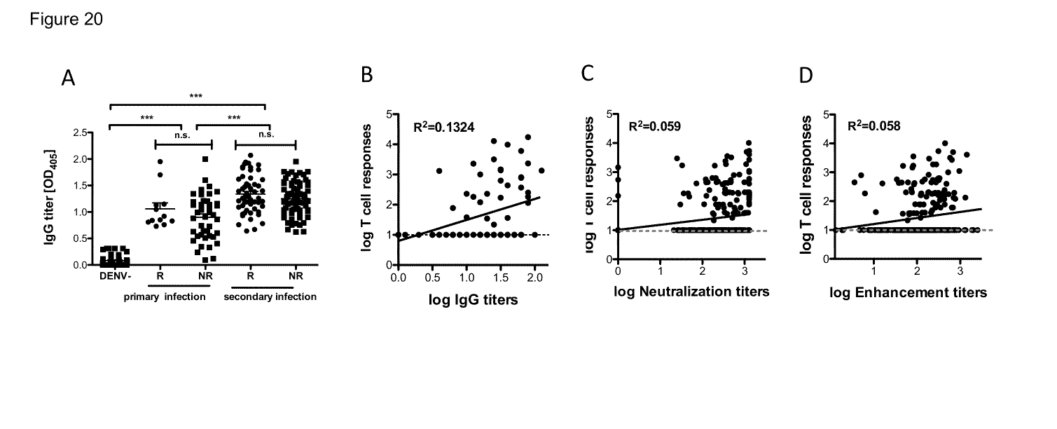

It was also addressed whether the T cell reactivity correlated with IgG titers. Significantly higher titers in secondary infection compared to primary infection, were observed, no difference has been detected between responding and non-responding donors in either cohort (FIG. 20). A subset of 80 donor sera samples randomly selected and also tested for the presence of enhancing antibodies. In these samples, IgG titers, enhancement and neutralization titers showed only weak correlations with paired T cell responses (FIG. 20, B-D, respectively).

TABLE 7
Overall T cell responses in the study population

| | | mean | | |
|---|---|---|---|---|
| DENV | | response/donor | frequency of | mean |
| infection | n | [SFC] | responders [%] | epitopes/donor |
| primary | 55 | 122 | 22 | 6 |
| secondary | 127 | 1004 | 43 | 11 |

| | |
|---|---|
| toal T cell responses detected: | 753 |
| total epitopes identified: | 408 |
| total antigenic regions identified: | 267 |

### Example 19

This example includes a description of studies investigating the Immunodominant regions of the DENV polyprotein

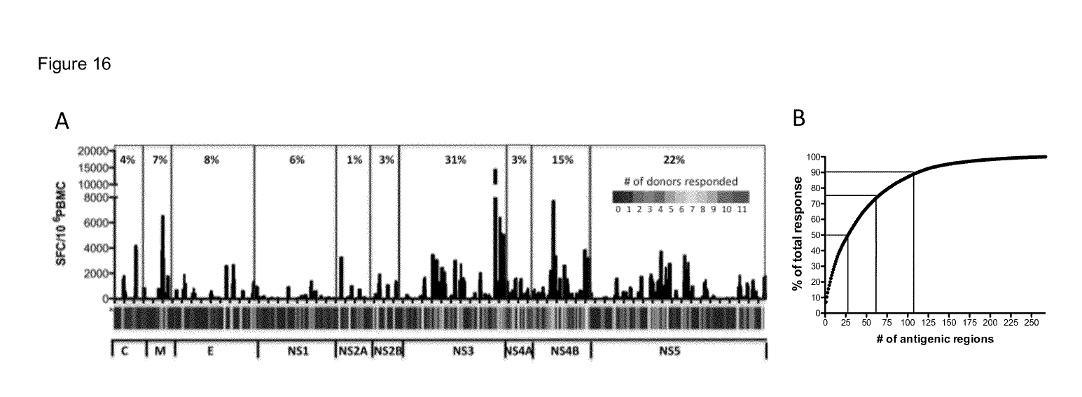

To investigate the relative immunodominance of different parts of the proteome, response magnitude (as SFC/10⁶ PBMC values) and frequency of responding donors (as a heat map) were plotted as a function of the genomic position of DENV encoded proteins (FIG. 16a). NS3, NS4B and NS5 were the most vigorously and frequently recognized proteins within DENV and accounted for more than two thirds of the total response observed. Conversely, proteins known to be main antibody targets (such as NS1 and E) were less prominently recognized at the level of the T cell responses.

It was next noted that reactivity appeared to cluster in discrete regions of the polyprotein. The Epitope Cluster Analysis tool from the IEDB website was used to cluster epitopes that share more than 80% sequence homology, resulting in the definition of a total of 267 antigenic regions (Table 7). When these antigenic regions were plotted as a function of the percentage of the total response (FIG. 16B) it was found that about 25 regions account for half of the total response. Sequences, proteome location, serotype affiliation, HLA restriction as well as frequency and magnitude of responses of these 25 most immunodominant antigenic regions are shown in Table 8 (SEQ ID NOs: 764-812). Several immunodominant regions contain epitopes derived from multiple serotypes and also restricted by a variety of different alleles.

TABLE 8
Immunodominant regions of the DENV polyprotein

| | Epitope | | | | | | |
|---|---|---|---|---|---|---|---|
| | (SEQ ID | | | | | | |
| | NOs: 764- | | | | | | |
| | 812, 1106, | | | | | | |
| | 888 in | | | | | T cell | |
| Antigenic | order of | Proteome | Sero- | HLA | # of | response | |
| Region | appearance) | location | type | allele | responders | [SFC] | |
| 1 | TPEGIIPAL | 1978 | 1, 4 | B*0702 | 1 | 183 | 10671 |
| | TPEGIIPALF | 1978 | 1, 4 | B*3501 | 9 | 2213 | |
| | TPEGIIPSM | 1978 | 2 | B*3501, | 5 | 2717 | |
| | | | | B*5301 | | | |
| | TPEGIIPSMF | 1978 | 2 | B*0702, | 7 | 1772 | |
| | | | | B*3501 | | | |
| | TPEGIIPTLF | 1978 | 4 | B*3501, | 11 | 2914 | |
| | | | | B*0702, | | | |
| | | | | B*5301 | | | |
| | YTPEGIIPTL | 1977 | 4 | A*0206 | 1 | 872 | |
| 2 | GEARKTFVDL | 2005 | 2 | B*4001 | 3 | 1405 | 5005 |
| | GEARKTFVEL | 2005 | 1 | B*4001 | 3 | 1330 | |
| | GEQRKTFVEL | 2005 | 4 | B*4001 | 2 | 1050 | |
| | GESRKTFVEL | 2005 | 3 | B*4001 | 3 | 1220 | |
| 3 | LPVWLAHKVA | 2020 | 3 | B*3501 | 1 | 23 | 4157 |
| | LPVWLAYKV | 2020 | 2 | B*5301, | 2 | 923 | |
| | | | | B*5101 | | | |
| | LPVWLAYKVA | 2020 | 2 | B*3501 | 7 | 2427 | |
| | LPVWLAYRVA | 2020 | 2 | B*5101 | 1 | 470 | |
| | LPVWLSYKV | 2020 | 1 | B*5101 | 1 | 313 | |
| 4 | HPGAGKTKRY | 1672 | 2 | B*3501 | 10 | 3047 | 3047 |
| 5 | NPEIEDDIF | 1653 | 2 | B*3501 | 10 | 3390 | 3390 |
| 6 | DTTPFGOOR | 2840 | 1, 2, | A*6801, | 8 | 3260 | 3260 |
| | | | 3, 4 | A*3301 | | | |
| 7 | MSFRDLGRVM | 1176 | 2 | B*3501 | 8 | 3250 | 3250 |
| 8 | ATGPILTLW | 2444 | 4 | B*5801 | 2 | 938 | 3089 |
| | ATGPISTLW | 2444 | 2 | B*5801 | 2 | 236 | |
| | ATGPITTLW | 2444 | 3 | B*5801 | 1 | 505 | |
| | ATGPLTTLW | 2444 | 1 | B*5801 | 1 | 275 | |
| | ATGPVLTLW | 2444 | 4 | B*5801 | 2 | 390 | |
| | LATGPVLTLW | 2443 | 4 | B*5301 | 1 | 745 | |
| 9 | CLTPTAMAF | 108 | 4 | B*1501 | 1 | 67 | 2815 |
| | MLIPTAMAF | 108 | 2 | B*3501 | 7 | 2748 | |
| 10 | VATTFVTPM | 2290 | 2 | B*3501 | 8 | 2777 | 2777 |
| 11 | KPRLCTREEF | 2885 | 1 | B*0702 | 1 | 340 | 2756 |
| | KPRLCTREEF | 2885 | 3 | B*0702 | 1 | 350 | |
| | NPRLCTREEF | 2885 | 4 | B*0702 | 1 | 365 | |
| | RPRLCTREEF | 2885 | 3 | B*0702 | 4 | 501 | |
| | TPRMCTREEF | 2885 | 2 | B*0702, | 5 | 1200 | |
| | | | | B*3501 | | | |
| 12 | VPLLAIGCY | 2338 | 2 | B*3501 | 7 | 1520 | 2603 |
| | VPLLAMGCY | 2338 | 4 | B*3501 | 1 | 1083 | |
| 13 | MSYSMCTGKF | 578 | 2 | B*3501 | 6 | 2553 | 2553 |
| 14 | DPASTAARGY | 1768 | 1, 2, | B*3501 | 9 | 2383 | 2383 |
| | | | 3 | | | | |
| 15 | IANOATVLM | 2315 | 2 | B*3501 | 7 | 1518 | 1518 |
| 16 | APTRVVAAEM | 1700 | 2, 3, | B*0702, | 8 | 1623 | 2230 |
| | | | 4 | B*3501 | | | |
| | APTRVVASEM | 1700 | 1 | B*0702 | 5 | 607 | |
| 17 | TPMLRHTIEN | 2296 | 3, 4 | B*0702 | 2 | 388 | 2123 |
| 18 | MLVTPSMTM | 274 | 3 | B*3501 | 8 | 1735 | 1735 |
| 19 | FTMRHKKATY | 2738 | 2 | B*3501 | 4 | 1723 | 1723 |
| 20 | FTILALFLAH | 248 | 3 | B*3501 | 8 | 1715 | 1715 |
| 21 | WHYDQDHPY | 2787 | 2 | B*3501 | 5 | 1628 | 1628 |
| 22 | MALKDFKEF | 2083 | 4 | B*3501 | 7 | 1497 | 1497 |
| 23 | LMKTTAEWLW | 2868 | 2 | B*5301 | 1 | 423 | 1417 |
| | MEITAEWLW | 2869 | 3 | B*5801 | 1 | 583 | |
| | VMGITAEWLW | 2868 | 3 | B*5301 | 1 | 410 | |
| 24 | TETTILDVDL | 2266 | 4 | B*4001 | 2 | 1413 | 1413 |
| 25 | GEFRLRGEOR | 1999 | 4 | B*4001 | 3 | 1373 | 1373 |

### Example 20

This example includes a description of studies investigating differences between serotype specific responses.

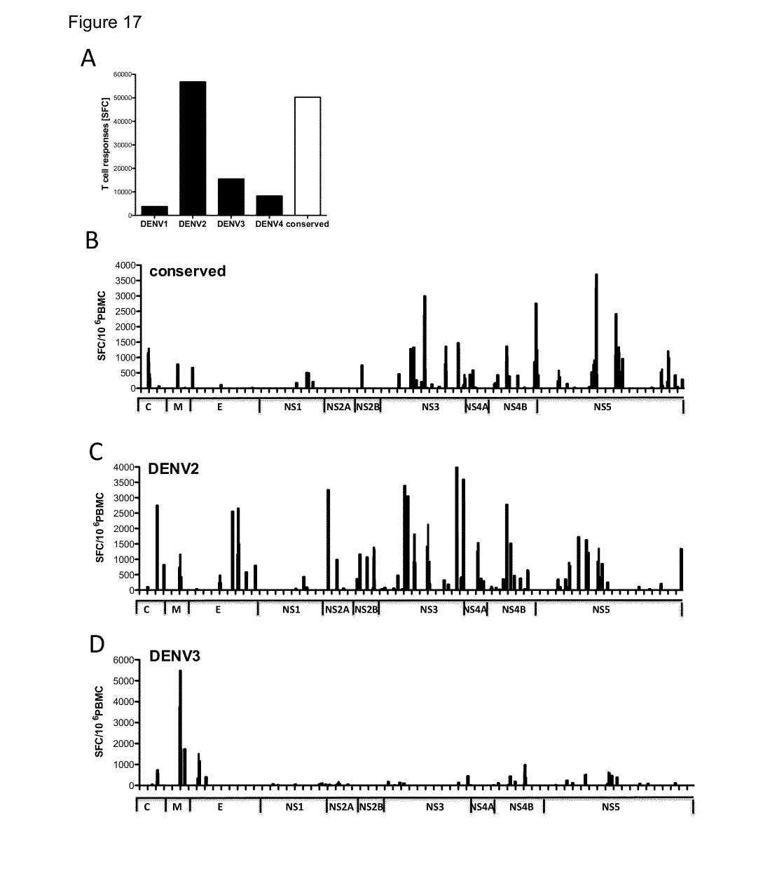

In the present section T cell reactivity was segregated as being directed against serotype specific sequences (found only in one serotype) or conserved/homologous sequences (sequences found in two or more serotypes, also allowing one residues substitutions to account for potential cross-reactivity of highly homologous sequences). Conserved sequences accounted for 37% of the overall responses (FIG. 17a). In terms of serotype specific responses, responses against DENV2 were by far most prevalent (42%), followed by DENV3 (12%), DENV4 (6%) and DENV1 (3%) (FIG. 17a).

Interestingly, the prominence of DENV2 responses is more marked at the level of T cell than at the level of antibodies (FIG. 15C). This may reflect that while DENV2 has historically been most prevalent in Sri Lanka, recent years have seen the appearance of new sub-strains of DEN3, and DENV1 has only recently appeared in this population.

To further investigate the dominance of DENV2 in the cohort serotype specific responses were aligned along the dengue polyprotein. Epitopes conserved between serotypes (FIG. 17b) were mostly derived from the highly conserved NS proteins. Interestingly, DENV2 specific responses were more evenly distributed across the polyprotein (FIG. 17c) while DENV3 specific responses mostly targeted the structural proteins (FIG. 17d).

### Example 21

This example includes a description of studies showing that Antigenic sin is not associated with differences in epitope avidity or multifunctionality.

The broad DENV2 and limited DENV3 specific response focused on surface proteins, might reflect lower immunogenicity of DENV3 as compared to DENV2. Alternatively, it might be that conserved epitopes dominate DENV3 responses, reflecting previous DENV2 infections and thus expansion of T cells recognizing conserved epitopes (antigenic sin).

To test this hypothesis, responses form donors either exhibiting responses against DENV3 (or DENV2) specific responses were compiled and used as an indicator of previous infection with either DENV2 (or DENV3). Overall, no difference in magnitude of responses was noted between the two groups if the total response to serotype specific and conserved epitopes per donor was compared. The average total response observed was 2819±1203 SFC for donors recognizing DENV2 specific epitopes and 2032±619 SFC for donors responding to DENV3 specific epitopes (p=0.61).

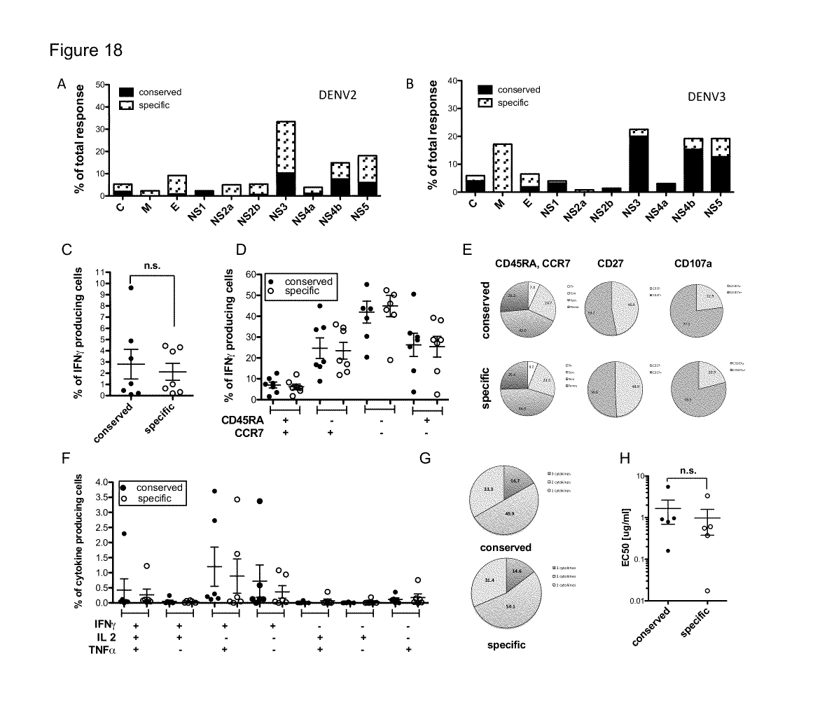

When responses were plotted as a function of the polyprotein position, consistent with the antigenic sin hypothesis it was noted that donors presumably infected by DENV2 responded utilizing both conserved and serotype-specific epitopes distributed along the entire sequence (FIG. 18A). In stark contrast, the lower serotype specific responses in donors presumably exposed to DENV3 were compensated by responses against conserved regions of the protein (FIG. 18B). These observations match the know historical prevalence of DENV serotypes circulating in Sri Lanka, and suggest that the differential focus of DENV2 versus DENV3 responses is the results of “antigenic sin”.

These observations allowed testing whether antigenic sin is associated with differential quality of responses. Accordingly, pools of epitopes corresponding to either serotype specific or conserved epitopes were tested by ICS assays in several representative donors. There was no appreciable difference in the magnitude, phenotype, and pattern of multi-functionality or avidity of the T cell responses between serotype specific and conserved responses (FIG. 18C-H, respectively).

### Example 22

This example includes a description of studies showing that low magnitude T cell responses are HLA-linked and associated with disease susceptibility.

Overall the results presented above suggest that antigenic sin does not significantly change or impair the quality of T cell responses in the general population of an endemic area. However, it possible that lower quality of responses might be present in the relatively few individuals that experience more severe clinical outcomes. Previous studies highlight that certain HLA alleles are associated with either increased or decreased risk of clinical manifestations. Those studies did not determine whether increased risk might be associated with a hyperactive T cell response, or rather a higher T cell response might have protective effects, leading to a decreased risk. In fact, since HLA class I restricted T cell responses have not been comprehensively mapped, it was unclear whether different HLA alleles were differentially associated with response frequency or magnitude.

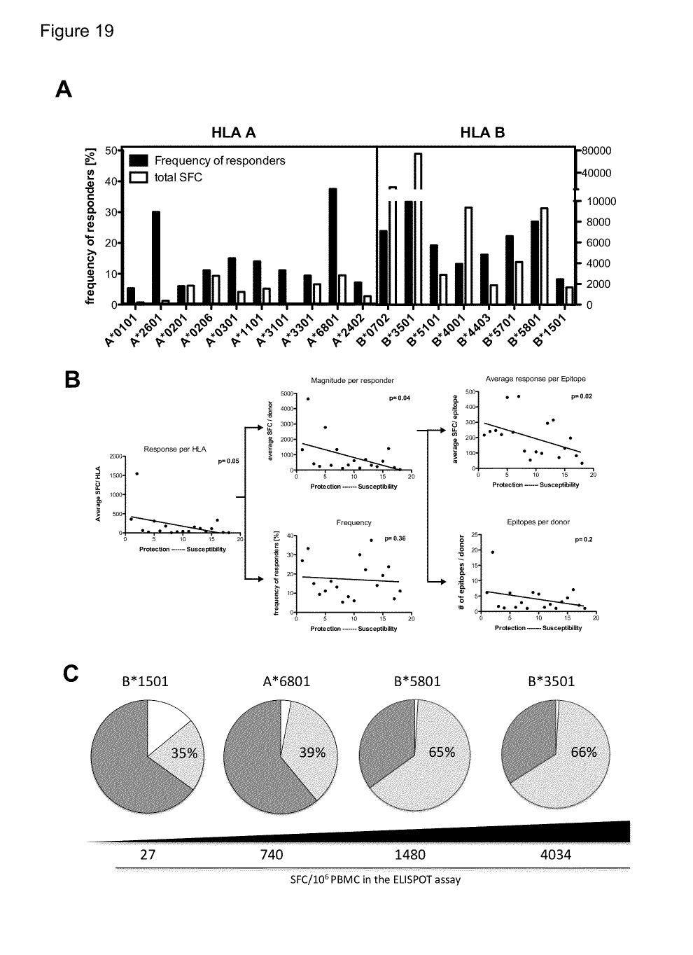

To address these points, HLA types expressed in our cohort were correlated with T cell responses. FIG. 19a shows the frequency (black bars) and the magnitude (white bars) of T cell responses sorted according to their restriction element. A wide variation in terms of frequency and magnitude was detected as a function of the different HLA alleles. Interestingly, certain alleles were associated with low response frequency and magnitude (A*0101, A*2401) while others were associated with high response frequency and magnitude (B*0702, B*3501). Still other alleles were associated with low frequency and high magnitude response (B*4001), while others were associated with high frequency and low magnitude response (A*2601). Overall HLA B restricted responses were of significantly higher magnitude but not frequency as compared to HLA A restricted responses.

To test whether T cell responses correlated with HLA associated disease susceptibility, all data available in the literature was compiled (15-21). For each of the studies, all investigated alleles were ranked according to their association with clinical manifestations (dengue fever [DF], dengue hemorrhagic fever [DHF] and dengue shock syndrome [DSS]; Table 9). A percentile ranking across all studies was calculated for the 18 alleles detected in significant frequencies in our own cohort and correlated the rankings with T cell responses (FIG. 19b).

When average magnitudes of HLA restricted responses were compared with disease susceptibility, weak T cell responses correlated with disease susceptibility (FIG. 19b, left panel; p=0.05). This correlation was accounted for, by the response magnitude rather than their frequency (FIG. 19b, middle panel, p=0.04). Further analysis revealed that the magnitude per epitope rather than the breadth of responses correlated best with disease susceptibility (FIG. 19b, right panel, p=0.02) and that low T cell responses are associated with disease susceptibility.

TABLE 9
Ranking of reported HLA associations from the literature

| Study | Malavige et al. Plos ONE 2011 | | | Stephens et al. Tissue Antigens 2002 | | | | Falcon et al 2009 Acta Tropica | | |
|---|---|---|---|---|---|---|---|---|---|---|
| Ethnic- | Sri Lankan | Sri Lankan | Sri Lankan | Thai | Thai | Thai | Thai | Mexican Mest | Mexican Mest | Mexican Mest |
| ity Pa- | Normal vs. | Normal vs | Normal vs. | Normal vs | Normal vs | Normal vs | Normal vs | Normal vs | Normal vs | Normal vs |
| rameter | acute DHF | PD/SD | DSS | PD_DF | PD_DHF | SD_DF | SD_DHF | DF/DHF | DF | DHF |
| Allele | Rank | Rank | Rank | Rank | Rank | Rank | Rank | Rank | Rank | Rank |
| A*01 | 23 | 7 | 10 | 6 | 1 | 5 | 5 | 9 | 7 | 6 |
| A*02 | 11 | 4 | 8 | 21 | 20 | 24 | 23 | 9 | 7 | 6 |
| A*03 | 21 | | | 2 | 1 | 4 | 3 | | | |
| A*11 | 15 | 3 | 9 | 6 | 21 | 7 | 22 | | | |
| A*23 | | | | | | | | | | |
| A*24 | 22 | 12 | 3 | 21 | 5 | 8 | 8 | 9 | 7 | 6 |
| A*25 | | | | | | | | | | |
| A*26 | 9 | 5 | 1 | | | | | | | |
| A*29 | | | | | | | | | | |
| A*30 | 5 | | | | | | | | | |
| A*31 | 6 | | 13 | | | | | | | |
| A*32 | | | | | | | | | | |
| A*33 | 13 | 6 | 4 | 5 | 23 | 8 | 6 | | | |
| A*34 | | | | | | | | | | |
| A*36 | | | | | | | | | | |
| A*66 | | | | | | | | | | |
| A*68 | 1 | | | | | | | 9 | 7 | 6 |
| A*74 | | | | | | | | | | |
| B*07 | 16 | 2 | 2 | 8 | 7 | 9 | 9 | 17 | 15 | 14 |
| B*08 | 17 | | | | | | | | | |
| B*13 | 26 | | | 8 | 7 | 9 | 9 | | | |
| B*14 | | | | | | | | 6 | 2 | 12 |
| B*15 | 14 | 11 | 11 | | | | | 5 | 5 | 2 |
| B*18 | 24 | | | 8 | 7 | 9 | 9 | 6 | 14 | |
| B*27 | 8 | | | 8 | 7 | 9 | 9 | | | |
| B*35 | 25 | 10 | 7 | 8 | 7 | 9 | 9 | 2 | 1 | 1 |
| B*37 | 20 | | | | | | | | | |
| B*38 | 3 | | | 8 | 7 | 9 | 9 | | | |
| B*39 | | | | 8 | 7 | 9 | 9 | 14 | 12 | 11 |
| B*40 | 7 | 8 | 5 | | | | | 4 | 4 | 4 |
| B*41 | | | | | | | | 18 | 18 | |
| B*42 | | | | | | | | | | |
| B*44 | 18 | 9 | | 4 | 22 | 6 | 3 | 3 | 3 | 4 |
| B*45 | | | | | | | | | | |
| B*46 | | | | 21 | 24 | 23 | 24 | | | |
| B*47 | | | | | | | | | | |
| B*48 | | | | | | | | 15 | 6 | 13 |
| B*49 | | | | | | | | 6 | 13 | |
| B*5 | | | | 25 | 25 | 25 | 25 | | | |
| B*50 | | | | | | | | | | |
| B*51 | 12 | 1 | 12 | | | | | 1 | | 2 |
| B*52 | 19 | | | | | | | 19 | 17 | 15 |
| B*53 | | | | | | | | | | |
| B*54 | | | | | | | | | | |
| B*55 | 4 | | | 8 | 7 | 9 | 9 | | | |
| B*56 | | | | 8 | 7 | 9 | 9 | | | |
| B*57 | 10 | | | 8 | 7 | 9 | 9 | 16 | 15 | |
| B*58 | 2 | | | 8 | 7 | 9 | 9 | | | |
| B*60 | | | | 8 | 7 | 9 | 9 | | | |
| B*62 | | | | 8 | 7 | 9 | 9 | | | |
| B*75 | | | | 8 | 7 | 9 | 9 | | | |
| B*76 | | | | 1 | 1 | 1 | 2 | | | |
| B*77 | | | | 3 | 1 | 2 | 1 | | | |
| B*78 | | | | | | | | | | |
| n = | 26 | 12 | 13 | 25 | 25 | 25 | 25 | 19 | 18 | 15 |

| | | | | | | Lan et al. | Sierra et al. | |
|---|---|---|---|---|---|---|---|---|
| | | | | | | PloS NTDs | Human | |
| Study | Loke et al. J | Appanna et al. PloS ONE 2010 | | | | 2008 | Imm 2007 | |
| Ethnic- | Infect Dis | total | Malay | Chinese | Indian | Vietnam | Cuba | Study |
| ity Pa- | 2001 | Normal vs | Normal vs | Normal vs | Normal vs | normal vs. | Normal vs. | Ethnicity |
| rameter | Vietnam | DF/DHF | DF/DHF | DF/DHF | DF/DHF | DHF/DSS | DENV2 | Parameter |
| Allele | Rank | Rank | Rank | Rank | Rank | Rank | Rank | Allele |
| A*01 | | 9 | 18 | 1 | 14 | 1 | 17 | A*01 |
| A*02 | 2 | 14 | 19 | 15 | 13 | 1 | 30 | A*02 |
| A*03 | | 3 | 5 | 1 | 12 | | 19 | A*03 |
| A*11 | 4 | 16 | 8 | 21 | 25 | 1 | 2 | A*11 |
| A*23 | | | | | | | 12 | A*23 |
| A*24 | 5 | 22 | 14 | 24 | 15 | 20 | 28 | A*24 |
| A*25 | | | | | | | 39 | A*25 |
| A*26 | | 28 | | | 15 | 1 | 39 | A*26 |
| A*29 | 3 | | | | 1 | 1 | 19 | A*29 |
| A*30 | | 33 | 23 | | | 1 | 35 | A*30 |
| A*31 | | 24 | | 8 | 29 | | 40 | A*31 |
| A*32 | | 11 | | | 1 | | 19 | A*32 |
| A*33 | 1 | 9 | 16 | 10 | 1 | 1 | 10 | A*33 |
| A*34 | | 28 | 20 | | | | 30 | A*34 |
| A*36 | | | | | | | 36 | A*36 |
| A*66 | | | | | | | 2 | A*66 |
| A*68 | | 28 | | | 24 | | 19 | A*68 |
| A*74 | | | | | | | 29 | A*74 |
| B*07 | | 18 | 16 | 16 | 15 | 1 | 12 | B*07 |
| B*08 | | | | | 1 | | 1 | B*08 |
| B*13 | | 28 | 26 | 12 | 27 | 1 | 12 | B*13 |
| B*14 | | 11 | | 16 | | | 7 | B*14 |
| B*15 | | 21 | 20 | 23 | 11 | 1 | 37 | B*15 |
| B*18 | | 1 | 1 | 16 | 1 | | 19 | B*18 |
| B*27 | | 7 | 1 | 25 | 1 | | 2 | B*27 |
| B*35 | | 6 | 6 | 11 | 15 | 1 | 12 | B*35 |
| B*37 | | 26 | 9 | | | | 7 | B*37 |
| B*38 | | 19 | 23 | 6 | | 1 | 19 | B*38 |
| B*39 | | 2 | 1 | 8 | 1 | | 37 | B*39 |
| B*40 | | 14 | 15 | 14 | 15 | 1 | 19 | B*40 |
| B*41 | | | | | | | 10 | B*41 |
| B*42 | | 1 | 1 | 1 | 1 | | 19 | B*42 |
| B*44 | | 5 | 9 | 1 | 15 | 1 | 17 | B*44 |
| B*45 | | | | | | | 30 | B*45 |
| B*46 | | 19 | 20 | 16 | | 1 | | B*46 |
| B*47 | | 11 | | 1 | | | | B*47 |
| B*48 | | 33 | 25 | | | | | B*48 |
| B*49 | | | | | | | 19 | B*49 |
| B*5 | | | | | | | | B*5 |
| B*50 | | | | | | | 30 | B*50 |
| B*51 | | 17 | 9 | 12 | 26 | 1 | 7 | B*51 |
| B*52 | | 23 | 9 | | 23 | | | B*52 |
| B*53 | | | | | | | 12 | B*53 |
| B*54 | | 32 | | 22 | | | | B*54 |
| B*55 | | 7 | 9 | 16 | 1 | 1 | | B*55 |
| B*56 | | 24 | 1 | | 15 | | | B*56 |
| B*57 | | 26 | | 1 | 27 | 1 | 30 | B*57 |
| B*58 | | 4 | 7 | 7 | 1 | 1 | 2 | B*58 |
| B*60 | | | | | | | | B*60 |
| B*62 | | | | | | | | B*62 |
| B*75 | | | | | | | | B*75 |
| B*76 | | | | | | | | B*76 |
| B*77 | | | | | | | | B*77 |
| B*78 | | | | | | | 1 | B*78 |
| n = | 5 | 34 | 26 | 25 | 29 | 20 | 40 | |

### Example 22

This example includes a description of studies showing that higher T cell responses are associated with multifunctional T cell responses.

It was reasoned that a possible explanation for the observations above would be that certain alleles and epitopes are associated with higher magnitude responses, which are in turn associated with higher degrees of multi-functionality, and thus might be most beneficial in protecting from disease. A detailed analysis of cytokines produced by DENV specific T cells revealed indeed that stronger responses are associated with multifunctional T cell responses (FIG. 19c).

### Example 23

This example includes a discussion of the foregoing data and conclusions based upon the data.

The role of CD8⁺ T cells in dengue infection is not yet fully understood. Several reports highlight how CD8⁺ T cell responses are detected in humans that have recovered from infection and disease. In animal models, CD8⁺ T cells are associated with protection from infection and disease (28, 29). According to one hypothesis, however, T cells can also be detrimental, because of antigenic sin (30, 31). While definitions vary somewhat, antigenic sin is usually defined as the imprint of responses associated with infection of a given serotype, which shapes and biases responses following infection with a different serotype (32). Herein, it is shown that while “antigenic sin” can be detected in the general population from the Colombo (Sri Lanka) endemic region, its functional consequence is not generating a less functional response. Rather it results in honing responses towards recognition of conserved viral sequences, by highly multifunctional CD8⁺ T cells.

It is further shown herein that different HLA alleles are associated with differential magnitude of anti DENV responses, and that HLA alleles known to be associated with increased risk of severe DENV disease (14, 32) are also associated with weaker CD8⁺ responses. Finally, also in this case the higher magnitude responses are associated with more poly-functional CD8⁺ T cells. Taken together, these data support a protective role for CD8⁺ T cells, and dispel the notion that DENV associated pathogenicity is resulting from “antigenic sin”.

A major obstacle to clearly elucidate the function of CD8⁺ T cell responses in humans has been the somewhat anectodical evidence available to date. Only a handful of epitopes have been defined, and lack of knowledge of T cell epitopes presented by common MHC alleles expressed by populations in endemic areas allowed only a very episodical evaluation of responses. In this study, the present inventors report for the first time a comprehensive ex vivo characterization of HLA restricted DENV specific T cell memory in the general population of Sri Lanka, where dengue is endemic. A total of 250 different individuals were studied and in 90% of the donors 3 or more of the HLA molecules expressed by atypical heterozygous individual at the A and B locus, were addressed by the present approach. These efforts led to the identification of 408 unique CD8⁺ T cell epitopes. The novelty and impact of these results can be appreciated by noting that previous studies, as compiled by the Immune Epitope Database (IEDB) (www.iedb.org) identified a total of 82 unique CD8⁺ T cell epitopes. This reflects an almost 6 fold expansion in knowledge about DENV specific T cell epitopes.

The present results are consistent with previous studies that have emphasized NS3 as a major T cell target (30, 33). However a more complete and through characterization demonstrates that T cell epitopes originate from all of the ten dengue virus proteins (capsid (C), pre-membrane (prM/M), envelope (E), and the seven non-structural proteins NS1, NS2A, NS2B, NS3, NS4A, NS4B and NS5). Besides NS3, other non-structural proteins such as NS4B and NS5 were also highly immunogenic. These results have implication in terms of DENV vaccination strategies, and specifically suggest that inclusion of NS3, NS4, and NS5 will be necessary to replicate the natural CD8⁺ adaptive immunity.

Likewise, to clearly monitor responses and study DENV-associated immunopathology, a broad set of epitopes and antigens is required. To map epitopes for the various MHC class I alleles, relatively large amounts of blood were necessary. The 408 epitopes identified herein provide a tool to further investigate the CD8⁺ responses in smaller sample volumes available from acute fever patients and/or children experiencing dengue fever. This will allow further characterization of CD8′ T cell responses in clinically defined cohorts.

Along those lines the present inventors have been able to demonstrate that reactivity clusters in specific regions of these proteins with some of them being recognized across serotypes and being restricted by multiple HLA alleles. This clustering of epitopes in small amino-acid stretches has also been shown in a previous smaller scale study conducted in Vietnamese adults for a certain region of the NS3 protein (34) suggesting that this phenomenon is not limited to a certain ethnicity. Clustering of about 50% of the response in about 25 main antigenic regions promises to simplify the development of reagents to be utilized for diagnostic purposes and for vaccine evaluations. A number of techniques are of potential interest, such as the use of epitopes pools in ICS assays, Q-dots and CyTOF (35, 36)

The epitopes were subdivided in those conserved or homologous amongst different serotypes, and those uniquely associated with one particular serotype, in that no identical or highly homologous sequence could be found in other serotypes. This allowed in turn identifying which donors in the general populations had been infected at some point in time by a given serotype. Consistent with the serological evidence derived from the same population, it was found that DENV2 and 3 were most prevalent, and that the magnitude of the responses did not differ appreciably between DENV 2 and DENV3 responses, defined for each individual as the sum total of responses directed against serotype specific and conserved/homologous epitopes.

According to the known epidemiologic history of DENV infection in Sri Lanka both DENV 2 and 3 are prevalent (25) and a new clade of DENV3 is associated with a more recent spread, associated with a recrudescence of severe DENV cases in Sri (26). Consistent with this data and with the antigenic sin notion, the present inventors see that while responses to both serotype specific and conserved epitopes are observed for DENV2, conserved epitopes dominate DENV3 responses, likely reflecting previous DENV2 infections and thus expansion of T cells recognizing conserved epitopes.

This observation provided a rare opportunity to test, at the level of a global population from an endemic region, whether antigenic sin was associated with a lower quality of responses. However no significant difference in the magnitude, phenotype, pattern of multi-functionality or avidity of the T cell responses between serotype specific and conserved/homologous responses. While to a certain extent a semantic issue, perhaps the term antigenic “sin” is misleading and because of an implicit negative connotation, inaccurate. The evidence revealed by the current study is more aptly described by maturation of the specificity of CD8⁺ T cell responses, induced by repeated stimulation with only partially cross-reactive antigens.

The present observations are in contrast to other studies that reported that humans who contract a secondary dengue infection may generate a CD8⁺ T cell response that reacts better with a previously encountered dengue serotype (31). This altered peptide ligand (APL) hypothesis is also in contrast with the observation that heterologous T cell responses are not needed to produce DHF in infants. Exactly the same severe vascular permeability clinical syndrome and the same concentrations of cytokines in blood are produced during primary dengue immune responses in infants, as are in children with secondary dengue infections (37). Another study has shown a temporal mismatch between the CD8⁺ T cell response and commencement of capillary leakage, suggesting that CD8⁺ T cells are not responsible for early triggering of capillary leakage in children with DHF (38). The present inventors were able to show that dengue specific T cells recognize a wide repertoire of epitopes. To explain the association with DHF and heterologous infection by the APLs hypothesis, would require that aberrant T cell responses would consistently occur across the whole dengue specific epitope repertoire in one donor, which seems unlikely. The present data relates to the feature of immunity present in the general population, and does not directly address the features present at the time of acute manifestations such as DHF and DHSS. While the present data demonstrates that secondary infection does not negatively affect the response at the level of the general population, without being limited to any particular theory it may be that it does so in the few individuals that develop DHF or DSS.

The present inventors next examined the responses associated with different HLA alleles present in the general population of Sri Lanka. Multiple HLA Class I alleles have been associated with either protection or susceptibility to dengue infection in several ethnicities so far (17, 18, 20, 21). The A*24 allele is associated with susceptibility to disease not only in secondary but also primary infections with dengue virus (17, 19) and a recent report associated the A*01 allele with susceptibility to DHF in the Brazilian population (39). In contrast the B*3501 allele was negatively associated with symptomatic disease in Mexican dengue fever patients (20) and one study reported that HLA A and not HLA B genes are associated with DHF (17). However, until now it was not clear whether this association reflected differences in the corresponding T cell responses. In the present study A*0101 and A*2402 restricted responses were amongst the lowest responses observed in terms of frequencies as well as magnitude, whereas B*3501 restricted responses were associated with high magnitude responses. More extensive correlations demonstrate that HLA class I responses are positively correlated with protection from severe disease. This data demonstrates that severe disease is not to be ascribed to an over-active CD8⁺ T cell response, and rather a strong HLA class I restricted response is a positive correlate of protection.

Regarding the mechanism that mediates the positive association of HLA class I with protection from severe disease, the present inventors found that the best correlate of protection is the magnitude of response associated with individual epitopes. In turn, strong responses were associated with multi-functionality, which has been shown to be a main predictor of immunity in other viral systems (40, 41). Higher frequencies of dengue virus specific IFNγ producing T cells are present in children who subsequently developed subclinical infection, compared with those who develop symptomatic secondary dengue virus infection (42). Furthermore, HIV specific HLA B*27 and HLA B*57 restricted T cell responses are not suppressed by regulatory T cells and thus maintain proliferative capacity over the course of chronic infection (43). It remains to be determined if similar mechanisms underlie the observed variations in HLA restricted responses against infection with dengue virus.

The data presented herein suggests a protective role of strong CD8⁺ T cell responses, and does not support a causative role in the induction of severe disease by heterologous infection. Thus, the mechanism underlying severe disease induction remains open. Without being limited to any particular theory, HLA class II responses might be linked to immunopathology. Serotype cross-reactive antibodies may exacerbate disease by antibody-dependent enhancement (ADE) of infection (11, 12). It is possible that ADE increases processing and presentation of structural proteins through the exogenous presentation pathway, which might increase CD4⁺ T cell responses and immunopathology associated with lymphokine production.

In conclusion, the present inventors present for the first time a comprehensive analysis of functional T cell memory against dengue virus and were able to correlate this with HLA alleles expressed in the very same donors. The results support a protective rather than pathogenetic role for DENV-specific, HLA class I restricted responses in humans.

Dengue T cell epitopes identified by the proteome-wide screen described herein are listed in Table 10 (SEQ ID NOs: 813-1163).

TABLE 10
Dengue Antigenic Regions

| | Sequence | | | | | | | | | |
|---|---|---|---|---|---|---|---|---|---|---|
| | (SEQ ID | | | | | | | | | |
| | NOs: 813- | # | | | | | | | Total | Total |
| | 1163, in | donors | | | | | Align- | Align- | Response | response |
| Cluster | order of | re- | Super- | HLA | | Sero- | ment | ment | per | per |
| No. | appearance) | sponded | typte | allele | length | type | start | end | epitope | Cluster |
| 1 | AAVLLLVTHY | 2 | B58 | HLA-B-5801 | 10 | DENV3 | 2356 | 2365 | 393.3 | 393.3 |
| 2 | ALRGLPIRY | 2 | A3 | HLA-A-0301 | 9 | DENV2 | 1712 | 1720 | 918.3 | 918.3 |
| 3 | AMALSIVSLF | 1 | B62 | HLA-B-1501 | 10 | DENV1 | 1297 | 1306 | 68.3 | 116.7 |
| 3 | MALSIVSLF | 1 | B7 | HLA-B-5101 | 9 | DENV1 | 1298 | 1306 | 48.3 | |
| 4 | APFESEGVL | 2 | B7 | HLA-B-0702 | 9 | DENV4 | 3387 | 3395 | 401.7 | 401.7 |
| 5 | APIMDEEREI | 1 | B7 | HLA-B-5301 | 10 | DENV2 | 1808 | 1817 | 206.7 | 206.7 |
| 6 | APTRVVAAEM | 8 | B7 | HLA-B-3501, | 10 | DENV2, | 1700 | 1709 | 1623.3 | 2230.0 |
| | | | | B*0702 | | 3, 4 | | | | |
| 6 | APTRVVASEM | 5 | B7 | HLA-B-0702 | 10 | DENV1 | 1700 | 1709 | 606.7 | |
| 7 | ASSMVNGVVK | 1 | B58 | HLA-B-5801 | 10 | DENV1 | 2812 | 2821 | 116.7 | 523.3 |
| 7 | ASSMVNGVVR | 2 | B58 | HLA-B-5701 | 10 | DENV1, | 2812 | 2821 | 406.7 | |
| | | | | | | 2 | | | | |
| 8 | ASSVLLWMAS | 1 | B58 | HLA-B-5801 | 10 | DENV1 | 2186 | 2195 | 68.3 | 68.3 |
| 9 | ATGPILTLW | 2 | B58 | HLA-B-5801 | 9 | DENV4 | 2444 | 2452 | 938.3 | 3088.8 |
| 9 | ATGPISTLW | 2 | B58 | HLA-B-5801 | 9 | DENV2 | 2444 | 2452 | 235.5 | |
| 9 | ATGPITTLW | 1 | B58 | HLA-B-5801 | 9 | DENV3 | 2444 | 2452 | 505.0 | |
| 9 | ATGPLTTLW | 1 | B58 | HLA-B-5801 | 9 | DENV1 | 2444 | 2452 | 275.0 | |
| 9 | ATGPVLTLW | 2 | B58 | HLA-B-5801 | 9 | DENV4 | 2444 | 2452 | 390.0 | |
| 9 | LATGPVLTLW | 1 | B7 | HLA-B-5301 | 10 | DENV4 | 2443 | 2452 | 745.0 | |
| 10 | ATYGWNLVK | 1 | A3 | HLA-A-1101 | 9 | DENV1, | 2612 | 2620 | 28.3 | 291.7 |
| | | | | | | 4 | | | | |
| 10 | MATYGWNLVK | 1 | A3 | HLA-A-1101 | 10 | DENV1 | 2611 | 2620 | 26.7 | |
| 10 | MSTYGWNIVK | 1 | A3 | HLA-A-1101 | 10 | DENV3, | 2611 | 2620 | 236.7 | |
| | | | | | | 1 | | | | |
| 11 | AVQTKPGLFK | 1 | A3 | HLA-A-1101 | 10 | DENV2 | 1585 | 1594 | 63.3 | 63.3 |
| 12 | CLIPTAMAF | 1 | B62 | HLA-B-1501 | 9 | DENV4 | 108 | 116 | 66.7 | 2815.0 |
| 12 | MLIPTAMAF | 7 | B7 | HLA-B-3501 | 9 | DENV2 | 108 | 116 | 2748.3 | |
| 13 | CLMMMLPATL | 1 | A2 | HLA-A-0201 | 10 | DENV3 | 105 | 114 | 78.3 | 78.3 |
| 14 | CPTQGEATL | 1 | B7 | HLA-B-3501 | 9 | DENV1 | 355 | 363 | 295.0 | 680.0 |
| 14 | CPTQGEAVL | 2 | B7 | HLA-B-3501 | 9 | DENV3 | 355 | 363 | 385.0 | |
| 15 | CPTQGEPSL | 1 | B7 | HLA-B-5301 | 9 | DENV2 | 355 | 363 | 35.0 | 35.0 |
| 16 | DPASIAARGY | 9 | B7 | HLA-B-3501 | 10 | DENV1, | 1768 | 1777 | 2383.3 | 2383.3 |
| | | | | | | 2, 3 | | | | |
| 17 | DPIPYDPKF | 1 | B7 | HLA-B-3501 | 9 | DENV2 | 2403 | 2411 | 38.3 | 38.3 |
| 18 | DPRRCLKPV | 2 | B7 | HLA-B-0702 | 9 | DENV1, | 1902 | 1910 | 556.7 | 556.7 |
| | | | | | | 3, 4 | | | | |
| 19 | DTTPFGQQR | 8 | A3 | HLA-A-6801, | 9 | DENV1, | 2840 | 2848 | 3260.0 | 3260.0 |
| | | | | A*3301 | | 2, 3, | | | | |
| | | | | | | 4 | | | | |
| 20 | DYMPSMKRFR | 1 | A3 | HLA-A-3301 | 10 | DENV2, | 3377 | 3386 | 285.0 | 285.0 |
| | | | | | | 3 | | | | |
| 21 | EAVEDSRFWE | 1 | B58 | HLA-B-5801 | 10 | DENV2 | 2919 | 2928 | 248.3 | 248.3 |
| 22 | EENMDVEIW | 1 | B44 | HLA-B-4403 | 9 | DENV1 | 2052 | 2060 | 448.3 | 448.3 |
| 23 | EERDIPERSW | 1 | B44 | HLA-B-4403 | 10 | DENV1, | 1813 | 1822 | 133.3 | 133.3 |
| | | | | | | 3 | | | | |
| 24 | EFKEFAAGR | 1 | A3 | HLA-A-3301 | 9 | DENV1 | 2087 | 2095 | 26.7 | 66.7 |
| 24 | EFKEFAAGRR | 1 | A3 | HLA-A-3301 | 10 | DENV1 | 2087 | 2096 | 40.0 | |
| 25 | ELMRRGDLPV | 1 | A2 | HLA-A-0201 | 10 | DENV1, | 2013 | 2022 | 123.3 | 123.3 |
| | | | | | | 3, 4 | | | | |
| 26 | EPDYEVDEDI | 1 | B7 | HLA-B-5301 | 10 | DENV4 | 1651 | 1660 | 78.3 | 78.3 |
| 27 | EPGQLKLNWF | 5 | B7 | HLA-B-3501 | 10 | DENV2 | 664 | 673 | 581.7 | 581.7 |
| 28 | EPGVVSPTSY | 1 | B7 | HLA-B-3501 | 10 | DENV3 | 2264 | 2274 | 431.7 | 431.7 |
| 29 | EPIEGKVVQY | 1 | B7 | HLA-B-3501 | 10 | DENV3 | 404 | 413 | 403.3 | 403.3 |
| 30 | EPISYDPKF | 1 | B7 | HLA-B-5301 | 9 | DENV4 | 2403 | 2411 | 340.0 | 340.0 |
| 31 | EPKEGTKKLM | 3 | B7 | HLA-B-3501 | 10 | DENV2 | 2860 | 2869 | 928.3 | 928.3 |
| 32 | ESSSNPTIEE | 1 | B58 | HLA-B-5801 | 10 | DENV4 | 2644 | 2653 | 408.3 | 408.3 |
| 33 | ETACLGKAY | 3 | A1 | HLA-A-2601 | 9 | DENV3, | 3246 | 3254 | 240.0 | 771.6 |
| | | | | | | 4 | | | | |
| 33 | ETACLGKAYA | 1 | A1 | HLA-A-2601 | 10 | DENV3, | 3246 | 3255 | 73.3 | |
| | | | | | | 4 | | | | |
| 33 | ETACLGKSY | 4 | A1 | HLA-A-2601 | 9 | DENV1, | 3246 | 3254 | 321.7 | |
| | | | | | | 2 | | | | |
| 33 | KETACLGKSY | 1 | B44 | HLA-B-4403 | 10 | DENV2 | 3245 | 3254 | 23.3 | |
| 33 | RETACLGKAY | 1 | B44 | HLA-B-4403 | 10 | DENV3 | 3245 | 3254 | 60.0 | |
| 33 | RETACLGKSY | 1 | B44 | HLA-B-4403 | 10 | DENV1 | 3245 | 3254 | 53.3 | |
| 34 | ETTHHAVSR | 1 | A3 | HLA-A-6801 | 9 | DENV3 | 2544 | 2552 | 30.0 | 80.0 |
| 34 | ETTKHAVSR | 1 | A3 | HLA-A-3301 | 9 | DENV1 | 2544 | 2552 | 50.0 | |
| 35 | FAGPVSQHNY | 3 | B7 | HLA-B-3501 | 10 | DENV2 | 1023 | 1032 | 428.3 | 428.3 |
| 36 | FASGRKSITL | 1 | B58 | HLA-B-5801 | 10 | DENV4 | 2091 | 2100 | 26.7 | 26.7 |
| 37 | FGAIYGAAF | 3 | B7 | HLA-B-3501 | 9 | DENV2 | 721 | 729 | 798.3 | 798.3 |
| 38 | FLMVLLIPEP | 1 | A2 | HLA-A-0201 | 10 | DENV1, | 2210 | 2219 | 28.3 | 28.3 |
| | | | | | | 4 | | | | |
| 39 | FMALVAFLRF | 1 | B7 | HLA-B-3501 | 10 | DENV2 | 48 | 57 | 105.0 | 105.0 |
| 40 | FMKDGRSLVV | 1 | B7 | HLA-B-5101 | 10 | DENV4 | 3213 | 3222 | 66.7 | 66.7 |
| 41 | FPQSNAPIMD | 4 | B7 | HLA-B-3501 | 10 | DENV2 | 1803 | 1812 | 716.7 | 716.7 |
| 42 | FPVSIPITAA | 1 | B7 | HLA-B-3501 | 10 | DENV2 | 1457 | 1466 | 71.7 | 71.7 |
| 43 | FSRENSLSGV | 1 | B7 | HLA-B-5101 | 10 | DENV1, | 2993 | 3002 | 101.7 | 101.7 |
| | | | | | | 2 | | | | |
| 44 | FTILALFLAH | 8 | B7 | HLA-B-3501 | 10 | DENV3 | 248 | 257 | 1715.0 | 1715.0 |
| 45 | FTIMAAILAY | 2 | B7 | HLA-B-3501 | 10 | DENV2 | 248 | 257 | 675.0 | 738.3 |
| 45 | TIMAAILAY | 1 | B7 | HLA-B-3501 | 9 | DENV2 | 249 | 257 | 20.0 | |
| 45 | TLMAAILAY | 1 | B62 | HLA-B-1501 | 9 | DENV2 | 249 | 257 | 43.3 | |
| 46 | FTMGVLCLAI | 1 | A2 | HLA-A-0201 | 10 | DENV3 | 1136 | 1145 | 58.3 | 58.3 |
| 47 | FTMRHKKATY | 4 | B7 | HLA-B-3501 | 10 | DENV2 | 2738 | 2747 | 1723.3 | 1723.3 |
| 48 | FTNMEAQLVR | 1 | B7 | HLA-B-3501 | 10 | DENV3 | 3107 | 3116 | 88.3 | 88.3 |
| 49 | GAMLFLISGK | 1 | A3 | HLA-A-1101 | 10 | DENV3 | 2163 | 2172 | 23.3 | 23.3 |
| 50 | GASKRSWPLN | 1 | B58 | HLA-B-5801 | 10 | DENV4 | 1343 | 1352 | 51.7 | 51.7 |
| 51 | GEARKTFVDL | 3 | B44 | HLA-B-4001 | 10 | DENV2 | 2005 | 2014 | 1405.0 | 5005.0 |
| 51 | GEARKTFVEL | 3 | B44 | HLA-B-4001 | 10 | DENV1 | 2005 | 2014 | 1330.0 | |
| 51 | GEQRKTFVEL | 2 | B44 | HLA-B-4001 | 10 | DENV4 | 2005 | 2014 | 1050.0 | |
| 51 | GESRKTFVEL | 3 | B44 | HLA-B-4001 | 10 | DENV3 | 2005 | 2014 | 1220.0 | |
| 52 | GEFRLRGEQR | 3 | B44 | HLA-B-4001 | 10 | DENV4 | 1999 | 2008 | 1373.3 | 1373.3 |
| 53 | GEGLHKLGY | 2 | B44 | HLA-B-4403 | 9 | DENV1, | 3004 | 3012 | 413.3 | 413.3 |
| | | | | | | 3 | | | | |
| 54 | GEVGAIALDF | 1 | B44 | HLA-B-4403 | 10 | DENV1 | 1598 | 1607 | 101.7 | 101.7 |
| 55 | GFFLRKLTSR | 1 | A3 | HLA-A-3301 | 10 | DENV3 | 1218 | 1227 | 81.7 | 81.7 |
| 56 | GGWRLSAQW | 1 | B58 | HLA-B-5701 | 9 | DENV3 | 1558 | 1566 | 20.0 | 20.0 |
| 57 | GMGEAAAIF | 1 | B62 | HLA-B-1501 | 9 | DENV1 | 1783 | 1791 | 23.3 | 23.3 |
| 58 | GPGHEEPIPM | 3 | B7 | HLA-B-3501 | 10 | DENV1, | 2602 | 2611 | 235.0 | 235.0 |
| | | | | | | 2, 4 | | | | |
| 59 | GPLVAGGLL | 3 | B7 | HLA-B-0702 | 9 | DENV2, | 1377 | 1385 | 748.3 | 748.3 |
| | | | | | | 3 | | | | |
| | | | | HLA-B-0702 | | | | | | |
| 60 | GPMKLVMAF | 2 | B7 | HLA-B-5301 | 9 | DENV1, | 43 | 51 | 789.7 | 1148.0 |
| | | | | | | 3 | | | | |
| 60 | GPMKLVMAFI | 1 | B7 | HLA-B-0702 | 10 | DENV1, | 43 | 52 | 358.3 | |
| | | | | | | 3 | | | | |
| 61 | GPSLRTTTV | 1 | B7 | HLA-B-0702 | 9 | DENV1, | 1071 | 1079 | 213.3 | 213.3 |
| | | | | | | 3 | | | | |
| 62 | GPWHLGKLEL | 2 | B7 | HLA-B-0702 | 10 | DENV1, | 1042 | 1051 | 495.0 | 585.0 |
| | | | | | | 3 | | | | |
| 62 | GPWHLGKLEM | 3 | B7 | HLA-B-3501 | 10 | DENV2 | 1042 | 1051 | 90.0 | |
| 63 | GSSKIRWIVE | 2 | B58 | HLA-B-5801 | 10 | DENV4 | 2553 | 2562 | 111.7 | 111.7 |
| 64 | GTSGSPIIDK | 3 | A3 | HLA-A-1101 | 10 | DENV2 | 1610 | 1619 | 475.0 | 516.7 |
| 64 | TSGSPIIDK | 1 | A3 | HLA-A-1101 | 9 | DENV2 | 1611 | 1619 | 41.7 | |
| 65 | GTSGSPIVNR | 1 | B58 | HLA-B-5701 | 10 | DENV1 | 1610 | 1619 | 28.3 | 28.3 |
| 66 | HALLATSIF | 2 | B7 | HLA-B-3501 | 9 | DENV1 | 3056 | 3064 | 80.0 | 80.0 |
| 67 | HMIAGVLFTF | 1 | B62 | HLA-B-1501 | 10 | DENV3 | 1159 | 1168 | 45.0 | 45.0 |
| | | | | HLA-B-3501 | | | | | | |
| 68 | HPASAWTLY | 3 | B7 | HLA-B-5301 | 9 | DENV1, | 2280 | 2288 | 281.7 | 810.0 |
| | | | | | | 3 | | | | |
| 68 | RPASAWTLYA | 2 | B7 | HLA-B-0702 | 10 | DENV1, | 2280 | 2289 | 528.3 | |
| | | | | | | 2, 3 | | | | |
| 69 | HPGAGKTKRY | 10 | B7 | HLA-B-3501 | 10 | DENV2 | 1672 | 1681 | 3046.7 | 3046.7 |
| 70 | HPGFTILALF | 9 | B7 | HLA-B-3501 | 10 | DENV3 | 245 | 254 | 3763.3 | 3763.3 |
| | | | A2 | HLA-A-0206 | | | | | | |
| 71 | HQLWATLLSL | 2 | B62 | HLA-B-1501 | 10 | DENV1 | 1271 | 1280 | 601.7 | 601.7 |
| 72 | IAFLRFLAI | 1 | B7 | HLA-B-5101 | 9 | DENV1 | 52 | 60 | 318.3 | 318.3 |
| 73 | IANQAAILM | 1 | B7 | HLA-B-3501 | 9 | DENV1 | 2315 | 2323 | 58.3 | 58.3 |
| 74 | IANQATVLM | 7 | B7 | HLA-B-3501 | 9 | DENV2 | 2315 | 2323 | 1518.3 | 1518.3 |
| | | | B7 | | | | | | | |
| 75 | IAVSMANIF | 6 | B58 | HLA-B-5801, | 9 | DENV1, | 2464 | 2472 | 1966.7 | 2381.7 |
| | | | | HLA-B-3501 | | 2, 3 | | | | |
| 75 | IAVSTANIF | 2 | B58 | HLA-B-5801 | 9 | DENV4 | 2464 | 2472 | 415.0 | |
| 76 | ICSAVPVHW | 1 | B58 | HLA-B-5801 | 9 | DENV3 | 3275 | 3283 | 115.0 | 115.0 |
| 77 | IGIGILLTW | 1 | B58 | HLA-B-5801 | 9 | DENV1 | 738 | 746 | 525.0 | 903.3 |
| 77 | IGIGVLLTW | 1 | B58 | HLA-B-5801 | 9 | DENV1 | 738 | 746 | 378.3 | |
| 78 | IGLTSRATW | 1 | B58 | HLA-B-5801 | 9 | DENV2, | 3347 | 3355 | 50.0 | 50.0 |
| | | | | | | 3 | | | | |
| 79 | IILEFFLMV | 1 | A2 | HLA-A-0201 | 9 | DENV1, | 2205 | 2213 | 143.3 | 143.3 |
| | | | | | | 4 | | | | |
| 80 | IPFEIMDLEK | 5 | B7 | HLA-B-3501 | 10 | DENV2 | 616 | 625 | 1500.0 | 1500.0 |
| 81 | IPITAAAWY | 5 | B7 | HLA-B-3501 | 9 | DENV2 | 1461 | 1469 | 1001.7 | 1001.7 |
| 82 | IPKAYAGPF | 1 | B7 | HLA-B-3501 | 9 | DENV4 | 1019 | 1027 | 920.0 | 920.0 |
| 83 | IPMATYGWNL | 2 | B7 | HLA-B-0702 | 10 | DENV1, | 2609 | 2618 | 456.7 | 456.7 |
| | | | | | | 4 | | | | |
| | | | B7 | HLA-B -0702 | | | | | | |
| 83 | IPMSTYGWNL | 3 | B7 | HLA-B-3501 | 10 | DENV2 | 2609 | 2618 | 320.0 | 320.0 |
| 84 | IPMTGPLVAG | 5 | B7 | HLA-B-3501 | 10 | DENV2 | 1373 | 1382 | 1160.0 | 1160.0 |
| 85 | IPQWEPSKGW | 1 | B7 | HLA-B-5301 | 10 | DENV1, | 3187 | 3196 | 26.7 | 26.7 |
| | | | | | | 4 | | | | |
| 86 | IPTVMAFHL | 1 | B7 | HLA-B-5301 | 9 | DENV2, | 110 | 118 | 70.0 | 70.0 |
| | | | | | | 4 | | | | |
| 87 | IQPFLALGF | 1 | A24 | HLA-A-2402 | 9 | DENV3 | 1211 | 1219 | 85.0 | 85.0 |
| 88 | ISSMLNIMNR | 1 | A3 | HLA-A-6801 | 10 | DENV1 | 89 | 98 | 30.0 | 30.0 |
| 89 | ITAAAWYLW | 1 | B7 | HLA-B-5301 | 9 | DENV2 | 1463 | 1471 | 310.0 | 310.0 |
| 90 | ITLLCLIPTV | 1 | A2 | HLA-A-0201 | 10 | DENV4 | 104 | 113 | 63.3 | 116.7 |
| 90 | TLLCLIPTV | 1 | A2 | HLA-A-0201 | 9 | DENV4 | 105 | 113 | 53.3 | |
| 91 | ITPMLRHTI | 1 | A24 | HLA-A-2402 | 9 | DENV3 | 2295 | 2303 | 190.0 | 190.0 |
| 92 | IVIGVGDSAL | 1 | B62 | HLA-B-1501 | 10 | DENV4 | 659 | 668 | 35.0 | 35.0 |
| 93 | KAKGSRAIW | 4 | B58 | HLA-B-5701 | 9 | DENV1, | 2962 | 2970 | 1070.0 | 1291.7 |
| | | | | | | 2, 3 | | | | |
| 93 | RAKGSRAIW | 2 | B58 | HLA-B-5701 | 9 | DENV4 | 2962 | 2970 | 221.7 | |
| 94 | KATREAQKRA | 1 | B58 | HLA-B-5801 | 10 | DENV2 | 2375 | 2384 | 383.3 | 383.3 |
| 95 | KAVHADMGYW | 1 | B58 | HLA-B-5801 | 10 | DENV1, | 968 | 977 | 136.7 | 180.0 |
| | | | | | | 2 | | | | |
| 95 | RAVHADMGYW | 1 | B58 | HLA-B-5801 | 10 | DENV2 | 968 | 977 | 43.3 | |
| 96 | KAYAQMWSL | 2 | B58 | HLA-B-5801, | 9 | DENV3 | 3252 | 3260 | 116.7 | 116.7 |
| | | | | B5701 | | | | | | |
| 97 | KEGVFHTMW | 1 | B44 | HLA-B-4403 | 9 | DENV3 | 1519 | 1527 | 178.3 | 178.3 |
| 98 | KGSRAIWYMW | 2 | B58 | HLA-B-5801, | 10 | DENV1, | 2964 | 2973 | 1345.0 | 1345.0 |
| | | | | B5701 | | 2, 3, | | | | |
| | | | | | | 4 | | | | |
| 99 | KPGTSGSPI | 1 | B7 | HLA-B-0702 | 9 | DENV1, | 1608 | 1616 | 345.0 | 516.7 |
| | | | | | | 3, 4 | | | | |
| 99 | KPGTSGSPII | 1 | B7 | HLA-B-0702 | 10 | DENV3, | 1608 | 1617 | 118.3 | |
| | | | | | | 4 | | | | |
| 99 | KPGTSGSPIV | 1 | B7 | HLA-B-0702 | 10 | DENV1 | 1608 | 1617 | 53.3 | |
| 100 | KPRICTREEF | 1 | B7 | HLA-B-0702 | 10 | DENV1 | 2885 | 2894 | 340.0 | 2756.3 |
| 100 | KPRLCTREEF | 1 | B7 | HLA-B-0702 | 10 | DENV3 | 2885 | 2894 | 350.0 | |
| 100 | NPRLCTREEF | 1 | B7 | HLA-B-0702 | 10 | DENV4 | 2885 | 2894 | 365.0 | |
| 100 | RPRLCTREEF | 4 | B7 | HLA-B-0702 | 10 | DENV3 | 2885 | 2894 | 501.3 | |
| 100 | TPRMCTREEF | 5 | B7 | HLA-B-0702, | 10 | DENV2 | 2885 | 2894 | 1200.0 | |
| | | | | B-3501 | | | | | | |
| 101 | KPRWLDARI | 1 | B7 | HLA-B-0702 | 9 | DENV2 | 2070 | 2078 | 70.0 | 70.0 |
| 102 | KPTLDFELI | 1 | B7 | HLA-B-5301 | 9 | DENV2, | 319 | 327 | 670.0 | 670.0 |
| | | | | | | 4 | | | | |
| 103 | KPWDVIPMV | 3 | B7 | HLA-B-5101 | 9 | DENV1 | 2825 | 2833 | 746.7 | 1298.3 |
| 103 | KPWDVVPMV | 2 | B7 | HLA-B-5101 | 9 | DENV2 | 2825 | 2833 | 551.7 | |
| 104 | KQIANELNY | 1 | B62 | HLA-B-1501 | 9 | DENV3 | 845 | 853 | 26.7 | 53.3 |
| 104 | KQISNELNY | 1 | B62 | HLA-B-1501 | 9 | DENV1 | 845 | 853 | 26.7 | |
| 105 | KSGAIKVLK | 1 | A3 | HLA-A-1101 | 9 | DENV3 | 75 | 83 | 48.3 | 48.3 |
| 106 | KSYAQMWQL | 1 | B58 | HLA-B-5801 | 9 | DENV1 | 3252 | 3260 | 140.0 | 140.0 |
| 107 | KTDFGFYQV | 1 | A2 | HLA-A-0206 | 9 | DENV4 | 2256 | 2264 | 235.0 | 235.0 |
| 108 | KTFVDLMRR | 1 | A3 | HLA-A-1101 | 9 | DENV2 | 2009 | 2017 | 38.3 | 38.3 |
| 109 | KTWAYHGSY | 1 | B62 | HLA-B-1501 | 9 | DENV1 | 2796 | 2804 | 25.0 | 25.0 |
| 110 | KVIQLSRKTF | 1 | B62 | HLA-B-1501 | 10 | DENV2 | 1859 | 1868 | 51.7 | 51.7 |
| 111 | KVLNPYMPSV | 1 | A2 | HLA-A-0201 | 10 | DENV2 | 2676 | 2685 | 63.3 | 105.0 |
| 111 | VLNPYMPSV | 1 | A2 | HLA-A-0201 | 9 | DENV2 | 2677 | 2685 | 41.7 | |
| 112 | KVRKDIQQW | 1 | B58 | HLA-B-5701 | 9 | DENV2 | 3182 | 3190 | 38.3 | 38.3 |
| 113 | LAYTIGTTHF | 2 | B7 | HLA-B-3501 | 10 | DENV2 | 255 | 264 | 421.7 | 421.7 |
| 114 | LEFEALGFLN | 1 | B44 | HLA-B-4001 | 10 | DENV2 | 2979 | 2988 | 610.0 | 1166.7 |
| 114 | LEFEALGFMN | 1 | B44 | HLA-B-4001 | 10 | DENV1 | 2979 | 2988 | 556.7 | |
| 115 | LETLMLVAL | 1 | B44 | HLA-B-4001 | 9 | DENV4 | 2148 | 2156 | 103.3 | 103.3 |
| 116 | LGFLNEDHW | 1 | B58 | HLA-B-5701 | 9 | DENV2, | 2984 | 2992 | 546.7 | 546.7 |
| | | | | | | 3, 4 | | | | |
| 117 | LIHQVFGTAY | 1 | B62 | HLA-B-1501 | 10 | DENV1 | 716 | 725 | 51.7 | 406.7 |
| 117 | LVHQIFGTAY | 1 | B62 | HLA-B-1501 | 10 | DENV1 | 716 | 725 | 355.0 | |
| 118 | LILCVTQVLM | 2 | B7 | HLA-B-3501 | 10 | DENV2 | 2421 | 2430 | 603.3 | 603.3 |
| 119 | LLMLVTPSM | 1 | B62 | HLA-B-1501 | 9 | DENV1 | 272 | 280 | 20.0 | 20.0 |
| 120 | LLSPVRVPNY | 1 | B62 | HLA-B-1501 | 10 | DENV1 | 1747 | 1756 | 36.7 | 36.7 |
| 121 | LMCHATFTM | 1 | B62 | HLA-B-1501 | 9 | DENV1 | 1737 | 1745 | 33.3 | 33.3 |
| 122 | LMKITAEWLW | 1 | B7 | HLA-B-5301 | 10 | DENV2 | 2868 | 2877 | 423.3 | 1416.7 |
| 122 | MEITAEWLW | 1 | B58 | HLA-B-5801 | 9 | DENV3 | 2869 | 2877 | 583.3 | |
| 122 | VMGITAEWLW | 1 | B7 | HLA-B-5301 | 10 | DENV3 | 2868 | 2877 | 410.0 | |
| 123 | LMMATIGIAL | 1 | B62 | HLA-B-1501 | 10 | DENV2 | 1229 | 1238 | 31.7 | 940.0 |
| 123 | MMATIGIAL | 6 | B7 | HLA-B-3501 | 9 | DENV2 | 1230 | 1238 | 908.3 | |
| 124 | LMMILPAAL | 1 | B62 | HLA-B-1501 | 9 | DENV3 | 106 | 114 | 46.7 | 46.7 |
| 125 | LMMMLPATLA | 1 | A2 | HLA-A-0201 | 10 | DENV3 | 106 | 115 | 61.7 | 61.7 |
| 126 | LMMTTIGVVL | 1 | B62 | HLA-B-1501 | 10 | DENV2 | 1229 | 1238 | 50.0 | 50.0 |
| 127 | LPAIVREAI | 3 | B7 | HLA-B-0702, | 9 | DENV1, | 1682 | 1690 | 1280.0 | 1280.0 |
| | | | | B-5301 | | 2, 3 | | | | |
| 128 | LPEEQDQNY | 5 | B7 | HLA-B-3501 | 9 | DENV3 | 363 | 371 | 1166.7 | 1166.7 |
| 129 | LPESLETLM | 1 | B7 | HLA-B-3501 | 9 | DENV4 | 2144 | 2152 | 208.3 | 208.3 |
| 130 | LPETLETLLL | 4 | B7 | HLA-B-3501, | 10 | DENV2 | 2144 | 2153 | 296.7 | 296.7 |
| | | | | B-5101 | | | | | | |
| 131 | LPGADTQGSN | 1 | B7 | HLA-B-3501 | 10 | DENV2 | 502 | 511 | 240.0 | 240.0 |
| 132 | LPIRYQTPA | 3 | B7 | HLA-B-0702, | 9 | DENV2 | 1716 | 1724 | 270.0 | 1164.3 |
| | | | | B-5101 | | | | | | |
| 132 | LPIRYQTPAI | 5 | B7 | HLA-B-3501 | 10 | DENV2 | 1716 | 1725 | 894.3 | |
| 133 | LPLPWLPGAD | 1 | B7 | HLA-B-3501 | 10 | DENV2 | 497 | 506 | 238.3 | 238.3 |
| 134 | LPLPWTSGA | 1 | B7 | HLA-B-5101 | 9 | DENV1 | 497 | 505 | 111.7 | 111.7 |
| 135 | LPTFMTQKAR | 8 | B7 | HLA-B-3501 | 10 | DENV2 | 2109 | 2118 | 1263.3 | 1263.3 |
| 136 | LPTYLSSRAK | 1 | B7 | HLA-B-3501 | 10 | DENV4 | 2109 | 2118 | 26.7 | 26.7 |
| 137 | LPVWLAHKVA | 1 | B7 | HLA-B-3501 | 10 | DENV3 | 2020 | 2029 | 23.3 | 4156.7 |
| 137 | LPVWLAYKV | 2 | B7 | HLA-B-5301, | 9 | DENV2 | 2020 | 2028 | 923.3 | |
| | | | | B-5101 | | | | | | |
| 137 | LPVWLAYKVA | 7 | B7 | HLA-B-3501 | 10 | DENV2 | 2020 | 2029 | 2426.7 | |
| 137 | LPVWLAYRVA | 1 | B7 | HLA-B-5101 | 10 | DENV2 | 2020 | 2029 | 470.0 | |
| 137 | LPVWLSYKV | 1 | B7 | HLA-B-5101 | 9 | DENV1 | 2020 | 2028 | 313.3 | |
| 138 | LRVLNLVENW | 1 | B7 | HLA-B-5301 | 10 | DENV2 | 2657 | 2666 | 351.7 | 351.7 |
| 139 | LSMGLITIAV | 1 | A2 | HLA-A-0206 | 10 | DENV4 | 2177 | 2186 | 571.7 | 571.7 |
| 140 | LSRNSTHEM | 1 | B62 | HLA-B-1501 | 9 | DENV1 | 2705 | 2713 | 26.7 | 26.7 |
| 141 | LTAAVLLLI | 1 | B58 | HLA-B-5801 | 9 | DENV3 | 2354 | 2362 | 545.0 | 611.7 |
| 141 | TLTAAVLLLV | 1 | A2 | HLA-A-0201 | 10 | DENV3 | 2353 | 2362 | 66.7 | |
| 142 | LWEGSPGKF | 1 | A24 | HLA-A-2402 | 9 | DENV1, | 2451 | 2459 | 25.0 | 25.0 |
| | | | | | | 3 | | | | |
| 143 | MAEIPLQWI | 1 | B7 | HLA-B-5101 | 9 | DENV3 | 2193 | 2201 | 96.7 | 96.7 |
| | | | B7 | | | | | | | |
| 144 | MAFIAFLRF | 2 | B62 | HLA-B-3501, | 9 | DENV1, | 49 | 57 | 146.7 | 146.7 |
| | | | | B-1501 | | 3 | | | | |
| 145 | MAILGDTAW | 1 | B7 | HLA-B-3501 | 9 | DENV1, | 693 | 701 | 21.7 | 21.7 |
| | | | | | | 2, 3 | | | | |
| 146 | MALKDFKEF | 7 | B7 | HLA-B-3501 | 9 | DENV4 | 2083 | 2091 | 1496.7 | 1496.7 |
| 147 | MAMTCIAVG | 1 | B7 | HLA-B-3501 | 9 | DENV4 | 756 | 764 | 123.3 | 123.3 |
| 148 | MANEMGFLEK | 1 | A3 | HLA-A-1101 | 10 | DENV2 | 2245 | 2254 | 21.7 | 21.7 |
| 149 | MANIFRGSY | 5 | B7 | HLA-B-3501 | 9 | DENV1, | 2468 | 2476 | 821.7 | 821.7 |
| | | | | | | 2, 3 | | | | |
| 150 | MASSALLWMA | 1 | B58 | HLA-B-5801 | 10 | DENV1 | 2185 | 2194 | 45.0 | 45.0 |
| 151 | MAVGMVSIL | 5 | B7 | HLA-B-3501 | 9 | DENV2 | 1356 | 1364 | 361.7 | 361.7 |
| 152 | MGYWIESAL | 1 | B7 | HLA-B-3501 | 9 | DENV2 | 974 | 982 | 53.3 | 53.3 |
| 153 | MIDKTPVHSW | 1 | B7 | HLA-B-5301 | 10 | DENV4 | 3320 | 3329 | 720.0 | 720.0 |
| 154 | MLLALIAVL | 1 | A2 | HLA-A-0201 | 9 | DENV1 | 2152 | 2160 | 81.7 | 81.7 |
| 155 | MLLILCVTQV | 1 | A2 | HLA-A-0201 | 10 | DENV2 | 2419 | 2428 | 43.3 | 43.3 |
| 156 | MLVTPSMTM | 8 | B7 | HLA-B-3501 | 9 | DENV3 | 274 | 282 | 1735.0 | 1735.0 |
| 157 | MLWMAEIPL | 1 | B7 | HLA-B-3501 | 9 | DENV3 | 2190 | 2198 | 21.7 | 21.7 |
| 158 | MMLKLLTDF | 1 | B62 | HLA-B-1501 | 9 | DENV1 | 1260 | 1268 | 50.0 | 50.0 |
| 159 | MMLPATLAF | 1 | B7 | HLA-B-3501 | 9 | DENV3 | 108 | 116 | 80.0 | 80.0 |
| 159 | MMMLPATLAF | 2 | B7 | HLA-B-3501 | 10 | DENV3 | 107 | 116 | 473.3 | 473.3 |
| 160 | MPGTFQTTTG | 1 | B7 | HLA-B-3501 | 10 | DENV3 | 1589 | 1598 | 136.7 | 136.7 |
| 161 | MPGTRKVMGI | 1 | B7 | HLA-B-5101 | 10 | DENV3 | 2862 | 2871 | 60.0 | 85.0 |
| 161 | MPGTRRVMGI | 1 | B7 | HLA-B-5101 | 10 | DENV3 | 2862 | 2871 | 25.0 | |
| 162 | MPLVMAWRTI | 1 | B7 | HLA-B-5101 | 10 | DENV4 | 1287 | 1296 | 56.7 | 56.7 |
| 163 | MPSMKRFRRE | 5 | B7 | HLA-B-5301, | 10 | DENV2 | 3379 | 3388 | 1336.7 | 1336.7 |
| | | | | B-3501 | | | | | | |
| 164 | MPSVIEKMET | 2 | B7 | HLA-B-3501, | 10 | DENV2 | 2682 | 2691 | 783.3 | 783.3 |
| | | | | B-5301 | | | | | | |
| 165 | MPVMKRYSAP | 1 | B7 | HLA-B-5101 | 10 | DENV4 | 3379 | 3388 | 36.7 | 36.7 |
| 166 | MPVTHSSAAQ | 2 | B7 | HLA-B-3501 | 10 | DENV2 | 1925 | 1934 | 183.3 | 235.0 |
| 166 | MPVTVASAAQ | 1 | B7 | HLA-B-3501 | 10 | DENV1 | 1925 | 1934 | 51.7 | |
| 167 | MSFRDLGRVM | 8 | B7 | HLA-B-3501 | 10 | DENV2 | 1176 | 1185 | 3250.0 | 3250.0 |
| 168 | MSSEGAWKHA | 1 | B58 | HLA-B-5701 | 10 | DENV2, | 226 | 235 | 778.3 | 778.3 |
| | | | | | | 4 | | | | |
| 169 | MSYSMCTGKF | 6 | B7 | HLA-B-3501 | 10 | DENV2 | 578 | 587 | 2553.3 | 2553.3 |
| 170 | MSYTMCSGK | 1 | A3 | HLA-A-1101 | 9 | DENV4 | 578 | 586 | 23.3 | 23.3 |
| 171 | MTQKARNAL | 2 | B7 | HLA-B-0702 | 9 | DENV2 | 2113 | 2121 | 268.3 | 268.3 |
| 172 | MTTTANWLW | 1 | B7 | HLA-B-5301 | 9 | DENV4 | 2869 | 2877 | 40.0 | 40.0 |
| 173 | MVSRLLLNR | 1 | A3 | HLA-A-1101 | 9 | DENV3 | 2729 | 2737 | 28.3 | 28.3 |
| 174 | NPAQEDDQY | 2 | B7 | HLA-B-3501 | 9 | DENV4 | 1942 | 1950 | 176.7 | 176.7 |
| 175 | NPEIEDDIF | 10 | B7 | HLA-B-3501 | 9 | DENV2 | 1653 | 1661 | 3390.0 | 3390.0 |
| 176 | NPITLTAAL | 1 | B7 | HLA-B-0702 | 9 | DENV2 | 2350 | 2358 | 20.0 | 20.0 |
| 177 | NPLTLTAAV | 2 | B7 | HLA-B-0702 | 9 | DENV1, | 2350 | 2358 | 415.0 | 415.0 |
| | | | | | | 3 | | | | |
| 178 | NPNMIDKTPV | 2 | B7 | HLA-B-0702 | 10 | DENV4 | 3317 | 3326 | 376.7 | 675.0 |
| 178 | NPWMEDKTH | 1 | B7 | HLA-B-3501 | 9 | DENV1 | 3317 | 3325 | 106.7 | |
| 178 | NPWMEDKTPV | 2 | B7 | HLA-B-0702 | 10 | DENV2, | 3317 | 3326 | 191.7 | |
| | | | | | | 3 | | | | |
| 179 | NPTVDGITV | 1 | B7 | HLA-B-5101 | 9 | DENV2 | 2391 | 2399 | 30.0 | 30.0 |
| 180 | NQLIYVILTI | 1 | A2 | HLA-A-0206 | 10 | DENV4 | 2228 | 2237 | 381.7 | 381.7 |
| 181 | PASAWTLYAV | 1 | B58 | HLA-B-5801 | 10 | DENV1, | 2281 | 2290 | 503.3 | 503.3 |
| | | | | | | 2, 3, | | | | |
| | | | | | | 4 | | | | |
| 182 | PASIAARGYI | 2 | B58 | HLA-B-5801 | 10 | DENV1, | 1769 | 1778 | 611.7 | 611.7 |
| | | | | | | 2, 3 | | | | |
| 183 | PTSRTTWSIH | 1 | B58 | HLA-B-5801 | 10 | DENV1 | 3285 | 3294 | 215.0 | 215.0 |
| 184 | QEGAMHSAL | 1 | B44 | HLA-B-4001 | 9 | DENV4 | 537 | 545 | 48.3 | 88.3 |
| 184 | QEGAMHTAL | 1 | B44 | HLA-B-4001 | 9 | DENV1, | 537 | 545 | 40.0 | |
| | | | | | | 2, 3 | | | | |
| 185 | QENPYRTWAY | 1 | B44 | HLA-B-4001 | 10 | DENV4 | 2791 | 2800 | 265.0 | 265.0 |
| 186 | QIAMTDTTPF | 1 | B62 | HLA-B-1501 | 10 | DENV1 | 2835 | 2844 | 28.3 | 28.3 |
| 187 | QPESNILDI | 1 | B7 | HLA-B-5301 | 9 | DENV2 | 2268 | 2277 | 351.7 | 351.7 |
| 188 | QPHWIAASI | 1 | B7 | HLA-B-5101 | 9 | DENV2 | 2197 | 2205 | 71.7 | 71.7 |
| 189 | QSGVDVFFTP | 1 | B58 | HLA-B-5801 | 10 | DENV2 | 2622 | 2631 | 103.3 | 103.3 |
| 190 | QYIFTGQPL | 1 | A24 | HLA-A-2402 | 9 | DENV3 | 1949 | 1957 | 138.3 | 138.3 |
| 191 | QYSDRRWCF | 1 | A24 | HLA-A-2402 | 9 | DENV1 | 2034 | 2042 | 31.7 | 31.7 |
| 192 | RAAVEDEEF | 1 | B58 | HLA-B-5801 | 9 | DENV3 | 2918 | 2926 | 381.7 | 381.7 |
| 193 | RASFIEVKTC | 2 | B58 | HLA-B-5801 | 10 | DENV1 | 990 | 999 | 290.0 | 290.0 |
| 194 | REDLWCGSL | 1 | B44 | HLA-B-4001 | 9 | DENV4 | 3338 | 3346 | 96.7 | 145.0 |
| 194 | REDQWCGSL | 1 | B44 | HLA-B-4001 | 9 | DENV1, | 3338 | 3346 | 48.3 | |
| | | | | | | 2, 3 | | | | |
| 195 | REIPERSWNT | 2 | B44 | HLA-B-4001 | 10 | DENV4 | 1815 | 1824 | 1278.3 | 1278.3 |
| 196 | REWCFTGERN | 2 | B44 | HLA-B-4001 | 10 | DENV4 | 2038 | 2047 | 1345.0 | 1345.0 |
| 197 | RFLEFEALGF | 1 | A24 | HLA-A-2402 | 10 | DENV1, | 2977 | 2986 | 181.7 | 358.3 |
| | | | | | | 2, 4 | | | | |
| 197 | RYLEFEALGF | 1 | A24 | HLA-A-2402 | 10 | DENV3 | 2977 | 2986 | 176.7 | |
| 198 | RLRGEARKTF | 1 | B62 | HLA-B-1501 | 10 | DENV1 | 2002 | 2011 | 21.7 | 21.7 |
| 199 | RPGYHTQTA | 2 | B7 | HLA-B-0702 | 9 | DENV2, | 1033 | 1041 | 501.7 | 501.7 |
| | | | | | | 3 | | | | |
| 200 | RPISEKEENM | 1 | B7 | HLA-B-3501 | 10 | DENV3 | 1112 | 1121 | 101.7 | 101.7 |
| 201 | RPRWLDART | 2 | B7 | HLA-B-0702 | 9 | DENV1, | 2070 | 2078 | 585.0 | 585.0 |
| | | | | | | 3 | | | | |
| 202 | RPTPKGAVM | 1 | B7 | HLA-B-0702 | 9 | DENV4 | 3077 | 3085 | 191.7 | 223.3 |
| 202 | RPTPKGTVM | 1 | B7 | HLA-B-0702 | 9 | DENV3 | 3077 | 3085 | 31.7 | |
| 203 | RQLANAIFK | 1 | A3 | HLA-A-1101 | 9 | DENV3 | 3057 | 3065 | 90.0 | 90.0 |
| 204 | RQMEGEGIF | 1 | B62 | HLA-B-1501 | 9 | DENV2 | 3116 | 3124 | 108.3 | 108.3 |
| 205 | RQMEGEGVL | 1 | B62 | HLA-B-1501 | 9 | DENV3 | 3116 | 3124 | 56.7 | 56.7 |
| 206 | RVIDPRRCL | 2 | B7 | HLA-B-0702 | 9 | DENV1, | 1899 | 1907 | 541.7 | 1368.3 |
| | | | | | | 3, 4 | | | | |
| 206 | RVIDPRRCLK | 3 | A3 | HLA-A-0301, | 10 | DENV1 | 1899 | 1908 | 258.3 | |
| | | | | A-1101 | | | | | | |
| 206 | RVIDPRRCM | 1 | B7 | HLA-B-0702 | 9 | DENV2 | 1899 | 1907 | 88.3 | |
| 206 | RVIDPRRCMK | 6 | A3 | HLA-A-3101, | 10 | DENV2 | 1899 | 1908 | 480.0 | |
| | | | | A-1101, | | | | | | |
| | | | | A-0301 | | | | | | |
| 207 | RVLKMVEPW | 1 | B58 | HLA-B-5801 | 9 | DENV1, | 2658 | 2666 | 150.0 | 150.0 |
| | | | | | | 3, 4 | | | | |
| 208 | RYMGEDGCWY | 1 | A24 | HLA-A-2402 | 10 | DENV3 | 1098 | 1107 | 66.7 | 66.7 |
| 209 | SEMAEALKGM | 1 | B44 | HLA-B-4001 | 10 | DENV1 | 1707 | 1716 | 30.0 | 30.0 |
| 210 | SPCKIPFEIM | 5 | B7 | HLA-B-3501 | 10 | DENV2 | 612 | 621 | 1156.0 | 1156.0 |
| 211 | SPGKFWNTTI | 3 | B7 | HLA-B-0702 | 10 | DENV1, | 2455 | 2464 | 826.7 | 826.7 |
| | | | | | | 2 | | | | |
| 212 | SPIINREGKV | 1 | B7 | HLA-B-0702 | 10 | DENV3 | 1614 | 1623 | 106.7 | 106.7 |
| 213 | SPILSITISE | 6 | B7 | HLA-B-3501 | 10 | DENV2 | 1418 | 1427 | 1068.3 | 1068.3 |
| 214 | SPKRLATAIA | 1 | B7 | HLA-B-0702 | 10 | DENV3 | 814 | 823 | 68.3 | 68.3 |
| 215 | SPSPTVEESR | 1 | B7 | HLA-B-0702 | 10 | DENV3 | 2646 | 2655 | 118.3 | 118.3 |
| 216 | SPVRVPNYNL | 1 | B7 | HLA-B-0702 | 10 | DENV2, | 1749 | 1758 | 173.3 | 173.3 |
| | | | | | | 3 | | | | |
| 217 | SQIGAGVYK | 1 | A3 | HLA-A-1101 | 9 | DENV2 | 1511 | 1519 | 40.0 | 40.0 |
| 218 | STYGWNLVR | 1 | A3 | HLA-A-1101 | 9 | DENV2 | 2612 | 2620 | 28.3 | 28.3 |
| 219 | SWMVRILIGF | 1 | A24 | HLA-A-2402 | 10 | DENV4 | 733 | 742 | 53.3 | 53.3 |
| 220 | SYAQMWTLMY | 1 | A1 | HLA-A-0101 | 10 | DENV2 | 3253 | 3262 | 151.7 | 578.3 |
| 220 | YAQMWQLMYF | 1 | B7 | HLA-B-3501 | 10 | DENV1 | 3254 | 3263 | 135.0 | |
| 220 | YAQMWSLMY | 1 | B62 | HLA-B-1501 | 9 | DENV2, | 3254 | 3262 | 38.3 | |
| | | | | | | 3, 4 | | | | |
| 220 | YAQMWSLMYF | 1 | B7 | HLA-B-3501 | 10 | DENV2, | 3254 | 3263 | 33.3 | |
| | | | | | | 3, 4 | | | | |
| 220 | YAQMWTLMY | 2 | B62 | HLA-B-1501 | 9 | DENV2 | 3254 | 3262 | 175.0 | |
| 220 | YAQMWTLMYF | 1 | B62 | HLA-B-1501 | 10 | DENV2 | 3254 | 3263 | 45.0 | |
| 221 | TAAVLLLITH | 1 | B58 | HLA-B-5801 | 10 | DENV3 | 2355 | 2364 | 118.3 | 118.3 |
| 222 | TAEAGGRAY | 4 | B7 | HLA-B-3501 | 9 | DENV2 | 2129 | 2137 | 371.7 | 371.7 |
| 223 | TETTILDVDL | 2 | B44 | HLA-B-4001 | 10 | DENV4 | 2266 | 2279 | 1413.3 | 1413.3 |
| 224 | TFKVPHAKR | 1 | A3 | HLA-A-3301 | 9 | DENV4 | 520 | 528 | 78.3 | 78.3 |
| 225 | TLMAMDLGEL | 1 | A2 | HLA-A-0201 | 10 | DENV2 | 150 | 159 | 823.3 | 823.3 |
| 226 | TLMLLALIAV | 1 | A2 | HLA-A-0201 | 10 | DENV1 | 2150 | 2159 | 31.7 | 31.7 |
| 227 | TPEAKNSTF | 1 | B7 | HLA-B-3501 | 9 | DENV4 | 901 | 909 | 860.0 | 915.0 |
| 227 | TPEARNSTF | 1 | B7 | HLA-B-3501 | 9 | DENV4 | 901 | 909 | 55.0 | |
| 228 | TPEGIIPAL | 1 | B7 | HLA-B-0702 | 9 | DENV1, | 1978 | 1986 | 183.3 | 10671.0 |
| | | | | | | 3 | | | | |
| 228 | TPEGIIPALF | 9 | B7 | HLA-B-3501 | 10 | DENV1, | 1978 | 1987 | 2213.3 | |
| | | | | | | 3 | | | | |
| 228 | TPEGIIPSM | 5 | B7 | HLA-B-3501, | 9 | DENV2 | 1978 | 1986 | 2716.7 | |
| | | | | B-5301 | | | | | | |
| 228 | TPEGIIPSMF | 7 | B7 | HLA-B-0702, | 10 | DENV2 | 1978 | 1987 | 1771.7 | |
| | | | | B-3501 | | | | | | |
| 228 | TPEGIIPTLF | 11 | B7 | HLA-B-3501, | 10 | DENV4 | 1978 | 1987 | 2914.3 | |
| | | | | B-0702, | | | | | | |
| | | | | B-5301 | | | | | | |
| 228 | YTPEGIIPTL | 1 | A2 | HLA-A-0206 | 10 | DENV4 | 1977 | 1986 | 871.7 | |
| 229 | TPFGQQRVF | 3 | B7 | HLA-B-3501 | 9 | DENV1, | 2842 | 2850 | 438.3 | |
| | | | | | | 2, 3, | | | | |
| | | | | | | 4 | | | | |
| 230 | TPKGAVMDII | 1 | B7 | HLA-B-0702 | 10 | DENV4 | 3079 | 3088 | 105.0 | 436.7 |
| 230 | TPRGTVMDII | 1 | B7 | HLA-B-0702 | 10 | DENV4 | 3079 | 3088 | 331.7 | |
| 231 | TPMLRHTIEN | 2 | B7 | HLA-B-0702 | 10 | DENV3, | 2296 | 2305 | 388.3 | 1805.0 |
| | | | | | | 4 | | | | |
| 232 | TPPGSRDPF | 7 | B7 | HLA-B-3501, | 9 | DENV2 | 1795 | 1803 | 1416.7 | |
| | | | | B-0702 | | | | | | |
| 233 | TPQDNQLAY | 1 | B7 | HLA-B-3501 | 9 | DENV1, | 2224 | 2232 | 25.0 | 506.7 |
| | | | | | | 3 | | | | |
| 233 | TPQDNQLAYV | 2 | B7 | HLA-B-0702 | 10 | DENV1, | 2224 | 2233 | 406.7 | |
| | | | | | | 3 | | | | |
| 233 | TPQDNQLTY | 1 | B7 | HLA-B-3501 | 9 | DENV2 | 2224 | 2232 | 75.0 | |
| 234 | TPRSMPGTRR | 1 | B7 | HLA-B-0702 | 10 | DENV3 | 2858 | 2867 | 55.0 | 55.0 |
| 235 | TPVHSWEDI | 1 | B7 | HLA-B-5101 | 9 | DENV4 | 3324 | 3332 | 63.3 | 63.3 |
| 236 | TTFSLHYAW | 1 | B58 | HLA-B-5801 | 9 | DENV1 | 1285 | 1293 | 38.3 | 38.3 |
| 237 | TTKRDLGMSK | 1 | A3 | HLA-A-1101 | 10 | DENV3 | 2254 | 2263 | 35.0 | 35.0 |
| 238 | TYLALIATF | 1 | A24 | HLA-A-2402 | 9 | DENV3 | 1201 | 1209 | 28.3 | 28.3 |
| 239 | VASGLLWVAE | 1 | B58 | HLA-B-5801 | 10 | DENV4 | 2186 | 2195 | 53.3 | 53.3 |
| 240 | VATTFVTPM | 8 | B7 | HLA-B-3501 | 9 | DENV2 | 2290 | 2298 | 2776.7 | 2776.7 |
| 241 | VLLLVTHYAI | 1 | A2 | HLA-A-0201 | 10 | DENV3 | 2358 | 2367 | 40.0 | 40.0 |
| 242 | VPLLAIGCY | 7 | B7 | HLA-B-3501 | 9 | DENV2 | 2338 | 2346 | 1520.0 | 2603.3 |
| 242 | VPLLAMGCY | 1 | B7 | HLA-B-3501 | 9 | DENV4 | 2338 | 2346 | 1083.3 | |
| 243 | VPMVTQMAM | 1 | B7 | HLA-B-0702 | 9 | DENV2, | 2830 | 2838 | 148.3 | 148.3 |
| | | | | | | 3 | | | | |
| 244 | VPNYNMIIM | 1 | B7 | HLA-B-5101 | 9 | DENV1 | 1753 | 1761 | 51.7 | 51.7 |
| 245 | VPYLGKREDQ | 2 | B7 | HLA-B-0702 | 10 | DENV1, | 3332 | 3341 | 423.3 | 423.3 |
| | | | | | | 2, 3 | | | | |
| 246 | VQADMGCVV | 1 | A2 | HLA-A-0206 | 9 | DENV4 | 774 | 782 | 190.0 | 190.0 |
| 247 | VSSVNMVSRL | 1 | B58 | HLA-B-5801 | 10 | DENV3 | 2724 | 2733 | 421.7 | 421.7 |
| 248 | VTIDLDPVVY | 1 | B62 | HLA-B-1501 | 10 | DENV1 | 2398 | 2407 | 38.3 | 38.3 |
| 249 | VTRGAVLMHK | 1 | A3 | HLA-A-1101 | 10 | DENV2 | 1529 | 1538 | 81.7 | 81.7 |
| 250 | VTYECPLLV | 1 | A2 | HLA-A-0201 | 9 | DENV4 | 164 | 172 | 31.7 | 31.7 |
| 251 | VYTQLCDHR | 1 | A3 | HLA-A-3301 | 9 | DENV3 | 950 | 958 | 35.0 | 56.7 |
| 251 | VYTQLCDHRL | 1 | A24 | HLA-A-2402 | 10 | DENV3 | 950 | 959 | 21.7 | |
| 252 | WALCESITL | 1 | B7 | HLA-B-3501 | 9 | DENV1 | 2435 | 2443 | 23.3 | 23.3 |
| 253 | WAYHGSYET | 3 | B7 | HLA-B-3501 | 9 | DENV2 | 2798 | 2806 | 1220.0 | 1245.0 |
| 253 | WAYHGSYEV | 1 | B7 | HLA-B-5101 | 9 | DENV1, | 3 | 2798 | 2806 | 25.0 |
| 254 | WHYDQDHPY | 5 | B7 | HLA-B-3501 | 9 | DENV2 | 2787 | 2795 | 1628.3 | 1628.3 |
| 255 | WSIHAHHQW | 3 | B58 | HLA-B-5801; | 9 | DENV1, | 3291 | 3299 | 950.0 | 950.0 |
| | | | | B5701 | | 3, 4 | | | | |
| 256 | WVAEIQPQW | 1 | B58 | HLA-B-5801 | 9 | DENV4 | 2192 | 2200 | 30.0 | 30.0 |
| 257 | YAQIQPHWI | 1 | B7 | HLA-B-5101 | 9 | DENV2 | 2193 | 2201 | 48.3 | 48.3 |
| 258 | YENLKYSVI | 1 | B44 | HLA-B-4402 | 9 | DENV1 | 413 | 421 | 381.7 | 381.7 |
| 259 | YGGPISQHNY | 1 | B7 | HLA-B-3501 | 10 | DENV1 | 1023 | 1032 | 33.3 | 33.3 |
| 260 | YGVLFSGVSW | 1 | B7 | HLA-B-5301 | 10 | DENV1 | 725 | 734 | 166.7 | 166.7 |
| 262 | YLAGAGLAF | 3 | B7 | HLA-B-0702, | 9 | DENV1, | 2476 | 2484 | 340.0 | 340.0 |
| | | | B62 | B-1501 | | 3 | | | | |
| 263 | YPKTKLTDW | 1 | B7 | HLA-B-5301 | 9 | DENV4 | 1872 | 1880 | 320.0 | 430.0 |
| 263 | YPKTKLTDWD | 1 | B7 | HLA-B-3501 | 10 | DENV4 | 1872 | 1881 | 110.0 | |
| 264 | YQLAVTITAI | 1 | B62 | HLA-B-1501 | 10 | DENV2 | 1271 | 1280 | 63.3 | 63.3 |
| 265 | YQLWTALISL | 1 | B62 | HLA-B-1501 | 10 | DENV3 | 1271 | 1280 | 55.0 | 55.0 |
| 266 | YQNKVVKVLR | 1 | A3 | HLA-A-3301 | 10 | DENV4 | 3068 | 3077 | 895.0 | 1235.0 |
| 266 | YQNKVVKVQR | 1 | A3 | HLA-A-3301 | 10 | DENV3 | 3068 | 3077 | 340.0 | |
| 267 | YVSAIAQTEK | 1 | A3 | HLA-A-1101 | 10 | DENV2 | 1638 | 1647 | 36.7 | 36.7 |

1. Fu, J., B. H. Tan, E. H. Yap, Y. C. Chan, and Y. H. Tan. 1992. Full-length cDNA sequence of dengue type 1 virus (Singapore strain S275/90). Virology 188:953-958.
2. Suaya, J. A., D. S. Shepard, J. B. Siqueira, C. T. Martelli, L. C. Lum, L. H. Tan, S. Kongsin, S. Jiamton, F. Gamido, R. Montoya, B. Armien, R. Huy, L. Castillo, M. Caram, B. K. Sah, R. Sughayyar, K. R. Tyo, and S. B. Halstead. 2009. Cost of dengue cases in eight countries in the Americas and Asia: a prospective study. The American journal of tropical medicine and hygiene 80:846-855.
3. Guzman, M. G., S. B. Halstead, H. Artsob, P. Buchy, J. Farrar, D. J. Gubler, E. Hunsperger, A. Kroeger, H. S. Margolis, E. Martinez, M. B. Nathan, J. L. Pelegrino, C. Simmons, S. Yoksan, and R. W. Peeling. 2010. Dengue: a continuing global threat. Nature reviews. Microbiology 8:S7-16.
4. Kyle, J. L., and E. Harris. 2008. Global spread and persistence of dengue. Annual review of microbiology 62:71-92.
5. Guzman, A., and R. E. Isturiz. 2010. Update on the global spread of dengue. International journal of antimicrobial agents 36 Suppl 1:S40-42.
6. Kyle, J. L., and E. Harris. 2008. Global spread and persistence of dengue. Annu Rev Microbiol 62:71-92.
7. Burke, D. S., A. Nisalak, D. E. Johnson, and R. M. Scott. 1988. A prospective study of dengue infections in Bangkok. The American journal of tropical medicine and hygiene 38:172-180.
8. Sangkawibha, N., S. Rojanasuphot, S. Ahandrik, S. Viriyapongse, S. Jatanasen, V. Salitul, B. Phanthumachinda, and S. B. Halstead. 1984. Risk factors in dengue shock syndrome: a prospective epidemiologic study in Rayong, Thailand. I. The 1980 outbreak. American journal of epidemiology 120:653-669.
9. Halstead, S. B. 1988. Pathogenesis of dengue: challenges to molecular biology. Science 239:476-481.
10. Heinz, F. X., and K. Stiasny. 2012. Flaviviruses and flavivirus vaccines. Vaccine 30:4301-4306.
11. Morens, D. M. 1994. Antibody-dependent enhancement of infection and the pathogenesis of viral disease. Clinical infectious diseases: an official publication of the Infectious Diseases Society of America 19:500-512.
12. Halstead, S. B., S. Mahalingam, M. A. Marovich, S. Ubol, and D. M. Mosser. 2010. Intrinsic antibody-dependent enhancement of microbial infection in macrophages: disease regulation by immune complexes. The Lancet infectious diseases 10:712-722.
13. Halstead, S. B., S. Rojanasuphot, and N. Sangkawibha. 1983. Original antigenic sin in dengue. The American journal of tropical medicine and hygiene 32:154-156.
14. Lan, N. T., and K. Hirayama. 2011. Host genetic susceptibility to severe dengue infection. Tropical medicine and health 39:73-81.
15. Malavige, G. N., T. Rostron, L. T. Rohanachandra, S. D. Jayaratne, N. Fernando, A. D. De Silva, M. Liyanage, and G. Ogg. 2011. HLA class I and class II associations in dengue viral infections in a Sri Lankan population. PloS one 6:e20581.
16. Stephens, H. A., R. Klaythong, M. Sirikong, D. W. Vaughn, S. Green, S. Kalayanarooj, T. P. Endy, D. H. Libraty, A. Nisalak, B. L. Innis, A. L. Rothman, F. A. Ennis, and D. Chandanayingyong. 2002. HLA-A and -B allele associations with secondary dengue virus infections correlate with disease severity and the infecting viral serotype in ethnic Thais. Tissue antigens 60:309-318.
17. Loke, H., D. B. Bethell, C. X. Phuong, M. Dung, J. Schneider, N. J. White, N. P. Day, J. Farrar, and A. V. Hill. 2001. Strong HLA class I-restricted T cell responses in dengue hemorrhagic fever: a double-edged sword? The Journal of infectious diseases 184:1369-1373.
18. Appanna, R., S. Ponnampalavanar, L. Lum Chai See, and S. D. Sekaran. 2010. Susceptible and protective HLA class 1 alleles against dengue fever and dengue hemorrhagic fever patients in a Malaysian population. PloS one 5.
19. Nguyen, T. P., M. Kikuchi, T. Q. Vu, Q. H. Do, T. T. Tran, D. T. Vo, M. T. Ha, V. T. Vo, T. P. Cao, V. D. Tran, T. Oyama, K. Morita, M. Yasunami, and K. Hirayama. 2008. Protective and enhancing HLA alleles, HLA-DRB1*0901 and HLA-A*24, for severe forms of dengue virus infection, dengue hemorrhagic fever and dengue shock syndrome. PLoS neglected tropical diseases 2:e304.
20. Falcon-Lezama, J. A., C. Ramos, J. Zuniga, L. Juarez-Palma, H. Rangel-Flores, A. R. Garcia-Trejo, V. Acunha-Alonzo, J. Granados, and G. Vargas-Alarcon. 2009. HLA class I and II polymorphisms in Mexican Mestizo patients with dengue fever. Acta tropica 112:193-197.
21. Sierra, B., R. Alegre, A. B. Perez, G. Garcia, K. Sturn-Ramirez, O. Obasanjo, E. Aguirre, M. Alvarez, R. Rodriguez-Roche, L. Valdes, P. Kanki, and M. G. Guzman. 2007. HLA-A, -B, -C, and -DRB1 allele frequencies in Cuban individuals with antecedents of dengue 2 disease: advantages of the Cuban population for HLA studies of dengue virus infection. Human immunology 68:531-540.
22. Robinson, J., K. Mistry, H. McWilliam, R. Lopez, P. Parham, and S. G. Marsh. 2011. The IMGT/HLA database. Nucleic acids research 39:D1171-1176.
23. King, N. J., B. Shrestha, and A. M. Kesson. 2003. Immune modulation by flaviviruses. Advances in virus research 60:121-155.
24. Sette, A., and J. Sidney. 1999. Nine major HLA class I supertypes account for the vast preponderance of HLA-A and -B polymorphism. Immunogenetics 50:201-212.
25. Messer, W. B., U. T. Vitarana, K. Sivananthan, J. Elvtigala, L. D. Preethimala, R. Ramesh, N. Withana, D. J. Gubler, and A. M. De Silva. 2002. Epidemiology of dengue in Sri Lanka before and after the emergence of epidemic dengue hemorrhagic fever. The American journal of tropical medicine and hygiene 66:765-773.
26. Kanakaratne, N., W. M. Wahala, W. B. Messer, H. A. Tissera, A. Shahani, N. Abeysinghe, A. M. de-Silva, and M. Gunasekera. 2009. Severe dengue epidemics in Sri Lanka, 2003-2006. Emerging infectious diseases 15:192-199.
27. Tissera, H. A., E. E. Ooi, D. J. Gubler, Y. Tan, B. Logendra, W. M. Wahala, A. M. de Silva, M. R. Abeysinghe, P. Palihawadana, S. Gunasena, C. C. Tam, A. Amarasinghe, G. W. Letson, H. S. Margolis, and A. D. De Silva. 2011. New dengue virus type 1 genotype in Colombo, Sri Lanka. Emerging infectious diseases 17:2053-2055.
28. Yauch, L. E., R. M. Zellweger, M. F. Kotturi, A. Qutubuddin, J. Sidney, B. Peters, T. R. Prestwood, A. Sette, and S. Shresta. 2009. A protective role for dengue virus-specific CD8+ T cells. Journal of immunology 182:4865-4873.
29. Zompi, S., B. H. Santich, P. R. Beatty, and E. Harris. 2012. Protection from secondary dengue virus infection in a mouse model reveals the role of serotype cross-reactive B and T cells. Journal of immunology 188:404-416.
30. Duangchinda, T., W. Dejnirattisai, S. Vasanawathana, W. Limpitikul, N. Tangthawornchaikul, P. Malasit, J. Mongkolsapaya, and G. Screaton. 2010. Immunodominant T-cell responses to dengue virus NS3 are associated with DHF. Proceedings of the National Academy of Sciences of the United States of America 107:16922-16927.
31. Mongkolsapaya, J., W. Dejnirattisai, X. N. Xu, S. Vasanawathana, N. Tangthawornchaikul, A. Chairunsri, S. Sawasdivorn, T. Duangchinda, T. Dong, S. Rowland-Jones, P. T. Yenchitsomanus, A. McMichael, P. Malasit, and G. Screaton. 2003. Original antigenic sin and apoptosis in the pathogenesis of dengue hemorrhagic fever. Nature medicine 9:921-927.
32. Rothman, A. L. 2011. Immunity to dengue virus: a tale of original antigenic sin and tropical cytokine storms. Nature reviews. Immunology 11:532-543.
33. Mathew, A., I. Kurane, A. L. Rothman, L. L. Zeng, M. A. Brinton, and F. A. Ennis. 1996. Dominant recognition by human CD8+ cytotoxic T lymphocytes of dengue virus nonstructural proteins NS3 and NS1.2a. The Journal of clinical investigation 98:1684-1691.
34. Simmons, C. P., T. Dong, N. V. Chau, N. T. Dung, T. N. Chau, T. T. Thao le, T. T. Hien, S. Rowland-Jones, and J. Farrar. 2005. Early T-cell responses to dengue virus epitopes in Vietnamese adults with secondary dengue virus infections. Journal of virology 79:5665-5675.
35. Hsieh, Y. H., S. J. Liu, H. W. Chen, Y. K. Lin, K. S. Liang, and L. J. Lai. 2010. Highly sensitive rare cell detection based on quantum dot probe fluorescence analysis. Analytical and bioanalytical chemistry 396:1135-1141.
36. Newell, E. W., N. Sigal, S. C. Bendall, G. P. Nolan, and M. M. Davis. 2012. Cytometry by time-of-flight shows combinatorial cytokine expression and virus-specific cell niches within a continuum of CD8+ T cell phenotypes. Immunity 36:142-152.
37. Nguyen, T. H., H. Y. Lei, T. L. Nguyen, Y. S. Lin, K. J. Huang, B. L. Le, C. F. Lin, T. M. Yeh, Q. H. Do, T. Q. Vu, L. C. Chen, J. H. Huang, T. M. Lam, C. C. Liu, and S. B. Halstead. 2004. Dengue hemorrhagic fever in infants: a study of clinical and cytokine profiles. The Journal of infectious diseases 189:221-232.
38. Dung, N. T., H. T. Duyen, N. T. Thuy, T. V. Ngoc, N. V. Chau, T. T. Hien, S. L. Rowland-Jones, T. Dong, J. Farrar, B. Wills, and C. P. Simmons. 2010. Timing of CD8+ T cell responses in relation to commencement of capillary leakage in children with dengue. Journal of immunology 184:7281-7287.
39. Monteiro, S. P., P. E. do Brasil, G. M. Cabello, R. V. de Souza, P. Brasil, I. Georg, P. H. Cabello, and L. De Castro. 2012. HLA-A*01 allele: a risk factor for dengue haemorrhagic fever in Brazil's population. Memorias do Instituto Oswaldo Cruz 107:224-230.
40. Almeida, J. R., D. Sauce, D. A. Price, L. Papagno, S. Y. Shin, A. Moris, M. Larsen, G. Pancino, D. C. Douek, B. Autran, A. Saez-Cirion, and V. Appay. 2009. Antigen sensitivity is a major determinant of CD8+ T-cell polyfunctionality and HIV-suppressive activity. Blood 113:6351-6360.
41. Park, S. H., E. C. Shin, S. Capone, L. Caggiari, V. De Re, A. Nicosia, A. Folgori, and B. Rehermann. 2012. Successful Vaccination Induces Multifunctional Memory T-Cell Precursors Associated with Early Control of Hepatitis C Virus. Gastroenterology.
42. Hatch, S., T. P. Endy, S. Thomas, A. Mathew, J. Potts, P. Pazoles, D. H. Libraty, R. Gibbons, and A. L. Rothman. 2011. Intracellular cytokine production by dengue virus-specific T cells correlates with subclinical secondary infection. The Journal of infectious diseases 203:1282-1291.
43. Elahi, S., W. L. Dinges, N. Lejarcegui, K. J. Laing, A. C. Collier, D. M. Koelle, M. J. McElrath, and H. Horton. 2011. Protective HIV-specific CD8⁺ T cells evade Treg cell suppression. Nature medicine 17:989-995.
44. Barrett, J. H., M. M. Iles, M. Harland, J. C. Taylor, J. F. Aitken, P. A. Andresen, L. A. Akslen, B. K. Armstrong, M. F. Avril, E. Azizi, B. Bakker, W. Bergman, G. Bianchi-Scarra, B. Bressac-de Paillerets, D. Calista, L. A. Cannon-Albright, E. Corda, A. E. Cust, T. Debniak, D. Duffy, A. M. Dunning, D. F. Easton, E. Friedman, P. Galan, P. Ghiorzo, G. G. Giles, J. Hansson, M. Hocevar, V. Hoiom, J. L. Hopper, C. Ingvar, B. Janssen, M. A. Jenkins, G. Jonsson, R. F. Kefford, G. Landi, M. T. Landi, J. Lang, J. Lubinski, R. Mackie, J. Malvehy, N. G. Martin, A. Molven, G. W. Montgomery, F. A. van Nieuwpoort, S, Novakovic, H. Olsson, L. Pastorino, S. Puig, J. A. Puig-Butille, J. Randerson-Moor, H. Snowden, R. Tuominen, P. Van Belle, N. van der Stoep, D. C. Whiteman, D. Zelenika, J. Han, S. Fang, J. E. Lee, Q. Wei, G. M. Lathrop, E. M. Gillanders, K. M. Brown, A. M. Goldstein, P. A. Kanetsky, G. J. Mann, S. Macgregor, D. E. Elder, C. I. Amos, N. K. Hayward, N. A. Gruis, F. Demenais, J. A. Bishop, and D. T. Bishop. 2011. Genome-wide association study identifies three new melanoma susceptibility loci. Nature genetics 43:1108-1113.
45. Kim, Y., J. Ponomarenko, Z. Zhu, D. Tamang, P. Wang, J. Greenbaum, C. Lundegaard, A. Sette, O. Lund, P. E. Bourne, M. Nielsen, and B. Peters. 2012. Immune epitope database analysis resource. Nucleic acids research.
46. Kraus, A. A., W. Messer, L. B. Haymore, and A. M. de Silva. 2007. Comparison of plaque- and flow cytometry-based methods for measuring dengue virus neutralization. J Clin Microbiol 45:3777-3780.

1. A peptide comprising or consisting of a subsequence, portion, or an amino acid modification of Dengue virus (DV) structural or non-structural (NS) polypeptide sequence comprising or consisting of a sequence set forth in Tables 1-4, 8 & 10 (SEQ ID NOs: 11-1163), or a subsequence thereof or a modification thereof, wherein the peptide elicits, stimulates, induces, promotes, increases, or enhances an anti-DV CD8+ T cell response or an anti-DV CD4+ T cell response.

2. The peptide of claim 1, wherein the peptide comprises or consists of a subsequence, portion, or an amino acid modification of Dengue virus (DV) structural core (C), membrane (M) or envelope (E) polypeptide sequence.

3. The peptide of claim 2, wherein the structural core (C), membrane (M) or envelope (E) polypeptide sequence is identical to or derived from a DENV1, DENV2, DENV3 or DENV4 serotype.

4. (canceled)

5. The peptide of claim 1, wherein the peptide comprises or consists of a subsequence, portion, or an amino acid modification of Dengue virus (DV) NS1, NS2A, NS2B, NS3, NS4A, NS4B or NS5 polypeptide sequence.

6. The peptide of claim 5, wherein the NS1, NS2A, NS2B, NS3, NS4A, NS4B or NS5 polypeptide sequence is identical to or derived from a DENV1, DENV2, DENV3 or DENV4 serotype.

7. (canceled)

8. (canceled)

9. The peptide of claim 1, wherein the peptide comprises or consists of a peptide sequence set forth as: GLFPVSLPITAAAWY (SEQ ID NO: 11); GKTKRYLPAIVREAI (SEQ ID NO: 12); GLPIRYQTPAIRAEH (SEQ ID NO: 13); or IGCYSQVNPITLTAA (SEQ ID NO: 14), or a subsequence thereof or a modification thereof.

10. The peptide of claim 1, wherein the peptide comprises or consists of a peptide sequence set forth as: TPEGIIPAL, TPEGIIPALF, TPEGIIPSM, TPEGIIPSMF, TPEGIIPTLF, YTPEGIIPTL, GEARKTFVDL, GEARKTFVEL, GEQRKTFVEL, GESRKTFVEL, LPVWLAHKVA, LPVWLAYKV, LPVWLAYKVA, LPVWLAYRVA, LPVWLSYKV, HPGAGKTKRY, NPEIEDDIF, DTTPFGQQR, MSFRDLGRVM, ATGPILTLW, ATGPISTLW, ATGPITTLW, ATGPLTTLW, ATGPVLTLW, LATGPVLTLW, CLIPTAMAF, MLIPTAMAF, VATTFVTPM, KPRICTREEF, KPRLCTREEF, NPRLCTREEF, RPRLCTREEF, TPRMCTREEF, VPLLAIGCY, VPLLAMGCY, MSYSMCTGKF, DPASIAARGY, IANQATVLM, APTRVVAAEM, APTRVVASEM, TPMLRHTIEN, MLVTPSMTM, FTMRHKKATY, FTILALFLAH, WHYDQDHPY, MALKDFKEF, LMKITAEWLW, MEITAEWLW, VMGITAEWLW, TETTILDVDL, GEFRLRGEQR (SEQ ID NOs: 764-812, 1106, 888) or a subsequence thereof or a modification thereof.

11. The peptide of claim 1, wherein said amino acid modification is 1, 2, 3, 4, 5 or 6 conservative, non-conservative, or conservative and non-conservative amino acid substitutions.

12. The peptide of claim 1, wherein the peptide is isolated or purified.

13. The peptide of claim 1, wherein said anti-DV CD8+ T cell response comprises increased IFN-gamma, TNF-alpha, IL-1alpha, IL-6 or IL-8 production by CD8+ T cells in the presence of the peptide.

14. The peptide of claim 1, wherein the CD4+ T cells produce IFN-gamma, TNF, IL-2, or CD40L in the presence of the peptide, or kill peptide-pulsed target cells.

15. (canceled)

16. (canceled)

17. A composition comprising the peptide of claim 1.

18. A pharmaceutical composition comprising the peptide of claim 1.

19. A composition comprising 2, 3, 4, 5, 6, 7, 8, 9, 10, 11, 12, 13, 14, 15, 16, 17, 18, 19, 20, 21, 22, 23, 24, 25, or more peptides selected from Tables 1-4, 8 & 10 (SEQ ID NOs: 11-1163), or a subsequence thereof or a modification thereof.

20. The composition of claim 19, wherein the composition comprises a pharmaceutical composition.

21. (canceled)

22. The composition of claim 17, further comprising an adjuvant.

23. (canceled)

24. (canceled)

25. (canceled)

26. (canceled)

27. A method of stimulating, inducing, promoting, increasing, or enhancing an immune response against Dengue virus (DV) in a subject, comprising administering to a subject an amount of a peptide of claim 1 sufficient to stimulate, induce, promote, increase, or enhance an immune response against Dengue virus (DV) in the subject.

28. The method of claim 27, wherein the immune response provides the subject with protection against a Dengue virus (DV) infection or pathology, or one or more physiological conditions, disorders, illness, diseases or symptoms caused by or associated with DV infection or pathology.

29. A method of treating a subject for a Dengue virus (DV) infection, comprising administering to a subject an amount of a peptide of claim 1 sufficient to treat the subject for the Dengue virus (DV) infection.

30. The method of claim 27, wherein the Dengue virus comprises a DENV1, DENV2, DENV3 or DENV4 serotype.

31. The method of claim 27, wherein the peptide comprises or consists of a Dengue virus (DV) T cell epitope.

32. The method of claim 31, wherein the T cell epitope elicits, stimulates, induces, promotes, increases, or enhances an anti-DV CD8+ T cell response or an anti-DV CD4+ T cell response.

33. The method of claim 31, wherein the Dengue virus (DV) T cell epitope is a structural or non-structural (NS) T cell epitope.

34. The method of claim 31, wherein the Dengue virus T cell epitope comprises or consists of a subsequence or portion of Dengue virus C, M or E protein.

35. (canceled)

36. The method of claim 31, wherein the Dengue virus T cell epitope comprises or consists of a subsequence or portion of NS1, NS2A, NS2B, NS3, NS4A, NS4B or NS5 protein.

37. The method of claim 29, wherein the Dengue virus (DV) infection is acute.

38. The method of claim 29, wherein the subject is a mammal.

39. The method of claim 29, wherein the subject is a human.

40. (canceled)

41. The method of claim 27, wherein the method reduces one or more adverse physiological conditions, disorders, illness, diseases, symptoms or complications caused by or associated with Dengue virus (DV) infection or pathology.

42. The method of claim 27, wherein the method improves one or more adverse physiological conditions, disorders, illness, diseases, symptoms or complications caused by or associated with Dengue virus (DV) infection or pathology.

43. (canceled)

44. The method of claim 27, wherein the method reduces or inhibits susceptibility to Dengue virus (DV) infection or pathology.

45. The method of claim 31, wherein the Dengue virus (DV) T cell epitope is administered prior to, substantially contemporaneously with or following exposure to or infection of the subject with Dengue virus (DV).

46. The method of claim 31, wherein a plurality of Dengue virus (DV) T cell epitopes are administered prior to, substantially contemporaneously with or following exposure to or infection of the subject with Dengue virus (DV).

47. (canceled)

48. (canceled)

49. (canceled)

50. (canceled)

51. (canceled)

52. (canceled)

53. (canceled)

54. The method of claim 27, wherein the peptide comprises or consists of a peptide sequence set forth as: GLFPVSLPITAAAWY (SEQ ID NO: 11); GKTKRYLPAIVREAI (SEQ ID NO: 12); GLPIRYQTPAIRAEH (SEQ ID NO: 13); or IGCYSQVNPITLTAA (SEQ ID NO: 14), or a subsequence thereof or a modification thereof.

55. The peptide of claim 1, wherein the peptide comprises or consists of a sequence set forth as any of: GPMKLVMAF, GPMKLVMAFI, MAFIAFLRF, KSGAIKVLK, ITLLCLIPTV, CLMMMLPATL, TLLCLIPTV, LMMMLPATL, LMMMLPATLA, MMMLPATLA, MMMLPATLAF, MLIPTAMAF, TLMAMDLGEL, VTYECPLLV, HPGFTILALF, FTIMAAILAY, FTILALFLAH, TIMAAILAY, MLVTPSMTM, CPTQGEATL, CPTQGEAVL, LPEEQDQNY, YENLKYSVI, QEGAMHSAL, QEGAMHTAL, MSYTMCSGK, MSYSMCTGKF, SPCKIPFEIM, IPFEIMDLEK, EPGQLKLNWF, FGAIYGAAF, SWMVRILIGF, IGIGILLTW, SPKRLATAIA, KQIANELNY, VYTQLCDHRL, VYTQLCDHR, KAVHADMGYW, RASFIEVKTC, FAGPVSQHNY, RPGYHTQTA, GPWHLGKLEL, GPWHLGKLEM, RYMGEDGCWY, FTMGVLCLAI, MSFRDLGRVM, TYLALIATF, IQPFLALGF, GFFLRKLTSR, MMATIGIAL, MALSIVSLF, MAVGMVSIL, IPMTGPLVAG, GPLVAGGLL, SPILSITISE, FPVSIPITAA, IPITAAAWY, MEGVFHTMW, MEGVFHTMWH, KPGTSGSPI, KPGTSGSPII, GTSGSPIIDK, SPIINREGKV, NPEIEDDIF, HPGAGKTKRY, LPAIVREAI, APTRVVAAEM, APTRVVASEM, APTRVVAAEM, SEMAEALKGM, LPIRYQTPA, LPIRYQTPAI, DPASIAARGY, PASIAARGYI, TPPGSRDPF, FPQSNAPIMD, FPQSNAPIM, EERDIPERSW, REIPERSWNT, YPKTKLTDWD, RVIDPRRCMK, RVIDPRRCLK, RVIDPRRCMK, RVIDPRRCL, RVIDPRRCMK, DPRRCLKPV, MPVTHSSAA, MPVTHSSAAQ, NPAQEDDQY, QYIFTGQPL, TPEGIIPSM, TPEGIIPSMF, TPEGIIPAL, TPEGIIPALF, TPEGIIPSM, TPEGIIPSMF, TPEGIIPTLF, TPEGIIPALF, GEFRLRGEQR, GEARKTFVEL, GEARKTFVDL, GESRKTFVEL, GEQRKTFVEL, ELMRRGDLPV, LPVWLAYKVA, YKVASAGISY, REWCFTGERN, RPRWLDART, MALKDFKEF, EFKEFAAGR, FASGRKSITL, LPTFMTQKAR, MTQKARNAL, TAEAGGRAY, LPETLETLLL, LETLMLVAL, LETLMLVALL, TLMLLALIAV, LMLLALIAVL, MLLALIAVL, GAMLFLISGK, SIILEFFLMV, IILEFFLMV, IILEFFLMVL, FLMVLLIPEP, TPQDNQLAYV, TPQDNQLTY, TTKRDLGMSK, TETTILDVDL, RPASAWTLY, RPASAWTLYA, HPASAWTLY, PASAWTLYAV, VATTFVTPM, ITPMLRHTI, TPMLRHTIEN, IANQATVLM, VPLLAIGCY, NPLTLTAAV, TLTAAVLLLV, AAVLLLVTHY, VLLLVTHYAI, DPIPYDPKF, MLLILCVTQV, ATGPLTTLW, ATGPISTLW, ATGPITTLW, ATGPILTLW, ATGPVLTLW, LWEGSPGKF, SPGKFWNTTI, IAVSMANIF, IAVSMANIF, IAVSTANIF, MANIFRGSY, YLAGAGLAF, GSSKIRWIVE, GPGHEEPIPM, IPMSTYGWNL, IPMATYGWNL, IPMSTYGWNL, MSTYGWNIVK, STYGWNIVK, QSGVDVFFTP, RVLKMVEPW, KVLNPYMPSV, VLNPYMPSV, MPSVIEKMET, VSSVNMVSRL, MVSRLLLNR, FTMRHKKATY, WHYDQDHPY, QENPYRTWAY, WAYHGSYET, WAYHGSYEV, DTTPFGQQR, TPFGQQRVF, EPKEGTKKLM, MEITAEWLW, KPRICTREEF, TPRMCTREEF, KPRLCTREEF, NPRLCTREEF, RPRLCTREEF, TPRMCTREEF, RAAVEDEEF, EAVEDSRFWE, KGSRAIWYMW, RYLEFEALGF, RFLEFEALGF, FSRENSLSGV, GEGLHKLGY, RQLANAIFK, TPRGTVMDII, TPKGAVMDII, RQMEGEGIF, RQMEGEGVL, KVRKDIQQW, YAQMWSLMY, YAQMWSLMYF, ICSAVPVHW, WSIHAHHQW, NPNMIDKTPV, NPWMEDKTPV, VPYLGKREDQ, REDLWCGSL, REDQWCGSL, MPSMKRFRRE, APFESEGVL (SEQ ID NOs: 555-763), or a subsequence or a modification thereof.

56. The peptide of claim 1, wherein the peptide comprises or consists of a sequence set forth as any of: ITEAELTGY, RSCTLPPLRY, MTDDIGMGV, LTDALALGM, VIDLDPIPY, YTDYMPSMK, RLITVNPIV, IMAVGMVSI, GLLTVCYVL, LLVISGLFPV, AAAWYLWEV, YLPAIVREA, DLMRRGDLPV, ALSELPETL, IILEFFLIV, KLAEAIFKL, SQIGAGVYK, GTSGSPIIDK, KTFDSEYVK, RIYSDPLALK, ATVLMGLGK, STYGWNLVR, TVMDIISRR, RQMEGEGVFK, RTTWSIHAK, RPTFAAGLLL, LPAIVREAI, APTRVVAAEM, VPNYNLIIM, APIMDEEREI, TPEGIIPSMF, KPRWLDARI, RPASAWTLYA, TPMLRHSI, SPNPTVEAGR, TPRMCTREEF, RPTPRGTVM, AFLRFLTIPPTAGIL, GVTYLALLAAFKVRP, MAVGMVSILASSLLK, TFTMRLLSPVRVPNY, SRAIWYMWLGARFLE (SEQ ID NOs: 15-56), or a subsequence or a modification thereof.

57. The peptide of claim 1, wherein the peptide comprises or consists of a sequence set forth as any of: ITEAELTGY, STEIQLTDY, TTEIQLTDY, TSEIQLIDY, TSEIQLTDY, IAEAELTGY, IAEAELTDY, ITDAELTGY, STEAELTGY, TTEAELTGY, IAEAELTDY, ITEAELTGY, TVEAVLLEY, TVEAVLPEY, TVEAILPEY, TAEAILPEY, THEALLPEY, ITEAILPEY, TTEVILPEY, TTEAILPEY, SVEVELPDY, SVEVKLPDY, RSCTLPPLRY, RSCTLPPLRF, RSCTLPPLRY, RSCTLPPLRY, RSCTMPPLRF, MTDDIGMGV, ASDRMGMGM, ASDMMGMGT, ASDKMGMGT, ASDNMGMGT, VSDRMGMGT, ASDRMGMGT, MADDIGMGV, MTDEMGMGV, ITDDIGMGV, MTDDIGMGV, ASDRTGMGV, ASDKMGMGV, ATDRMGMGV, ASDRMGMGV, LTDALALGM, LGDGLAIGI, LGDGFAMGI, LGDGLAMGI, LTDAIALGI, LTDAWALGM, LTDALALGI, LTDALALGM, MANGVALGL, MANGIALGL, LISGISLGL, FIDGLSLGL, LIDGISLGL, LIDGIALGL, FIDGISLGL, VIDLDPIPY, TIDLDPVVY, AIDLDPVVY, VIDLEPIPY, VIDLDPIPY, TIDLDSVIF, TIDLDPVIY, TIALDPVIY, VIDLEPISY, YTDYMPSMK, YSDYMTSMK, YLDYMASMK, YIDYMTSMK, YLDFMTSMK, YLDYMTSMK, YLDYMISMK, YIDYMPSMK, YMDYMPSMK, YTDYMPSMK, FLDYMPSMK, YADYMPVMK, YMDYMPVMK, YVDYMPAMK, YVDYMPVMR, YVDYMPVMK, RLITVNPIV, RVITANPIV, RLVTANPIV, RLITANPIV, RLITVNPVV, RLITVNPII, RLITVNPIV, RLTTVNPIV, RLITANPIV, RLITANPVV, RVISATPLA, RVISSTPLA, RIISSTPLA, RVISSTPFA, RIISSTPFA, RIISSIPFA, IMAVGMVSI, IMAVGVVSI, VMAVGIVSI, IMAIGIVSI, IMAVGIVSI, VMAVGMVSI, IMAVGMVSI, VMAIGLVSI, VMAVGLVSI, MMAVGLVSL, IMAVGLVSL, GLLTVCYVL, GMLITCYVI, GMLIACYVI, GPLTVCYVL, GLLTVCYVL, GMLIACYVI, GLLIACYVI, GLLLAAYMM, GLLLAAYVM, RIYSDPLALK, RTYSDPQALR, RTYSDPLALR, RTYSDPLALK, RIYSDPLTLK, KIYSDPLALK, RIYSEPRALK, RIYSDPLALK, RTYSDPLAPK, RTYSDPLALK, RIYSDPLALK, RVYADPMALQ, RVYADPMALK, ATVLMGLGK, AAILMGLDK, ATVLMGLGK, ATVLMGLGR, AVVLMGLNK, AVVLMGLDK, AAVLMGLGK, STYGWNLVR, AAYGWNLVK, ATYGWNLVK, STYGWNLVR, STYGWNLVK, STYGWNVVK, STYGWNIVK, ATYGWNLVK, TVMDIISRR, TVMDIISRR, TVMDVISRR, TVLDIISRR, TVMDIISRK, TVMDIISRR, TVMDIISRK, AVMDIISRK, RQMEGEGVFK, RQMESEEIFS, RQMESEGIVS, RQMESEGIFF, RQMESEGIIL, RQMESEGIFS, RQMESEGIFL, RQMEGEGVFR, RQMEGEGIFR, RQMEGEGLFK, RQMEGEEVFK, RQMEGEGVFK, RQMEGEGIFK, RQMEGEGVLT, RQMEGEGVLS, RQMEGEDVLS, RQMEAEGVIT, RTTWSIHAK, RTTWSIHAH, RTTWSIHAR, RTTWSIHAT, RTTWSIHAS, RTTWSIHAK, RTTWSIHAH, RTTWSIHAH, RPTFAAGLLL, RPMLAVGLLF, RPMFAMGLLF, RPMFAVGLLI, RPMFAVGLLF, RPTFAAGLFL, RPTFAVGLVL, RPTFAVGLLL, RPTFAAGLLL, QPFLALGFFM, QPFLTLGFFL, QPFLALGFFL, SPRYVLGVFL, SPGYVLGVFL, SPGYVLGIFL, LPAIVREAI, LPAIIREAI, LPAIVREAI, LPAMVREAI, LPAIVREAI, LPTIVREAI, LPAVVREAI, LPAIVREAI, LPAIIREAI, LPSIVREAL, APTRVVAAEM, APTRVVASET, APTRVVAAEM, APTRVVASEM, APPRVVPAEM, APTRVVAAEM, APTRVVAAEM, APTRVVAAEM, VPNYNLIIM, VPNYNMIIV, VPNYNMIIM, VPNYNMIVM, VPNYNLIIM, VPNYNLIVM, VPNYNLVVM, VPNYNLVIM, VSNYNLIIM, VPNYNLIIM, VPNYNLIVM, APIMDEEREI, AIIQDEERDI, AVIQDEEKDI, AAIQDEERDI, AVIQDEERDI, APIMDDEREI, APIIDEEREI, APIVDEEREI, APIMDEEREI, APIQDEEKDI, SPIQDEERDI, APIQDEERDI, APIQDKERDI, SPIEDIEREI, TPEGIIPSMF, TPEGIIPALY, TPEGIIPALF, TPEGIIPSLF, TPEGIIPSMF, TPEGIIPALF, TPEGIIPTLF, LLVISGLFPV, LLAISGVYPL, LLAVSGMYPL, LLAVSGVYPL, LLVISGVYPM, LLAVSGVYPI, LLAASGVYPM, LLAISGVYPM, LLAVSGVYPM, LLVVSGLFPV, LLVISGLFPA, LLVISGLFPI, LLVISGVFPV, LLVISGLFPV, LLIVSGIFPC, LLIVSGIFPY, LLIVSGVFPY, LITVSGLYPL, AAAWYLWEV, LFVWCFWQK, LFLWYFWQK, LFVWHFWQK, FFVWYFWQK, PFVWYFWQK, LFVWYFWQK, AAAWYLWET, AAAWYLWEA, AAAWYLWEV, LLVWHAWQK, MLVWHTWQK, LLVWHTWQK, MALWYIWQV, MTLWYMWQV, MALWYMWQV, YLPAIVREA, YLPAIIREA, YLPAIVREA, YLPAMVREA, SLPAIVREA, YLPAIVREA, YLPTIVREA, YLPAVVREA, YLPAIVREA, YLPAIIREA, ILPSIVREA, DLMRRGDLPV, DLLRRGDLPV, ELMRRGDLPV, DLMKRGDLPV, ELMRRGDLPV, DLMRRGDLPV, ELMRRGHLPV, ELMRRGDLPV, ELMKRGDLPV, ELMRRGDLPV, ALSELPETL, ALEELPDTI, AVEELPDTI, AMEELPDTI, ALSELAETL, ALGELPETL, ALSELPETL, AVEELPETM, ALNELTESL, ALNELPESL, IILEFFLIV, IILKFFLMV, IILEFLLMV, IMLEFFLMV, IILEFFLMV, IILEFFLMV, IILEFFLIV, IILEFFMMV, IVLEFFMMV, IILEFFLMV, KLAEAIFKL, LLAKAIFKL, QLAKSIFKL, LLATSVFKL, LLAKSIFKL, LLATAIFKL, LLATSIFKL, LLAKSIFKL, KLAEAIFRL, RLAEAIFKL, KLAEAVFKL, KLAEAIFKL, QLASAIFKL, LLANAIFKL, RLANAIFKL, QLANAIFKL, TLAKAIFKL, ILAKAIFKL, SQIGAGVYK, SQVGVGVFQ, SQIGAGVYR, SQIGTGVYK, SQIGVGVYK, SQIGAGVYK, TQVGVGIQK, TQVGVGVHK, TQVGVGVQK, TQVGVGIHI, TQVGVGIHT, TQVGVGIHM, TQVGVGVHV, GTSGSPIIDK, GTSGSPIVSR, GTSGSPIVNR, GTSGSPIIDK, GTSGSPIADK, GTSGSPIVDR, GTSGSPIVDK, GTSGSPIINK, GTSGSPIINR, GSSGSPIINR, GTSGSPIVNR, GTSGSPIINK, GTSGSPIINR, KTFDSEYVK, KTFDTEYQK, KTFDTEYTK, KTFDTEYIK, KTFDFEYIK, KTFDSEYIK, KTFDSEYAK, KTFDSEYVK, KTFDTEYQR, KTFNTEYQK, KTFDTEYQK, KTFDTEYPK, KPRWLDARI, RPRWLDART, KPRWLDART, KPRWLDAKI, KPRWLDPRI, KPRWLDARI, RPRWLDART, RPRWLDARI, RPRWLDARV, RPKWLDARV, RPASAWTLYA, HPASAWTLYA, RPASAWTLYA, RPASAWTLYA, HPASAWILYA, HPASAWTLYA, RPASAWTLYA, TPMLRHSI, TPMLRHTI, TPMMRHTI, TPMLRHSI, TPMLRHTI, TPMLRHTI, SPNPTVEAGR, SPNPTIEEGR, SPSPTVEAGR, SPNPTVDAGR, SPNPTVEAGP, SPNPTIEAGR, SPNPTVEAGR, SPSPTVEEGR, SPSLTVEESR, SPSPIVEESR, SPSPTVEESR, SSNPTIEEGR, TPRMCTREEF, KPRICTREEF, RPRICTRAEF, KPRICTRAEF, TRRMCTREEF, TPRICTREEF, IPRMCTREEF, TPRMCTREEF, KPRLCPREEF, KPRLCTREEF, RPRLCTREEF, NPRLCTKEEF, SPRLCTREEF, TPRLCTREEF, SPRLCTKEEF, NPRLCTREEF, KPRLCTREEF, RPTPRGTVM, RPVKNGTVM, RPARNGTVM, RPAKNGTVM, RPAKSGTVM, RPTPRGTVL, RPTPKGTVM, RPTPIGTVM, RPTPRGTVM, RPTPKGTVM, RPTPTGTVM, RPTPRGAVM, RPTPKGAVM, AFLRFLTIPPTAGIL, AFLRFLAIPPTAGIV, ALLRFLAIPPTAGIL, AFLTFLAIPPTAGIL, AFLRFLAIPPTAGIL, AFLRFLTISPTAGIL, AFLRFLTIPPTVGIL, AFLRFLTIPPTAGIL, AFLRFLAIPPTAGIL, AFLRFLAIPPTAGVL, TFLRVLSIPPTAGIL, GVTYLALLAAFKVRP, GTTYLALMATFRMRP, GMTYLALMATFKMRP, GTTYLALMATLKMRP, GTTHLALMATFKMRP, GTTYLALMATFKMRP, GVTYLALLATFKVRP, GVTYLALLAAYKVRP, GVTYLALLAAFRVRP, GVTYLALLAAFKVRP, GVTYLALIATFEIQP, GVTCLALIATFKIQP, GVTYLALIATFKVQP, GVTYLALIATFKIQP, GQTHLAIMAVFKMSP, GQIHLAIMAVFKMSP, GQTHLAIMIVFKMSP, GQVHLAIMAVFKMSP, GQIHLAIMTMFKMSP, MAVGMVSILASSLLK, MAVGVVSILLSSLLK, MAIGIVSILLSSLLK, MAVGIVSILLSSLLK, MAVGMVSILASSLLK, MAVGLVSILASSFLR, MAIGLVSILASSLLR, MAVGLVSILASSLLR, MAVGLVSLLGSALLK, TFTMRLLSPVRVPNY, TFTMRLLSPVRVPNY, PFTMRLLSPVRVPNY, TFTMRLLSPIRVPNY, TFTMRLLSPVRVPNY, TFTMRLLSPVRVSNY, PFTMRLLSPVRVPNY, TFTMRLLSPVRVPNY, TFTTKLLSSTRVPNY, TFTTRLLSSTRVPNY, SRAIWYMWLGARFLE, SRAIWYVWLGARFLE, SRAIWYMWLGAAFLE, SRAIWYMWLGARFLE, SRAIWYMWLGARFLE, SRAIWYMWLGARFLE, SRAIWYMWLGVRYLE, SRAIWYMWLGARYLE, SRAIWYMWLGARFLE (SEQ ID NOs: 57-554), or a subsequence or a modification thereof.

58. The peptide of claim 1, wherein the peptide comprises or consists of a sequence set forth as any of: AAVLLLVTHY, ALRGLPIRY, AMALSIVSLF, MALSIVSLF, APFESEGVL, APIMDEEREI, APTRVVAAEM, APTRVVASEM, ASSMVNGVVK, ASSMVNGVVR, ASSVLLWMAS, ATGPILTLW, ATGPISTLW, ATGPITTLW, ATGPLTTLW, ATGPVLTLW, LATGPVLTLW, ATYGWNLVK, MATYGWNLVK, MSTYGWNIVK, AVQTKPGLFK, CLIPTAMAF, MLIPTAMAF, CLMMMLPATL, CPTQGEATL, CPTQGEAVL, CPTQGEPSL, DPASIAARGY, DPIPYDPKF, DPRRCLKPV, DTTPFGQQR, DYMPSMKRFR, EAVEDSRFWE, EENMDVEIW, EERDIPERSW, EFKEFAAGR, EFKEFAAGRR, ELMRRGDLPV, EPDYEVDEDI, EPGQLKLNWF, EPGVVSPTSY, EPIEGKVVQY, EPISYDPKF, EPKEGTKKLM, ESSSNPTIEE, ETACLGKAY, ETACLGKAYA, ETACLGKSY, KETACLGKSY, RETACLGKAY, RETACLGKSY, ETTHHAVSR, ETTKHAVSR, FAGPVSQHNY, FASGRKSITL, FGAIYGAAF, FLMVLLIPEP, FMALVAFLRF, FMKDGRSLVV, FPQSNAPIMD, FPVSIPITAA, FSRENSLSGV, FTILALFLAH, FTIMAAILAY, TIMAAILAY, TLMAAILAY, FTMGVLCLAI, FTMRHKKATY, FTNMEAQLVR, GAMLFLISGK, GASKRSWPLN, GEARKTFVDL, GEARKTFVEL, GEQRKTFVEL, GESRKTFVEL, GEFRLRGEQR, GEGLHKLGY, GEVGAIALDF, GFFLRKLTSR, GGWRLSAQW, GMGEAAAIF, GPGHEEPIPM, GPLVAGGLL, GPMKLVMAF, GPMKLVMAFI, GPSLRTTTV, GPWHLGKLEL, GPWHLGKLEM, GSSKIRWIVE, GTSGSPIIDK, TSGSPIIDK, GTSGSPIVNR, HALLATSIF, HMIAGVLFTF, HPASAWTLY, RPASAWTLYA, HPGAGKTKRY, HPGFTILALF, HQLWATLLSL, IAFLRFLAI, IANQAAILM, IANQATVLM, IAVSMANIF, IAVSTANIF, ICSAVPVHW, IGIGILLTW, IGIGVLLTW, IGLTSRATW, IILEFFLMV, IPFEIMDLEK, IPITAAAWY, IPKAYAGPF, IPMATYGWNL, IPMSTYGWNL, IPMTGPLVAG, IPQWEPSKGW, IPTVMAFHL, IQPFLALGF, ISSMLNIMNR, ITAAAWYLW, ITLLCLIPTV, TLLCLIPTV, ITPMLRHTI, IVIGVGDSAL, KAKGSRAIW, RAKGSRAIW, KATREAQKRA, KAVHADMGYW, RAVHADMGYW, KAYAQMWSL, KEGVFHTMW, KGSRAIWYMW, KPGTSGSPI, KPGTSGSPII, KPGTSGSPIV, KPRICTREEF, KPRLCTREEF, NPRLCTREEF, RPRLCTREEF, TPRMCTREEF, KPRWLDARI, KPTLDFELI, KPWDVIPMV, KPWDVVPMV, KQIANELNY, KQISNELNY, KSGAIKVLK, KSYAQMWQL, KTDFGFYQV, KTFVDLMRR, KTWAYHGSY, KVIQLSRKTF, KVLNPYMPSV, VLNPYMPSV, KVRKDIQQW, LAYTIGTTHF, LEFEALGFLN, LEFEALGFMN, LETLMLVAL, LGFLNEDHW, LIHQVFGTAY, LVHQIFGTAY, LILCVTQVLM, LLMLVTPSM, LLSPVRVPNY, LMCHATFTM, LMKITAEWLW, MEITAEWLW, VMGITAEWLW, LMMATIGIAL, MMATIGIAL, LMMILPAAL, LMMMLPATLA, LMMTTIGVVL, LPAIVREAI, LPEEQDQNY, LPESLETLM, LPETLETLLL, LPGADTQGSN, LPIRYQTPA, LPIRYQTPAI, LPLPWLPGAD, LPLPWTSGA, LPTFMTQKAR, LPTYLSSRAK, LPVWLAHKVA, LPVWLAYKV, LPVWLAYKVA, LPVWLAYRVA, LPVWLSYKV, LRVLNLVENR, LSMGLITIAV, LSRNSTHEM, LTAAVLLLI, TLTAAVLLLV, LWEGSPGKF, MAEIPLQWI, MAFIAFLRF, MAILGDTAW, MALKDFKEF, MAMTCIAVG, MANEMGFLEK, MANIFRGSY, MASSALLWMA, MAVGMVSIL, MGYWIESAL, MIDKTPVHSW, MLLALIAVL, MLLILCVTQV, MLVTPSMTM, MLWMAEIPL, MMLKLLTDF, MMLPATLAF, MMMLPATLAF, MPGTFQTTTG, MPGTRKVMGI, MPGTRRVMGI, MPLVMAWRTI, MPSMKRFRRE, MPSVIEKMET, MPVMKRYSAP, MPVTHSSAAQ, MPVTVASAAQ, MSFRDLGRVM, MSSEGAWKHA, MSYSMCTGKF, MSYTMCSGK, MTQKARNAL, MTTTANWLW, MVSRLLLNR, NPAQEDDQY, NPEIEDDIF, NPITLTAAL, NPLTLTAAV, NPNMIDKTPV, NPWMEDKTH, NPWMEDKTPV, NPTVDGITV, NQLIYVILTI, PASAWTLYAV, PASIAARGYI, PTSRTTWSIH, QEGAMHSAL, QEGAMHTAL, QENPYRTWAY, QIAMTDTTPF, QPESNILDI, QPHWIAASI, QSGVDVFFTP, QYIFTGQPL, QYSDRRCF, RAAVEDEEF, RASFIEVKTC, REDLWCGSL, REDQWCGSL, REIPERSWNT, REWCFTGERN, RFLEFEALGF, RYLEFEALGF, RLRGEARKTF, RPGYHTQTA, RPISEKEENM, RPRWLDART, RPTPKGAVM, RPTPKGTVM, RQLANAIFK, RQMEGEGIF, RQMEGEGVL, RVIDPRRCL, RVIDPRRCLK, RVIDPRRCM, RVIDPRRCMK, RVLKMVEPW, RYMGEDGCWY, SEMAEALKGM, SPCKIPFEIM, SPGKFWNTTI, SPIINREGKV, SPILSITISE, SPKRLATAIA, SPSPTVEESR, SPVRVPNYNL, SQIGAGVYK, STYGWNLVR, SWMVRILIGF, SYAQMWTLMY, YAQMWQLMYF, YAQMWSLMY, YAQMWSLMYF, YAQMWTLMY, YAQMWTLMYF, TAAVLLLITH, TAEAGGRAY, TETTILDVDL, TFKVPHAKR, TLMAMDLGEL, TLMLLALIAV, TPEAKNSTF, TPEARNSTF, TPEGIIPAL, TPEGIIPALF, TPEGIIPSM, TPEGIIPSMF, TPEGIIPTLF, YTPEGIIPTL, TPFGQQRVF, TPKGAVMDII, TPRGTVMDII, TPMLRHTIEN, TPPGSRDPF, TPQDNQLAY, TPQDNQLAYV, TPQDNQLTY, TPRSMPGTRR, TPVHSWEDI, TTFSLHYAW, TTKRDLGMSK, TYLALIATF, VASGLLWVAE, VATTFVTPM, VLLLVTHYAI, VPLLAIGCY, VPLLAMGCY, VPMVTQMAM, VPNYNMIIM, VPYLGKREDQ, VQADMGCVV, VSSVNMVSRL, VTIDLDPVVY, VTRGAVLMHK, VTYECPLLV, VYTQLCDHR, VYTQLCDHRL, WALCESITL, WAYHGSYET, WAYHGSYEV, WHYDQDHPY, WSIHAHHQW, WVAEIQPQW, YAQIQPHWI, YENLKYSVI, YGGPISQHNY, YGVLFSGVSW, YKVASAGISY, YLAGAGLAF, YPKTKLTDW, YPKTKLTDWD, YQLAVTITAI, YQLWTALISL, YQNKVVKVLR, YQNKVVKVQR, YVSAIAQTEK (SEQ ID NOs: 813-1163), or a subsequence thereof or a modification thereof.

59. The method of claim 27, wherein the peptide comprises or consists of a sequence set forth as any of: TPEGIIPAL, TPEGIIPALF, TPEGIIPSM, TPEGIIPSMF, TPEGIIPTLF, YTPEGIIPTL, GEARKTFVDL, GEARKTFVEL, GEQRKTFVEL, GESRKTFVEL, LPVWLAHKVA, LPVWLAYKV, LPVWLAYKVA, LPVWLAYRVA, LPVWLSYKV, HPGAGKTKRY, NPEIEDDIF, DTTPFGQQR, MSFRDLGRVM, ATGPILTLW, ATGPISTLW, ATGPITTLW, ATGPLTTLW, ATGPVLTLW, LATGPVLTLW, CLIPTAMAF, MLIPTAMAF, VATTFVTPM, KPRICTREEF, KPRLCTREEF, NPRLCTREEF, RPRLCTREEF, TPRMCTREEF, VPLLAIGCY, VPLLAMGCY, MSYSMCTGKF, DPASIAARGY, IANQATVLM, APTRVVAAEM, APTRVVASEM, TPMLRHTIEN, MLVTPSMTM, FTMRHKKATY, FTILALFLAH, WHYDQDHPY, MALKDFKEF, LMKITAEWLW, MEITAEWLW, VMGITAEWLW, TETTILDVDL, GEFRLRGEQR (SEQ ID NOs: 764-812, 1106, 888) or a subsequence thereof or a modification thereof.

60. (canceled)

61. (canceled)

62. (canceled)

63. (canceled)

64. (canceled)

65. (canceled)

66. The method of claim 27 wherein the peptide comprises or consists of a sequence set forth as any of: GPMKLVMAF, GPMKLVMAFI, MAFIAFLRF, KSGAIKVLK, ITLLCLIPTV, CLMMMLPATL, TLLCLIPTV, LMMMLPATL, LMMMLPATLA, MMMLPATLA, MMMLPATLAF, MLIPTAMAF, TLMAMDLGEL, VTYECPLLV, HPGFTILALF, FTIMAAILAY, FTILALFLAH, TIMAAILAY, MLVTPSMTM, CPTQGEATL, CPTQGEAVL, LPEEQDQNY, YENLKYSVI, QEGAMHSAL, QEGAMHTAL, MSYTMCSGK, MSYSMCTGKF, SPCKIPFEIM, IPFEIMDLEK, EPGQLKLNWF, FGAIYGAAF, SWMVRILIGF, IGIGILLTW, SPKRLATAIA, KQIANELNY, VYTQLCDHRL, VYTQLCDHR, KAVHADMGYW, RASFIEVKTC, FAGPVSQHNY, RPGYHTQTA, GPWHLGKLEL, GPWHLGKLEM, RYMGEDGCWY, FTMGVLCLAI, MSFRDLGRVM, TYLALIATF, IQPFLALGF, GFFLRKLTSR, MMATIGIAL, MALSIVSLF, MAVGMVSIL, IPMTGPLVAG, GPLVAGGLL, SPILSITISE, FPVSIPITAA, IPITAAAWY, MEGVFHTMW, MEGVFHTMWH, KPGTSGSPI, KPGTSGSPII, GTSGSPIIDK, SPIINREGKV, NPEIEDDIF, HPGAGKTKRY, LPAIVREAI, APTRVVAAEM, APTRVVASEM, APTRVVAAEM, SEMAEALKGM, LPIRYQTPA, LPIRYQTPAI, DPASIAARGY, PASIAARGYI, TPPGSRDPF, FPQSNAPIMD, FPQSNAPIM, EERDIPERSW, REIPERSWNT, YPKTKLTDWD, RVIDPRRCMK, RVIDPRRCLK, RVIDPRRCMK, RVIDPRRCL, RVIDPRRCMK, DPRRCLKPV, MPVTHSSAA, MPVTHSSAAQ, NPAQEDDQY, QYIFTGQPL, TPEGIIPSM, TPEGIIPSMF, TPEGIIPAL, TPEGIIPALF, TPEGIIPSM, TPEGIIPSMF, TPEGIIPTLF, TPEGIIPALF, GEFRLRGEQR, GEARKTFVEL, GEARKTFVDL, GESRKTFVEL, GEQRKTFVEL, ELMRRGDLPV, LPVWLAYKVA, YKVASAGISY, REWCFTGERN, RPRWLDART, MALKDFKEF, EFKEFAAGR, FASGRKSITL, LPTFMTQKAR, MTQKARNAL, TAEAGGRAY, LPETLETLLL, LETLMLVAL, LETLMLVALL, TLMLLALIAV, LMLLALIAVL, MLLALIAVL, GAMLFLISGK, SIILEFFLMV, IILEFFLMV, IILEFFLMVL, FLMVLLIPEP, TPQDNQLAYV, TPQDNQLTY, TTKRDLGMSK, TETTILDVDL, RPASAWTLY, RPASAWTLYA, HPASAWTLY, PASAWTLYAV, VATTFVTPM, ITPMLRHTI, TPMLRHTIEN, IANQATVLM, VPLLAIGCY, NPLTLTAAV, TLTAAVLLLV, AAVLLLVTHY, VLLLVTHYAI, DPIPYDPKF, MLLILCVTQV, ATGPLTTLW, ATGPISTLW, ATGPITTLW, ATGPILTLW, ATGPVLTLW, LWEGSPGKF, SPGKFWNTTI, IAVSMANIF, IAVSMANIF, IAVSTANIF, MANIFRGSY, YLAGAGLAF, GSSKIRWIVE, GPGHEEPIPM, IPMSTYGWNL, IPMATYGWNL, IPMSTYGWNL, MSTYGWNIVK, STYGWNIVK, QSGVDVFFTP, RVLKMVEPW, KVLNPYMPSV, VLNPYMPSV, MPSVIEKMET, VSSVNMVSRL, MVSRLLLNR, FTMRHKKATY, WHYDQDHPY, QENPYRTWAY, WAYHGSYET, WAYHGSYEV, DTTPFGQQR, TPFGQQRVF, EPKEGTKKLM, MEITAEWLW, KPRICTREEF, TPRMCTREEF, KPRLCTREEF, NPRLCTREEF, RPRLCTREEF, TPRMCTREEF, RAAVEDEEF, EAVEDSRFWE, KGSRAIWYMW, RYLEFEALGF, RFLEFEALGF, FSRENSLSGV, GEGLHKLGY, RQLANAIFK, TPRGTVMDII, TPKGAVMDII, RQMEGEGIF, RQMEGEGVL, KVRKDIQQW, YAQMWSLMY, YAQMWSLMYF, ICSAVPVHW, WSIHAHHQW, NPNMIDKTPV, NPWMEDKTPV, VPYLGKREDQ, REDLWCGSL, REDQWCGSL, MPSMKRFRRE, APFESEGVL, (SEQ ID NOs: 555-763) or a subsequence or a modification thereof.

67. The method of claim 27 wherein the peptide comprises or consists of a sequence set forth as any of: ITEAELTGY, RSCTLPPLRY, MTDDIGMGV, LTDALALGM, VIDLDPIPY, YTDYMPSMK, RLITVNPIV, IMAVGMVSI, GLLTVCYVL, LLVISGLFPV, AAAWYLWEV, YLPAIVREA, DLMRRGDLPV, ALSELPETL, IILEFFLIV, KLAEAIFKL, SQIGAGVYK, GTSGSPIIDK, KTFDSEYVK, RIYSDPLALK, ATVLMGLGK, STYGWNLVR, TVMDIISRR, RQMEGEGVFK, RTTWSIHAK, RPTFAAGLLL, LPAIVREAI, APTRVVAAEM, VPNYNLIIM, APIMDEEREI, TPEGIIPSMF, KPRWLDARI, RPASAWTLYA, TPMLRHSI, SPNPTVEAGR, TPRMCTREEF, RPTPRGTVM, AFLRFLTIPPTAGIL, GVTYLALLAAFKVRP, MAVGMVSILASSLLK, TFTMRLLSPVRVPNY, SRAIWYMWLGARFLE, (SEQ ID NOs: 15-56) or a subsequence or a modification thereof.

68. The method of claim 27 wherein the peptide comprises or consists of a sequence set forth as any of: ITEAELTGY, STEIQLTDY, TTEIQLTDY, TSEIQLIDY, TSEIQLTDY, IAEAELTGY, IAEAELTDY, ITDAELTGY, STEAELTGY, TTEAELTGY, ISEAELTDY, ITEAELTGY, TVEAVLLEY, TVEAVLPEY, TVEAILPEY, TAEAILPEY, THEALLPEY, ITEAILPEY, TTEVILPEY, TTEAILPEY, SVEVELPDY, SVEVKLPDY, RSCTLPPLRY, RSCTLPPLRF, RSCTLPPLRY, RSCTLPPLRY, RSCTMPPLRF, MTDDIGMGV, ASDRMGMGM, ASDMMGMGT, ASDKMGMGT, ASDNMGMGT, VSDRMGMGT, ASDRMGMGT, MADDIGMGV, MTDEMGMGV, ITDDIGMGV, MTDDIGMGV, ASDRTGMGV, ASDKMGMGV, ATDRMGMGV, ASDRMGMGV, LTDALALGM, LGDGLAIGI, LGDGFAMGI, LGDGLAMGI, LTDAIALGI, LTDAWALGM, LTDALALGI, LTDALALGM, MANGVALGL, MANGIALGL, LISGISLGL, FIDGLSLGL, LIDGISLGL, LIDGIALGL, FIDGISLGL, VIDLDPIPY, TIDLDPVVY, AIDLDPVVY, VIDLEPIPY, VIDLDPIPY, TIDLDSVIF, TIDLDPVIY, TIALDPVIY, VIDLEPISY, YTDYMPSMK, YSDYMTSMK, YLDYMASMK, YIDYMTSMK, YLDFMTSMK, YLDYMTSMK, YLDYMISMK, YIDYMPSMK, YMDYMPSMK, YTDYMPSMK, FLDYMPSMK, YADYMPVMK, YMDYMPVMK, YVDYMPAMK, YVDYMPVMR, YVDYMPVMK, RLITVNPIV, RVITANPIV, RLVTANPIV, RLITANPIV, RLITVNPVV, RLITVNPII, RLITVNPIV, RLTTVNPIV, RLITANPIV, RLITANPVV, RVISATPLA, RVISSTPLA, RIISSTPLA, RVISSTPFA, RIISSTPFA, RIISSIPFA, IMAVGMVSI, IMAVGVVSI, VMAVGIVSI, IMAIGIVSI, IMAVGIVSI, VMAVGMVSI, IMAVGMVSI, VMAIGLVSI, VMAVGLVSI, MMAVGLVSL, IMAVGLVSL, GLLTVCYVL, GMLITCYVI, GMLIACYVI, GPLTVCYVL, GLLTVCYVL, GMLIACYVI, GLLIACYVI, GLLLAAYMM, GLLLAAYVM, RIYSDPLALK, RTYSDPQALR, RTYSDPLALR, RTYSDPLALK, RIYSDPLTLK, KIYSDPLALK, RIYSEPRALK, RIYSDPLALK, RTYSDPLAPK, RTYSDPLALK, RIYSDPLALK, RVYADPMALQ, RVYADPMALK, ATVLMGLGK, AAILMGLDK, ATVLMGLGK, ATVLMGLGR, AVVLMGLNK, AVVLMGLDK, AAVLMGLGK, STYGWNLVR, AAYGWNLVK, ATYGWNLVK, STYGWNLVR, STYGWNLVK, STYGWNVVK, STYGWNIVK, ATYGWNLVK, TVMDIISRR, TVMDIISRR, TVMDVISRR, TVLDIISRR, TVMDIISRK, TVMDIISRR, TVMDIISRK, AVMDIISRK, RQMEGEGVFK, RQMESEEIFS, RQMESEGIVS, RQMESEGIFF, RQMESEGIIL, RQMESEGIFS, RQMESEGIFL, RQMEGEGVFR, RQMEGEGIFR, RQMEGEGLFK, RQMEGEEVFK, RQMEGEGVFK, RQMEGEGIFK, RQMEGEGVLT, RQMEGEGVLS, RQMEGEDVLS, RQMEAEGVIT, RTTWSIHAK, RTTWSIHAH, RTTWSIHAR, RTTWSIHAT, RTTWSIHAS, RTTWSIHAK, RTTWSIHAH, RTTWSIHAH, RPTFAAGLLL, RPMLAVGLLF, RPMFAMGLLF, RPMFAVGLLI, RPMFAVGLLF, RPTFAAGLFL, RPTFAVGLVL, RPTFAVGLLL, RPTFAAGLLL, QPFLALGFFM, QPFLTLGFFL, QPFLALGFFL, SPRYVLGVFL, SPGYVLGVFL, SPGYVLGIFL, LPAIVREAI, LPAIIREAI, LPAIVREAI, LPAMVREAI, LPAIVREAI, LPTIVREAI, LPAVVREAI, LPAIVREAI, LPAIIREAI, LPSIVREAL, APTRVVAAEM, APTRVVASET, APTRVVAAEM, APTRVVASEM, APPRVVPAEM, APTRVVAAEM, APTRVVAAEM, APTRVVAAEM, VPNYNLIIM, VPNYNMIIV, VPNYNMIIM, VPNYNMIVM, VPNYNLIIM, VPNYNLIVM, VPNYNLVVM, VPNYNLVIM, VSNYNLIIM, VPNYNLIIM, VPNYNLIVM, APIMDEEREI, AIIQDEERDI, AVIQDEEKDI, AAIQDEERDI, AVIQDEERDI, APIMDDEREI, APIIDEEREI, APIVDEEREI, APIMDEEREI, APIQDEEKDI, SPIQDEERDI, APIQDEERDI, APIQDKERDI, SPIEDIEREI, TPEGIIPSMF, TPEGIIPALY, TPEGIIPALF, TPEGIIPSLF, TPEGIIPSMF, TPEGIIPALF, TPEGIIPTLF, LLVISGLFPV, LLAISGVYPL, LLAVSGMYPL, LLAVSGVYPL, LLVISGVYPM, LLAVSGVYPI, LLAASGVYPM, LLAISGVYPM, LLAVSGVYPM, LLVVSGLFPV, LLVISGLFPA, LLVISGLFPI, LLVISGVFPV, LLVISGLFPV, LLIVSGIFPC, LLIVSGIFPY, LLIVSGVFPY, LITVSGLYPL, AAAWYLWEV, LFVWCFWQK, LFLWYFWQK, LFVWHFWQK, FFVWYFWQK, PFVWYFWQK, LFVWYFWQK, AAAWYLWET, AAAWYLWEA, AAAWYLWEV, LLVWHAWQK, MLVWHTWQK, LLVWHTWQK, MALWYIWQV, MTLWYMWQV, MALWYMWQV, YLPAIVREA, YLPAIIREA, YLPAIVREA, YLPAMVREA, SLPAIVREA, YLPAIVREA, YLPTIVREA, YLPAVVREA, YLPAIVREA, YLPAIIREA, ILPSIVREA, DLMRRGDLPV, DLLRRGDLPV, ELMRRGDLPV, DLMKRGDLPV, ELMRRGDLPV, DLMRRGDLPV, ELMRRGHLPV, ELMRRGDLPV, ELMKRGDLPV, ELMRRGDLPV, ALSELPETL, ALEELPDTI, AVEELPDTI, AMEELPDTI, ALSELAETL, ALGELPETL, ALSELPETL, AVEELPETM, ALNELTESL, ALNELPESL, IILEFFLIV, IILKFFLMV, IILEFLLMV, IMLEFFLMV, IILEFFLMV, IILEFFLMV, IILEFFLIV, IILEFFMMV, IVLEFFMMV, IILEFFLMV, KLAEAIFKL, LLAKAIFKL, QLAKSIFKL, LLATSVFKL, LLAKSIFKL, LLATAIFKL, LLATSIFKL, LLASSIFKL, KLAEAIFRL, RLAEAIFKL, KLAEAVFKL, KLAEAIFKL, QLASAIFKL, LLANAIFKL, RLANAIFKL, QLANAIFKL, TLAKAIFKL, ILAKAIFKL, SQIGAGVYK, SQVGVGVFQ, SQIGAGVYR, SQIGTGVYK, SQIGVGVYK, SQIGAGVYK, TQVGVGIQK, TQVGVGVHK, TQVGVGVQK, TQVGVGIHI, TQVGVGIHT, TQVGVGIHM, TQVGVGVHV, GTSGSPIIDK, GTSGSPIVSR, GTSGSPIVNR, GTSGSPIIDK, GTSGSPIADK, GTSGSPIVDR, GTSGSPIVDK, GTSGSPIINK, GTSGSPIINR, GSSGSPIINR, GTSGSPIVNR, GTSGSPIINK, GTSGSPIINR, KTFDSEYVK, KTFDTEYQK, KTFDTEYTK, KTFDTEYIK, KTFDFEYIK, KTFDSEYIK, KTFDSEYAK, KTFDSEYVK, KTFDTEYQR, KTFNTEYQK, KTFDTEYQK, KTFDTEYPK, KPRWLDARI, RPRWLDART, KPRWLDART, KPRWLDAKI, KPRWLDPRI, KPRWLDARI, RPRWLDART, RPRWLDARI, RPRWLDARV, RPKWLDARV, RPASAWTLYA, HPASAWTLYA, RPASAWTLYA, RPASAWTLYA, HPASAWILYA, HPASAWTLYA, RPASAWTLYA, TPMLRHSI, TPMLRHTI, TPMMRHTI, TPMLRHSI, TPMLRHTI, TPMLRHTI, SPNPTVEAGR, SPNPTIEEGR, SPSPTVEAGR, SPNPTVDAGR, SPNPTVEAGP, SPNPTIEAGR, SPNPTVEAGR, SPSPTVEEGR, SPSLTVEESR, SPSPIVEESR, SPSPTVEESR, SSNPTIEEGR, TPRMCTREEF, KPRICTREEF, RPRICTRAEF, KPRICTRAEF, TRRMCTREEF, TPRICTREEF, IPRMCTREEF, TPRMCTREEF, KPRLCPREEF, KPRLCTREEF, RPRLCTREEF, NPRLCTKEEF, SPRLCTREEF, TPRLCTREEF, SPRLCTKEEF, NPRLCTREEF, KPRLCTREEF, RPTPRGTVM, RPVKNGTVM, RPARNGTVM, RPAKNGTVM, RPAKSGTVM, RPTPRGTVL, RPTPKGTVM, RPTPIGTVM, RPTPRGTVM, RPTPKGTVM, RPTPTGTVM, RPTPRGAVM, RPTPKGAVM, AFLRFLTIPPTAGIL, AFLRFLAIPPTAGIV, ALLRFLAIPPTAGIL, AFLTFLAIPPTAGIL, AFLRFLAIPPTAGIL, AFLRFLTISPTAGIL, AFLRFLTIPPTVGIL, AFLRFLTIPPTAGIL, AFLRFLAIPPTAGIL, AFLRFLAIPPTAGVL, TFLRVLSIPPTAGIL, GVTYLALLAAFKVRP, GTTYLALMATFRMRP, GMTYLALMATFKMRP, GTTYLALMATLKMRP, GTTHLALMATFKMRP, GTTYLALMATFKMRP, GVTYLALLATFKVRP, GVTYLALLAAYKVRP, GVTYLALLAAFRVRP, GVTYLALLAAFKVRP, GVTYLALIATFEIQP, GVTCLALIATFKIQP, GVTYLALIATFKVQP, GVTYLALIATFKIQP, GQTHLAIMAVFKMSP, GQIHLAIMAVFKMSP, GQTHLAIMIVFKMSP, GQVHLAIMAVFKMSP, GQIHLAIMTMFKMSP, MAVGMVSILASSLLK, MAVGVVSILLSSLLK, MAIGIVSILLSSLLK, MAVGIVSILLSSLLK, MAVGMVSILASSLLK, MAVGLVSILASSFLR, MAIGLVSILASSLLR, MAVGLVSILASSLLR, MAVGLVSLLGSALLK, TFTMRLLSPVRVPNY, TFTMRLLSPVRVPNY, PFTMRLLSPVRVPNY, TFTMRLLSPIRVPNY, TFTMRLLSPVRVPNY, TFTMRLLSPVRVSNY, PFTMRLLSPVRVPNY, TFTMRLLSPVRVPNY, TFTTKLLSSTRVPNY, TFTTRLLSSTRVPNY, SRAIWYMWLGARFLE, SRAIWYVWLGARFLE, SRAIWYMWLGAAFLE, SRAIWYMWLGARFLE, SRAIWYMWLGARFLE, SRAIWYMWLGARFLE, SRAIWYMWLGVRYLE, SRAIWYMWLGARYLE, SRAIWYMWLGARFLE, (SEQ ID NOs: 57-554) or a subsequence or a modification thereof.

69. The method of claim 27 wherein the peptide comprises or consists of a sequence set forth as any of: AAVLLLVTHY, ALRGLPIRY, AMALSIVSLF, MALSIVSLF, APFESEGVL, APIMDEEREI, APTRVVAAEM, APTRVVASEM, ASSMVNGVVK, ASSMVNGVVR, ASSVLLWMAS, ATGPILTLW, ATGPISTLW, ATGPITTLW, ATGPLTTLW, ATGPVLTLW, LATGPVLTLW, ATYGWNLVK, MATYGWNLVK, MSTYGWNIVK, AVQTKPGLFK, CLIPTAMAF, MLIPTAMAF, CLMMMLPATL, CPTQGEATL, CPTQGEAVL, CPTQGEPSL, DPASIAARGY, DPIPYDPKF, DPRRCLKPV, DTTPFGQQR, DYMPSMKRFR, EAVEDSRFWE, EENMDVEIW, EERDIPERSW, EFKEFAAGR, EFKEFAAGRR, ELMRRGDLPV, EPDYEVDEDI, EPGQLKLNWF, EPGVVSPTSY, EPIEGKVVQY, EPISYDPKF, EPKEGTKKLM, ESSSNPTIEE, ETACLGKAY, ETACLGKAYA, ETACLGKSY, KETACLGKSY, RETACLGKAY, RETACLGKSY, ETTHHAVSR, ETTKHAVSR, FAGPVSQHNY, FASGRKSITL, FGAIYGAAF, FLMVLLIPEP, FMALVAFLRF, FMKDGRSLVV, FPQSNAPIMD, FPVSIPITAA, FSRENSLSGV, FTILALFLAH, FTIMAAILAY, TIMAAILAY, TLMAAILAY, FTMGVLCLAI, FTMRHKKATY, FTNMEAQLVR, GAMLFLISGK, GASKRSWPLN, GEARKTFVDL, GEARKTFVEL, GEQRKTFVEL, GESRKTFVEL, GEFRLRGEQR, GEGLHKLGY, GEVGAIALDF, GFFLRKLTSR, GGWRLSAQW, GMGEAAAIF, GPGHEEPIPM, GPLVAGGLL, GPMKLVMAF, GPMKLVMAFI, GPSLRTTTV, GPWHLGKLEL, GPWHLGKLEM, GSSKIRWIVE, GTSGSPIIDK, TSGSPIIDK, GTSGSPIVNR, HALLATSIF, HMIAGVLFTF, HPASAWTLY, RPASAWTLYA, HPGAGKTKRY, HPGFTILALF, HQLWATLLSL, IAFLRFLAI, IANQAAILM, IANQATVLM, IAVSMANIF, IAVSTANIF, ICSAVPVHW, IGIGILLTW, IGIGVLLTW, IGLTSRATW, IILEFFLMV, IPFEIMDLEK, IPITAAAWY, IPKAYAGPF, IPMATYGWNL, IPMSTYGWNL, IPMTGPLVAG, IPQWEPSKGW, IPTVMAFHL, IQPFLALGF, ISSMLNIMNR, ITAAAWYLW, ITLLCLIPTV, TLLCLIPTV, ITPMLRHTI, IVIGVGDSAL, KAKGSRAIW, RAKGSRAIW, KATREAQKRA, KAVHADMGYW, RAVHADMGYW, KAYAQMWSL, KEGVFHTMW, KGSRAIWYMW, KPGTSGSPI, KPGTSGSPII, KPGTSGSPIV, KPRICTREEF, KPRLCTREEF, NPRLCTREEF, RPRLCTREEF, TPRMCTREEF, KPRWLDARI, KPTLDFELI, KPWDVIPMV, KPWDVVPMV, KQIANELNY, KQISNELNY, KSGAIKVLK, KSYAQMWQL, KTDFGFYQV, KTFVDLMRR, KTWAYHGSY, KVIQLSRKTF, KVLNPYMPSV, VLNPYMPSV, KVRKDIQQW, LAYTIGTTHF, LEFEALGFLN, LEFEALGFMN, LETLMLVAL, LGFLNEDHW, LIHQVFGTAY, LVHQIFGTAY, LILCVTQVLM, LLMLVTPSM, LLSPVRVPNY, LMCHATFTM, LMKITAEWLW, MEITAEWLW, VMGITAEWLW, LMMATIGIAL, MMATIGIAL, LMMILPAAL, LMMMLPATLA, LMMTTIGVVL, LPAIVREAI, LPEEQDQNY, LPESLETLM, LPETLETLLL, LPGADTQGSN, LPIRYQTPA, LPIRYQTPAI, LPLPWLPGAD, LPLPWTSGA, LPTFMTQKAR, LPTYLSSRAK, LPVWLAHKVA, LPVWLAYKV, LPVWLAYKVA, LPVWLAYRVA, LPVWLSYKV, LRVLNLVENR, LSMGLITIAV, LSRNSTHEM, LTAAVLLLI, TLTAAVLLLV, LWEGSPGKF, MAEIPLQWI, MAFIAFLRF, MAILGDTAW, MALKDFKEF, MAMTCIAVG, MANEMGFLEK, MANIFRGSY, MASSALLWMA, MAVGMVSIL, MGYWIESAL, MIDKTPVHSW, MLLALIAVL, MLLILCVTQV, MLVTPSMTM, MLWMAEIPL, MMLKLLTDF, MMLPATLAF, MMMLPATLAF, MPGTFQTTTG, MPGTRKVMGI, MPGTRRVMGI, MPLVMAWRTI, MPSMKRFRRE, MPSVIEKMET, MPVMKRYSAP, MPVTHSSAAQ, MPVTVASAAQ, MSFRDLGRVM, MSSEGAWKHA, MSYSMCTGKF, MSYTMCSGK, MTQKARNAL, MTTTANWLW, MVSRLLLNR, NPAQEDDQY, NPEIEDDIF, NPITLTAAL, NPLTLTAAV, NPNMIDKTPV, NPWMEDKTH, NPWMEDKTPV, NPTVDGITV, NQLIYVILTI, PASAWTLYAV, PASIAARGYI, PTSRTTWSIH, QEGAMHSAL, QEGAMHTAL, QENPYRTWAY, QIAMTDTTPF, QPESNILDI, QPHWIAASI, QSGVDVFFTP, QYIFTGQPL, QYSDRRCF, RAAVEDEEF, RASFIEVKTC, REDLWCGSL, REDQWCGSL, REIPERSWNT, REWCFTGERN, RFLEFEALGF, RYLEFEALGF, RLRGEARKTF, RPGYHTQTA, RPISEKEENM, RPRWLDART, RPTPKGAVM, RPTPKGTVM, RQLANAIFK, RQMEGEGIF, RQMEGEGVL, RVIDPRRCL, RVIDPRRCLK, RVIDPRRCM, RVIDPRRCMK, RVLKMVEPW, RYMGEDGCWY, SEMAEALKGM, SPCKIPFEIM, SPGKFWNTTI, SPIINREGKV, SPILSITISE, SPKRLATAIA, SPSPTVEESR, SPVRVPNYNL, SQIGAGVYK, STYGWNLVR, SWMVRILIGF, SYAQMWTLMY, YAQMWQLMYF, YAQMWSLMY, YAQMWQLMYF, YAQMWTLMY, YAQMWTLMYF, TAAVLLLITH, TAEAGGRAY, TETTILDVDL, TFKVPHAKR, TLMAMDLGEL, TLMLLALIAV, TPEAKNSTF, TPEARNSTF, TPEGIIPAL, TPEGIIPALF, TPEGIIPSM, TPEGIIPSMF, TPEGIIPTLF, YTPEGIIPTL, TPFGQQRVF, TPKGAVMDII, TPRGTVMDII, TPMLRHTIEN, TPPGSRDPF, TPQDNQLAY, TPQDNQLAYV, TPQDNQLTY, TPRSMPGTRR, TPVHSWEDI, TTFSLHYAW, TTKRDLGMSK, TYLALIATF, VASGLLWVAE, VATTFVTPM, VLLLVTHYAI, VPLLAIGCY, VPLLAMGCY, VPMVTQMAM, VPNYNMIIM, VPYLGKREDQ, VQADMGCVV, VSSVNMVSRL, VTIDLDPVVY, VTRGAVLMHK, VTYECPLLV, VYTQLCDHR, VYTQLCDHRL, WALCESITL, WAYHGSYET, WAYHGSYEV, WHYDQDHPY, WSIHAHHQW, WVAEIQPQW, YAQIQPHWI, YENLKYSVI, YGGPISQHNY, YGVLFSGVSW, YKVASAGISY, YLAGAGLAF, YPKTKLTDW, YPKTKLTDWD, YQLAVTITAI, YQLWTALISL, YQNKVVKVLR, YQNKVVKVQR, YVSAIAQTEK (SEQ ID NOs: 813-1163) or a subsequence thereof or a modification thereof.

70. The method of claim 29 wherein the Dengue virus comprises a DENV1, DENV2, DENV3 or DENV4 serotype.

71. The method of claim 29, wherein the peptide comprises or consists of a Dengue virus (DV) T cell epitope.

72. The method of claim 71, wherein the Dengue virus (DV) T cell epitope is a structural or non-structural (NS) T cell epitope.

73. The method of claim 71, wherein the Dengue virus T cell epitope comprises or consists of a subsequence or portion of Dengue virus C, M or E protein.

74. The method of claim 71, wherein the Dengue virus T cell epitope comprises or consists of a subsequence or portion of NS1, NS2A, NS2B, NS3, NS4A, NS4B or NS5 protein.

75. The method of claim 29, wherein the method reduces one or more adverse physiological conditions, disorders, illness, diseases, symptoms or complications caused by or associated with Dengue virus (DV) infection or pathology.

76. The method of claim 29, wherein the method improves one or more adverse physiological conditions, disorders, illness, diseases, symptoms or complications caused by or associated with Dengue virus (DV) infection or pathology.

77. The method of claim 29, wherein the method reduces or inhibits susceptibility to Dengue virus (DV) infection or pathology.

78. The method of claim 71, wherein the Dengue virus (DV) T cell epitope is administered prior to, substantially contemporaneously with or following exposure to or infection of the subject with Dengue virus (DV).

79. The method of claim 71, wherein a plurality of Dengue virus (DV) T cell epitopes are administered prior to, substantially contemporaneously with or following exposure to or infection of the subject with Dengue virus (DV).

80. The method of claim 29, wherein the peptide comprises or consists of a peptide sequence set forth as: GLFPVSLPITAAAWY (SEQ ID NO: 11); GKTKRYLPAIVREAI (SEQ ID NO: 12); GLPIRYQTPAIRAEH (SEQ ID NO: 13); or IGCYSQVNPITLTAA (SEQ ID NO: 14), or a subsequence thereof or a modification thereof.

81. The method of claim 29, wherein the peptide comprises or consists of a peptide sequence set forth as any one of: GPMKLVMAF, GPMKLVMAFI, MAFIAFLRF, KSGAIKVLK, ITLLCLIPTV, CLMMMLPATL, TLLCLIPTV, LMMMLPATL, LMMMLPATLA, MMMLPATLA, MMMLPATLAF, MLIPTAMAF, TLMAMDLGEL, VTYECPLLV, HPGFTILALF, FTIMAAILAY, FTILALFLAH, TIMAAILAY, MLVTPSMTM, CPTQGEATL, CPTQGEAVL, LPEEQDQNY, YENLKYSVI, QEGAMHSAL, QEGAMHTAL, MSYTMCSGK, MSYSMCTGKF, SPCKIPFEIM, IPFEIMDLEK, EPGQLKLNWF, FGAIYGAAF, SWMVRILIGF, IGIGILLTW, SPKRLATAIA, KQIANELNY, VYTQLCDHRL, VYTQLCDHR, KAVHADMGYW, RASFIEVKTC, FAGPVSQHNY, RPGYHTQTA, GPWHLGKLEL, GPWHLGKLEM, RYMGEDGCWY, FTMGVLCLAI, MSFRDLGRVM, TYLALIATF, IQPFLALGF, GFFLRKLTSR, MMATIGIAL, MALSIVSLF, MAVGMVSIL, IPMTGPLVAG, GPLVAGGLL, SPILSITISE, FPVSIPITAA, IPITAAAWY, MEGVFHTMW, MEGVFHTMWH, KPGTSGSPI, KPGTSGSPII, GTSGSPIIDK, SPIINREGKV, NPEIEDDIF, HPGAGKTKRY, LPAIVREAI, APTRVVAAEM, APTRVVASEM, APTRVVAAEM, SEMAEALKGM, LPIRYQTPA, LPIRYQTPAI, DPASIAARGY, PASIAARGYI, TPPGSRDPF, FPQSNAPIMD, FPQSNAPIM, EERDIPERSW, REIPERSWNT, YPKTKLTDWD, RVIDPRRCMK, RVIDPRRCLK, RVIDPRRCMK, RVIDPRRCL, RVIDPRRCMK, DPRRCLKPV, MPVTHSSAA, MPVTHSSAAQ, NPAQEDDQY, QYIFTGQPL, TPEGIIPSM, TPEGIIPSMF, TPEGIIPAL, TPEGIIPALF, TPEGIIPSM, TPEGIIPSMF, TPEGIIPTLF, TPEGIIPALF, GEFRLRGEQR, GEARKTFVEL, GEARKTFVDL, GESRKTFVEL, GEQRKTFVEL, ELMRRGDLPV, LPVWLAYKVA, YKVASAGISY, REWCFTGERN, RPRWLDART, MALKDFKEF, EFKEFAAGR, FASGRKSITL, LPTFMTQKAR, MTQKARNAL, TAEAGGRAY, LPETLETLLL, LETLMLVAL, LETLMLVALL, TLMLLALIAV, LMLLALIAVL, MLLALIAVL, GAMLFLISGK, SIILEFFLMV, IILEFFLMV, IILEFFLMVL, FLMVLLIPEP, TPQDNQLAYV, TPQDNQLTY, TTKRDLGMSK, TETTILDVDL, RPASAWTLY, RPASAWTLYA, HPASAWTLY, PASAWTLYAV, VATTFVTPM, ITPMLRHTI, TPMLRHTIEN, IANQATVLM, VPLLAIGCY, NPLTLTAAV, TLTAAVLLLV, AAVLLLVTHY, VLLLVTHYAI, DPIPYDPKF, MLLILCVTQV, ATGPLTTLW, ATGPISTLW, ATGPITTLW, ATGPILTLW, ATGPVLTLW, LWEGSPGKF, SPGKFWNTTI, IAVSMANIF, IAVSMANIF, IAVSTANIF, MANIFRGSY, YLAGAGLAF, GSSKIRWIVE, GPGHEEPIPM, IPMSTYGWNL, IPMATYGWNL, IPMSTYGWNL, MSTYGWNIVK, STYGWNIVK, QSGVDVFFTP, RVLKMVEPW, KVLNPYMPSV, VLNPYMPSV, MPSVIEKMET, VSSVNMVSRL, MVSRLLLNR, FTMRHKKATY, WHYDQDHPY, QENPYRTWAY, WAYHGSYET, WAYHGSYEV, DTTPFGQQR, TPFGQQRVF, EPKEGTKKLM, MEITAEWLW, KPRICTREEF, TPRMCTREEF, KPRLCTREEF, NPRLCTREEF, RPRLCTREEF, TPRMCTREEF, RAAVEDEEF, EAVEDSRFWE, KGSRAIWYMW, RYLEFEALGF, RFLEFEALGF, FSRENSLSGV, GEGLHKLGY, RQLANAIFK, TPRGTVMDII, TPKGAVMDII, RQMEGEGIF, RQMEGEGVL, KVRKDIQQW, YAQMWSLMY, YAQMWSLMYF, ICSAVPVHW, WSIHAHHQW, NPNMIDKTPV, NPWMEDKTPV, VPYLGKREDQ, REDLWCGSL, REDQWCGSL, MPSMKRFRRE, APFESEGVL, (SEQ ID NOs: 555-763) or a subsequence or a modification thereof.

82. The method of claim 29, wherein the peptide comprises or consists of a peptide sequence set forth as any one of: ITEAELTGY, RSCTLPPLRY, MTDDIGMGV, LTDALALGM, VIDLDPIPY, YTDYMPSMK, RLITVNPIV, IMAVGMVSI, GLLTVCYVL, LLVISGLFPV, AAAWYLWEV, YLPAIVREA, DLMRRGDLPV, ALSELPETL, IILEFFLIV, KLAEAIFKL, SQIGAGVYK, GTSGSPIIDK, KTFDSEYVK, RIYSDPLALK, ATVLMGLGK, STYGWNLVR, TVMDIISRR, RQMEGEGVFK, RTTWSIHAK, RPTFAAGLLL, LPAIVREAI, APTRVVAAEM, VPNYNLIIM, APIMDEEREI, TPEGIIPSMF, KPRWLDARI, RPASAWTLYA, TPMLRHSI, SPNPTVEAGR, TPRMCTREEF, RPTPRGTVM, AFLRFLTIPPTAGIL, GVTYLALLAAFKVRP, MAVGMVSILASSLLK, TFTMRLLSPVRVPNY, SRAIWYMWLGARFLE, (SEQ ID NOs: 15-56) or a subsequence or a modification thereof.

83. The method of claim 29, wherein the peptide comprises or consists of a peptide sequence set forth as any one of: ITEAELTGY, STEIQLTDY, TTEIQLTDY, TSEIQLIDY, TSEIQLTDY, IAEAELTGY, IAEAELTDY, ITDAELTGY, STEAELTGY, TTEAELTGY, ISEAELTDY, ITEAELTGY, TVEAVLLEY, TVEAVLPEY, TVEAILPEY, TAEAILPEY, THEALLPEY, ITEAILPEY, TTEVILPEY, TTEAILPEY, SVEVELPDY, SVEVKLPDY, RSCTLPPLRY, RSCTLPPLRF, RSCTLPPLRY, RSCTLPPLRY, RSCTMPPLRF, MTDDIGMGV, ASDRMGMGM, ASDMMGMGT, ASDKMGMGT, ASDNMGMGT, VSDRMGMGT, ASDRMGMGT, MADDIGMGV, MTDEMGMGV, ITDDIGMGV, MTDDIGMGV, ASDRTGMGV, ASDKMGMGV, ATDRMGMGV, ASDRMGMGV, LTDALALGM, LGDGLAIGI, LGDGFAMGI, LGDGLAMGI, LTDAIALGI, LTDAWALGM, LTDALALGI, LTDALALGM, MANGVALGL, MANGIALGL, LISGISLGL, FIDGLSLGL, LIDGISLGL, LIDGIALGL, FIDGISLGL, VIDLDPIPY, TIDLDPVVY, AIDLDPVVY, VIDLEPIPY, VIDLDPIPY, TIDLDSVIF, TIDLDPVIY, TIALDPVIY, VIDLEPISY, YTDYMPSMK, YSDYMTSMK, YLDYMASMK, YIDYMTSMK, YLDFMTSMK, YLDYMTSMK, YLDYMISMK, YIDYMPSMK, YMDYMPSMK, YTDYMPSMK, FLDYMPSMK, YADYMPVMK, YMDYMPVMK, YVDYMPAMK, YVDYMPVMR, YVDYMPVMK, RLITVNPIV, RVITANPIV, RLVTANPIV, RLITANPIV, RLITVNPVV, RLITVNPII, RLITVNPIV, RLTTVNPIV, RLITANPIV, RLITANPVV, RVISATPLA, RVISSTPLA, RIISSTPLA, RVISSTPFA, RIISSTPFA, RIISSIPFA, IMAVGMVSI, IMAVGVVSI, VMAVGIVSI, IMAIGIVSI, IMAVGIVSI, VMAVGMVSI, IMAVGMVSI, VMAIGLVSI, VMAVGLVSI, MMAVGLVSL, IMAVGLVSL, GLLTVCYVL, GMLITCYVI, GMLIACYVI, GPLTVCYVL, GLLTVCYVL, GMLIACYVI, GLLIACYVI, GLLLAAYMM, GLLLAAYVM, RIYSDPLALK, RTYSDPQALR, RTYSDPLALR, RTYSDPLALK, RIYSDPLTLK, KIYSDPLALK, RIYSEPRALK, RIYSDPLALK, RTYSDPLAPK, RTYSDPLALK, RIYSDPLALK, RVYADPMALQ, RVYADPMALK, ATVLMGLGK, AAILMGLDK, ATVLMGLGK, ATVLMGLGR, AVVLMGLNK, AVVLMGLDK, AAVLMGLGK, STYGWNLVR, AAYGWNLVK, ATYGWNLVK, STYGWNLVR, STYGWNLVK, STYGWNVVK, STYGWNIVK, ATYGWNLVK, TVMDIISRR, TVMDIISRR, TVMDVISRR, TVLDIISRR, TVMDIISRK, TVMDIISRR, TVMDIISRK, AVMDIISRK, RQMEGEGVFK, RQMESEEIFS, RQMESEGIVS, RQMESEGIFF, RQMESEGIIL, RQMESEGIFS, RQMESEGIFL, RQMEGEGVFR, RQMEGEGIFR, RQMEGEGLFK, RQMEGEEVFK, RQMEGEGVFK, RQMEGEGIFK, RQMEGEGVLT, RQMEGEGVLS, RQMEGEDVLS, RQMEAEGVIT, RTTWSIHAK, RTTWSIHAH, RTTWSIHAR, RTTWSIHAT, RTTWSIHAS, RTTWSIHAK, RTTWSIHAH, RTTWSIHAH, RPTFAAGLLL, RPMLAVGLLF, RPMFAMGLLF, RPMFAVGLLI, RPMFAVGLLF, RPTFAAGLFL, RPTFAVGLVL, RPTFAVGLLL, RPTFAAGLLL, QPFLALGFFM, QPFLTLGFFL, QPFLALGFFL, SPRYVLGVFL, SPGYVLGVFL, SPGYVLGIFL, LPAIVREAI, LPAIIREAI, LPAIVREAI, LPAMVREAI, LPAIVREAI, LPTIVREAI, LPAVVREAI, LPAIVREAI, LPAIIREAI, LPSIVREAL, APTRVVAAEM, APTRVVASET, APTRVVAAEM, APTRVVASEM, APPRVVPAEM, APTRVVAAEM, APTRVVAAEM, APTRVVAAEM, VPNYNLIIM, VPNYNMIIV, VPNYNMIIM, VPNYNMIVM, VPNYNLIIM, VPNYNLIVM, VPNYNLVVM, VPNYNLVIM, VSNYNLIIM, VPNYNLIIM, VPNYNLIVM, APIMDEEREI, AIIQDEERDI, AVIQDEEKDI, AAIQDEERDI, AVIQDEERDI, APIMDDEREI, APIIDEEREI, APIVDEEREI, APIMDEEREI, APIQDEEKDI, SPIQDEERDI, APIQDEERDI, APIQDKERDI, SPIEDIEREI, TPEGIIPSMF, TPEGIIPALY, TPEGIIPALF, TPEGIIPSLF, TPEGIIPSMF, TPEGIIPALF, TPEGIIPTLF, LLVISGLFPV, LLAISGVYPL, LLAVSGMYPL, LLAVSGVYPL, LLVISGVYPM, LLAVSGVYPI, LLAASGVYPM, LLAISGVYPM, LLAVSGVYPM, LLVVSGLFPV, LLVISGLFPA, LLVISGLFPI, LLVISGVFPV, LLVISGLFPV, LLIVSGIFPC, LLIVSGIFPY, LLIVSGVFPY, LITVSGLYPL, AAAWYLWEV, LFVWCFWQK, LFLWYFWQK, LFVWHFWQK, FFVWYFWQK, PFVWYFWQK, LFVWYFWQK, AAAWYLWET, AAAWYLWEA, AAAWYLWEV, LLVWHAWQK, MLVWHTWQK, LLVWHTWQK, MALWYIWQV, MTLWYMWQV, MALWYMWQV, YLPAIVREA, YLPAIIREA, YLPAIVREA, YLPAMVREA, SLPAIVREA, YLPAIVREA, YLPTIVREA, YLPAVVREA, YLPAIVREA, YLPAIIREA, ILPSIVREA, DLMRRGDLPV, DLLRRGDLPV, ELMRRGDLPV, DLMKRGDLPV, ELMRRGDLPV, DLMRRGDLPV, ELMRRGHLPV, ELMRRGDLPV, ELMKRGDLPV, ELMRRGDLPV, ALSELPETL, ALEELPDTI, AVEELPDTI, AMEELPDTI, ALSELAETL, ALGELPETL, ALSELPETL, AVEELPETM, ALNELTESL, ALNELPESL, IILEFFLIV, IILKFFLMV, IILEFLLMV, IMLEFFLMV, IILEFFLMV, IILEFFLMV, IILEFFLIV, IILEFFMMV, IVLEFFMMV, IILEFFLMV, KLAEAIFKL, LLAKAIFKL, QLAKSIFKL, LLATSVFKL, LLAKSIFKL, LLATAIFKL, LLATSIFKL, LLASSIFKL, KLAEAIFRL, RLAEAIFKL, KLAEAVFKL, KLAEAIFKL, QLASAIFKL, LLANAIFKL, RLANAIFKL, QLANAIFKL, TLAKAIFKL, ILAKAIFKL, SQIGAGVYK, SQVGVGVFQ, SQIGAGVYR, SQIGTGVYK, SQIGVGVYK, SQIGAGVYK, TQVGVGIQK, TQVGVGVHK, TQVGVGVQK, TQVGVGIHI, TQVGVGIHT, TQVGVGIHM, TQVGVGVHV, GTSGSPIIDK, GTSGSPIVSR, GTSGSPIVNR, GTSGSPIIDK, GTSGSPIADK, GTSGSPIVDR, GTSGSPIVDK, GTSGSPIINK, GTSGSPIINR, GSSGSPIINR, GTSGSPIVNR, GTSGSPIINK, GTSGSPIINR, KTFDSEYVK, KTFDTEYQK, KTFDTEYTK, KTFDTEYIK, KTFDFEYIK, KTFDSEYIK, KTFDSEYAK, KTFDSEYVK, KTFDTEYQR, KTFNTEYQK, KTFDTEYQK, KTFDTEYPK, KPRWLDARI, RPRWLDART, KPRWLDART, KPRWLDAKI, KPRWLDPRI, KPRWLDARI, RPRWLDART, RPRWLDARI, RPRWLDARV, RPKWLDARV, RPASAWTLYA, HPASAWTLYA, RPASAWTLYA, RPASAWTLYA, HPASAWILYA, HPASAWTLYA, RPASAWTLYA, TPMLRHSI, TPMLRHTI, TPMMRHTI, TPMLRHSI, TPMLRHTI, TPMLRHTI, SPNPTVEAGR, SPNPTIEEGR, SPSPTVEAGR, SPNPTVDAGR, SPNPTVEAGP, SPNPTIEAGR, SPNPTVEAGR, SPSPTVEEGR, SPSLTVEESR, SPSPIVEESR, SPSPTVEESR, SSNPTIEEGR, TPRMCTREEF, KPRICTREEF, RPRICTRAEF, KPRICTRAEF, TRRMCTREEF, TPRICTREEF, IPRMCTREEF, TPRMCTREEF, KPRLCPREEF, KPRLCTREEF, RPRLCTREEF, NPRLCTKEEF, SPRLCTREEF, TPRLCTREEF, SPRLCTKEEF, NPRLCTREEF, KPRLCTREEF, RPTPRGTVM, RPVKNGTVM, RPARNGTVM, RPAKNGTVM, RPAKSGTVM, RPTPRGTVL, RPTPKGTVM, RPTPIGTVM, RPTPRGTVM, RPTPKGTVM, RPTPTGTVM, RPTPRGAVM, RPTPKGAVM, AFLRFLTIPPTAGIL, AFLRFLAIPPTAGIV, ALLRFLAIPPTAGIL, AFLTFLAIPPTAGIL, AFLRFLAIPPTAGIL, AFLRFLTISPTAGIL, AFLRFLTIPPTVGIL, AFLRFLTIPPTAGIL, AFLRFLAIPPTAGIL, AFLRFLAIPPTAGVL, TFLRVLSIPPTAGIL, GVTYLALLAAFKVRP, GTTYLALMATFRMRP, GMTYLALMATFKMRP, GTTYLALMATLKMRP, GTTHLALMATFKMRP, GTTYLALMATFKMRP, GVTYLALLATFKVRP, GVTYLALLAAYKVRP, GVTYLALLAAFRVRP, GVTYLALLAAFKVRP, GVTYLALIATFEIQP, GVTCLALIATFKIQP, GVTYLALIATFKVQP, GVTYLALIATFKIQP, GQTHLAIMAVFKMSP, GQIHLAIMAVFKMSP, GQTHLAIMIVFKMSP, GQVHLAIMAVFKMSP, GQIHLAIMTMFKMSP, MAVGMVSILASSLLK, MAVGVVSILLSSLLK, MAIGIVSILLSSLLK, MAVGIVSILLSSLLK, MAVGMVSILASSLLK, MAVGLVSILASSFLR, MAIGLVSILASSLLR, MAVGLVSILASSLLR, MAVGLVSLLGSALLK, TFTMRLLSPVRVPNY, TFTMRLLSPVRVPNY, PFTMRLLSPVRVPNY, TFTMRLLSPIRVPNY, TFTMRLLSPVRVPNY, TFTMRLLSPVRVSNY, PFTMRLLSPVRVPNY, TFTMRLLSPVRVPNY, TFTTKLLSSTRVPNY, TFTTRLLSSTRVPNY, SRAIWYMWLGARFLE, SRAIWYVWLGARFLE, SRAIWYMWLGAAFLE, SRAIWYMWLGARFLE, SRAIWYMWLGARFLE, SRAIWYMWLGARFLE, SRAIWYMWLGVRYLE, SRAIWYMWLGARYLE, SRAIWYMWLGARFLE, (SEQ ID NOs: 57-554) or a subsequence or a modification thereof.

84. The method of claim 29, wherein the peptide comprises or consists of a peptide sequence set forth as any one of: AAVLLLVTHY, ALRGLPIRY, AMALSIVSLF, MALSIVSLF, APFESEGVL, APIMDEEREI, APTRVVAAEM, APTRVVASEM, ASSMVNGVVK, ASSMVNGVVR, ASSVLLWMAS, ATGPILTLW, ATGPISTLW, ATGPITTLW, ATGPLTTLW, ATGPVLTLW, LATGPVLTLW, ATYGWNLVK, MATYGWNLVK, MSTYGWNIVK, AVQTKPGLFK, CLIPTAMAF, MLIPTAMAF, CLMMMLPATL, CPTQGEATL, CPTQGEAVL, CPTQGEPSL, DPASIAARGY, DPIPYDPKF, DPRRCLKPV, DTTPFGQQR, DYMPSMKRFR, EAVEDSRFWE, EENMDVEIW, EERDIPERSW, EFKEFAAGR, EFKEFAAGRR, ELMRRGDLPV, EPDYEVDEDI, EPGQLKLNWF, EPGVVSPTSY, EPIEGKVVQY, EPISYDPKF, EPKEGTKKLM, ESSSNPTIEE, ETACLGKAY, ETACLGKAYA, ETACLGKSY, KETACLGKSY, RETACLGKAY, RETACLGKSY, ETTHHAVSR, ETTKHAVSR, FAGPVSQHNY, FASGRKSITL, FGAIYGAAF, FLMVLLIPEP, FMALVAFLRF, FMKDGRSLVV, FPQSNAPIMD, FPVSIPITAA, FSRENSLSGV, FTILALFLAH, FTIMAAILAY, TIMAAILAY, TLMAAILAY, FTMGVLCLAI, FTMRHKKATY, FTNMEAQLVR, GAMLFLISGK, GASKRSWPLN, GEARKTFVDL, GEARKTFVEL, GEQRKTFVEL, GESRKTFVEL, GEFRLRGEQR, GEGLHKLGY, GEVGAIALDF, GFFLRKLTSR, GGWRLSAQW, GMGEAAAIF, GPGHEEPIPM, GPLVAGGLL, GPMKLVMAF, GPMKLVMAFI, GPSLRTTTV, GPWHLGKLEL, GPWHLGKLEM, GSSKIRWIVE, GTSGSPIIDK, TSGSPIIDK, GTSGSPIVNR, HALLATSIF, HMIAGVLFTF, HPASAWTLY, RPASAWTLYA, HPGAGKTKRY, HPGFTILALF, HQLWATLLSL, IAFLRFLAI, IANQAAILM, IANQATVLM, IAVSMANIF, IAVSTANIF, ICSAVPVHW, IGIGILLTW, IGIGVLLTW, IGLTSRATW, IILEFFLMV, IPFEIMDLEK, IPITAAAWY, IPKAYAGPF, IPMATYGWNL, IPMSTYGWNL, IPMTGPLVAG, IPQWEPSKGW, IPTVMAFHL, IQPFLALGF, ISSMLNIMNR, ITAAAWYLW, ITLLCLIPTV, TLLCLIPTV, ITPMLRHTI, IVIGVGDSAL, KAKGSRAIW, RAKGSRAIW, KATREAQKRA, KAVHADMGYW, RAVHADMGYW, KAYAQMWSL, KEGVFHTMW, KGSRAIWYMW, KPGTSGSPI, KPGTSGSPII, KPGTSGSPIV, KPRICTREEF, KPRLCTREEF, NPRLCTREEF, RPRLCTREEF, TPRMCTREEF, KPRWLDARI, KPTLDFELI, KPWDVIPMV, KPWDVVPMV, KQIANELNY, KQISNELNY, KSGAIKVLK, KSYAQMWQL, KTDFGFYQV, KTFVDLMRR, KTWAYHGSY, KVIQLSRKTF, KVLNPYMPSV, VLNPYMPSV, KVRKDIQQW, LAYTIGTTHF, LEFEALGFLN, LEFEALGFMN, LETLMLVAL, LGFLNEDHW, LIHQVFGTAY, LVHQIFGTAY, LILCVTQVLM, LLMLVTPSM, LLSPVRVPNY, LMCHATFTM, LMKITAEWLW, MEITAEWLW, VMGITAEWLW, LMMATIGIAL, MMATIGIAL, LMMILPAAL, LMMMLPATLA, LMMTTIGVVL, LPAIVREAI, LPEEQDQNY, LPESLETLM, LPETLETLLL, LPGADTQGSN, LPIRYQTPA, LPIRYQTPAI, LPLPWLPGAD, LPLPWTSGA, LPTFMTQKAR, LPTYLSSRAK, LPVWLAHKVA, LPVWLAYKV, LPVWLAYKVA, LPVWLAYRVA, LPVWLSYKV, LRVLNLVENR, LSMGLITIAV, LSRNSTHEM, LTAAVLLLI, TLTAAVLLLV, LWEGSPGKF, MAEIPLQWI, MAFIAFLRF, MAILGDTAW, MALKDFKEF, MAMTCIAVG, MANEMGFLEK, MANIFRGSY, MASSALLWMA, MAVGMVSIL, MGYWIESAL, MIDKTPVHSW, MLLALIAVL, MLLILCVTQV, MLVTPSMTM, MLWMAEIPL, MMLKLLTDF, MMLPATLAF, MMMLPATLAF, MPGTFQTTTG, MPGTRKVMGI, MPGTRRVMGI, MPLVMAWRTI, MPSMKRFRRE, MPSVIEKMET, MPVMKRYSAP, MPVTHSSAAQ, MPVTVASAAQ, MSFRDLGRVM, MSSEGAWKHA, MSYSMCTGKF, MSYTMCSGK, MTQKARNAL, MTTTANWLW, MVSRLLLNR, NPAQEDDQY, NPEIEDDIF, NPITLTAAL, NPLTLTAAV, NPNMIDKTPV, NPWMEDKTH, NPWMEDKTPV, NPTVDGITV, NQLIYVILTI, PASAWTLYAV, PASIAARGYI, PTSRTTWSIH, QEGAMHSAL, QEGAMHTAL, QENPYRTWAY, QIAMTDTTPF, QPESNILDI, QPHWIAASI, QSGVDVFFTP, QYIFTGQPL, QYSDRRCF, RAAVEDEEF, RASFIEVKTC, REDLWCGSL, REDQWCGSL, REIPERSWNT, REWCFTGERN, RFLEFEALGF, RYLEFEALGF, RLRGEARKTF, RPGYHTQTA, RPISEKEENM, RPRWLDART, RPTPKGAVM, RPTPKGTVM, RQLANAIFK, RQMEGEGIF, RQMEGEGVL, RVIDPRRCL, RVIDPRRCLK, RVIDPRRCM, RVIDPRRCMK, RVLKMVEPW, RYMGEDGCWY, SEMAEALKGM, SPCKIPFEIM, SPGKFWNTTI, SPIINREGKV, SPILSITISE, SPKRLATAIA, SPSPTVEESR, SPVRVPNYNL, SQIGAGVYK, STYGWNLVR, SWMVRILIGF, SYAQMWTLMY, YAQMWQLMYF, YAQMWSLMY, YAQMWSLMYF, YAQMWTLMY, YAQMWTLMYF, TAAVLLLITH, TAEAGGRAY, TETTILDVDL, TFKVPHAKR, TLMAMDLGEL, TLMLLALIAV, TPEAKNSTF, TPEARNSTF, TPEGIIPAL, TPEGIIPALF, TPEGIIPSM, TPEGIIPSMF, TPEGIIPTLF, YTPEGIIPTL, TPFGQQRVF, TPKGAVMDII, TPRGTVMDII, TPMLRHTIEN, TPPGSRDPF, TPQDNQLAY, TPQDNQLAYV, TPQDNQLTY, TPRSMPGTRR, TPVHSWEDI, TTFSLHYAW, TTKRDLGMSK, TYLALIATF, VASGLLWVAE, VATTFVTPM, VLLLVTHYAI, VPLLAIGCY, VPLLAMGCY, VPMVTQMAM, VPNYNMIIM, VPYLGKREDQ, VQADMGCVV, VSSVNMVSRL, VTIDLDPVVY, VTRGAVLMHK, VTYECPLLV, VYTQLCDHR, VYTQLCDHRL, WALCESITL, WAYHGSYET, WAYHGSYEV, WHYDQDHPY, WSIHAHHQW, WVAEIQPQW, YAQIQPHWI, YENLKYSVI, YGGPISQHNY, YGVLFSGVSW, YKVASAGISY, YLAGAGLAF, YPKTKLTDW, YPKTKLTDWD, YQLAVTITAI, YQLWTALISL, YQNKVVKVLR, YQNKVVKVQR, YVSAIAQTEK (SEQ ID NOs: 813-1163) or a subsequence thereof or a modification thereof.

85. The method of claim 29, wherein the peptide comprises or consists of a peptide sequence set forth as any one of: TPEGIIPAL, TPEGIIPALF, TPEGIIPSM, TPEGIIPSMF, TPEGIIPTLF, YTPEGIIPTL, GEARKTFVDL, GEARKTFVEL, GEQRKTFVEL, GESRKTFVEL, LPVWLAHKVA, LPVWLAYKV, LPVWLAYKVA, LPVWLAYRVA, LPVWLSYKV, HPGAGKTKRY, NPEIEDDIF, DTTPFGQQR, MSFRDLGRVM, ATGPILTLW, ATGPISTLW, ATGPITTLW, ATGPLTTLW, ATGPVLTLW, LATGPVLTLW, CLIPTAMAF, MLIPTAMAF, VATTFVTPM, KPRICTREEF, KPRLCTREEF, NPRLCTREEF, RPRLCTREEF, TPRMCTREEF, VPLLAIGCY, VPLLAMGCY, MSYSMCTGKF, DPASIAARGY, IANQATVLM, APTRVVAAEM, APTRVVASEM, TPMLRHTIEN, MLVTPSMTM, FTMRHKKATY, FTILALFLAH, WHYDQDHPY, MALKDFKEF, LMKITAEWLW, MEITAEWLW, VMGITAEWLW, TETTILDVDL, GEFRLRGEQR (SEQ ID NOs: 764-812, 1106, 888) or a subsequence thereof or a modification thereof.


SEQUENCE LISTING (1163 sequences)

SEQ ID NO: 1 - protein, 114 aa, Dengue virus
MNNQRKKARNTPFNMLKRERNRVSTVQQLTKRFSLGMLQGRGPLKLFMALVAFLRFLTIPPTAGILKRWGTIKKSKAINVLRGFRKEIGRMLNILNRRRRTAGMIIMLIPTVMA

SEQ ID NO: 2 - protein, 166 aa, Dengue virus
FHLTTRNGEPHMIVSRQEKGKSLLFKTGDGVNMCTLMAMDLGELCEDTITYKCPLLRQNEPEDIDCWCNSTSTWVTYGTCTTTGEHRREKRSVALVPHVGMGLETRTETWMSSEGAWKHAQRIETWILRHPGFTIMAAILAYTIGTTHFQRALIFILLTAVAPSMT

SEQ ID NO: 3 - protein, 495 aa, Dengue virus
MRCIGISNRDFVEGVSGGSWVDIVLEHGSCVTTMAKNKPTLDFELIKTEAKQSATLRKYCIEAKLTNTTTESRCPTQGEPSLNEEQDKRFVCKHSMVDRGWGNGCGLFGKGGIVTCAMFTCKKNMKGKVVQPENLEYTIVITPHSGEEHAVGNDTGKHGKEIKITPQSSITEAELTGYGTVTMECSPRTGLDFNEMVLLQMENKAWLVHRQWFLDLPLPWLPGADTQGSNWIQKETLVTFKNPHAKKQDVVVLGSQEGAMHTALTGATEIQMSSGNLLFTGHLKCRLRMDKLQLKGMSYSMCTGKFKVVKEIAETQHGTIVIRVQYEGDGSPCKIPFEIMDLEKRHVLGRLITVNPIVTEKDSPVNIEAEPPFGDSYIIIGVEPGQLKLNWFKKGSSIGQMLETTMRGAKRMAILGDTAWDFGSLGGVFTSIGKALHQVFGAIYGAAFSGVSWTMKILIGVIITWIGMNSRSTSLSVSLVLVGVVTLYLGVMVQA

SEQ ID NO: 4 - protein, 353 aa, Dengue virus
ADSGCVVSWKNKELKCGSGIFITDNVHTWTEQYKFQPESPSKLASAIQKAHEEGICGIRSVTRLENLMWKQITPELNHILSENEVKLTIMTGDIKGIMQAGKRSLRPQPTELKYSWKTWGKAKMLSTESHNQTFLIDGPETAECPNTNRAWNSLEVEDYGFGVFTTNIWLKLREKQDVFCDSKLMSAAIKDNRAVHADMGYWIESALNDTWKIEKASFIEVKSCHWPKSHTLWSNEVLESEMIIPKNFAGPVSQHNYRPGYHTQTAGPWHLGKLEMDFDFCEGTTVVVTEDCGNRGPSLRTTTASGKLITEWCCRSCTLPPLRYRGEDGCWYGMEIRPLKEKEENLVNSLVTA

SEQ ID NO: 5 - protein, 218 aa, Dengue virus
GHGQIDNFSLGVLGMALFLEEMLRTRVGTKHAILLVAVSFVTLITGNMSFRDLGRVMVMVGATMTDDIGMGVTYLALLAAFKVRPTFAAGLLLRKLTSKELMMTTIGIVLLSQSTIPETILELTDALALGMMVLKMVRKMEKYQLAVTIMAILCVPNAVILQNAWKVSCTILAVVSVSPLFLTSSQQKADWIPLALTIKGLNPTAIFLTTLSRTNKKR

SEQ ID NO: 6 - protein, 130 aa, Dengue virus
SWPLNEAIMAVGMVSILASSLLKNDIPMTGPLVAGGLLTVCYVLTGRSADLELERAADVKWEDQAEISGSSPILSITISEDGSMSIKNEEEEQTLTILIRTGLLVISGLFPVSLPITAAAWYLWEVKKQR

SEQ ID NO: 7 - protein, 618 aa, Dengue virus
AGVLWDVPSPPPVGKAELEDGAYRIKQKGILGYSQIGAGVYKEGTFHTMWHVTRGAVLMHKGKRIEPSWADVKKDLISYGGGWKLEGEWKEGEEVQVLALEPGKNPRAVQTKPGLFKTNAGTIGAVSLDFSPGTSGSPIIDKKGKVVGLYGNGVVTRSGAYVSAIAQTEKSIEDNPEIEDDIFRKRKLTIMDLHPGAGKTKRYLPAIVREAIKRGLRTLILAPTRVVAAEMEEALRGLPIRYQTPAIRAEHTGREIVDLMCHATFTMRLLSPVRVPNYNLIIMDEAHFTDPASIAARGYISTRVEMGEAAGIFMTATPPGSRDPFPQSNAPIMDEEREIPERSWSSGHEWVTDFKGKTVWFVPSIKAGNDIAACLRKNGKKVIQLSRKTFDSEYVKTRTNDWDFVVTTDISEMGANFKAERVIDPRRCMKPVILTDGEERVILAGPMPVTHSSAAQRRGRIGRNPKNENDQYIYMGEPLENDEDCAHWKEAKMLLDNINTPEGIIPSMFEPEREKVDAIDGEYRLRGEARKTFVDLMRRGDLPVWLAYRVAAEGINYADRRWCFDGIKNNQILEENVEVEIWTKEGERKKLKPRWLDARIYSDPLALKEFKEFAAGRK

SEQ ID NO: 8 - protein, 150 aa, Dengue virus
SLTLSLITEMGRLPTFMTQKARDALDNLAVLHTAEAGGRAYNHALSELPETLETLLLLTLLATVTGGIFLFLMSGRGIGKMTLGMCCIITASILLWYAQIQPHWIAASIILEFFLIVLLIPEPEKQRTPQDNQLTYVVIAILTVVAATMA

SEQ ID NO: 9 - protein, 248 aa, Dengue virus
NEMGFLEKTKKDLGLGSITTQQPESNILDIDLRPASAWTLYAVATTFVTPMLRHSIENSSVNVSLTAIANQATVLMGLGKGWPLSKMDIGVPLLAIGCYSQVNPITLTAALFLLVAHYAIIGPGLQAKATREAQKRAAAGIMKNPTVDGITVIDLDPIPYDPKFEKQLGQVMLLVLCVTQVLMMRTTWALCEALTLATGPISTLWEGNPGRFWNTTIAVSMANIFRGSYLAGAGLLFSIMKNTTNTRR

SEQ ID NO: 10 - protein, 900 aa, Dengue virus
GTGNIGETLGEKWKSRLNALGKSEFQIYKKSGIQEVDRTLAKEGIKRGETDHHAVSRGSAKLRWFVERNMVTPEGKVVDLGCGRGGWSYYCGGLKNVREVKGLTKGGPGHEEPIPMSTYGWNLVRLQSGVDVFFTPPEKCDTLLCDIGESSPNPTVEAGRTLRVLNLVENWLNNNTQFCIKVLNPYMPSVIEKMEALQRKYGGALVRNPLSRNSTHEMYWVSNASGNIVSSVNMISRMLINRFTMRHKKATYEPDVDLGSGTRNIGIESEIPNLDIIGKRIEKIKQEHETSWHYDQDHPYKTWAYHGSYETKQTGSASSMVNGVVRLLTKPWDVVPMVTQMAMTDTTPFGQQRVFKEKVDTRTQEPKEGTKKLMKITAEWLWKELGKKKTPRMCTREEFTRKVRSNAALGAIFTDENKWKSAREAVEDSRFWELVDKERNLHLEGKCETCVYNMMGKREKKLGEFGKAKGSRAIWYMWLGARFLEFEALGFLNEDHWFSRENSLSGVEGEGLHKLGYILRDVSKKEGGAMYADDTAGWDTRITLEDLKNEEMVTNHMEGEHKKLAEAIFKLTYQNKVVRVQRPTPRGTVMDIISRRDQRGSGQVGTYGLNTFTNMEAQLIRQMEGEGVFKSIQHLTVTEEIAVQNWLARVGRERLSRMAISGDDCVVKPLDDRFASALTALNDMGKVRKDIQQWEPSRGWNDWTQVPFCSHHFHELIMKDGRVLVVPCRNQDELIGRARISQGAGWSLRETACLGKSYAQMWSLMYFHRRDLRLAANAICSAVPSHWVPTSRTTWSIHAKHEWMTAEDMLTVWNRVWIQENPWMEDKTPVESWEEIPYLGKREDQWCGSLIGLTSRATWAKNIQTAINQVRSLIGNEEYTDYMPSMKRFRREEEEAGVLW

SEQ ID NO: 11 - protein, 15 aa, Dengue virus
GLFPVSLPITAAAWY

SEQ ID NO: 12 - protein, 15 aa, Dengue virus
GKTKRYLPAIVREAI

SEQ ID NO: 13 - protein, 15 aa, Dengue virus
GLPIRYQTPAIRAEH

SEQ ID NO: 14 - protein, 15 aa, Dengue virus
IGCYSQVNPITLTAA

SEQ ID NO: 15 - protein, 9 aa, Dengue virus
ITEAELTGY

SEQ ID NO: 16 - protein, 10 aa, Dengue virus
RSCTLPPLRY

SEQ ID NO: 17 - protein, 9 aa, Dengue virus
MTDDIGMGV

SEQ ID NO: 18 - protein, 9 aa, Dengue virus
LTDALALGM

SEQ ID NO: 19 - protein, 9 aa, Dengue virus
VIDLDPIPY

SEQ ID NO: 20 - protein, 9 aa, Dengue virus
YTDYMPSMK

SEQ ID NO: 21 - protein, 9 aa, Dengue virus
RLITVNPIV

SEQ ID NO: 22 - protein, 9 aa, Dengue virus
IMAVGMVSI

SEQ ID NO: 23 - protein, 9 aa, Dengue virus
GLLTVCYVL

SEQ ID NO: 24 - protein, 10 aa, Dengue virus
LLVISGLFPV

SEQ ID NO: 25 - protein, 9 aa, Dengue virus
AAAWYLWEV

SEQ ID NO: 26 - protein, 9 aa, Dengue virus
YLPAIVREA

SEQ ID NO: 27 - protein, 10 aa, Dengue virus
DLMRRGDLPV

SEQ ID NO: 28 - protein, 9 aa, Dengue virus
ALSELPETL

SEQ ID NO: 29 - protein, 9 aa, Dengue virus
IILEFFLIV

SEQ ID NO: 30 - protein, 9 aa, Dengue virus
KLAEAIFKL

SEQ ID NO: 31 - protein, 9 aa, Dengue virus
SQIGAGVYK

SEQ ID NO: 32 - protein, 10 aa, Dengue virus
GTSGSPIIDK

SEQ ID NO: 33 - protein, 9 aa, Dengue virus
KTFDSEYVK

SEQ ID NO: 34 - protein, 10 aa, Dengue virus
RIYSDPLALK

SEQ ID NO: 35 - protein, 9 aa, Dengue virus
ATVLMGLGK

SEQ ID NO: 36 - protein, 9 aa, Dengue virus
STYGWNLVR

SEQ ID NO: 37 - protein, 9 aa, Dengue virus
TVMDIISRR

SEQ ID NO: 38 - protein, 10 aa, Dengue virus
RQMEGEGVFK

SEQ ID NO: 39 - protein, 9 aa, Dengue virus
RTTWSIHAK

SEQ ID NO: 40 - protein, 10 aa, Dengue virus
RPTFAAGLLL

SEQ ID NO: 41 - protein, 9 aa, Dengue virus
LPAIVREAI

SEQ ID NO: 42 - protein, 10 aa, Dengue virus
APTRVVAAEM

SEQ ID NO: 43 - protein, 9 aa, Dengue virus
VPNYNLIIM

SEQ ID NO: 44 - protein, 10 aa, Dengue virus
APIMDEEREI

SEQ ID NO: 45 - protein, 10 aa, Dengue virus
TPEGIIPSMF

SEQ ID NO: 46 - protein, 9 aa, Dengue virus
KPRWLDARI

SEQ ID NO: 47 - protein, 10 aa, Dengue virus
RPASAWTLYA

SEQ ID NO: 48 - protein, 8 aa, Dengue virus
TPMLRHSI

SEQ ID NO: 49 - protein, 10 aa, Dengue virus
SPNPTVEAGR

SEQ ID NO: 50 - protein, 10 aa, Dengue virus
TPRMCTREEF

SEQ ID NO: 51 - protein, 9 aa, Dengue virus
RPTPRGTVM

SEQ ID NO: 52 - protein, 15 aa, Dengue virus
AFLRFLTIPPTAGIL

SEQ ID NO: 53 - protein, 15 aa, Dengue virus
GVTYLALLAAFKVRP

SEQ ID NO: 54 - protein, 15 aa, Dengue virus
MAVGMVSILASSLLK

SEQ ID NO: 55 - protein, 15 aa, Dengue virus
TFTMRLLSPVRVPNY

SEQ ID NO: 56 - protein, 15 aa, Dengue virus
SRAIWYMWLGARFLE

SEQ ID NO: 57 - protein, 9 aa, Dengue virus
ITEAELTGY

SEQ ID NO: 58 - protein, 9 aa, Dengue virus
STEIQLTDY

SEQ ID NO: 59 - protein, 9 aa, Dengue virus
TTEIQLTDY

SEQ ID NO: 60 - protein, 9 aa, Dengue virus
TSEIQLIDY

SEQ ID NO: 61 - protein, 9 aa, Dengue virus
TSEIQLTDY

SEQ ID NO: 62 - protein, 9 aa, Dengue virus
IAEAELTGY

SEQ ID NO: 63 - protein, 9 aa, Dengue virus
IAEAELTDY

SEQ ID NO: 64 - protein, 9 aa, Dengue virus
ITDAELTGY

SEQ ID NO: 65 - protein, 9 aa, Dengue virus
STEAELTGY

SEQ ID NO: 66 - protein, 9 aa, Dengue virus
TTEAELTGY

SEQ ID NO: 67 - protein, 9 aa, Dengue virus
ISEAELTDY

SEQ ID NO: 68 - protein, 9 aa, Dengue virus
ITEAELTGY

SEQ ID NO: 69 - protein, 9 aa, Dengue virus
TVEAVLLEY

SEQ ID NO: 70 - protein, 9 aa, Dengue virus
TVEAVLPEY

SEQ ID NO: 71 - protein, 9 aa, Dengue virus
TVEAILPEY

SEQ ID NO: 72 - protein, 9 aa, Dengue virus
TAEAILPEY

SEQ ID NO: 73 - protein, 9 aa, Dengue virus
THEALLPEY

SEQ ID NO: 74 - protein, 9 aa, Dengue virus
ITEAILPEY

SEQ ID NO: 75 - protein, 9 aa, Dengue virus
TTEVILPEY

SEQ ID NO: 76 - protein, 9 aa, Dengue virus
TTEAILPEY

SEQ ID NO: 77 - protein, 9 aa, Dengue virus
SVEVELPDY

SEQ ID NO: 78 - protein, 9 aa, Dengue virus
SVEVKLPDY

SEQ ID NO: 79 - protein, 10 aa, Dengue virus
RSCTLPPLRY

SEQ ID NO: 80 - protein, 10 aa, Dengue virus
RSCTLPPLRF

SEQ ID NO: 81 - protein, 10 aa, Dengue virus
RSCTLPPLRY

SEQ ID NO: 82 - protein, 10 aa, Dengue virus
RSCTLPPLRY

SEQ ID NO: 83 - protein, 10 aa, Dengue virus
RSCTMPPLRF

SEQ ID NO: 84 - protein, 9 aa, Dengue virus
MTDDIGMGV

SEQ ID NO: 85 - protein, 9 aa, Dengue virus
ASDRMGMGM

SEQ ID NO: 86 - protein, 9 aa, Dengue virus
ASDMMGMGT

SEQ ID NO: 87 - protein, 9 aa, Dengue virus
ASDKMGMGT

SEQ ID NO: 88 - protein, 9 aa, Dengue virus
ASDNMGMGT

SEQ ID NO: 89 - protein, 9 aa, Dengue virus
VSDRMGMGT

SEQ ID NO: 90 - protein, 9 aa, Dengue virus
ASDRMGMGT

SEQ ID NO: 91 - protein, 9 aa, Dengue virus
MADDIGMGV

SEQ ID NO: 92 - protein, 9 aa, Dengue virus
MTDEMGMGV

SEQ ID NO: 93 - protein, 9 aa, Dengue virus
ITDDIGMGV

SEQ ID NO: 94 - protein, 9 aa, Dengue virus
MTDDIGMGV

SEQ ID NO: 95 - protein, 9 aa, Dengue virus
ASDRTGMGV

SEQ ID NO: 96 - protein, 9 aa, Dengue virus
ASDKMGMGV

SEQ ID NO: 97 - protein, 9 aa, Dengue virus
ATDRMGMGV

SEQ ID NO: 98 - protein, 9 aa, Dengue virus
ASDRMGMGV

SEQ ID NO: 99 - protein, 9 aa, Dengue virus
LTDALALGM

SEQ ID NO: 100 - protein, 9 aa, Dengue virus
LGDGLAIGI

SEQ ID NO: 101 - protein, 9 aa, Dengue virus
LGDGFAMGI

SEQ ID NO: 102 - protein, 9 aa, Dengue virus
LGDGLAMGI

SEQ ID NO: 103 - protein, 9 aa, Dengue virus
LTDAIALGI

SEQ ID NO: 104 - protein, 9 aa, Dengue virus
LTDAWALGM

SEQ ID NO: 105 - protein, 9 aa, Dengue virus
LTDALALGI

SEQ ID NO: 106 - protein, 9 aa, Dengue virus
LTDALALGM

SEQ ID NO: 107 - protein, 9 aa, Dengue virus
MANGVALGL

SEQ ID NO: 108 - protein, 9 aa, Dengue virus
MANGIALGL

SEQ ID NO: 109 - protein, 9 aa, Dengue virus
LISGISLGL

SEQ ID NO: 110 - protein, 9 aa, Dengue virus
FIDGLSLGL

SEQ ID NO: 111 - protein, 9 aa, Dengue virus
LIDGISLGL

SEQ ID NO: 112 - protein, 9 aa, Dengue virus
LIDGIALGL

SEQ ID NO: 113 - protein, 9 aa, Dengue virus
FIDGISLGL

SEQ ID NO: 114 - protein, 9 aa, Dengue virus
VIDLDPIPY

SEQ ID NO: 115 - protein, 9 aa, Dengue virus
TIDLDPVVY

SEQ ID NO: 116 - protein, 9 aa, Dengue virus
AIDLDPVVY

SEQ ID NO: 117 - protein, 9 aa, Dengue virus
VIDLEPIPY

SEQ ID NO: 118 - protein, 9 aa, Dengue virus
VIDLDPIPY

SEQ ID NO: 119 - protein, 9 aa, Dengue virus
TIDLDSVIF

SEQ ID NO: 120 - protein, 9 aa, Dengue virus
TIDLDPVIY

SEQ ID NO: 121 - protein, 9 aa, Dengue virus
TIALDPVIY

SEQ ID NO: 122 - protein, 9 aa, Dengue virus
VIDLEPISY

SEQ ID NO: 123 - protein, 9 aa, Dengue virus
YTDYMPSMK

SEQ ID NO: 124 - protein, 9 aa, Dengue virus
YSDYMTSMK

SEQ ID NO: 125 - protein, 9 aa, Dengue virus
YLDYMASMK

SEQ ID NO: 126 - protein, 9 aa, Dengue virus
YIDYMTSMK

SEQ ID NO: 127 - protein, 9 aa, Dengue virus
YLDFMTSMK

SEQ ID NO: 128 - protein, 9 aa, Dengue virus
YLDYMTSMK

SEQ ID NO: 129 - protein, 9 aa, Dengue virus
YLDYMISMK

SEQ ID NO: 130 - protein, 9 aa, Dengue virus
YIDYMPSMK

SEQ ID NO: 131 - protein, 9 aa, Dengue virus
YMDYMPSMK

SEQ ID NO: 132 - protein, 9 aa, Dengue virus
YTDYMPSMK

SEQ ID NO: 133 - protein, 9 aa, Dengue virus
FLDYMPSMK

SEQ ID NO: 134 - protein, 9 aa, Dengue virus
YADYMPVMK

SEQ ID NO: 135 - protein, 9 aa, Dengue virus
YMDYMPVMK

SEQ ID NO: 136 - protein, 9 aa, Dengue virus
YVDYMPAMK

SEQ ID NO: 137 - protein, 9 aa, Dengue virus
YVDYMPVMR

SEQ ID NO: 138 - protein, 9 aa, Dengue virus
YVDYMPVMK

SEQ ID NO: 139 - protein, 9 aa, Dengue virus
RLITVNPIV

SEQ ID NO: 140 - protein, 9 aa, Dengue virus
RVITANPIV

SEQ ID NO: 141 - protein, 9 aa, Dengue virus
RLVTANPIV

SEQ ID NO: 142 - protein, 9 aa, Dengue virus
RLITANPIV

SEQ ID NO: 143 - protein, 9 aa, Dengue virus
RLITVNPVV

SEQ ID NO: 144 - protein, 9 aa, Dengue virus
RLITVNPII

SEQ ID NO: 145 - protein, 9 aa, Dengue virus
RLITVNPIV

SEQ ID NO: 146 - protein, 9 aa, Dengue virus
RLTTVNPIV

SEQ ID NO: 147 - protein, 9 aa, Dengue virus
RLITANPIV

SEQ ID NO: 148 - protein, 9 aa, Dengue virus
RLITANPVV

SEQ ID NO: 149 - protein, 9 aa, Dengue virus
RVISATPLA

SEQ ID NO: 150 - protein, 9 aa, Dengue virus
RVISSTPLA

SEQ ID NO: 151 - protein, 9 aa, Dengue virus
RIISSTPLA

SEQ ID NO: 152 - protein, 9 aa, Dengue virus
RVISSTPFA

SEQ ID NO: 153 - protein, 9 aa, Dengue virus
RIISSTPFA

SEQ ID NO: 154 - protein, 9 aa, Dengue virus
RIISSIPFA

SEQ ID NO: 155 - protein, 9 aa, Dengue virus
IMAVGMVSI

SEQ ID NO: 156 - protein, 9 aa, Dengue virus
IMAVGVVSI

SEQ ID NO: 157 - protein, 9 aa, Dengue virus
VMAVGIVSI

SEQ ID NO: 158 - protein, 9 aa, Dengue virus
IMAIGIVSI

SEQ ID NO: 159 - protein, 9 aa, Dengue virus
IMAVGIVSI

SEQ ID NO: 160 - protein, 9 aa, Dengue virus
VMAVGMVSI

SEQ ID NO: 161 - protein, 9 aa, Dengue virus
IMAVGMVSI

SEQ ID NO: 162 - protein, 9 aa, Dengue virus
VMAIGLVSI

SEQ ID NO: 163 - protein, 9 aa, Dengue virus
VMAVGLVSI

SEQ ID NO: 164 - protein, 9 aa, Dengue virus
MMAVGLVSL

SEQ ID NO: 165 - protein, 9 aa, Dengue virus
IMAVGLVSL

SEQ ID NO: 166 - protein, 9 aa, Dengue virus
GLLTVCYVL

SEQ ID NO: 167 - protein, 9 aa, Dengue virus
GMLITCYVI

SEQ ID NO: 168 - protein, 9 aa, Dengue virus
GMLIACYVI

SEQ ID NO: 169 - protein, 9 aa, Dengue virus
GPLTVCYVL

SEQ ID NO: 170 - protein, 9 aa, Dengue virus
GLLTVCYVL

SEQ ID NO: 171 - protein, 9 aa, Dengue virus
GMLIACYVI

SEQ ID NO: 172 - protein, 9 aa, Dengue virus
GLLIACYVI

SEQ ID NO: 173 - protein, 9 aa, Dengue virus
GLLLAAYMM

SEQ ID NO: 174 - protein, 9 aa, Dengue virus
GLLLAAYVM

SEQ ID NO: 175 - protein, 10 aa, Dengue virus
RIYSDPLALK

SEQ ID NO: 176 - protein, 10 aa, Dengue virus
RTYSDPQALR

SEQ ID NO: 177 - protein, 10 aa, Dengue virus
RTYSDPLALR

SEQ ID NO: 178 - protein, 10 aa, Dengue virus
RTYSDPLALK

SEQ ID NO: 179 - protein, 10 aa, Dengue virus
RIYSDPLTLK

SEQ ID NO: 180 - protein, 10 aa, Dengue virus
KIYSDPLALK

SEQ ID NO: 181 - protein, 10 aa, Dengue virus
RIYSEPRALK

SEQ ID NO: 182 - protein, 10 aa, Dengue virus
RIYSDPLALK

SEQ ID NO: 183 - protein, 10 aa, Dengue virus
RTYSDPLAPK

SEQ ID NO: 184 - protein, 10 aa, Dengue virus
RTYSDPLALK

SEQ ID NO: 185 - protein, 10 aa, Dengue virus
RIYSDPLALK

SEQ ID NO: 186 - protein, 10 aa, Dengue virus
RVYADPMALQ

SEQ ID NO: 187 - protein, 10 aa, Dengue virus
RVYADPMALK

SEQ ID NO: 188 - protein, 9 aa, Dengue virus
ATVLMGLGK

SEQ ID NO: 189 - protein, 9 aa, Dengue virus
AAILMGLDK

SEQ ID NO: 190 - protein, 9 aa, Dengue virus
ATVLMGLGK

SEQ ID NO: 191 - protein, 9 aa, Dengue virus
ATVLMGLGR

SEQ ID NO: 192 - protein, 9 aa, Dengue virus
AVVLMGLNK

SEQ ID NO: 193 - protein, 9 aa, Dengue virus
AVVLMGLDK

SEQ ID NO: 194 - protein, 9 aa, Dengue virus
AAVLMGLGK

SEQ ID NO: 195 - protein, 9 aa, Dengue virus
STYGWNLVR

SEQ ID NO: 196 - protein, 9 aa, Dengue virus
AAYGWNLVK

SEQ ID NO: 197 - protein, 9 aa, Dengue virus
ATYGWNLVK

SEQ ID NO: 198 - protein, 9 aa, Dengue virus
STYGWNLVR

SEQ ID NO: 199 - protein, 9 aa, Dengue virus
STYGWNLVK

SEQ ID NO: 200 - protein, 9 aa, Dengue virus
STYGWNVVK

SEQ ID NO: 201 - protein, 9 aa, Dengue virus
STYGWNIVK

SEQ ID NO: 202 - protein, 9 aa, Dengue virus
ATYGWNLVK

SEQ ID NO: 203 - protein, 9 aa, Dengue virus
TVMDIISRR

SEQ ID NO: 204 - protein, 9 aa, Dengue virus
TVMDIISRR

SEQ ID NO: 205 - protein, 9 aa, Dengue virus
TVMDVISRR

SEQ ID NO: 206 - protein, 9 aa, Dengue virus
TVLDIISRR

SEQ ID NO: 207 - protein, 9 aa, Dengue virus
TVMDIISRK

SEQ ID NO: 208 - protein, 9 aa, Dengue virus
TVMDIISRR

SEQ ID NO: 209 - protein, 9 aa, Dengue virus
TVMDIISRK

SEQ ID NO: 210 - protein, 9 aa, Dengue virus
AVMDIISRK

SEQ ID NO: 211 - protein, 10 aa, Dengue virus
RQMEGEGVFK

SEQ ID NO: 212 - protein, 10 aa, Dengue virus
RQMESEEIFS

SEQ ID NO: 213 - protein, 10 aa, Dengue virus
RQMESEGIVS

SEQ ID NO: 214 - protein, 10 aa, Dengue virus
RQMESEGIFF

SEQ ID NO: 215 - protein, 10 aa, Dengue virus
RQMESEGIIL

SEQ ID NO: 216 - protein, 10 aa, Dengue virus
RQMESEGIFS

SEQ ID NO: 217 - protein, 10 aa, Dengue virus
RQMESEGIFL

SEQ ID NO: 218 - protein, 10 aa, Dengue virus
RQMEGEGVFR

SEQ ID NO: 219 - protein, 10 aa, Dengue virus
RQMEGEGIFR

SEQ ID NO: 220 - protein, 10 aa, Dengue virus
RQMEGEGLFK

SEQ ID NO: 221 - protein, 10 aa, Dengue virus
RQMEGEEVFK

SEQ ID NO: 222 - protein, 10 aa, Dengue virus
RQMEGEGVFK

SEQ ID NO: 223 - protein, 10 aa, Dengue virus
RQMEGEGIFK

SEQ ID NO: 224 - protein, 10 aa, Dengue virus
RQMEGEGVLT

SEQ ID NO: 225 - protein, 10 aa, Dengue virus
RQMEGEGVLS

SEQ ID NO: 226 - protein, 10 aa, Dengue virus
RQMEGEDVLS

SEQ ID NO: 227 - protein, 10 aa, Dengue virus
RQMEAEGVIT

SEQ ID NO: 228 - protein, 9 aa, Dengue virus
RTTWSIHAK

SEQ ID NO: 229 - protein, 9 aa, Dengue virus
RTTWSIHAH

SEQ ID NO: 230 - protein, 9 aa, Dengue virus
RTTWSIHAR

SEQ ID NO: 231 - protein, 9 aa, Dengue virus
RTTWSIHAT

SEQ ID NO: 232 - protein, 9 aa, Dengue virus
RTTWSIHAS

SEQ ID NO: 233 - protein, 9 aa, Dengue virus
RTTWSIHAK

SEQ ID NO: 234 - protein, 9 aa, Dengue virus
RTTWSIHAH

SEQ ID NO: 235 - protein, 9 aa, Dengue virus
RTTWSIHAH

SEQ ID NO: 236 - protein, 10 aa, Dengue virus
RPTFAAGLLL

SEQ ID NO: 237 - protein, 10 aa, Dengue virus
RPMLAVGLLF

SEQ ID NO: 238 - protein, 10 aa, Dengue virus
RPMFAMGLLF

SEQ ID NO: 239 - protein, 10 aa, Dengue virus
RPMFAVGLLI

SEQ ID NO: 240 - protein, 10 aa, Dengue virus
RPMFAVGLLF

SEQ ID NO: 241 - protein, 10 aa, Dengue virus
RPTFAAGLFL

SEQ ID NO: 242 - protein, 10 aa, Dengue virus
RPTFAVGLVL

SEQ ID NO: 243 - protein, 10 aa, Dengue virus
RPTFAVGLLL

SEQ ID NO: 244 - protein, 10 aa, Dengue virus
RPTFAAGLLL

SEQ ID NO: 245 - protein, 10 aa, Dengue virus
QPFLALGFFM

SEQ ID NO: 246 - protein, 10 aa, Dengue virus
QPFLTLGFFL

SEQ ID NO: 247 - protein, 10 aa, Dengue virus
QPFLALGFFL

SEQ ID NO: 248 - protein, 10 aa, Dengue virus
SPRYVLGVFL

SEQ ID NO: 249 - protein, 10 aa, Dengue virus
SPGYVLGVFL

SEQ ID NO: 250 - protein, 10 aa, Dengue virus
SPGYVLGIFL

SEQ ID NO: 251 - protein, 9 aa, Dengue virus
LPAIVREAI

SEQ ID NO: 252 - protein, 9 aa, Dengue virus
LPAIIREAI

SEQ ID NO: 253 - protein, 9 aa, Dengue virus
LPAIVREAI

SEQ ID NO: 254 - protein, 9 aa, Dengue virus
LPAMVREAI

SEQ ID NO: 255 - protein, 9 aa, Dengue virus
LPAIVREAI

SEQ ID NO: 256 - protein, 9 aa, Dengue virus
LPTIVREAI

SEQ ID NO: 257 - protein, 9 aa, Dengue virus
LPAVVREAI

SEQ ID NO: 258 - protein, 9 aa, Dengue virus
LPAIVREAI

SEQ ID NO: 259 - protein, 9 aa, Dengue virus
LPAIIREAI

SEQ ID NO: 260 - protein, 9 aa, Dengue virus
LPSIVREAL

SEQ ID NO: 261 - protein, 10 aa, Dengue virus
APTRVVAAEM

SEQ ID NO: 262 - protein, 10 aa, Dengue virus
APTRVVASET

SEQ ID NO: 263 - protein, 10 aa, Dengue virus
APTRVVAAEM

SEQ ID NO: 264 - protein, 10 aa, Dengue virus
APTRVVASEM

SEQ ID NO: 265 - protein, 10 aa, Dengue virus
APPRVVPAEM

SEQ ID NO: 266 - protein, 10 aa, Dengue virus
APTRVVAAEM

SEQ ID NO: 267 - protein, 10 aa, Dengue virus
APTRVVAAEM

SEQ ID NO: 268 - protein, 10 aa, Dengue virus
APTRVVAAEM

SEQ ID NO: 269 - protein, 9 aa, Dengue virus
VPNYNLIIM

SEQ ID NO: 270 - protein, 9 aa, Dengue virus
VPNYNMIIV

SEQ ID NO: 271 - protein, 9 aa, Dengue virus
VPNYNMIIM

SEQ ID NO: 272 - protein, 9 aa, Dengue virus
VPNYNMIVM

SEQ ID NO: 273 - protein, 9 aa, Dengue virus
VPNYNLIIM

SEQ ID NO: 274 - protein, 9 aa, Dengue virus
VPNYNLIVM

SEQ ID NO: 275 - protein, 9 aa, Dengue virus
VPNYNLVVM

SEQ ID NO: 276 - protein, 9 aa, Dengue virus
VPNYNLVIM

SEQ ID NO: 277 - protein, 9 aa, Dengue virus
VSNYNLIIM

SEQ ID NO: 278 - protein, 9 aa, Dengue virus
VPNYNLIIM

SEQ ID NO: 279 - protein, 9 aa, Dengue virus
VPNYNLIVM

SEQ ID NO: 280 - protein, 10 aa, Dengue virus
APIMDEEREI

SEQ ID NO: 281 - protein, 10 aa, Dengue virus
AIIQDEERDI

SEQ ID NO: 282 - protein, 10 aa, Dengue virus
AVIQDEEKDI

SEQ ID NO: 283 - protein, 10 aa, Dengue virus
AAIQDEERDI

SEQ ID NO: 284 - protein, 10 aa, Dengue virus
AVIQDEERDI

SEQ ID NO: 285 - protein, 10 aa, Dengue virus
APIMDDEREI

SEQ ID NO: 286 - protein, 10 aa, Dengue virus
APIIDEEREI

SEQ ID NO: 287 - protein, 10 aa, Dengue virus
APIVDEEREI

SEQ ID NO: 288 - protein, 10 aa, Dengue virus
APIMDEEREI

SEQ ID NO: 289 - protein, 10 aa, Dengue virus
APIQDEEKDI

SEQ ID NO: 290 - protein, 10 aa, Dengue virus
SPIQDEERDI

SEQ ID NO: 291 - protein, 10 aa, Dengue virus
APIQDEERDI

SEQ ID NO: 292 - protein, 10 aa, Dengue virus
APIQDKERDI

SEQ ID NO: 293 - protein, 10 aa, Dengue virus
SPIEDIEREI

SEQ ID NO: 294 - protein, 10 aa, Dengue virus
TPEGIIPSMF

SEQ ID NO: 295 - protein, 10 aa, Dengue virus
TPEGIIPALY

SEQ ID NO: 296 - protein, 10 aa, Dengue virus
TPEGIIPALF

SEQ ID NO: 297 - protein, 10 aa, Dengue virus
TPEGIIPSLF

SEQ ID NO: 298 - protein, 10 aa, Dengue virus
TPEGIIPSMF

SEQ ID NO: 299 - protein, 10 aa, Dengue virus
TPEGIIPALF

SEQ ID NO: 300 - protein, 10 aa, Dengue virus
TPEGIIPTLF

SEQ ID NO: 301 - protein, 10 aa, Dengue virus
LLVISGLFPV

SEQ ID NO: 302 - protein, 10 aa, Dengue virus
LLAISGVYPL

SEQ ID NO: 303 - protein, 10 aa, Dengue virus
LLAVSGMYPL

SEQ ID NO: 304 - protein, 10 aa, Dengue virus
LLAVSGVYPL

SEQ ID NO: 305 - protein, 10 aa, Dengue virus
LLVISGVYPM

SEQ ID NO: 306 - protein, 10 aa, Dengue virus
LLAVSGVYPI

SEQ ID NO: 307 - protein, 10 aa, Dengue virus
LLAASGVYPM

SEQ ID NO: 308 - protein, 10 aa, Dengue virus
LLAISGVYPM

SEQ ID NO: 309 - protein, 10 aa, Dengue virus
LLAVSGVYPM

SEQ ID NO: 310 - protein, 10 aa, Dengue virus
LLVVSGLFPV

SEQ ID NO: 311 - protein, 10 aa, Dengue virus
LLVISGLFPA

SEQ ID NO: 312 - protein, 10 aa, Dengue virus
LLVISGLFPI

SEQ ID NO: 313 - protein, 10 aa, Dengue virus
LLVISGVFPV

SEQ ID NO: 314 - protein, 10 aa, Dengue virus
LLVISGLFPV

SEQ ID NO: 315 - protein, 10 aa, Dengue virus
LLIVSGIFPC

SEQ ID NO: 316 - protein, 10 aa, Dengue virus
LLIVSGIFPY

SEQ ID NO: 317 - protein, 10 aa, Dengue virus
LLIVSGVFPY

SEQ ID NO: 318 - protein, 10 aa, Dengue virus
LITVSGLYPL

SEQ ID NO: 319 - protein, 9 aa, Dengue virus
AAAWYLWEV

SEQ ID NO: 320 - protein, 9 aa, Dengue virus
LFVWCFWQK

SEQ ID NO: 321 - protein, 9 aa, Dengue virus
LFLWYFWQK

SEQ ID NO: 322 - protein, 9 aa, Dengue virus
LFVWHFWQK

SEQ ID NO: 323 - protein, 9 aa, Dengue virus
FFVWYFWQK

SEQ ID NO: 324 - protein, 9 aa, Dengue virus
PFVWYFWQK

SEQ ID NO: 325 - protein, 9 aa, Dengue virus
LFVWYFWQK

SEQ ID NO: 326 - protein, 9 aa, Dengue virus
AAAWYLWET

SEQ ID NO: 327 - protein, 9 aa, Dengue virus
AAAWYLWEA

SEQ ID NO: 328 - protein, 9 aa, Dengue virus
AAAWYLWEV

SEQ ID NO: 329 - protein, 9 aa, Dengue virus
LLVWHAWQK

SEQ ID NO: 330 - protein, 9 aa, Dengue virus
MLVWHTWQK

SEQ ID NO: 331 - protein, 9 aa, Dengue virus
LLVWHTWQK

SEQ ID NO: 332 - protein, 9 aa, Dengue virus
MALWYIWQV

SEQ ID NO: 333 - protein, 9 aa, Dengue virus
MTLWYMWQV

SEQ ID NO: 334 - protein, 9 aa, Dengue virus
MALWYMWQV

SEQ ID NO: 335 - protein, 9 aa, Dengue virus
YLPAIVREA

SEQ ID NO: 336 - protein, 9 aa, Dengue virus
YLPAIIREA

SEQ ID NO: 337 - protein, 9 aa, Dengue virus
YLPAIVREA

SEQ ID NO: 338 - protein, 9 aa, Dengue virus
YLPAMVREA

SEQ ID NO: 339 - protein, 9 aa, Dengue virus
SLPAIVREA

SEQ ID NO: 340 - protein, 9 aa, Dengue virus
YLPAIVREA

SEQ ID NO: 341 - protein, 9 aa, Dengue virus
YLPTIVREA

SEQ ID NO: 342 - protein, 9 aa, Dengue virus
YLPAVVREA

SEQ ID NO: 343 - protein, 9 aa, Dengue virus
YLPAIVREA

SEQ ID NO: 344 - protein, 9 aa, Dengue virus
YLPAIIREA

SEQ ID NO: 345 - protein, 9 aa, Dengue virus
ILPSIVREA

SEQ ID NO: 346 - protein, 10 aa, Dengue virus
DLMRRGDLPV

SEQ ID NO: 347 - protein, 10 aa, Dengue virus
DLLRRGDLPV

SEQ ID NO: 348 - protein, 10 aa, Dengue virus
ELMRRGDLPV

SEQ ID NO: 349 - protein, 10 aa, Dengue virus
DLMKRGDLPV

SEQ ID NO: 350 - protein, 10 aa, Dengue virus
ELMRRGDLPV

SEQ ID NO: 351 - protein, 10 aa, Dengue virus
DLMRRGDLPV

SEQ ID NO: 352 - protein, 10 aa, Dengue virus
ELMRRGHLPV

SEQ ID NO: 353 - protein, 10 aa, Dengue virus
ELMRRGDLPV

SEQ ID NO: 354 - protein, 10 aa, Dengue virus
ELMKRGDLPV

SEQ ID NO: 355 - protein, 10 aa, Dengue virus
ELMRRGDLPV

SEQ ID NO: 356 - protein, 9 aa, Dengue virus
ALSELPETL

SEQ ID NO: 357 - protein, 9 aa, Dengue virus
ALEELPDTI

SEQ ID NO: 358 - protein, 9 aa, Dengue virus
AVEELPDTI

SEQ ID NO: 359 - protein, 9 aa, Dengue virus
AMEELPDTI

SEQ ID NO: 360 - protein, 9 aa, Dengue virus
ALSELAETL

SEQ ID NO: 361 - protein, 9 aa, Dengue virus
ALGELPETL

SEQ ID NO: 362 - protein, 9 aa, Dengue virus
ALSELPETL

SEQ ID NO: 363 - protein, 9 aa, Dengue virus
AVEELPETM

SEQ ID NO: 364 - protein, 9 aa, Dengue virus
ALNELTESL

SEQ ID NO: 365 - protein, 9 aa, Dengue virus
ALNELPESL

SEQ ID NO: 366 - protein, 9 aa, Dengue virus
IILEFFLIV

SEQ ID NO: 367 - protein, 9 aa, Dengue virus
IILKFFLMV

SEQ ID NO: 368 - protein, 9 aa, Dengue virus
IILEFLLMV

SEQ ID NO: 369 - protein, 9 aa, Dengue virus
IMLEFFLMV

SEQ ID NO: 370 - protein, 9 aa, Dengue virus
IILEFFLMV

SEQ ID NO: 371 - protein, 9 aa, Dengue virus
IILEFFLMV

SEQ ID NO: 372 - protein, 9 aa, Dengue virus
IILEFFLIV

SEQ ID NO: 373 - protein, 9 aa, Dengue virus
IILEFFMMV

SEQ ID NO: 374 - protein, 9 aa, Dengue virus
IVLEFFMMV

SEQ ID NO: 375 - protein, 9 aa, Dengue virus
IILEFFLMV

SEQ ID NO: 376 - protein, 9 aa, Dengue virus
KLAEAIFKL

SEQ ID NO: 377 - protein, 9 aa, Dengue virus
LLAKAIFKL

SEQ ID NO: 378 - protein, 9 aa, Dengue virus
QLAKSIFKL

SEQ ID NO: 379 - protein, 9 aa, Dengue virus
LLATSVFKL

SEQ ID NO: 380 - protein, 9 aa, Dengue virus
LLAKSIFKL

SEQ ID NO: 381 - protein, 9 aa, Dengue virus
LLATAIFKL

SEQ ID NO: 382 - protein, 9 aa, Dengue virus
LLATSIFKL

SEQ ID NO: 383 - protein, 9 aa, Dengue virus
LLASSIFKL

SEQ ID NO: 384 - protein, 9 aa, Dengue virus
KLAEAIFRL

SEQ ID NO: 385 - protein, 9 aa, Dengue virus
RLAEAIFKL

SEQ ID NO: 386 - protein, 9 aa, Dengue virus
KLAEAVFKL

SEQ ID NO: 387 - protein, 9 aa, Dengue virus
KLAEAIFKL

SEQ ID NO: 388 - protein, 9 aa, Dengue virus
QLASAIFKL

SEQ ID NO: 389 - protein, 9 aa, Dengue virus
LLANAIFKL

SEQ ID NO: 390 - protein, 9 aa, Dengue virus
RLANAIFKL

SEQ ID NO: 391 - protein, 9 aa, Dengue virus
QLANAIFKL

SEQ ID NO: 392 - protein, 9 aa, Dengue virus
TLAKAIFKL

SEQ ID NO: 393 - protein, 9 aa, Dengue virus
ILAKAIFKL

SEQ ID NO: 394 - protein, 9 aa, Dengue virus
SQIGAGVYK

SEQ ID NO: 395 - protein, 9 aa, Dengue virus
SQVGVGVFQ

SEQ ID NO: 396 - protein, 9 aa, Dengue virus
SQIGAGVYR

SEQ ID NO: 397 - protein, 9 aa, Dengue virus
SQIGTGVYK

SEQ ID NO: 398 - protein, 9 aa, Dengue virus
SQIGVGVYK

SEQ ID NO: 399 - protein, 9 aa, Dengue virus
SQIGAGVYK

SEQ ID NO: 400 - protein, 9 aa, Dengue virus
TQVGVGIQK

SEQ ID NO: 401 - protein, 9 aa, Dengue virus
TQVGVGVHK

SEQ ID NO: 402 - protein, 9 aa, Dengue virus
TQVGVGVQK

SEQ ID NO: 403 - protein, 9 aa, Dengue virus
TQVGVGIHI

SEQ ID NO: 404 - protein, 9 aa, Dengue virus
TQVGVGIHT

SEQ ID NO: 405 - protein, 9 aa, Dengue virus
TQVGVGIHM

SEQ ID NO: 406 - protein, 9 aa, Dengue virus
TQVGVGVHV

SEQ ID NO: 407 - protein, 10 aa, Dengue virus
GTSGSPIIDK

SEQ ID NO: 408 - protein, 10 aa, Dengue virus
GTSGSPIVSR

SEQ ID NO: 409 - protein, 10 aa, Dengue virus
GTSGSPIVNR

SEQ ID NO: 410 - protein, 10 aa, Dengue virus
GTSGSPIIDK

SEQ ID NO: 411 - protein, 10 aa, Dengue virus
GTSGSPIADK

SEQ ID NO: 412 - protein, 10 aa, Dengue virus
GTSGSPIVDR

SEQ ID NO: 413 - protein, 10 aa, Dengue virus
GTSGSPIVDK

SEQ ID NO: 414 - protein, 10 aa, Dengue virus
GTSGSPIINK

SEQ ID NO: 415 - protein, 10 aa, Dengue virus
GTSGSPIINR

SEQ ID NO: 416 - protein, 10 aa, Dengue virus
GSSGSPIINR

SEQ ID NO: 417 - protein, 10 aa, Dengue virus
GTSGSPIVNR

SEQ ID NO: 418 - protein, 10 aa, Dengue virus
GTSGSPIINK

SEQ ID NO: 419 - protein, 10 aa, Dengue virus
GTSGSPIINR

SEQ ID NO: 420 - protein, 9 aa, Dengue virus
KTFDSEYVK

SEQ ID NO: 421 - protein, 9 aa, Dengue virus
KTFDTEYQK

SEQ ID NO: 422 - protein, 9 aa, Dengue virus
KTFDTEYTK

SEQ ID NO: 423 - protein, 9 aa, Dengue virus
KTFDTEYIK

SEQ ID NO: 424 - protein, 9 aa, Dengue virus
KTFDFEYIK

SEQ ID NO: 425 - protein, 9 aa, Dengue virus
KTFDSEYIK

SEQ ID NO: 426 - protein, 9 aa, Dengue virus
KTFDSEYAK

SEQ ID NO: 427 - protein, 9 aa, Dengue virus
KTFDSEYVK

SEQ ID NO: 428 - protein, 9 aa, Dengue virus
KTFDTEYQR

SEQ ID NO: 429 - protein, 9 aa, Dengue virus
KTFNTEYQK

SEQ ID NO: 430 - protein, 9 aa, Dengue virus
KTFDTEYQK

SEQ ID NO: 431 - protein, 9 aa, Dengue virus
KTFDTEYPK

SEQ ID NO: 432 - protein, 9 aa, Dengue virus
KPRWLDARI

SEQ ID NO: 433 - protein, 9 aa, Dengue virus
RPRWLDART

SEQ ID NO: 434 - protein, 9 aa, Dengue virus
KPRWLDART

SEQ ID NO: 435 - protein, 9 aa, Dengue virus
KPRWLDAKI

SEQ ID NO: 436 - protein, 9 aa, Dengue virus
KPRWLDPRI

SEQ ID NO: 437 - protein, 9 aa, Dengue virus
KPRWLDARI

SEQ ID NO: 438 - protein, 9 aa, Dengue virus
RPRWLDART

SEQ ID NO: 439 - protein, 9 aa, Dengue virus
RPRWLDARI

SEQ ID NO: 440 - protein, 9 aa, Dengue virus
RPRWLDARV

SEQ ID NO: 441 - protein, 9 aa, Dengue virus
RPKWLDARV

SEQ ID NO: 442 - protein, 10 aa, Dengue virus
RPASAWTLYA

SEQ ID NO: 443 - protein, 10 aa, Dengue virus
HPASAWTLYA

SEQ ID NO: 444 - protein, 10 aa, Dengue virus
RPASAWTLYA

SEQ ID NO: 445 - protein, 10 aa, Dengue virus
RPASAWTLYA

SEQ ID NO: 446 - protein, 10 aa, Dengue virus
HPASAWILYA

SEQ ID NO: 447 - protein, 10 aa, Dengue virus
HPASAWTLYA

SEQ ID NO: 448 - protein, 10 aa, Dengue virus
RPASAWTLYA

SEQ ID NO: 449 - protein, 8 aa, Dengue virus
TPMLRHSI

SEQ ID NO: 450 - protein, 8 aa, Dengue virus
TPMLRHTI

SEQ ID NO: 451 - protein, 8 aa, Dengue virus
TPMMRHTI

SEQ ID NO: 452 - protein, 8 aa, Dengue virus
TPMLRHSI

SEQ ID NO: 453 - protein, 8 aa, Dengue virus
TPMLRHTI

SEQ ID NO: 454 - protein, 8 aa, Dengue virus
TPMLRHTI

SEQ ID NO: 455 - protein, 10 aa, Dengue virus
SPNPTVEAGR

SEQ ID NO: 456 - protein, 10 aa, Dengue virus
SPNPTIEEGR

SEQ ID NO: 457 - protein, 10 aa, Dengue virus
SPSPTVEAGR

SEQ ID NO: 458 - protein, 10 aa, Dengue virus
SPNPTVDAGR

SEQ ID NO: 459 - protein, 10 aa, Dengue virus
SPNPTVEAGP

SEQ ID NO: 460 - protein, 10 aa, Dengue virus
SPNPTIEAGR

SEQ ID NO: 461 - protein, 10 aa, Dengue virus
SPNPTVEAGR

SEQ ID NO: 462 - protein, 10 aa, Dengue virus
SPSPTVEEGR

SEQ ID NO: 463 - protein, 10 aa, Dengue virus
SPSLTVEESR

SEQ ID NO: 464 - protein, 10 aa, Dengue virus
SPSPIVEESR

SEQ ID NO: 465 - protein, 10 aa, Dengue virus
SPSPTVEESR

SEQ ID NO: 466 - protein, 10 aa, Dengue virus
SSNPTIEEGR

SEQ ID NO: 467 - protein, 10 aa, Dengue virus
TPRMCTREEF

SEQ ID NO: 468 - protein, 10 aa, Dengue virus
KPRICTREEF

SEQ ID NO: 469 - protein, 10 aa, Dengue virus
RPRICTRAEF

SEQ ID NO: 470 - protein, 10 aa, Dengue virus
KPRICTRAEF

SEQ ID NO: 471 - protein, 10 aa, Dengue virus
TRRMCTREEF

SEQ ID NO: 472 - protein, 10 aa, Dengue virus
TPRICTREEF

SEQ ID NO: 473 - protein, 10 aa, Dengue virus
IPRMCTREEF

SEQ ID NO: 474 - protein, 10 aa, Dengue virus
TPRMCTREEF

SEQ ID NO: 475 - protein, 10 aa, Dengue virus
KPRLCPREEF

SEQ ID NO: 476 - protein, 10 aa, Dengue virus
KPRLCTREEF

SEQ ID NO: 477 - protein, 10 aa, Dengue virus
RPRLCTREEF

SEQ ID NO: 478 - protein, 10 aa, Dengue virus
NPRLCTKEEF

SEQ ID NO: 479 - protein, 10 aa, Dengue virus
SPRLCTREEF

SEQ ID NO: 480 - protein, 10 aa, Dengue virus
TPRLCTREEF

SEQ ID NO: 481 - protein, 10 aa, Dengue virus
SPRLCTKEEF

SEQ ID NO: 482 - protein, 10 aa, Dengue virus
NPRLCTREEF

SEQ ID NO: 483 - protein, 10 aa, Dengue virus
KPRLCTREEF

SEQ ID NO: 484 - protein, 9 aa, Dengue virus
RPTPRGTVM

SEQ ID NO: 485 - protein, 9 aa, Dengue virus
RPVKNGTVM

SEQ ID NO: 486 - protein, 9 aa, Dengue virus
RPARNGTVM

SEQ ID NO: 487 - protein, 9 aa, Dengue virus
RPAKNGTVM

SEQ ID NO: 488 - protein, 9 aa, Dengue virus
RPAKSGTVM

SEQ ID NO: 489 - protein, 9 aa, Dengue virus
RPTPRGTVL

SEQ ID NO: 490 - protein, 9 aa, Dengue virus
RPTPKGTVM

SEQ ID NO: 491 - protein, 9 aa, Dengue virus
RPTPIGTVM

SEQ ID NO: 492 - protein, 9 aa, Dengue virus
RPTPRGTVM

SEQ ID NO: 493 - protein, 9 aa, Dengue virus
RPTPKGTVM

SEQ ID NO: 494 - protein, 9 aa, Dengue virus
RPTPTGTVM

SEQ ID NO: 495 - protein, 9 aa, Dengue virus
RPTPRGAVM

SEQ ID NO: 496 - protein, 9 aa, Dengue virus
RPTPKGAVM

SEQ ID NO: 497 - protein, 15 aa, Dengue virus
AFLRFLTIPPTAGIL

SEQ ID NO: 498 - protein, 15 aa, Dengue virus
AFLRFLAIPPTAGIV

SEQ ID NO: 499 - protein, 15 aa, Dengue virus
ALLRFLAIPPTAGIL

SEQ ID NO: 500 - protein, 15 aa, Dengue virus
AFLTFLAIPPTAGIL

SEQ ID NO: 501 - protein, 15 aa, Dengue virus
AFLRFLAIPPTAGIL

SEQ ID NO: 502 - protein, 15 aa, Dengue virus
AFLRFLTISPTAGIL

SEQ ID NO: 503 - protein, 15 aa, Dengue virus
AFLRFLTIPPTVGIL

SEQ ID NO: 504 - protein, 15 aa, Dengue virus
AFLRFLTIPPTAGIL

SEQ ID NO: 505 - protein, 15 aa, Dengue virus
AFLRFLAIPPTAGIL

SEQ ID NO: 506 - protein, 15 aa, Dengue virus
AFLRFLAIPPTAGVL

SEQ ID NO: 507 - protein, 15 aa, Dengue virus
TFLRVLSIPPTAGIL

SEQ ID NO: 508 - protein, 15 aa, Dengue virus
GVTYLALLAAFKVRP

SEQ ID NO: 509 - protein, 15 aa, Dengue virus
GTTYLALMATFRMRP

SEQ ID NO: 510 - protein, 15 aa, Dengue virus
GMTYLALMATFKMRP

SEQ ID NO: 511 - protein, 15 aa, Dengue virus
GTTYLALMATLKMRP

SEQ ID NO: 512 - protein, 15 aa, Dengue virus
GTTHLALMATFKMRP

SEQ ID NO: 513 - protein, 15 aa, Dengue virus
GTTYLALMATFKMRP

SEQ ID NO: 514 - protein, 15 aa, Dengue virus
GVTYLALLATFKVRP

SEQ ID NO: 515 - protein, 15 aa, Dengue virus
GVTYLALLAAYKVRP

SEQ ID NO: 516 - protein, 15 aa, Dengue virus
GVTYLALLAAFRVRP

SEQ ID NO: 517 - protein, 15 aa, Dengue virus
GVTYLALLAAFKVRP

SEQ ID NO: 518 - protein, 15 aa, Dengue virus
GVTYLALIATFEIQP

SEQ ID NO: 519 - protein, 15 aa, Dengue virus
GVTCLALIATFKIQP

SEQ ID NO: 520 - protein, 15 aa, Dengue virus
GVTYLALIATFKVQP

SEQ ID NO: 521 - protein, 15 aa, Dengue virus
GVTYLALIATFKIQP

SEQ ID NO: 522 - protein, 15 aa, Dengue virus
GQTHLAIMAVFKMSP

SEQ ID NO: 523 - protein, 15 aa, Dengue virus
GQIHLAIMAVFKMSP

SEQ ID NO: 524 - protein, 15 aa, Dengue virus
GQTHLAIMIVFKMSP

SEQ ID NO: 525 - protein, 15 aa, Dengue virus
GQVHLAIMAVFKMSP

SEQ ID NO: 526 - protein, 15 aa, Dengue virus
GQIHLAIMTMFKMSP

SEQ ID NO: 527 - protein, 15 aa, Dengue virus
MAVGMVSILASSLLK

SEQ ID NO: 528 - protein, 15 aa, Dengue virus
MAVGVVSILLSSLLK

SEQ ID NO: 529 - protein, 15 aa, Dengue virus
MAIGIVSILLSSLLK

SEQ ID NO: 530 - protein, 15 aa, Dengue virus
MAVGIVSILLSSLLK

SEQ ID NO: 531 - protein, 15 aa, Dengue virus
MAVGMVSILASSLLK

SEQ ID NO: 532 - protein, 15 aa, Dengue virus
MAVGLVSILASSFLR

SEQ ID NO: 533 - protein, 15 aa, Dengue virus
MAIGLVSILASSLLR

SEQ ID NO: 534 - protein, 15 aa, Dengue virus
MAVGLVSILASSLLR

SEQ ID NO: 535 - protein, 15 aa, Dengue virus
MAVGLVSLLGSALLK

SEQ ID NO: 536 - protein, 15 aa, Dengue virus
TFTMRLLSPVRVPNY

SEQ ID NO: 537 - protein, 15 aa, Dengue virus
TFTMRLLSPVRVPNY

SEQ ID NO: 538 - protein, 15 aa, Dengue virus
PFTMRLLSPVRVPNY

SEQ ID NO: 539 - protein, 15 aa, Dengue virus
TFTMRLLSPIRVPNY

SEQ ID NO: 540 - protein, 15 aa, Dengue virus
TFTMRLLSPVRVPNY

SEQ ID NO: 541 - protein, 15 aa, Dengue virus
TFTMRLLSPVRVSNY

SEQ ID NO: 542 - protein, 15 aa, Dengue virus
PFTMRLLSPVRVPNY

SEQ ID NO: 543 - protein, 15 aa, Dengue virus
TFTMRLLSPVRVPNY

SEQ ID NO: 544 - protein, 15 aa, Dengue virus
TFTTKLLSSTRVPNY

SEQ ID NO: 545 - protein, 15 aa, Dengue virus
TFTTRLLSSTRVPNY

SEQ ID NO: 546 - protein, 15 aa, Dengue virus
SRAIWYMWLGARFLE

SEQ ID NO: 547 - protein, 15 aa, Dengue virus
SRAIWYVWLGARFLE

SEQ ID NO: 548 - protein, 15 aa, Dengue virus
SRAIWYMWLGAAFLE

SEQ ID NO: 549 - protein, 15 aa, Dengue virus
SRAIWYMWLGARFLE

SEQ ID NO: 550 - protein, 15 aa, Dengue virus
SRAIWYMWLGARFLE

SEQ ID NO: 551 - protein, 15 aa, Dengue virus
SRAIWYMWLGARFLE

SEQ ID NO: 552 - protein, 15 aa, Dengue virus
SRAIWYMWLGVRYLE

SEQ ID NO: 553 - protein, 15 aa, Dengue virus
SRAIWYMWLGARYLE

SEQ ID NO: 554 - protein, 15 aa, Dengue virus
SRAIWYMWLGARFLE

SEQ ID NO: 555 - protein, 9 aa, Dengue virus
GPMKLVMAF

SEQ ID NO: 556 - protein, 10 aa, Dengue virus
GPMKLVMAFI

SEQ ID NO: 557 - protein, 9 aa, Dengue virus
MAFIAFLRF

SEQ ID NO: 558 - protein, 9 aa, Dengue virus
KSGAIKVLK

SEQ ID NO: 559 - protein, 10 aa, Dengue virus
ITLLCLIPTV

SEQ ID NO: 560 - protein, 10 aa, Dengue virus
CLMMMLPATL

SEQ ID NO: 561 - protein, 9 aa, Dengue virus
TLLCLIPTV

SEQ ID NO: 562 - protein, 9 aa, Dengue virus
LMMMLPATL

SEQ ID NO: 563 - protein, 10 aa, Dengue virus
LMMMLPATLA

SEQ ID NO: 564 - protein, 9 aa, Dengue virus
MMMLPATLA

SEQ ID NO: 565 - protein, 10 aa, Dengue virus
MMMLPATLAF

SEQ ID NO: 566 - protein, 9 aa, Dengue virus
MLIPTAMAF

SEQ ID NO: 567 - protein, 10 aa, Dengue virus
TLMAMDLGEL

SEQ ID NO: 568 - protein, 9 aa, Dengue virus
VTYECPLLV

SEQ ID NO: 569 - protein, 10 aa, Dengue virus
HPGFTILALF

SEQ ID NO: 570 - protein, 10 aa, Dengue virus
FTIMAAILAY

SEQ ID NO: 571 - protein, 10 aa, Dengue virus
FTILALFLAH

SEQ ID NO: 572 - protein, 9 aa, Dengue virus
TIMAAILAY

SEQ ID NO: 573 - protein, 9 aa, Dengue virus
MLVTPSMTM

SEQ ID NO: 574 - protein, 9 aa, Dengue virus
CPTQGEATL

SEQ ID NO: 575 - protein, 9 aa, Dengue virus
CPTQGEAVL

SEQ ID NO: 576 - protein, 9 aa, Dengue virus
LPEEQDQNY

SEQ ID NO: 577 - protein, 9 aa, Dengue virus
YENLKYSVI

SEQ ID NO: 578 - protein, 9 aa, Dengue virus
QEGAMHSAL

SEQ ID NO: 579 - protein, 9 aa, Dengue virus
QEGAMHTAL

SEQ ID NO: 580 - protein, 9 aa, Dengue virus
MSYTMCSGK

SEQ ID NO: 581 - protein, 10 aa, Dengue virus
MSYSMCTGKF

SEQ ID NO: 582 - protein, 10 aa, Dengue virus
SPCKIPFEIM

SEQ ID NO: 583 - protein, 10 aa, Dengue virus
IPFEIMDLEK

SEQ ID NO: 584 - protein, 10 aa, Dengue virus
EPGQLKLNWF

SEQ ID NO: 585 - protein, 9 aa, Dengue virus
FGAIYGAAF

SEQ ID NO: 586 - protein, 10 aa, Dengue virus
SWMVRILIGF

SEQ ID NO: 587 - protein, 9 aa, Dengue virus
IGIGILLTW

SEQ ID NO: 588 - protein, 10 aa, Dengue virus
SPKRLATAIA

SEQ ID NO: 589 - protein, 9 aa, Dengue virus
KQIANELNY

SEQ ID NO: 590 - protein, 10 aa, Dengue virus
VYTQLCDHRL

SEQ ID NO: 591 - protein, 9 aa, Dengue virus
VYTQLCDHR

SEQ ID NO: 592 - protein, 10 aa, Dengue virus
KAVHADMGYW

SEQ ID NO: 593 - protein, 10 aa, Dengue virus
RASFIEVKTC

SEQ ID NO: 594 - protein, 10 aa, Dengue virus
FAGPVSQHNY

SEQ ID NO: 595 - protein, 9 aa, Dengue virus
RPGYHTQTA

SEQ ID NO: 596 - protein, 10 aa, Dengue virus
GPWHLGKLEL

SEQ ID NO: 597 - protein, 10 aa, Dengue virus
GPWHLGKLEM

SEQ ID NO: 598 - protein, 10 aa, Dengue virus
RYMGEDGCWY

SEQ ID NO: 599 - protein, 10 aa, Dengue virus
FTMGVLCLAI

SEQ ID NO: 600 - protein, 10 aa, Dengue virus
MSFRDLGRVM

SEQ ID NO: 601 - protein, 9 aa, Dengue virus
TYLALIATF

SEQ ID NO: 602 - protein, 9 aa, Dengue virus
IQPFLALGF

SEQ ID NO: 603 - protein, 10 aa, Dengue virus
GFFLRKLTSR

SEQ ID NO: 604 - protein, 9 aa, Dengue virus
MMATIGIAL

SEQ ID NO: 605 - protein, 9 aa, Dengue virus
MALSIVSLF

SEQ ID NO: 606 - protein, 9 aa, Dengue virus
MAVGMVSIL

SEQ ID NO: 607 - protein, 10 aa, Dengue virus
IPMTGPLVAG

SEQ ID NO: 608 - protein, 9 aa, Dengue virus
GPLVAGGLL

SEQ ID NO: 609 - protein, 10 aa, Dengue virus
SPILSITISE

SEQ ID NO: 610 - protein, 10 aa, Dengue virus
FPVSIPITAA

SEQ ID NO: 611 - protein, 9 aa, Dengue virus
IPITAAAWY

SEQ ID NO: 612 - protein, 9 aa, Dengue virus
MEGVFHTMW

SEQ ID NO: 613 - protein, 10 aa, Dengue virus
MEGVFHTMWH

SEQ ID NO: 614 - protein, 9 aa, Dengue virus
KPGTSGSPI

SEQ ID NO: 615 - protein, 10 aa, Dengue virus
KPGTSGSPII

SEQ ID NO: 616 - protein, 10 aa, Dengue virus
GTSGSPIIDK

SEQ ID NO: 617 - protein, 10 aa, Dengue virus
SPIINREGKV

SEQ ID NO: 618 - protein, 9 aa, Dengue virus
NPEIEDDIF

SEQ ID NO: 619 - protein, 10 aa, Dengue virus
HPGAGKTKRY

SEQ ID NO: 620 - protein, 9 aa, Dengue virus
LPAIVREAI

SEQ ID NO: 621 - protein, 10 aa, Dengue virus
APTRVVAAEM

SEQ ID NO: 622 - protein, 10 aa, Dengue virus
APTRVVASEM

SEQ ID NO: 623 - protein, 10 aa, Dengue virus
APTRVVAAEM

SEQ ID NO: 624 - protein, 10 aa, Dengue virus
SEMAEALKGM

SEQ ID NO: 625 - protein, 9 aa, Dengue virus
LPIRYQTPA

SEQ ID NO: 626 - protein, 10 aa, Dengue virus
LPIRYQTPAI

SEQ ID NO: 627 - protein, 10 aa, Dengue virus
DPASIAARGY

SEQ ID NO: 628 - protein, 10 aa, Dengue virus
PASIAARGYI

SEQ ID NO: 629 - protein, 9 aa, Dengue virus
TPPGSRDPF

SEQ ID NO: 630 - protein, 10 aa, Dengue virus
FPQSNAPIMD

SEQ ID NO: 631 - protein, 9 aa, Dengue virus
FPQSNAPIM

SEQ ID NO: 632 - protein, 10 aa, Dengue virus
EERDIPERSW

SEQ ID NO: 633 - protein, 10 aa, Dengue virus
REIPERSWNT

SEQ ID NO: 634 - protein, 10 aa, Dengue virus
YPKTKLTDWD

SEQ ID NO: 635 - protein, 10 aa, Dengue virus
RVIDPRRCMK

SEQ ID NO: 636 - protein, 10 aa, Dengue virus
RVIDPRRCLK

SEQ ID NO: 637 - protein, 10 aa, Dengue virus
RVIDPRRCMK

SEQ ID NO: 638 - protein, 9 aa, Dengue virus
RVIDPRRCL

SEQ ID NO: 639 - protein, 10 aa, Dengue virus
RVIDPRRCMK

SEQ ID NO: 640 - protein, 9 aa, Dengue virus
DPRRCLKPV

SEQ ID NO: 641 - protein, 9 aa, Dengue virus
MPVTHSSAA

SEQ ID NO: 642 - protein, 10 aa, Dengue virus
MPVTHSSAAQ

SEQ ID NO: 643 - protein, 9 aa, Dengue virus
NPAQEDDQY

SEQ ID NO: 644 - protein, 9 aa, Dengue virus
QYIFTGQPL

SEQ ID NO: 645 - protein, 9 aa, Dengue virus
TPEGIIPSM

SEQ ID NO: 646 - protein, 10 aa, Dengue virus
TPEGIIPSMF

SEQ ID NO: 647 - protein, 9 aa, Dengue virus
TPEGIIPAL

SEQ ID NO: 648 - protein, 10 aa, Dengue virus
TPEGIIPALF

SEQ ID NO: 649 - protein, 9 aa, Dengue virus
TPEGIIPSM

SEQ ID NO: 650 - protein, 10 aa, Dengue virus
TPEGIIPSMF

SEQ ID NO: 651 - protein, 10 aa, Dengue virus
TPEGIIPTLF

SEQ ID NO: 652 - protein, 10 aa, Dengue virus
TPEGIIPALF

SEQ ID NO: 653 - protein, 10 aa, Dengue virus
GEFRLRGEQR

SEQ ID NO: 654 - protein, 10 aa, Dengue virus
GEARKTFVEL

SEQ ID NO: 655 - protein, 10 aa, Dengue virus
GEARKTFVDL

SEQ ID NO: 656 - protein, 10 aa, Dengue virus
GESRKTFVEL

SEQ ID NO: 657 - protein, 10 aa, Dengue virus
GEQRKTFVEL

SEQ ID NO: 658 - protein, 10 aa, Dengue virus
ELMRRGDLPV

SEQ ID NO: 659 - protein, 10 aa, Dengue virus
LPVWLAYKVA

SEQ ID NO: 660 - protein, 10 aa, Dengue virus
YKVASAGISY

SEQ ID NO: 661 - protein, 10 aa, Dengue virus
REWCFTGERN

SEQ ID NO: 662 - protein, 9 aa, Dengue virus
RPRWLDART

SEQ ID NO: 663 - protein, 9 aa, Dengue virus
MALKDFKEF

SEQ ID NO: 664 - protein, 9 aa, Dengue virus
EFKEFAAGR

SEQ ID NO: 665 - protein, 10 aa, Dengue virus
FASGRKSITL

SEQ ID NO: 666 - protein, 10 aa, Dengue virus
LPTFMTQKAR

SEQ ID NO: 667 - protein, 9 aa, Dengue virus
MTQKARNAL

SEQ ID NO: 668 - protein, 9 aa, Dengue virus
TAEAGGRAY

SEQ ID NO: 669 - protein, 10 aa, Dengue virus
LPETLETLLL

SEQ ID NO: 670 - protein, 9 aa, Dengue virus
LETLMLVAL

SEQ ID NO: 671 - protein, 10 aa, Dengue virus
LETLMLVALL

SEQ ID NO: 672 - protein, 10 aa, Dengue virus
TLMLLALIAV

SEQ ID NO: 673 - protein, 10 aa, Dengue virus
LMLLALIAVL

SEQ ID NO: 674 - protein, 9 aa, Dengue virus
MLLALIAVL

SEQ ID NO: 675 - protein, 10 aa, Dengue virus
GAMLFLISGK

SEQ ID NO: 676 - protein, 10 aa, Dengue virus
SIILEFFLMV

SEQ ID NO: 677 - protein, 9 aa, Dengue virus
IILEFFLMV

SEQ ID NO: 678 - protein, 10 aa, Dengue virus
IILEFFLMVL

SEQ ID NO: 679 - protein, 10 aa, Dengue virus
FLMVLLIPEP

SEQ ID NO: 680 - protein, 10 aa, Dengue virus
TPQDNQLAYV

SEQ ID NO: 681 - protein, 9 aa, Dengue virus
TPQDNQLTY

SEQ ID NO: 682 - protein, 10 aa, Dengue virus
TTKRDLGMSK

SEQ ID NO: 683 - protein, 10 aa, Dengue virus
TETTILDVDL

SEQ ID NO: 684 - protein, 9 aa, Dengue virus
RPASAWTLY

SEQ ID NO: 685 - protein, 10 aa, Dengue virus
RPASAWTLYA

SEQ ID NO: 686 - protein, 9 aa, Dengue virus
HPASAWTLY

SEQ ID NO: 687 - protein, 10 aa, Dengue virus
PASAWTLYAV

SEQ ID NO: 688 - protein, 9 aa, Dengue virus
VATTFVTPM

SEQ ID NO: 689 - protein, 9 aa, Dengue virus
ITPMLRHTI

SEQ ID NO: 690 - protein, 10 aa, Dengue virus
TPMLRHTIEN

SEQ ID NO: 691 - protein, 9 aa, Dengue virus
IANQATVLM

SEQ ID NO: 692 - protein, 9 aa, Dengue virus
VPLLAIGCY

SEQ ID NO: 693 - protein, 9 aa, Dengue virus
NPLTLTAAV

SEQ ID NO: 694 - protein, 10 aa, Dengue virus
TLTAAVLLLV

SEQ ID NO: 695 - protein, 10 aa, Dengue virus
AAVLLLVTHY

SEQ ID NO: 696 - protein, 10 aa, Dengue virus
VLLLVTHYAI

SEQ ID NO: 697 - protein, 9 aa, Dengue virus
DPIPYDPKF

SEQ ID NO: 698 - protein, 10 aa, Dengue virus
MLLILCVTQV

SEQ ID NO: 699 - protein, 9 aa, Dengue virus
ATGPLTTLW

SEQ ID NO: 700 - protein, 9 aa, Dengue virus
ATGPISTLW

SEQ ID NO: 701 - protein, 9 aa, Dengue virus
ATGPITTLW

SEQ ID NO: 702 - protein, 9 aa, Dengue virus
ATGPILTLW

SEQ ID NO: 703 - protein, 9 aa, Dengue virus
ATGPVLTLW

SEQ ID NO: 704 - protein, 9 aa, Dengue virus
LWEGSPGKF

SEQ ID NO: 705 - protein, 10 aa, Dengue virus
SPGKFWNTTI

SEQ ID NO: 706 - protein, 9 aa, Dengue virus
IAVSMANIF

SEQ ID NO: 707 - protein, 9 aa, Dengue virus
IAVSMANIF

SEQ ID NO: 708 - protein, 9 aa, Dengue virus
IAVSTANIF

SEQ ID NO: 709 - protein, 9 aa, Dengue virus
MANIFRGSY

SEQ ID NO: 710 - protein, 9 aa, Dengue virus
YLAGAGLAF

SEQ ID NO: 711 - protein, 10 aa, Dengue virus
GSSKIRWIVE

SEQ ID NO: 712 - protein, 10 aa, Dengue virus
GPGHEEPIPM

SEQ ID NO: 713 - protein, 10 aa, Dengue virus
IPMSTYGWNL

SEQ ID NO: 714 - protein, 10 aa, Dengue virus
IPMATYGWNL

SEQ ID NO: 715 - protein, 10 aa, Dengue virus
IPMSTYGWNL

SEQ ID NO: 716 - protein, 10 aa, Dengue virus
MSTYGWNIVK

SEQ ID NO: 717 - protein, 9 aa, Dengue virus
STYGWNIVK

SEQ ID NO: 718 - protein, 10 aa, Dengue virus
QSGVDVFFTP

SEQ ID NO: 719 - protein, 9 aa, Dengue virus
RVLKMVEPW

SEQ ID NO: 720 - protein, 10 aa, Dengue virus
KVLNPYMPSV

SEQ ID NO: 721 - protein, 9 aa, Dengue virus
VLNPYMPSV

SEQ ID NO: 722 - protein, 10 aa, Dengue virus
MPSVIEKMET

SEQ ID NO: 723 - protein, 10 aa, Dengue virus
VSSVNMVSRL

SEQ ID NO: 724 - protein, 9 aa, Dengue virus
MVSRLLLNR

SEQ ID NO: 725 - protein, 10 aa, Dengue virus
FTMRHKKATY

SEQ ID NO: 726 - protein, 9 aa, Dengue virus
WHYDQDHPY

SEQ ID NO: 727 - protein, 10 aa, Dengue virus
QENPYRTWAY

SEQ ID NO: 728 - protein, 9 aa, Dengue virus
WAYHGSYET

SEQ ID NO: 729 - protein, 9 aa, Dengue virus
WAYHGSYEV

SEQ ID NO: 730 - protein, 9 aa, Dengue virus
DTTPFGQQR

SEQ ID NO: 731 - protein, 9 aa, Dengue virus
TPFGQQRVF

SEQ ID NO: 732 - protein, 10 aa, Dengue virus
EPKEGTKKLM

SEQ ID NO: 733 - protein, 9 aa, Dengue virus
MEITAEWLW

SEQ ID NO: 734 - protein, 10 aa, Dengue virus
KPRICTREEF

SEQ ID NO: 735 - protein, 10 aa, Dengue virus
TPRMCTREEF

SEQ ID NO: 736 - protein, 10 aa, Dengue virus
KPRLCTREEF

SEQ ID NO: 737 - protein, 10 aa, Dengue virus
NPRLCTREEF

SEQ ID NO: 738 - protein, 10 aa, Dengue virus
RPRLCTREEF

SEQ ID NO: 739 - protein, 10 aa, Dengue virus
TPRMCTREEF

SEQ ID NO: 740 - protein, 9 aa, Dengue virus
RAAVEDEEF

SEQ ID NO: 741 - protein, 10 aa, Dengue virus
EAVEDSRFWE

SEQ ID NO: 742 - protein, 10 aa, Dengue virus
KGSRAIWYMW

SEQ ID NO: 743 - protein, 10 aa, Dengue virus
RYLEFEALGF

SEQ ID NO: 744 - protein, 10 aa, Dengue virus
RFLEFEALGF

SEQ ID NO: 745 - protein, 10 aa, Dengue virus
FSRENSLSGV

SEQ ID NO: 746 - protein, 9 aa, Dengue virus
GEGLHKLGY

SEQ ID NO: 747 - protein, 9 aa, Dengue virus
RQLANAIFK

SEQ ID NO: 748 - protein, 10 aa, Dengue virus
TPRGTVMDII

SEQ ID NO: 749 - protein, 10 aa, Dengue virus
TPKGAVMDII

SEQ ID NO: 750 - protein, 9 aa, Dengue virus
RQMEGEGIF

SEQ ID NO: 751 - protein, 9 aa, Dengue virus
RQMEGEGVL

SEQ ID NO: 752 - protein, 9 aa, Dengue virus
KVRKDIQQW

SEQ ID NO: 753 - protein, 9 aa, Dengue virus
YAQMWSLMY

SEQ ID NO: 754 - protein, 10 aa, Dengue virus
YAQMWSLMYF

SEQ ID NO: 755 - protein, 9 aa, Dengue virus
ICSAVPVHW

SEQ ID NO: 756 - protein, 9 aa, Dengue virus
WSIHAHHQW

SEQ ID NO: 757 - protein, 10 aa, Dengue virus
NPNMIDKTPV

SEQ ID NO: 758 - protein, 10 aa, Dengue virus
NPWMEDKTPV

SEQ ID NO: 759 - protein, 10 aa, Dengue virus
VPYLGKREDQ

SEQ ID NO: 760 - protein, 9 aa, Dengue virus
REDLWCGSL

SEQ ID NO: 761 - protein, 9 aa, Dengue virus
REDQWCGSL

SEQ ID NO: 762 - protein, 10 aa, Dengue virus
MPSMKRFRRE

SEQ ID NO: 763 - protein, 9 aa, Dengue virus
APFESEGVL

SEQ ID NO: 764 - protein, 9 aa, Dengue virus
TPEGIIPAL

SEQ ID NO: 765 - protein, 10 aa, Dengue virus
TPEGIIPALF

SEQ ID NO: 766 - protein, 9 aa, Dengue virus
TPEGIIPSM

SEQ ID NO: 767 - protein, 10 aa, Dengue virus
TPEGIIPSMF

SEQ ID NO: 768 - protein, 10 aa, Dengue virus
TPEGIIPTLF

SEQ ID NO: 769 - protein, 10 aa, Dengue virus
YTPEGIIPTL

SEQ ID NO: 770 - protein, 10 aa, Dengue virus
GEARKTFVDL

SEQ ID NO: 771 - protein, 10 aa, Dengue virus
GEARKTFVEL

SEQ ID NO: 772 - protein, 10 aa, Dengue virus
GEQRKTFVEL

SEQ ID NO: 773 - protein, 10 aa, Dengue virus
GESRKTFVEL

SEQ ID NO: 774 - protein, 10 aa, Dengue virus
LPVWLAHKVA

SEQ ID NO: 775 - protein, 9 aa, Dengue virus
LPVWLAYKV

SEQ ID NO: 776 - protein, 10 aa, Dengue virus
LPVWLAYKVA

SEQ ID NO: 777 - protein, 10 aa, Dengue virus
LPVWLAYRVA

SEQ ID NO: 778 - protein, 9 aa, Dengue virus
LPVWLSYKV

SEQ ID NO: 779 - protein, 10 aa, Dengue virus
HPGAGKTKRY

SEQ ID NO: 780 - protein, 9 aa, Dengue virus
NPEIEDDIF

SEQ ID NO: 781 - protein, 9 aa, Dengue virus
DTTPFGQQR

SEQ ID NO: 782 - protein, 10 aa, Dengue virus
MSFRDLGRVM

SEQ ID NO: 783 - protein, 9 aa, Dengue virus
ATGPILTLW

SEQ ID NO: 784 - protein, 9 aa, Dengue virus
ATGPISTLW

SEQ ID NO: 785 - protein, 9 aa, Dengue virus
ATGPITTLW

SEQ ID NO: 786 - protein, 9 aa, Dengue virus
ATGPLTTLW

SEQ ID NO: 787 - protein, 9 aa, Dengue virus
ATGPVLTLW

SEQ ID NO: 788 - protein, 10 aa, Dengue virus
LATGPVLTLW

SEQ ID NO: 789 - protein, 9 aa, Dengue virus
CLIPTAMAF

SEQ ID NO: 790 - protein, 9 aa, Dengue virus
MLIPTAMAF

SEQ ID NO: 791 - protein, 9 aa, Dengue virus
VATTFVTPM

SEQ ID NO: 792 - protein, 10 aa, Dengue virus
KPRICTREEF

SEQ ID NO: 793 - protein, 10 aa, Dengue virus
KPRLCTREEF

SEQ ID NO: 794 - protein, 10 aa, Dengue virus
NPRLCTREEF

SEQ ID NO: 795 - protein, 10 aa, Dengue virus
RPRLCTREEF

SEQ ID NO: 796 - protein, 10 aa, Dengue virus
TPRMCTREEF

SEQ ID NO: 797 - protein, 9 aa, Dengue virus
VPLLAIGCY

SEQ ID NO: 798 - protein, 9 aa, Dengue virus
VPLLAMGCY

SEQ ID NO: 799 - protein, 10 aa, Dengue virus
MSYSMCTGKF

SEQ ID NO: 800 - protein, 10 aa, Dengue virus
DPASIAARGY

SEQ ID NO: 801 - protein, 9 aa, Dengue virus
IANQATVLM

SEQ ID NO: 802 - protein, 10 aa, Dengue virus
APTRVVAAEM

SEQ ID NO: 803 - protein, 10 aa, Dengue virus
APTRVVASEM

SEQ ID NO: 804 - protein, 10 aa, Dengue virus
TPMLRHTIEN

SEQ ID NO: 805 - protein, 9 aa, Dengue virus
MLVTPSMTM

SEQ ID NO: 806 - protein, 10 aa, Dengue virus
FTMRHKKATY

SEQ ID NO: 807 - protein, 10 aa, Dengue virus
FTILALFLAH

SEQ ID NO: 808 - protein, 9 aa, Dengue virus
WHYDQDHPY

SEQ ID NO: 809 - protein, 9 aa, Dengue virus
MALKDFKEF

SEQ ID NO: 810 - protein, 10 aa, Dengue virus
LMKITAEWLW

SEQ ID NO: 811 - protein, 9 aa, Dengue virus
MEITAEWLW

SEQ ID NO: 812 - protein, 10 aa, Dengue virus
VMGITAEWLW

SEQ ID NO: 813 - protein, 10 aa, Dengue virus
AAVLLLVTHY

SEQ ID NO: 814 - protein, 9 aa, Dengue virus
ALRGLPIRY

SEQ ID NO: 815 - protein, 10 aa, Dengue virus
AMALSIVSLF

SEQ ID NO: 816 - protein, 9 aa, Dengue virus
MALSIVSLF

SEQ ID NO: 817 - protein, 9 aa, Dengue virus
APFESEGVL

SEQ ID NO: 818 - protein, 10 aa, Dengue virus
APIMDEEREI

SEQ ID NO: 819 - protein, 10 aa, Dengue virus
APTRVVAAEM

SEQ ID NO: 820 - protein, 10 aa, Dengue virus
APTRVVASEM

SEQ ID NO: 821 - protein, 10 aa, Dengue virus
ASSMVNGVVK

SEQ ID NO: 822 - protein, 10 aa, Dengue virus
ASSMVNGVVR

SEQ ID NO: 823 - protein, 10 aa, Dengue virus
ASSVLLWMAS

SEQ ID NO: 824 - protein, 9 aa, Dengue virus
ATGPILTLW

SEQ ID NO: 825 - protein, 9 aa, Dengue virus
ATGPISTLW

SEQ ID NO: 826 - protein, 9 aa, Dengue virus
ATGPITTLW

SEQ ID NO: 827 - protein, 9 aa, Dengue virus
ATGPLTTLW

SEQ ID NO: 828 - protein, 9 aa, Dengue virus
ATGPVLTLW

SEQ ID NO: 829 - protein, 10 aa, Dengue virus
LATGPVLTLW

SEQ ID NO: 830 - protein, 9 aa, Dengue virus
ATYGWNLVK

SEQ ID NO: 831 - protein, 10 aa, Dengue virus
MATYGWNLVK

SEQ ID NO: 832 - protein, 10 aa, Dengue virus
MSTYGWNIVK

SEQ ID NO: 833 - protein, 10 aa, Dengue virus
AVQTKPGLFK

SEQ ID NO: 834 - protein, 9 aa, Dengue virus
CLIPTAMAF

SEQ ID NO: 835 - protein, 9 aa, Dengue virus
MLIPTAMAF

SEQ ID NO: 836 - protein, 10 aa, Dengue virus
CLMMMLPATL

SEQ ID NO: 837 - protein, 9 aa, Dengue virus
CPTQGEATL

SEQ ID NO: 838 - protein, 9 aa, Dengue virus
CPTQGEAVL

SEQ ID NO: 839 - protein, 9 aa, Dengue virus
CPTQGEPSL

SEQ ID NO: 840 - protein, 10 aa, Dengue virus
DPASIAARGY

SEQ ID NO: 841 - protein, 9 aa, Dengue virus
DPIPYDPKF

SEQ ID NO: 842 - protein, 9 aa, Dengue virus
DPRRCLKPV

SEQ ID NO: 843 - protein, 9 aa, Dengue virus
DTTPFGQQR

SEQ ID NO: 844 - protein, 10 aa, Dengue virus
DYMPSMKRFR

SEQ ID NO: 845 - protein, 10 aa, Dengue virus
EAVEDSRFWE

SEQ ID NO: 846 - protein, 9 aa, Dengue virus
EENMDVEIW

SEQ ID NO: 847 - protein, 10 aa, Dengue virus
EERDIPERSW

SEQ ID NO: 848 - protein, 9 aa, Dengue virus
EFKEFAAGR

SEQ ID NO: 849 - protein, 10 aa, Dengue virus
EFKEFAAGRR

SEQ ID NO: 850 - protein, 10 aa, Dengue virus
ELMRRGDLPV

SEQ ID NO: 851 - protein, 10 aa, Dengue virus
EPDYEVDEDI

SEQ ID NO: 852 - protein, 10 aa, Dengue virus
EPGQLKLNWF

SEQ ID NO: 853 - protein, 10 aa, Dengue virus
EPGVVSPTSY

SEQ ID NO: 854 - protein, 10 aa, Dengue virus
EPIEGKVVQY

SEQ ID NO: 855 - protein, 9 aa, Dengue virus
EPISYDPKF

SEQ ID NO: 856 - protein, 10 aa, Dengue virus
EPKEGTKKLM

SEQ ID NO: 857 - protein, 10 aa, Dengue virus
ESSSNPTIEE

SEQ ID NO: 858 - protein, 9 aa, Dengue virus
ETACLGKAY

SEQ ID NO: 859 - protein, 10 aa, Dengue virus
ETACLGKAYA

SEQ ID NO: 860 - protein, 9 aa, Dengue virus
ETACLGKSY

SEQ ID NO: 861 - protein, 10 aa, Dengue virus
KETACLGKSY

SEQ ID NO: 862 - protein, 10 aa, Dengue virus
RETACLGKAY

SEQ ID NO: 863 - protein, 10 aa, Dengue virus
RETACLGKSY

SEQ ID NO: 864 - protein, 9 aa, Dengue virus
ETTHHAVSR

SEQ ID NO: 865 - protein, 9 aa, Dengue virus
ETTKHAVSR

SEQ ID NO: 866 - protein, 10 aa, Dengue virus
FAGPVSQHNY

SEQ ID NO: 867 - protein, 10 aa, Dengue virus
FASGRKSITL

SEQ ID NO: 868 - protein, 9 aa, Dengue virus
FGAIYGAAF

SEQ ID NO: 869 - protein, 10 aa, Dengue virus
FLMVLLIPEP

SEQ ID NO: 870 - protein, 10 aa, Dengue virus
FMALVAFLRF

SEQ ID NO: 871 - protein, 10 aa, Dengue virus
FMKDGRSLVV

SEQ ID NO: 872 - protein, 10 aa, Dengue virus
FPQSNAPIMD

SEQ ID NO: 873 - protein, 10 aa, Dengue virus
FPVSIPITAA

SEQ ID NO: 874 - protein, 10 aa, Dengue virus
FSRENSLSGV

SEQ ID NO: 875 - protein, 10 aa, Dengue virus
FTILALFLAH

SEQ ID NO: 876 - protein, 10 aa, Dengue virus
FTIMAAILAY

SEQ ID NO: 877 - protein, 9 aa, Dengue virus
TIMAAILAY

SEQ ID NO: 878 - protein, 9 aa, Dengue virus
TLMAAILAY

SEQ ID NO: 879 - protein, 10 aa, Dengue virus
FTMGVLCLAI

SEQ ID NO: 880 - protein, 10 aa, Dengue virus
FTMRHKKATY

SEQ ID NO: 881 - protein, 10 aa, Dengue virus
FTNMEAQLVR

SEQ ID NO: 882 - protein, 10 aa, Dengue virus
GAMLFLISGK

SEQ ID NO: 883 - protein, 10 aa, Dengue virus
GASKRSWPLN

SEQ ID NO: 884 - protein, 10 aa, Dengue virus
GEARKTFVDL

SEQ ID NO: 885 - protein, 10 aa, Dengue virus
GEARKTFVEL

SEQ ID NO: 886 - protein, 10 aa, Dengue virus
GEQRKTFVEL

SEQ ID NO: 887 - protein, 10 aa, Dengue virus
GESRKTFVEL

SEQ ID NO: 888 - protein, 10 aa, Dengue virus
GEFRLRGEQR

SEQ ID NO: 889 - protein, 9 aa, Dengue virus
GEGLHKLGY

SEQ ID NO: 890 - protein, 10 aa, Dengue virus
GEVGAIALDF

SEQ ID NO: 891 - protein, 10 aa, Dengue virus
GFFLRKLTSR

SEQ ID NO: 892 - protein, 9 aa, Dengue virus
GGWRLSAQW

SEQ ID NO: 893 - protein, 9 aa, Dengue virus
GMGEAAAIF

SEQ ID NO: 894 - protein, 10 aa, Dengue virus
GPGHEEPIPM

SEQ ID NO: 895 - protein, 9 aa, Dengue virus
GPLVAGGLL

SEQ ID NO: 896 - protein, 9 aa, Dengue virus
GPMKLVMAF

SEQ ID NO: 897 - protein, 10 aa, Dengue virus
GPMKLVMAFI

SEQ ID NO: 898 - protein, 9 aa, Dengue virus
GPSLRTTTV

SEQ ID NO: 899 - protein, 10 aa, Dengue virus
GPWHLGKLEL

SEQ ID NO: 900 - protein, 10 aa, Dengue virus
GPWHLGKLEM

SEQ ID NO: 901 - protein, 10 aa, Dengue virus
GSSKIRWIVE

SEQ ID NO: 902 - protein, 10 aa, Dengue virus
GTSGSPIIDK

SEQ ID NO: 903 - protein, 9 aa, Dengue virus
TSGSPIIDK

SEQ ID NO: 904 - protein, 10 aa, Dengue virus
GTSGSPIVNR

SEQ ID NO: 905 - protein, 9 aa, Dengue virus
HALLATSIF

SEQ ID NO: 906 - protein, 10 aa, Dengue virus
HMIAGVLFTF

SEQ ID NO: 907 - protein, 9 aa, Dengue virus
HPASAWTLY

SEQ ID NO: 908 - protein, 10 aa, Dengue virus
RPASAWTLYA

SEQ ID NO: 909 - protein, 10 aa, Dengue virus
HPGAGKTKRY

SEQ ID NO: 910 - protein, 10 aa, Dengue virus
HPGFTILALF

SEQ ID NO: 911 - protein, 10 aa, Dengue virus
HQLWATLLSL

SEQ ID NO: 912 - protein, 9 aa, Dengue virus
IAFLRFLAI

SEQ ID NO: 913 - protein, 9 aa, Dengue virus
IANQAAILM

SEQ ID NO: 914 - protein, 9 aa, Dengue virus
IANQATVLM

SEQ ID NO: 915 - protein, 9 aa, Dengue virus
IAVSMANIF

SEQ ID NO: 916 - protein, 9 aa, Dengue virus
IAVSTANIF

SEQ ID NO: 917 - protein, 9 aa, Dengue virus
ICSAVPVHW

SEQ ID NO: 918 - protein, 9 aa, Dengue virus
IGIGILLTW

SEQ ID NO: 919 - protein, 9 aa, Dengue virus
IGIGVLLTW

SEQ ID NO: 920 - protein, 9 aa, Dengue virus
IGLTSRATW

SEQ ID NO: 921 - protein, 9 aa, Dengue virus
IILEFFLMV

SEQ ID NO: 922 - protein, 10 aa, Dengue virus
IPFEIMDLEK

SEQ ID NO: 923 - protein, 9 aa, Dengue virus
IPITAAAWY

SEQ ID NO: 924 - protein, 9 aa, Dengue virus
IPKAYAGPF

SEQ ID NO: 925 - protein, 10 aa, Dengue virus
IPMATYGWNL

SEQ ID NO: 926 - protein, 10 aa, Dengue virus
IPMSTYGWNL

SEQ ID NO: 927 - protein, 10 aa, Dengue virus
IPMTGPLVAG

SEQ ID NO: 928 - protein, 10 aa, Dengue virus
IPQWEPSKGW

SEQ ID NO: 929 - protein, 9 aa, Dengue virus
IPTVMAFHL

SEQ ID NO: 930 - protein, 9 aa, Dengue virus
IQPFLALGF

SEQ ID NO: 931 - protein, 10 aa, Dengue virus
ISSMLNIMNR

SEQ ID NO: 932 - protein, 9 aa, Dengue virus
ITAAAWYLW

SEQ ID NO: 933 - protein, 10 aa, Dengue virus
ITLLCLIPTV

SEQ ID NO: 934 - protein, 9 aa, Dengue virus
TLLCLIPTV

SEQ ID NO: 935 - protein, 9 aa, Dengue virus
ITPMLRHTI

SEQ ID NO: 936 - protein, 10 aa, Dengue virus
IVIGVGDSAL

SEQ ID NO: 937 - protein, 9 aa, Dengue virus
KAKGSRAIW

SEQ ID NO: 938 - protein, 9 aa, Dengue virus
RAKGSRAIW

SEQ ID NO: 939 - protein, 10 aa, Dengue virus
KATREAQKRA

SEQ ID NO: 940 - protein, 10 aa, Dengue virus
KAVHADMGYW

SEQ ID NO: 941 - protein, 10 aa, Dengue virus
RAVHADMGYW

SEQ ID NO: 942 - protein, 9 aa, Dengue virus
KAYAQMWSL

SEQ ID NO: 943 - protein, 9 aa, Dengue virus
KEGVFHTMW

SEQ ID NO: 944 - protein, 10 aa, Dengue virus
KGSRAIWYMW

SEQ ID NO: 945 - protein, 9 aa, Dengue virus
KPGTSGSPI

SEQ ID NO: 946 - protein, 10 aa, Dengue virus
KPGTSGSPII

SEQ ID NO: 947 - protein, 10 aa, Dengue virus
KPGTSGSPIV

SEQ ID NO: 948 - protein, 10 aa, Dengue virus
KPRICTREEF

SEQ ID NO: 949 - protein, 10 aa, Dengue virus
KPRLCTREEF

SEQ ID NO: 950 - protein, 10 aa, Dengue virus
NPRLCTREEF

SEQ ID NO: 951 - protein, 10 aa, Dengue virus
RPRLCTREEF

SEQ ID NO: 952 - protein, 10 aa, Dengue virus
TPRMCTREEF

SEQ ID NO: 953 - protein, 9 aa, Dengue virus
KPRWLDARI

SEQ ID NO: 954 - protein, 9 aa, Dengue virus
KPTLDFELI

SEQ ID NO: 955 - protein, 9 aa, Dengue virus
KPWDVIPMV

SEQ ID NO: 956 - protein, 9 aa, Dengue virus
KPWDVVPMV

SEQ ID NO: 957 - protein, 9 aa, Dengue virus
KQIANELNY

SEQ ID NO: 958 - protein, 9 aa, Dengue virus
KQISNELNY

SEQ ID NO: 959 - protein, 9 aa, Dengue virus
KSGAIKVLK

SEQ ID NO: 960 - protein, 9 aa, Dengue virus
KSYAQMWQL

SEQ ID NO: 961 - protein, 9 aa, Dengue virus
KTDFGFYQV

SEQ ID NO: 962 - protein, 9 aa, Dengue virus
KTFVDLMRR

SEQ ID NO: 963 - protein, 9 aa, Dengue virus
KTWAYHGSY

SEQ ID NO: 964 - protein, 10 aa, Dengue virus
KVIQLSRKTF

SEQ ID NO: 965 - protein, 10 aa, Dengue virus
KVLNPYMPSV

SEQ ID NO: 966 - protein, 9 aa, Dengue virus
VLNPYMPSV

SEQ ID NO: 967 - protein, 9 aa, Dengue virus
KVRKDIQQW

SEQ ID NO: 968 - protein, 10 aa, Dengue virus
LAYTIGTTHF

SEQ ID NO: 969 - protein, 10 aa, Dengue virus
LEFEALGFLN

SEQ ID NO: 970 - protein, 10 aa, Dengue virus
LEFEALGFMN

SEQ ID NO: 971 - protein, 9 aa, Dengue virus
LETLMLVAL

SEQ ID NO: 972 - protein, 9 aa, Dengue virus
LGFLNEDHW

SEQ ID NO: 973 - protein, 10 aa, Dengue virus
LIHQVFGTAY

SEQ ID NO: 974 - protein, 10 aa, Dengue virus
LVHQIFGTAY

SEQ ID NO: 975 - protein, 10 aa, Dengue virus
LILCVTQVLM

SEQ ID NO: 976 - protein, 9 aa, Dengue virus
LLMLVTPSM

SEQ ID NO: 977 - protein, 10 aa, Dengue virus
LLSPVRVPNY

SEQ ID NO: 978 - protein, 9 aa, Dengue virus
LMCHATFTM

SEQ ID NO: 979 - protein, 10 aa, Dengue virus
LMKITAEWLW

SEQ ID NO: 980 - protein, 9 aa, Dengue virus
MEITAEWLW

SEQ ID NO: 981 - protein, 10 aa, Dengue virus
VMGITAEWLW

SEQ ID NO: 982 - protein, 10 aa, Dengue virus
LMMATIGIAL

SEQ ID NO: 983 - protein, 9 aa, Dengue virus
MMATIGIAL

SEQ ID NO: 984 - protein, 9 aa, Dengue virus
LMMILPAAL

SEQ ID NO: 985 - protein, 10 aa, Dengue virus
LMMMLPATLA

SEQ ID NO: 986 - protein, 10 aa, Dengue virus
LMMTTIGVVL

SEQ ID NO: 987 - protein, 9 aa, Dengue virus
LPAIVREAI

SEQ ID NO: 988 - protein, 9 aa, Dengue virus
LPEEQDQNY

SEQ ID NO: 989 - protein, 9 aa, Dengue virus
LPESLETLM

SEQ ID NO: 990 - protein, 10 aa, Dengue virus
LPETLETLLL

SEQ ID NO: 991 - protein, 10 aa, Dengue virus
LPGADTQGSN

SEQ ID NO: 992 - protein, 9 aa, Dengue virus
LPIRYQTPA

SEQ ID NO: 993 - protein, 10 aa, Dengue virus
LPIRYQTPAI

SEQ ID NO: 994 - protein, 10 aa, Dengue virus
LPLPWLPGAD

SEQ ID NO: 995 - protein, 9 aa, Dengue virus
LPLPWTSGA

SEQ ID NO: 996 - protein, 10 aa, Dengue virus
LPTFMTQKAR

SEQ ID NO: 997 - protein, 10 aa, Dengue virus
LPTYLSSRAK

SEQ ID NO: 998 - protein, 10 aa, Dengue virus
LPVWLAHKVA

SEQ ID NO: 999 - protein, 9 aa, Dengue virus
LPVWLAYKV

SEQ ID NO: 1000 - protein, 10 aa, Dengue virus
LPVWLAYKVA

SEQ ID NO: 1001 - protein, 10 aa, Dengue virus
LPVWLAYRVA

SEQ ID NO: 1002 - protein, 9 aa, Dengue virus
LPVWLSYKV

SEQ ID NO: 1003 - protein, 10 aa, Dengue virus
LRVLNLVENW

SEQ ID NO: 1004 - protein, 10 aa, Dengue virus
LSMGLITIAV

SEQ ID NO: 1005 - protein, 9 aa, Dengue virus
LSRNSTHEM

SEQ ID NO: 1006 - protein, 9 aa, Dengue virus
LTAAVLLLI

SEQ ID NO: 1007 - protein, 10 aa, Dengue virus
TLTAAVLLLV

SEQ ID NO: 1008 - protein, 9 aa, Dengue virus
LWEGSPGKF

SEQ ID NO: 1009 - protein, 9 aa, Dengue virus
MAEIPLQWI

SEQ ID NO: 1010 - protein, 9 aa, Dengue virus
MAFIAFLRF

SEQ ID NO: 1011 - protein, 9 aa, Dengue virus
MAILGDTAW

SEQ ID NO: 1012 - protein, 9 aa, Dengue virus
MALKDFKEF

SEQ ID NO: 1013 - protein, 9 aa, Dengue virus
MAMTCIAVG

SEQ ID NO: 1014 - protein, 10 aa, Dengue virus
MANEMGFLEK

SEQ ID NO: 1015 - protein, 9 aa, Dengue virus
MANIFRGSY

SEQ ID NO: 1016 - protein, 10 aa, Dengue virus
MASSALLWMA

SEQ ID NO: 1017 - protein, 9 aa, Dengue virus
MAVGMVSIL

SEQ ID NO: 1018 - protein, 9 aa, Dengue virus
MGYWIESAL

SEQ ID NO: 1019 - protein, 10 aa, Dengue virus
MIDKTPVHSW

SEQ ID NO: 1020 - protein, 9 aa, Dengue virus
MLLALIAVL

SEQ ID NO: 1021 - protein, 10 aa, Dengue virus
MLLILCVTQV

SEQ ID NO: 1022 - protein, 9 aa, Dengue virus
MLVTPSMTM

SEQ ID NO: 1023 - protein, 9 aa, Dengue virus
MLWMAEIPL

SEQ ID NO: 1024 - protein, 9 aa, Dengue virus
MMLKLLTDF

SEQ ID NO: 1025 - protein, 9 aa, Dengue virus
MMLPATLAF

SEQ ID NO: 1026 - protein, 10 aa, Dengue virus
MMMLPATLAF

SEQ ID NO: 1027 - protein, 10 aa, Dengue virus
MPGTFQTTTG

SEQ ID NO: 1028 - protein, 10 aa, Dengue virus
MPGTRKVMGI

SEQ ID NO: 1029 - protein, 10 aa, Dengue virus
MPGTRRVMGI

SEQ ID NO: 1030 - protein, 10 aa, Dengue virus
MPLVMAWRTI

SEQ ID NO: 1031 - protein, 10 aa, Dengue virus
MPSMKRFRRE

SEQ ID NO: 1032 - protein, 10 aa, Dengue virus
MPSVIEKMET

SEQ ID NO: 1033 - protein, 10 aa, Dengue virus
MPVMKRYSAP

SEQ ID NO: 1034 - protein, 10 aa, Dengue virus
MPVTHSSAAQ

SEQ ID NO: 1035 - protein, 10 aa, Dengue virus
MPVTVASAAQ

SEQ ID NO: 1036 - protein, 10 aa, Dengue virus
MSFRDLGRVM

SEQ ID NO: 1037 - protein, 10 aa, Dengue virus
MSSEGAWKHA

SEQ ID NO: 1038 - protein, 10 aa, Dengue virus
MSYSMCTGKF

SEQ ID NO: 1039 - protein, 9 aa, Dengue virus
MSYTMCSGK

SEQ ID NO: 1040 - protein, 9 aa, Dengue virus
MTQKARNAL

SEQ ID NO: 1041 - protein, 9 aa, Dengue virus
MTTTANWLW

SEQ ID NO: 1042 - protein, 9 aa, Dengue virus
MVSRLLLNR

SEQ ID NO: 1043 - protein, 9 aa, Dengue virus
NPAQEDDQY

SEQ ID NO: 1044 - protein, 9 aa, Dengue virus
NPEIEDDIF

SEQ ID NO: 1045 - protein, 9 aa, Dengue virus
NPITLTAAL

SEQ ID NO: 1046 - protein, 9 aa, Dengue virus
NPLTLTAAV

SEQ ID NO: 1047 - protein, 10 aa, Dengue virus
NPNMIDKTPV

SEQ ID NO: 1048 - protein, 9 aa, Dengue virus
NPWMEDKTH

SEQ ID NO: 1049 - protein, 10 aa, Dengue virus
NPWMEDKTPV

SEQ ID NO: 1050 - protein, 9 aa, Dengue virus
NPTVDGITV

SEQ ID NO: 1051 - protein, 10 aa, Dengue virus
NQLIYVILTI

SEQ ID NO: 1052 - protein, 10 aa, Dengue virus
PASAWTLYAV

SEQ ID NO: 1053 - protein, 10 aa, Dengue virus
PASIAARGYI

SEQ ID NO: 1054 - protein, 10 aa, Dengue virus
PTSRTTWSIH

SEQ ID NO: 1055 - protein, 9 aa, Dengue virus
QEGAMHSAL

SEQ ID NO: 1056 - protein, 9 aa, Dengue virus
QEGAMHTAL

SEQ ID NO: 1057 - protein, 10 aa, Dengue virus
QENPYRTWAY

SEQ ID NO: 1058 - protein, 10 aa, Dengue virus
QIAMTDTTPF

SEQ ID NO: 1059 - protein, 9 aa, Dengue virus
QPESNILDI

SEQ ID NO: 1060 - protein, 9 aa, Dengue virus
QPHWIAASI

SEQ ID NO: 1061 - protein, 10 aa, Dengue virus
QSGVDVFFTP

SEQ ID NO: 1062 - protein, 9 aa, Dengue virus
QYIFTGQPL

SEQ ID NO: 1063 - protein, 9 aa, Dengue virus
QYSDRRWCF

SEQ ID NO: 1064 - protein, 9 aa, Dengue virus
RAAVEDEEF

SEQ ID NO: 1065 - protein, 10 aa, Dengue virus
RASFIEVKTC

SEQ ID NO: 1066 - protein, 9 aa, Dengue virus
REDLWCGSL

SEQ ID NO: 1067 - protein, 9 aa, Dengue virus
REDQWCGSL

SEQ ID NO: 1068 - protein, 10 aa, Dengue virus
REIPERSWNT

SEQ ID NO: 1069 - protein, 10 aa, Dengue virus
REWCFTGERN

SEQ ID NO: 1070 - protein, 10 aa, Dengue virus
RFLEFEALGF

SEQ ID NO: 1071 - protein, 10 aa, Dengue virus
RYLEFEALGF

SEQ ID NO: 1072 - protein, 10 aa, Dengue virus
RLRGEARKTF

SEQ ID NO: 1073 - protein, 9 aa, Dengue virus
RPGYHTQTA

SEQ ID NO: 1074 - protein, 10 aa, Dengue virus
RPISEKEENM

SEQ ID NO: 1075 - protein, 9 aa, Dengue virus
RPRWLDART

SEQ ID NO: 1076 - protein, 9 aa, Dengue virus
RPTPKGAVM

SEQ ID NO: 1077 - protein, 9 aa, Dengue virus
RPTPKGTVM

SEQ ID NO: 1078 - protein, 9 aa, Dengue virus
RQLANAIFK

SEQ ID NO: 1079 - protein, 9 aa, Dengue virus
RQMEGEGIF

SEQ ID NO: 1080 - protein, 9 aa, Dengue virus
RQMEGEGVL

SEQ ID NO: 1081 - protein, 9 aa, Dengue virus
RVIDPRRCL

SEQ ID NO: 1082 - protein, 10 aa, Dengue virus
RVIDPRRCLK

SEQ ID NO: 1083 - protein, 9 aa, Dengue virus
RVIDPRRCM

SEQ ID NO: 1084 - protein, 10 aa, Dengue virus
RVIDPRRCMK

SEQ ID NO: 1085 - protein, 9 aa, Dengue virus
RVLKMVEPW

SEQ ID NO: 1086 - protein, 10 aa, Dengue virus
RYMGEDGCWY

SEQ ID NO: 1087 - protein, 10 aa, Dengue virus
SEMAEALKGM

SEQ ID NO: 1088 - protein, 10 aa, Dengue virus
SPCKIPFEIM

SEQ ID NO: 1089 - protein, 10 aa, Dengue virus
SPGKFWNTTI

SEQ ID NO: 1090 - protein, 10 aa, Dengue virus
SPIINREGKV

SEQ ID NO: 1091 - protein, 10 aa, Dengue virus
SPILSITISE

SEQ ID NO: 1092 - protein, 10 aa, Dengue virus
SPKRLATAIA

SEQ ID NO: 1093 - protein, 10 aa, Dengue virus
SPSPTVEESR

SEQ ID NO: 1094 - protein, 10 aa, Dengue virus
SPVRVPNYNL

SEQ ID NO: 1095 - protein, 9 aa, Dengue virus
SQIGAGVYK

SEQ ID NO: 1096 - protein, 9 aa, Dengue virus
STYGWNLVR

SEQ ID NO: 1097 - protein, 10 aa, Dengue virus
SWMVRILIGF

SEQ ID NO: 1098 - protein, 10 aa, Dengue virus
SYAQMWTLMY

SEQ ID NO: 1099 - protein, 10 aa, Dengue virus
YAQMWQLMYF

SEQ ID NO: 1100 - protein, 9 aa, Dengue virus
YAQMWSLMY

SEQ ID NO: 1101 - protein, 10 aa, Dengue virus
YAQMWSLMYF

SEQ ID NO: 1102 - protein, 9 aa, Dengue virus
YAQMWTLMY

SEQ ID NO: 1103 - protein, 10 aa, Dengue virus
YAQMWTLMYF

SEQ ID NO: 1104 - protein, 10 aa, Dengue virus
TAAVLLLITH

SEQ ID NO: 1105 - protein, 9 aa, Dengue virus
TAEAGGRAY

SEQ ID NO: 1106 - protein, 10 aa, Dengue virus
TETTILDVDL

SEQ ID NO: 1107 - protein, 9 aa, Dengue virus
TFKVPHAKR

SEQ ID NO: 1108 - protein, 10 aa, Dengue virus
TLMAMDLGEL

SEQ ID NO: 1109 - protein, 10 aa, Dengue virus
TLMLLALIAV

SEQ ID NO: 1110 - protein, 9 aa, Dengue virus
TPEAKNSTF

SEQ ID NO: 1111 - protein, 9 aa, Dengue virus
TPEARNSTF

SEQ ID NO: 1112 - protein, 9 aa, Dengue virus
TPEGIIPAL

SEQ ID NO: 1113 - protein, 10 aa, Dengue virus
TPEGIIPALF

SEQ ID NO: 1114 - protein, 9 aa, Dengue virus
TPEGIIPSM

SEQ ID NO: 1115 - protein, 10 aa, Dengue virus
TPEGIIPSMF

SEQ ID NO: 1116 - protein, 10 aa, Dengue virus
TPEGIIPTLF

SEQ ID NO: 1117 - protein, 10 aa, Dengue virus
YTPEGIIPTL

SEQ ID NO: 1118 - protein, 9 aa, Dengue virus
TPFGQQRVF

SEQ ID NO: 1119 - protein, 10 aa, Dengue virus
TPKGAVMDII

SEQ ID NO: 1120 - protein, 10 aa, Dengue virus
TPRGTVMDII

SEQ ID NO: 1121 - protein, 10 aa, Dengue virus
TPMLRHTIEN

SEQ ID NO: 1122 - protein, 9 aa, Dengue virus
TPPGSRDPF

SEQ ID NO: 1123 - protein, 9 aa, Dengue virus
TPQDNQLAY

SEQ ID NO: 1124 - protein, 10 aa, Dengue virus
TPQDNQLAYV

SEQ ID NO: 1125 - protein, 9 aa, Dengue virus
TPQDNQLTY

SEQ ID NO: 1126 - protein, 10 aa, Dengue virus
TPRSMPGTRR

SEQ ID NO: 1127 - protein, 9 aa, Dengue virus
TPVHSWEDI

SEQ ID NO: 1128 - protein, 9 aa, Dengue virus
TTFSLHYAW

SEQ ID NO: 1129 - protein, 10 aa, Dengue virus
TTKRDLGMSK

SEQ ID NO: 1130 - protein, 9 aa, Dengue virus
TYLALIATF

SEQ ID NO: 1131 - protein, 10 aa, Dengue virus
VASGLLWVAE

SEQ ID NO: 1132 - protein, 9 aa, Dengue virus
VATTFVTPM

SEQ ID NO: 1133 - protein, 10 aa, Dengue virus
VLLLVTHYAI

SEQ ID NO: 1134 - protein, 9 aa, Dengue virus
VPLLAIGCY

SEQ ID NO: 1135 - protein, 9 aa, Dengue virus
VPLLAMGCY

SEQ ID NO: 1136 - protein, 9 aa, Dengue virus
VPMVTQMAM

SEQ ID NO: 1137 - protein, 9 aa, Dengue virus
VPNYNMIIM

SEQ ID NO: 1138 - protein, 10 aa, Dengue virus
VPYLGKREDQ

SEQ ID NO: 1139 - protein, 9 aa, Dengue virus
VQADMGCVV

SEQ ID NO: 1140 - protein, 10 aa, Dengue virus
VSSVNMVSRL

SEQ ID NO: 1141 - protein, 10 aa, Dengue virus
VTIDLDPVVY

SEQ ID NO: 1142 - protein, 10 aa, Dengue virus
VTRGAVLMHK

SEQ ID NO: 1143 - protein, 9 aa, Dengue virus
VTYECPLLV

SEQ ID NO: 1144 - protein, 9 aa, Dengue virus
VYTQLCDHR

SEQ ID NO: 1145 - protein, 10 aa, Dengue virus
VYTQLCDHRL

SEQ ID NO: 1146 - protein, 9 aa, Dengue virus
WALCESITL

SEQ ID NO: 1147 - protein, 9 aa, Dengue virus
WAYHGSYET

SEQ ID NO: 1148 - protein, 9 aa, Dengue virus
WAYHGSYEV

SEQ ID NO: 1149 - protein, 9 aa, Dengue virus
WHYDQDHPY

SEQ ID NO: 1150 - protein, 9 aa, Dengue virus
WSIHAHHQW

SEQ ID NO: 1151 - protein, 9 aa, Dengue virus
WVAEIQPQW

SEQ ID NO: 1152 - protein, 9 aa, Dengue virus
YAQIQPHWI

SEQ ID NO: 1153 - protein, 9 aa, Dengue virus
YENLKYSVI

SEQ ID NO: 1154 - protein, 10 aa, Dengue virus
YGGPISQHNY

SEQ ID NO: 1155 - protein, 10 aa, Dengue virus
YGVLFSGVSW

SEQ ID NO: 1156 - protein, 9 aa, Dengue virus
YLAGAGLAF

SEQ ID NO: 1157 - protein, 9 aa, Dengue virus
YPKTKLTDW

SEQ ID NO: 1158 - protein, 10 aa, Dengue virus
YPKTKLTDWD

SEQ ID NO: 1159 - protein, 10 aa, Dengue virus
YQLAVTITAI

SEQ ID NO: 1160 - protein, 10 aa, Dengue virus
YQLWTALISL

SEQ ID NO: 1161 - protein, 10 aa, Dengue virus
YQNKVVKVLR

SEQ ID NO: 1162 - protein, 10 aa, Dengue virus
YQNKVVKVQR

SEQ ID NO: 1163 - protein, 10 aa, Dengue virus
YVSAIAQTEK